(19) United States
(12) Patent Application Publication
(10) Pub. No.: US 2013/0102485 A1
(43) Pub. Date: Apr. 25, 2013

(54) Method of Determining a Diseased State in a Subject

(71) Applicant: Inhan Lee, Ann Arbor, MI (US)

(72) Inventor: Inhan Lee, Ann Arbor, MI (US)

(21) Appl. No.: 13/656,201

(22) Filed: Oct. 19, 2012

Related U.S. Application Data
(60) Provisional application No. 61/627,856, filed on Oct. 19, 2011.

(51) Int. Cl. C40B 30/04; C12Q 1/68

(52) U.S. Cl. 506/9; 435/6.1

(57) ABSTRACT

A method includes a step of identifying candidate miRNA-mRNA complexes where an mRNA sequence from a set of mRNA sequences stably hybridizes to a miRNA from a set of miRNA sequences. The candidate miRNA-mRNA complexes have stably hybridizing sub-regions of a downstream region to portions of a 5′ miRNA section and stably hybridizing sub-regions of an upstream region to portions of a 3′ miRNA section. Candidate mRNA and/or miRNA sequences are identified as sequences that form candidate microRNA-mRNA complexes. Differences between expression levels between candidate mRNA and/or miRNA sequences in subjects having a disease and subjects not having the disease are determined for each candidate mRNA and/or miRNA sequence to identify candidate mRNA sequences and/or miRNA sequences. The expression levels of each candidate RNA disease markers are compared to controls such that deviation of expression levels of the candidate RNA disease markers from the controls indicates presence of the disease.

### CROSS-REFERENCE TO RELATED APPLICATIONS

This application claims the benefit of U.S. provisional Application No. 61/627,856, filed Oct. 19, 2011, the disclosure of which is incorporated in its entirety by reference herein.

### SEQUENCE LISTING

The text file is mirc_ST25.txt, created Oct. 19, 2012, and of size 461 KB, filed therewith, is hereby incorporated by reference.

### TECHNICAL FIELD

The present invention relates to methods of determining mRNA and miRNA associated with a disease state, determining whether a subject has a disease state, and monitoring the subject's disease state progression. Such methods find use in research, diagnostic, and therapeutic setting (e.g., to discover targets, drugs, diagnostic products, etc.).

### BACKGROUND

Identifying biomarkers from large collective datasets drawn from subjects with similar symptoms is essential to personalized medicine. Though a systems biology approach is needed to explore beyond genes with known functions, differentiating non-marking entities from biomarkers poses a significant challenge due to the vast noisy data. With advances in technology producing vast amounts of data daily, the development of efficient analytical methods is urgently needed to identify biomarkers for the systems in interest. mRNA data also require such method.

Microarray platforms have generated an enormous amount of data since its early technical development. However, microarray technology has problems such as background fluorescence, different responses among fluorescent molecules, platform variances, and batch variances. Combining the accumulated data points of various samples can provide information that individual datasets cannot.

However, even after stringent statistical analysis or large meta-analysis, hundreds to thousands of genes are left to researchers simply as being up- or down-regulated in their systems of interest. Making sense of these gene lists remains difficult, even with various enrichment analyses or features integrated from other databases related to signaling pathway, gene ontology, protein-protein interaction mapping, and natural language processing for literature searches. The number of statistically significant mRNAs is still too great to be readily understood.

MicroRNAs (miRNAs) are a class of post-transcriptional regulators. miRNAs are short nucleotide RNA sequences that may range in size from 16-25 nucleotides that bind to complementary sequences in the 3′ UTR of multiple target mRNAs, usually resulting in mRNA silencing. miRNAs are processed from hairpin-structured precursors (pre-miRNA) by the enzyme Dicer. miRNAs recognize complementary sites in the 3′ untranslated region (UTR) of target mRNAs in miRNP (miRNA-RiboNucleoProtein complex) and repress gene expression posttranscriptionally by affecting mRNA stability or protein translation.

So far, more than a thousand miRNAs have been identified in humans (http://www.mirbase.org), some of them potentially regulating about 200 genes simultaneously. miRNAs target ˜60% of all human genes and are abundantly present in all human cells. miRNA ability to target many mRNAs, coupled with their conservation in disparate organisms, suggest miRNAs are a vital part of genetic regulation with ancient origins.

As with other endogenous genes with regulatory roles, such as transcription factors, expression of each miRNA is under strict control and exhibits characteristic patterns based on biological context, as in tissue and developmental stages. In line with the critical roles of miRNAs in biological processes, their deregulation is frequently observed in many diseases. For example, miRNA expression profiles predict cancer type, stage, and other clinical variables more accurately than mRNA expression profiles. As some miRNAs are oncogenes and others are tumor suppressors, they can be therapeutic targets. miRNAs are also being developed for diagnosis and therapy in several other diseases, including cardiovascular disorders, Alzheimer's disease, and diseases related to viral infection. Hence, miRNAs hold promise as biomarkers for diagnostic and therapeutic purposes in various diseases.

Though miRNA expression data are accumulating, they are still far fewer than mRNA data. Given that miRNAs regulate the expression of target mRNAs, it is possible to reasonably predict miRNA expression based on mRNA expression. Considering the vast amount of mRNA data available, this possibility holds great promise in terms of saving money and efficiently utilizing available resources. Also, given the miRNA deregulation in disease development, such predictions may lead to biomarkers with superior resolution, potentially revealing master regulators. To realize these benefits, accurate miRNA target identification is needed.

While other areas of miRNA research have undergone continuous development, target prediction has lagged behind. This is partly due to the imperfect miRNA-mRNA bindings found in animals. As experimental and evolutionary evidence indicates that the 5′-end of miRNAs (the nucleotide position 1-8 at the 5′-end, also called the seed sequence) is important for recognition of target sequences in 3′-UTRs, many computational algorithms utilize only 6-8 nucleotides of the ˜22 mer miRNA to predict target mRNAs, resulting in a large number of false positives among the predicted targets, thus challenging the miRNA-mRNA correlation.

As with transcription factors and other endogenous genes with regulatory roles, expression of each miRNA is strictly controlled and exhibits characteristic patterns based on biological context, such as tissue type or developmental stages. In line with the critical roles of miRNAs in biological processes, their deregulation is frequently observed in many diseases, including Parkinson's disease and neuroblastoma.

Parkinson's disease is a disorder of the brain that leads to shaking (tremors) and difficulty with walking, movement, and coordination. Parkinson's disease is one of the most common nervous system disorders of the elderly; most often develops after the age of 50. Sometimes Parkinson's disease occurs in younger adults. It affects both men and women. In some cases, Parkinson's disease runs in families, suggesting a genetic predisposition. When a young person is affected, it is usually because of a form of the disease that runs in families.

Nerve cells use a brain chemical called dopamine to help control muscle movement. Parkinson's disease occurs when the nerve cells in the brain that make dopamine are slowly destroyed. Without dopamine, the nerve cells in that part of the brain cannot properly send messages. This leads to the loss of muscle function. The damage gets worse with time. Exactly why these brain cells waste away is unknown. Parkinson's is rare in children. It may occur because the nerves are not as sensitive to dopamine.

The term “parkinsonism” refers to any condition that involves the types of movement changes seen in Parkinson's disease. Parkinsonism may be caused by other disorders (called secondary parkinsonism) or certain medications.

Because Parkinson's disease is a progressive disease, symptoms may worsen as time goes on. And because Parkinson's disease is a progressive disease, it's important to begin treatment as soon as possible. Thus, early detection, treatment, and monitoring of a subject with Parkinson's disease or showing Parkinson-like symptoms may have enormous benefits to the subject's short-term and long-term health.

There is no known cure for Parkinson's disease. The goal of treatment is to control symptoms. Medications control symptoms, mostly by increasing the levels of dopamine in the brain. Medications used to treat movement-related symptoms of Parkinson's disease include: Levodopa (L-dopa), Sinemet, levodopa and carbidopa (Atamet), Pramipexole (Mirapex), ropinirole (Requip), bromocriptine (Parlodel), Selegiline (Eldepryl, Deprenyl), rasagiline (Azilect), Amantadine or anticholinergic medications to reduce early or mild tremors, and Entacapone. Other medications may include: Memantine, rivastigmine, galantamine for cognitive difficulties, Antidepressants for mood disorders, Gabapentin, duloxetine for pain, Fludrocortisone, midodrine, botox, sidenafil for autonomic dysfunction, Armodafinil, clonazepam, and zolpidem for sleep disorders.

Neuroblastoma is a solid cancer that arises from precursor cells of the sympathetic nervous system. It is known for its extreme heterogeneity, ranging from spontaneous regression to rapid progression to metastasis. Prognostic factors include age and genetic abnormalities, with greater likelihood of survival in infants, suggesting that neuroblastoma is a developmental disorder. Considering that neuroblastoma accounts for 15% of all cancer deaths among children, novel therapeutic targets are clearly needed. Furthermore, early differentiation of children who will not develop advanced-stage neuroblastoma would reduce long-term side effects due to treatment, improving quality of life of survivors. Since neuroblastoma arises from precursor cells, understanding the mechanism by which it becomes advanced-stage cancer might also provide insight into how normal stem cells transform into cancer stem cells.

Common genetic abnormalities in neuroblastoma include the genetic amplification (multiple DNA copies) of MYCN, chromosome 1p36 and 11q23 deletion, and the gain of chromosome 17q22. Since all of these genetic abnormalities correlate with poor clinical outcome, they have been investigated as potential driving factors in advanced stage neuroblastoma. However, as advanced neuroblastoma also occurs without such genetic abnormalities, the signatures of advanced neuroblastoma must be determined to understand the mechanism of its progression. For example, though amplification of transcription factor MYCN was linked early on with a poor outcome, MYCN status cannot fully account for all advanced-stage cases. In addition to DNA-level abnormalities, expression level changes of transcripts in advanced stage have been studied to accurately predict the disease outcome. However, MYCN transcript levels could not dependably identify advanced stage neuroblastoma either. It is clear that multiple gene regulation should be considered in deciphering neuroblastoma progression. To differentiate the underlying mechanism of progression, several studies have assessed DNA abnormality and transcription level changes together, comparing, for example, all transcription levels of advanced-stage neuroblastoma with and without MYCN amplification. Recent research on transcript-level changes has incorporated microRNA expression changes.

As shown in FIG. 1, Neuroblastoma-related miRNAs have been researched mainly in regard to two biological processes, anti-apoptosis (tumorigenesis), and epithelial mesenchymal transition (EMT: metastasis related). TP53 is a well-known transcription factor that induces apoptosis and miR-34 family transcription. In neuroblastoma, TP53 mutation is uncommon, whereas deletion on chromosome 1p36 (where mir-34a is located) frequently occurs. Another frequent deletion site of 11q23 coincides with mir-34b and mir-34c chromosomal positions, implying the importance of the mir-34 family in neuroblastoma. On the other hand, MYCN amplification is frequent in neuroblastoma and several miRNAs are induced by MYCN, including the mir-17-92 cluster, which has been shown to be significantly upregulated in MYCN-amplified cells and has been identified with a direct anti-proliferation function. In neuroblastoma, upregulation of the mir-17-92 cluster is correlated with the obstruction of the TGF-beta signaling pathway, thereby preventing apoptosis. MYCN also achieves anti-apoptotic function by downregulating other miRNAs which prevent cell proliferation. Low levels of MYCN allow for the over-expression of miR-184, which has been shown to reduce neuroblastoma tumor growth. While MYCN affects transcripton of certain miRNAs, some miRNAs can down-regulate MYCN translation, such as miR-34. let-7 and miR-101 are also found to target MYCN and inhibit proliferation of MYCN-amplified neuroblastoma cells. Similarly, miR-10a and miR-10b have been shown to indirectly down-regulate MYCN by targeting NCOR2, though the intermediate processes between NCOR2 and MYCN need further study. Therefore, miR-34 family deletion and MYCN amplification in neuroblastoma together respond to tumorigenesis through anti-apoptotic process. Metastasis may also be brought on by altered levels of miRNA. A more specific study showed that over-expression of miR-524-5p decreases the invasive potential of neuroblastoma cells (Bray et al., 2011). Recently, the miR-34 family has been identified as blocking EMT by downregulating SNAIL (Kim et al., 2011), showing its dual function of blocking tumorigenesis and metastasis. miR-92 has been found to down-regulate tumor suppressor DKK3, which normally inhibits the Wnt signaling pathway. The canonical Wnt signaling can induce EMT mostly by degrading E-cadherin. All this implies that a large network stretching across miR-34, MYCN, and the miR-17-92 cluster is responsible for both tumorogenesis and metastasis in neuroblastoma (See, FIG. 1). Hence, miRNAs hold promise as biomarkers for diagnostic and therapeutic purposes.

Accordingly, there is a need for improved methods of diagnosing and monitoring the progression of diseases such are neuroblastoma and Parkinson's disease.

### SUMMARY OF THE INVENTION

The present invention solves one or more problems of the prior art by providing in at least one embodiment, a method of determining a disease state in a subject. The method includes a step of identifying candidate miRNA-mRNA complexes in which an mRNA sequence from a set of mRNA sequences stably hybridizes to a miRNA from a set of miRNA sequences. Each mRNA sequence has an upstream region that is upstream of a translation start site and a downstream region that is downstream of a translation stop site. Each miRNA has a 5′ miRNA section and a 3′ miRNA section. The candidate miRNA-mRNA complexes have stably hybridizing sub-regions of the downstream region to portions of the 5′ miRNA section and stably hybridizing sub-regions of the upstream region to portions of the 3′ miRNA section. Candidate mRNA sequences are identified as mRNA sequences that form candidate microRNA-mRNA complexes. Differences between expression levels between candidate mRNA sequences in subjects having a disease and subjects not having the disease are optionally determined for each candidate mRNA sequence. Candidate RNA disease markers are identified as candidate mRNA sequences and/or miRNA sequences in which the expression levels in subjects having a disease are different from subjects not having the disease. For example, candidate RNA disease markers are identified as candidate mRNA sequences and/or candidate miRNA sequences that have differences that are greater than a predetermined value. A biological sample is obtained from a test subject. The expression levels of each candidate RNA disease marker are determined from the biological sample. The expression level of each candidate RNA disease marker is compared to a control such that deviation of expression levels of at least three candidate RNA disease markers from the control indicates presence of the disease.

In another embodiment, a method for evaluating neuroblastoma in a subject is provided. The method includes a step of obtaining a biological sample from a subject. Expression levels of candidate RNA disease markers from the biological sample are determined. The candidate RNA disease markers are selected from the group consisting of AGPAT4 having NCBI number NM_{—}020133 (SEQ ID NO: 1), BTBD9 having NCBI number NM_{—}001172418 (SEQ ID NO: 2), BTBD9 having NCBI number NM_{—}152733 (SEQ ID NO: 3), BTBD9 having NCBI number NM_{—}001099272 (SEQ ID NO: 4), BTBD9 having NCBI number NM_{—}052893 (SEQ ID NO: 5), GABBR1 having NCBI number NM_{—}001470 (SEQ ID NO: 6), GABBR1 having NCBI number NM_{—}021904 (SEQ ID NO: 7), GABBR1 having NCBI number NM_{—}021903 (SEQ ID NO: 8), KCNK10 having NCBI number NM_{—}021161 (SEQ ID NO: 9), KCNK10 having NCBI number NM_{—}138318 (SEQ ID NO: 10), KCNK10 having NCBI number NM_{—}138317 (SEQ ID NO: 11), LRRTM4 having NCBI number NM_{—}024993 (SEQ ID NO: 12), LRRTM4 having NCBI number NM_{—}001134745 (SEQ ID NO: 13), S100PBP having NCBI number NM_{—}022753 (SEQ ID NO: 14), S100PBP having NCBI number NM_{—}001256121 (SEQ ID NO: 15), and combinations thereof.

A method for evaluating Parkinson's disease in a subject is provided. The method includes a step of obtaining a biological sample from the subject. Expression levels of candidate RNA disease markers from the biological sample are determined. Candidate RNA disease markers are selected from the group consisting AMMECR1 having NCBI number NM_{—}001025580 (SEQ ID NO: 32), AMMECR1 having NCBI number NM_{—}001171689 (SEQ ID NO: 33), AMMECR1 having NCBI number NM_{—}015365 (SEQ ID NO: 34), CASZ1 having NCBI number NM_{—}001079843 (SEQ ID NO: 35), CASZ1 having NCBI number NM_{—}017766 (SEQ ID NO: 36), CCNG2 having NCBI number NM_{—}004354 (SEQ ID NO: 37), FBXO41 having NCBI number NM_{—}001080410 (SEQ ID NO: 38), FOXP1 having NCBI number NM_{—}001244813 (SEQ ID NO: 39), FOXP1 having NCBI number NM_{—}001244814 (SEQ ID NO: 40), FOXP1 having NCBI number NM_{—}001244815 (SEQ ID NO: 41), FOXP1 having NCBI number NM_{—}001012505 (SEQ ID NO: 42), FOXP1 having NCBI number NM_{—}001244808 (SEQ ID NO: 43), FOXP1 having NCBI number NM_{—}001244810 (SEQ ID NO: 44), FOXP1 having NCBI number NM_{—}001244812 (SEQ ID NO: 45), FOXP1 having NCBI number NM_{—}001244816 (SEQ ID NO: 46), FOXP1 having NCBI number NM_{—}032682 (SEQ ID NO: 47), JRK having NCBI number NM_{—}001077527 (SEQ ID NO: 48), JRK having NCBI number NM_{—}003724 (SEQ ID NO: 49), MAPT having NCBI number NM_{—}001123066 (SEQ ID NO: 50), MAPT having NCBI number NM_{—}001123067 (SEQ ID NO: 51), MAPT having NCBI number NM_{—}001203251 (SEQ ID NO: 52), MAPT having NCBI number NM_{—}001203252 (SEQ ID NO: 53), MAPT having NCBI number NM_{—}005910 (SEQ ID NO: 54), MAPT having NCBI number NM_{—}016834 (SEQ ID NO: 55), MAPT having NCBI number NM_{—}016835 (SEQ ID NO: 56), MAPT having NCBI number NM_{—}016841 (SEQ ID NO: 57), NFYC having NCBI number NM_{—}001142587 (SEQ ID NO: 58), NFYC having NCBI number NM_{—}001142588 (SEQ ID NO: 59), NFYC having NCBI number NM_{—}001142589 (SEQ ID NO: 60), NFYC having NCBI number NM_{—}001142590 (SEQ ID NO: 61), NFYC having NCBI number NM_{—}014223 (SEQ ID NO: 62), QKI having NCBI number NM_{—}006775 (SEQ ID NO: 63), QKI having NCBI number NM_{—}206853 (SEQ ID NO: 64), QKI having NCBI number NM_{—}206854 (SEQ ID NO: 65), QKI having NCBI number NM_{—}206855 (SEQ ID NO: 66), RAB1A having NCBI number NM_{—}004161 (SEQ ID NO: 67), RAB1A having NCBI number NM_{—}015543 (SEQ ID NO: 68), RAPGEF3 having NCBI number NM_{—}001098531 (SEQ ID NO: 69), RAPGEF3 having NCBI number NM_{—}001098532 (SEQ ID NO: 70), RAPGEF3 having NCBI number NM_{—}006105 (SEQ ID NO: 71), STAT2 having NCBI number NM_{—}005419 (SEQ ID NO: 72), STAT2 having NCBI number NM_{—}198332 (SEQ ID NO: 73), VASH1 having NCBI number NM_{—}014909 (SEQ ID NO: 74), hsa-miR-1184 (SEQ ID NO: 75), hsa-miR-221 (SEQ ID NO: 23), hsa-miR-1207-5p (SEQ ID NO: 76), hsa-miR-760 (SEQ ID NO: 77), and combinations thereof.

### BRIEF DESCRIPTION OF THE DRAWINGS

FIG. 1 provides a schematic diagram of a miRNA network in neuroblastoma tumorigenesis and metastasis, including miR-9 from this study. The dotted box represents our six predicted targets in this study.

FIGS. 2A AND 2B provide a schematic flowchart for a method of evaluating the presence of a disease in a subject.

FIG. 3 provides an illustration of a miRNA molecule interacting with a mRNA molecule;

FIG. 4 is a schematic illustration of a computer system that at least partially implements a method of determining a disease state;

FIG. 5. Box plots of miR-9 (A), miR-18a* (B), miR-92 (C), miR-125b (D), miR-137 (E), and miR-149 (F) expression between advanced and low-risk stage neuroblastoma patient samples;

FIG. 6. Histogram of Pearson correlation values between miR-9 and gene expressions. The black bars show miR-9 target genes found in the down-regulated genes; the open rectangular bars represent all genes measured in the microarray experiments (total 1,4159 genes excluding ones with blank data >5 among samples);

FIG. 7. Scatter plots showing miR-9 and target gene expressions for 30 patient samples. Pearson correlation coefficient of miR-9 and 5100PBP is −0.53 (A) and that of miR-9 and LRRTM4 is −0.48 (B); and

FIG. 8. Box plots of miR-9 (A), miR-758 (B), miR-885-5p (C), miR-22 (D), miR-302a (E), and miR-886-3p (F) expressions of neuroblastoma between short- and long-term survivors in advanced stage patients.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

The term “amplification” refers to an increase in the amount of a nucleic acid sequence wherein the sequences produced are the same as a starting sequence.

Neuroblastoma, as used herein also includes hereditary neuroblastoma and ALK-related neuroblastoma and related conditions ganglioneuroblastoma, ganglioneuroma, or Opsoclonus Myoclonus Syndrome (OMS).

Parkinson's disease (PD), as used herein also includes Parkinsonism-plus syndromes such as, but not limited to, Progressive supranuclear palsy (PSP), Multiple system atrophy (MSA), Cortical-basal ganglionic degeneration (CBGD), Diffuse Lewy body disease (DLBD), Parkinson-dementia-ALS complex of Guam, and Progressive pallidal atrophy. Further, Heredodegenerative disorders are contemplated for a disease state such as, but not limited to, Alzheimer's disease, Wilson disease, Huntington disease, Frontotemporal dementia on chromosome 17, and X-linked dystonia-parkinsonism.

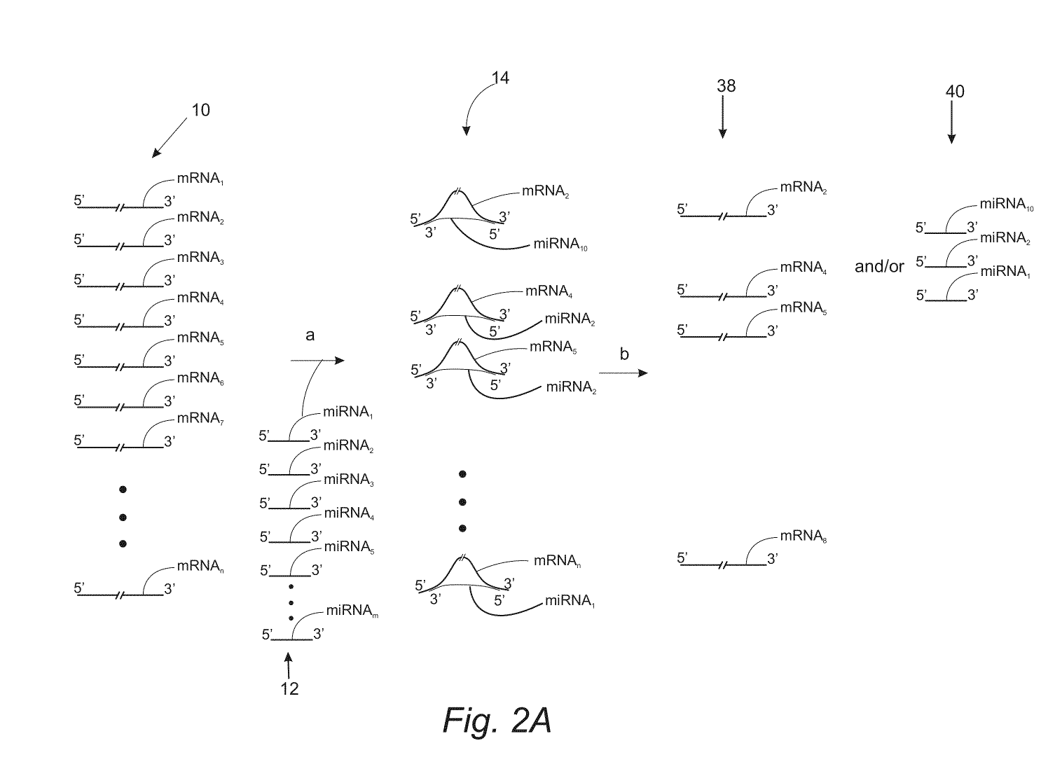

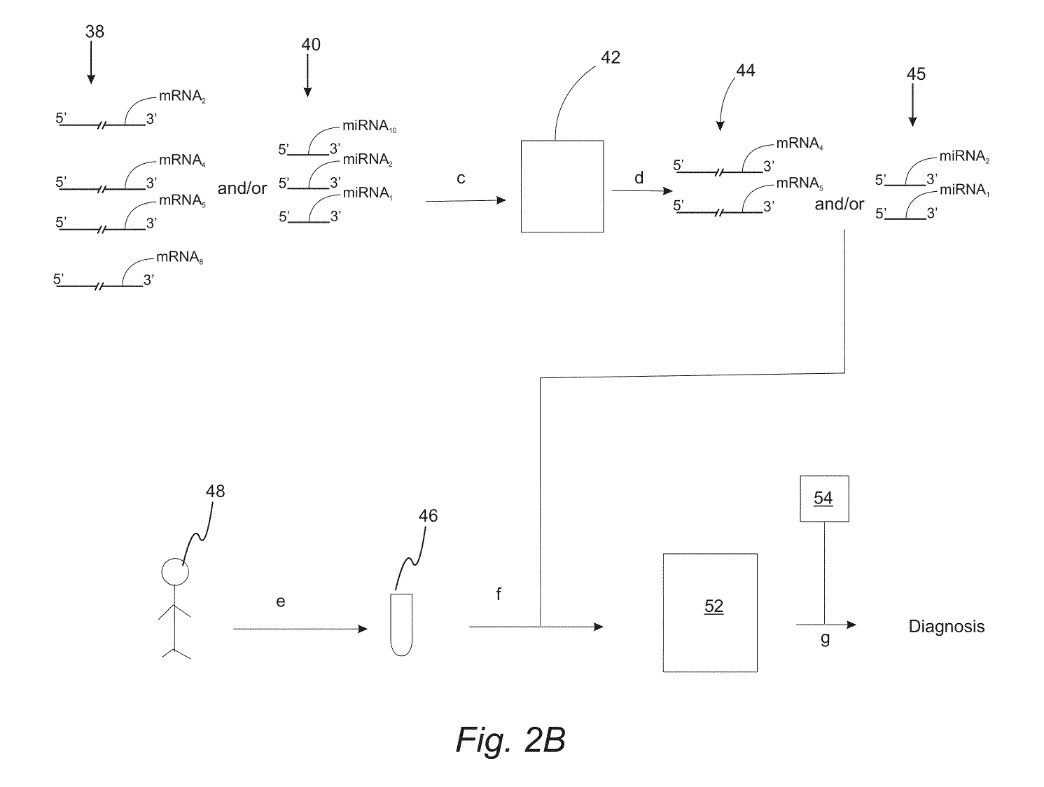

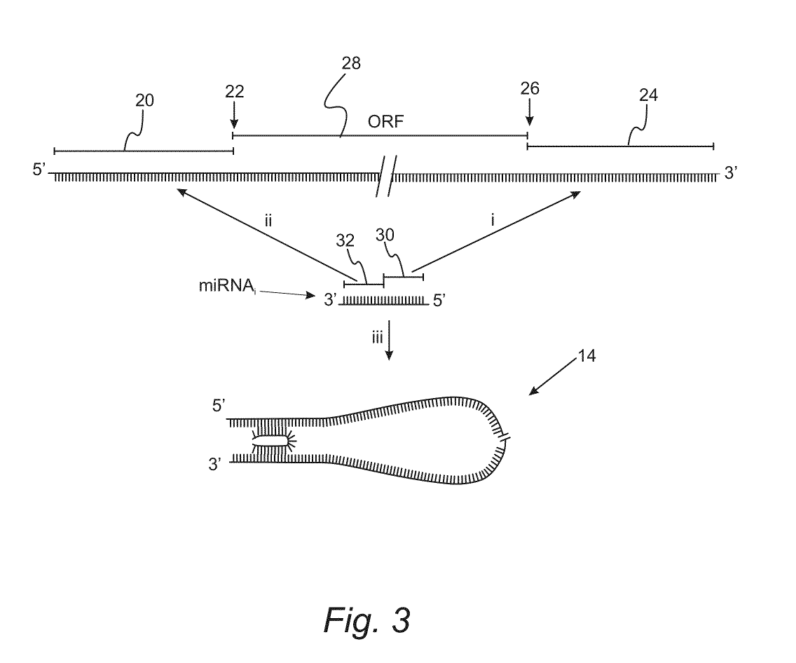

With reference to FIGS. 2A, 2B, and 3, a schematic flowchart for a method of evaluating the presence of a disease in a subject is provided. In a step a), a set of mRNA sequences 10 and a set of miRNA sequences 12 are used to identify candidate miRNA-mRNA complexes 14 in which an mRNA sequence from the set of mRNA sequences 10 stably hybridizes to a miRNA from the set of miRNA sequences 12. Each mRNA sequence has an upstream region 20 that is upstream of a translation start site 22 and a downstream region 24 that is downstream of a translation stop site 26. Each miRNA has a 5′ miRNA section 30 and a 3′ miRNA section 32. The candidate miRNA-mRNA complexes have sub-regions of the downstream region 24 that stably hybridize to portions of 5′ miRNA section 30 and sub-regions of the upstream region 20 that stably hybridize to portions of the 3′ miRNA section 32. In step b), candidate mRNA sequences 38 are identified as mRNA sequences that form candidate microRNA-mRNA complexes. Additionally or alternatively, candidate miRNA sequences 40 are identified as miRNA sequences that form candidate microRNA-mRNA complexes. It is next determined whether expression levels between candidate mRNA sequences in subjects having a disease and subjects not having the disease for each candidate mRNA sequence are different, and in particularly, statistically different with a p value less than or equal to 0.05 as set forth below. In addition or alternatively, it is determined whether expression levels between candidate miRNA sequences in subjects having a disease and subjects not having the disease for each candidate miRNA sequence are different and in particularly, statistically different with a p value less than or equal to 0.05 as set forth below.

In step c), differences 42 between expression levels between candidate mRNA sequences in subjects having a disease and subjects not having the disease for each candidate mRNA sequence are determined. In additional, or alternatively, differences between expression levels between candidate miRNA sequences in subjects having a disease and subjects not having the disease for each candidate miRNA sequence are determined. In step d), candidate RNA disease markers 44 are identified as candidate mRNA sequences and/or candidate miRNA sequences 45 that have differences determined in step c) that are greater than a predetermined value. In a variation, candidate RNA disease markers (mRNA or miRNA) are selected such that candidate miRNA sequences predominately up regulates or predominately down regulates a group of candidate mRNA sequences. That is candidate RNA disease markers are mostly associated with up regulation or down regulation but not both. In a refinement, the candidate miRNA sequences and/or candidate mRNA sequences are identified as candidate RNA disease markers if greater than 50 percent of the candidate mRNA sequences forming complexes with the candidate miRNA are up regulated. In another refinement, the candidate miRNA sequences and/or candidate mRNA sequences are identified as candidate RNA disease markers if greater than 70 percent of the candidate mRNA sequences forming complexes with the candidate miRNA are up regulated. In still another refinement, the candidate miRNA sequences and/or candidate mRNA sequences are identified as candidate RNA disease markers if greater than 80 percent of the candidate mRNA sequences forming complexes with the candidate miRNA are up regulated. In a refinement, the candidate miRNA sequences and/or candidate mRNA sequences are identified as candidate RNA disease markers if greater than 50 percent of the candidate mRNA sequences forming complexes with the candidate miRNA are down regulated. In another refinement, the candidate miRNA sequences and/or candidate mRNA sequences are identified as candidate RNA disease markers if greater than 70 percent of the candidate mRNA sequences forming complexes with the candidate miRNA are down regulated. In still another refinement, the candidate miRNA sequences and/or candidate mRNA sequences are identified as candidate RNA disease markers if greater than 80 percent of the candidate mRNA sequences forming complexes with the candidate miRNA are down regulated.

Still referring to FIGS. 2A, 2B, and 3, biological sample 46 is obtained from a test subject 48 in step e). Examples of biological samples include, but are not limited to, blood, plasma, cell free plasma, serum, CSF, urine, feces, saliva, biopsy samples, tissue, skin, hair, tumor, PAP smears, moles, warts, and the like. In step f), expression levels 52 of each candidate RNA disease marker from the biological sample is determined. In step g), the expression level of each of the candidate RNA disease markers 44 is compared to a control 54 such that deviation of expression levels of at least one candidate RNA disease marker from the control indicates presence of the disease. In one refinement, the control includes the expression levels of mRNA and/or miRNA sequences (i.e., candidate miRNA and candidate mRNA sequences) from a plurality of subjects not having the disease.

In a variation of the present embodiment, deviation of expression levels of at least three candidate mRNA sequences indicates presence of the disease. In another variation of the present embodiment, deviation of expression levels of at least six candidate mRNA sequences indicates presence of the disease.

In another variation, the subject will be monitored for disease progression when the subject has the expression level of at least one candidate RNA disease marker that deviates from the control. In this variation, the progression of the diseased state in the subject is monitored. This step includes again obtaining a biological sample 46 from test subject 48, determining the expression levels 52 of each candidate RNA disease marker from the biological sample, and comparing the expression level of each of the candidate RNA disease markers 44 to a control 54 such that deviation of expression levels of at least three candidate RNA disease markers from the control indicates presence of the disease. One skilled in the art would appreciate that a subject who was determined not to have the diseased state may also be monitored using the above method as a prophylactic measure or to parallel results from other diagnostic tests. Progression herein may refer to the disease state of the subject worsening, such as the incidence of new symptoms which may include, but not limited to physiological, mental, or psychological characteristics. Still further in another embodiment of the present invention a deviation of expression levels of at least ten candidate mRNA sequences indicates progression of diseased state in the subject. In a refinement, a deviation of expression levels of 2-3, 4-6, 7-10, 11-15, or 16-30 candidate mRNA sequences are used to identify a subject as having the disease.

In a variation, the method further includes a step of increasing an amount of a therapeutic agent administered to the subject if progression of the diseased state is determined.

In one particular variation, the present method is used to monitor neuroblastoma. Useful mRNA sequences that function as neuroblastoma biomarkers include, but are not limited to, mRNA sequences selected from the group consisting of AGPAT4 having NCBI (National Center for Biotechnology Information) number NM_{—}020133 (SEQ ID No: 1), BTBD9 having NCBI number NM_{—}001172418 (SEQ ID NO: 2), BTBD9 having NCBI number NM_{—}152733 (SEQ ID NO: 3), BTBD9 having NCBI number NM_{—}001099272 (SEQ ID NO: 4), BTBD9 having NCBI number NM_{—}052893 (SEQ ID NO: 5), GABBR1 having NCBI number NM_{—}001470 (SEQ ID NO: 6), GABBR1 having NCBI number NM_{—}021904 (SEQ ID NO: 7), GABBR1 having NCBI number NM_{—}021903 (SEQ ID NO: 8), KCNK10 having NCBI number NM_{—}021161 (SEQ ID NO: 9), KCNK10 having NCBI number NM_{—}138318 (SEQ ID NO: 10), KCNK10 having NCBI number NM_{—}138317 (SEQ ID NO: 11), LRRTM4 having NCBI number NM_{—}024993 (SEQ ID NO: 12), LRRTM4 having NCBI number NM_{—}001134745 (SEQ ID NO: 13), S100PBP having NCBI number NM_{—}022753 (SEQ ID NO: 14), S100PBP having NCBI number NM_{—}001256121 (SEQ ID NO: 15), and combinations thereof. In a further refinement, useful RNA biomarkers for neuroblastoma include an miRNA sequence or miRNA sequences selected from the group consisting of hsa-miR-9 (SEQ ID NO: 16), hsa-miR-18a* (SEQ ID NO: 17), hsa-miR-136 (SEQ ID NO: 18), hsa-miR-152 (SEQ ID NO: 19), hsa-miR-185 (SEQ ID NO: 20), hsa-miR-205 (SEQ ID NO: 21), hsa-miR-214 (SEQ ID NO: 22), hsa-miR-221 (SEQ ID NO: 23), hsa-miR-324-3p (SEQ ID NO: 24), hsa-miR-326 (SEQ ID NO: 25), hsa-miR-328 (SEQ ID NO: 26), hsa-miR-346 (SEQ ID NO: 27), hsa-miR-489 (SEQ ID NO: 28), hsa-miR-500a (SEQ ID NO: 29), hsa-miR-610 (SEQ ID NO: 30), hsa-miR-650 (SEQ ID NO: 31), and combinations thereof. In this variation, the therapeutic agent is an anti-cancer drug such as doxorubicin hydrochloride, cyclophosphamide, or vincristine sulfate. Note that for the NCBI numbers, “T” has been replaced by “U” as these are RNAs.

In another particular variation, the present method is used to monitor Parkinson's disease. In a refinement, useful RNA biomarkers include an mRNA sequence or mRNA sequences selected from the group consisting of AMMECR1 having NCBI number NM_{—}001025580 (SEQ ID NO: 32), AMMECR1 having NCBI number NM_{—}001171689 (SEQ ID NO: 33), AMMECR1 having NCBI number NM_{—}015365 (SEQ ID NO: 34), CASZ1 having NCBI number NM_{—}001079843 (SEQ ID NO: 35), CASZ1 having NCBI number NM_{—}017766 (SEQ ID NO: 36), CCNG2 having NCBI number NM_{—}004354 (SEQ ID NO: 37), FBXO41 having NCBI number NM_{—}001080410 (SEQ ID NO: 38), FOXP1 having NCBI number NM_{—}001244813 (SEQ ID NO: 39), FOXP1 having NCBI number NM_{—}001244814 (SEQ ID NO: 40), FOXP1 having NCBI number NM_{—}001244815 (SEQ ID NO: 41), FOXP1 having NCBI number NM_{—}001012505 (SEQ ID NO: 42), FOXP1 having NCBI number NM_{—}001244808 (SEQ ID NO: 43), FOXP1 having NCBI number NM_{—}001244810 (SEQ ID NO: 44), FOXP1 having NCBI number NM_{—}001244812 (SEQ ID NO: 45), FOXP1 having NCBI number NM_{—}001244816 (SEQ ID NO: 46), FOXP1 having NCBI number NM_{—}032682 (SEQ ID NO: 47), JRK having NCBI number NM_{—}001077527 (SEQ ID NO: 48), JRK having NCBI number NM_{—}003724 (SEQ ID NO: 49), MAPT having NCBI number NM_{—}001123066 (SEQ ID NO: 50), MAPT having NCBI number NM_{—}001123067 (SEQ ID NO: 51), MAPT having NCBI number NM_{—}001203251 (SEQ ID NO: 52), MAPT having NCBI number NM_{—}001203252 (SEQ ID NO: 53), MAPT having NCBI number NM_{—}005910 (SEQ ID NO: 54), MAPT having NCBI number NM_{—}016834 (SEQ ID NO: 55), MAPT having NCBI number NM_{—}016835 (SEQ ID NO: 56), MAPT having NCBI number NM_{—}016841 (SEQ ID NO: 57), NFYC having NCBI number NM_{—}001142587 (SEQ ID NO: 58), NFYC having NCBI number NM_{—}001142588 (SEQ ID NO: 59), NFYC having NCBI number NM_{—}001142589 (SEQ ID NO: 60), NFYC having NCBI number NM_{—}001142590 (SEQ ID NO: 61), NFYC having NCBI number NM_{—}014223 (SEQ ID NO: 62), QKI having NCBI number NM_{—}006775 (SEQ ID NO: 63), QKI having NCBI number NM_{—}206853 (SEQ ID NO: 64), QKI having NCBI number NM_{—}206854 (SEQ ID NO: 65), QKI having NCBI number NM_{—}206855 (SEQ ID NO: 66), RAB1A having NCBI number NM_{—}004161 (SEQ ID NO: 67), RAB1A having NCBI number NM_{—}015543 (SEQ ID NO: 68), RAPGEF3 having NCBI number NM_{—}001098531 (SEQ ID NO: 69), RAPGEF3 having NCBI number NM_{—}001098532 (SEQ ID NO: 70), RAPGEF3 having NCBI number NM_{—}006105 (SEQ ID NO: 71), STAT2 having NCBI number NM_{—}005419 (SEQ ID NO: 72), STAT2 having NCBI number NM_{—}198332 (SEQ ID NO: 73), VASH1 having NCBI number NM_{—}014909 (SEQ ID NO: 74), and combinations thereof. In a further refinement, useful RNA biomarkers for Parkinson's disease include an miRNA sequence or miRNA sequences selected from the group consisting of hsa-miR-1184 (SEQ ID NO: 75), hsa-miR-221 (SEQ ID NO: 23), hsa-miR-1207-5p (SEQ ID NO: 76), hsa-miR-760 (SEQ ID NO: 77), and combinations thereof. In this variation, the therapeutic agent is an anti-Parkinson's disease drug such as levodopa, selegiline, rasagiline, bromocriptine, pramipexole, ropinirole, transdermal rotigotine, apomorphine, tolcapone, entacapone, trihexyphenidyl, benztropine, orphenadrine, procyclidine, biperiden, and amantadine.

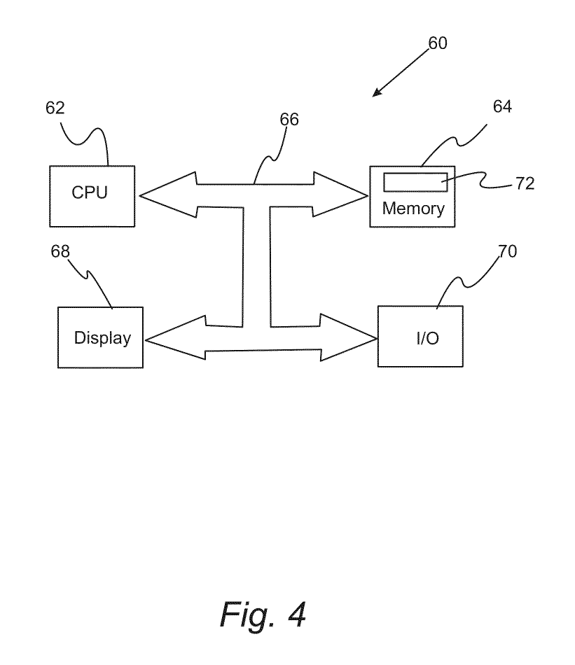

With reference to FIGS. 3 and 4, steps a) through d) at least partially apply the protocol set forth in U.S. Pat. Pub. No. 2012/0015351, filed Feb. 22, 2011 and U.S. patent application Ser. No. 13/579,896 filed Aug. 17, 2012, the entire disclosures of which is hereby incorporated by reference. In particular, as illustrated in FIG. 4, a computer system 60 is used for these steps. Computer system 60 of the present invention includes central processing unit (CPU) 62, memory 64, and input/output interface 66. Computer system 60 communicates with display 68 and input devices 70 such as a keyboard and mouse via interface 66. In one variation, memory 64 includes one or more of the following: random access memory (RAM), read only memory (ROM), CDROM, DVD, disk drive, tape drive. The method of various embodiments is implemented by routine 72 that is stored in memory 64 and executed by the CPU 62. The method implemented by routine 72 includes a step of receiving data identifying a set of mRNA sequences 10 representing a gene or portions thereof. Characteristically, the nucleotide sequence has a region 20 that is upstream of translation start site 22, a section 24 that is downstream of translation stop site 26, and an open reading frame 28.

The method also includes a step of receiving data identifying a set of miRNA sequences 12. This set of miRNA sequences can be downloaded from http://mirbase.org/ftp.shtml. Each miRNA sequence has 5′ miRNA section 30 and a 3′ miRNA section 32. In step i), section 24 is evaluated for sub-regions that are capable of stable hybridizing to at least of a portion of 5′ miRNA section 30. In general, one or more portions of section 30 and one or more sub-sections of section 24 are evaluated. In one refinement, stable hybridization is determined by the degree of complementariness of miRNA section 30 to a sub-region of section 24 with perfect complementary sub-regions of section 24 being the most stable. In another refinement, stable hybridization is determined by thermodynamic criteria. Specifically, the change AG in Gibbs free energy for the interaction of a portion of 5′ miRNA section 30 with sub-regions of section 24 is evaluated with interactions having AG less than a predetermined value being identified as candidate sites for in vivo interactions. In a further refinement, AG for these hybridizations is less than about −10 kcal/mol. In still a further refinement, AG for these hybridizations is less than about −13 kcal/mol. The thermodynamic calculation may be carried out using the RNAhybrid™ software available from http://bibiserv.techfak.uni-bielefeld.de/rnahybrid/.

Still referring to FIG. 3, in step ii), section 20 is evaluated for sub-regions that are capable of stable hybridizing to at least a portion of 3′ miRNA section 32. In general, one or more portions of 3′ miRNA section 32 and one or more sub-sections of section 20 are evaluated. In one refinement, stable hybridization is determined by the degree of complementariness of 3′miRNA section 32 to a sub-region of section 20 with perfect complementary sub-regions of section 32 being the most stable. In another refinement, stable hybridization is determined by thermodynamic criteria. Specifically, the change AG in Gibbs free energy for the interaction of a portion of 3′ miRNA section 32 with sub-regions of section 20 is evaluated with interactions having AG less than a predetermined value being identified as candidate sites for in vivo interactions. In a further refinement, AG for these hybridizations is less than about −10 kcal/mol. In still a further refinement, AG for these hybridizations is less than about −13 kcal/mol. In yet another refinement, section 20 includes an AUG motif that interacts with one or more portions of 3′ miRNA section 32. In step iii), combinations of stably hybridizing sub-regions of section 24 to portions of 5′ miRNA section 30 and stably hybridizing sub-regions of section 20 to portions of 3′ miRNA section 32 are used to identify candidates for microRNA-mRNA complexes 14. In another variation, a computer readable medium embodying a program of instructions executable by a processor is used to perform the method steps a)-d) of FIGS. 2A, 2B, and 3. Specifically, the computer readable medium is encoded with instructions for the steps of the methods of the invention. Examples of useful computer readable media include, but are not limited to, hard drives, floppy drives, CDROM, DVD, optical drives, random access medium, and the like.

As set forth in the present invention utilizing mRNA and miRNA data, such expression can be determined using four separate approaches: (1) quantitative reverse transcriptase polymerase chain reaction (qRT-PCR), (2) microarray, (3) high throughput sequencing technologies, and (4) fluorescence in situ hybridization (FISH). Expression data of mRNA and miRNA can be determined using quantitative reverse transcriptase polymerase chain reaction (qRT-PCR) utilizing the following equipment: (i) TaqMan® Probe-based expression analysis in single tubes (Life Technologies Corportation, Carlsbad, Calif.); (ii) TaqMan® Probe-based multi-well plates (Life Technologies Corportation, Carlsbad, Calif.); (iii) TaqMan® Probe-based multi-well microfluidic cards (Life Technologies Corportation, Carlsbad, Calif.); (iv) SYBR Green real-time PCR (QIAGEN Inc., Valencia, Calif.); (v) RT2 Profiler PCR Array (QIAGEN Inc., Valencia, Calif.); and, (vi) miRCURY LNATM Universal RT microRNA PCR (Exiqon, Inc., Woburn, Mass.). Ct (threshold cycle) is the intersection between an amplification curve and a threshold line. The Ct value represents the expression value. It is a relative measure of the concentration of target in the PCR reaction.

Expression data of mRNA and miRNA can be determined using microarray utilizing the following equipment: (i) Human Genome U133 (HG-U133) Plus 2.0 Array (Affymetrix, Inc., Santa Clara, Calif.); (ii) GeneChip Human Exon 1.0 ST Array (Affymetrix, Inc., Santa Clara, Calif.); and, (iii) miRCURY LNATM microRNA array (Exiqon, Inc., Woburn, Mass.). The fluorescence signal intensity is determined and compared for each sample. Expression data of mRNA and miRNA can be determined using high throughput sequencing technologies utilizing the following equipment: (i) HiSeq systems (Illumina, Inc., San Diego, Calif.); (ii) MiSeq systems (Illumina, Inc., San Diego, Calif.); and, (iii) Ion Personal Genome Machine™ Sequencer (Life Technology Corporation, Carlsbad, Calif.). The number of reads represents expression.

Changes in expression values between disease and control samples can be calculated from comparing representing values between disease and control samples from each technology. Statistical significance (such as a two tail student t-test, p value equal to or less than 5%) was determined if differences between the two values are significant. Significance was classified into several categories of up expression, down expression, and the expression fold change to determine whether the test sample is significantly different from the representative control group.

Identifying biomarkers, differentially expressed mRNA and miRNA, between genome-wide data of biological samples from subjects with a diseased state, and/or subjects with an increased risk of the diseased state was compared to normal controls. Applying the miRNA:mRNA binding model as already outlined, predicted miRNA that bind simultaneously to the target mRNA's 5′ and 3′ UTR are further triaged to biomarkers that are differentially expressed. These biomarkers, differentially expressed mRNA and/or miRNA, were further triaged with biomarkers, proteins, and enzymes known to be associated with the diseased state. Predictors are developed from identifying biomarkers associated with the diseased state based on differential expression from controls. These biomarker predictors were then applied to multiple independent subject sets from biological samples to predict, identify, and monitor subjects with or suspected of having the diseased state.

Biomarker predictors are determined from differences of the expression of level of diseased state biological samples and a control set of miRNA and/or mRNA expression levels. Triaged biomarkers may be selected using several approaches, including, but not limited to, (i) the percentage or fold change between the control set and diseased state expression levels of each mRNA and/or miRNA; and (ii) a two tail Student's T-test with a p value cut off. Therefore, in a refinement, candidate RNA disease markers are candidate mRNA sequences in which the difference between the expression level between subjects having a disease and subjects not having the disease is greater than 20 percent or candidate miRNA sequences in which the difference between the expression level between subjects having a disease and subjects not having the disease is greater than 20 percent. In another refinement, candidate RNA disease markers are candidate mRNA sequences in which the difference between the expression level between subjects having a disease and subjects not having the disease is greater than 50 percent or candidate miRNA sequences in which the difference between the expression level between subjects having a disease and subjects not having the disease is greater than 50 percent. In still another refinement, candidate RNA disease markers are candidate mRNA sequences in which the difference between the expression level between subjects having a disease and subjects not having the disease is greater than 100 percent or candidate miRNA sequences in which the difference between the expression level between subjects having a disease and subjects not having the disease is greater than 100 percent. In yet another refinement, candidate RNA disease markers are candidate mRNA sequences in which the difference between the expression level between subjects having a disease and subjects not having the disease is between 20 and 300 percent or candidate miRNA sequences in which the difference between the expression level between subjects having a disease and subjects not having the disease is between 20 and 300 percent. In another variation, candidate RNA disease markers are candidate mRNA sequences and/or candidate miRNA sequences in which expression levels between subjects having a disease and subjects not having the disease are statistically different. A Student's T test is a statistical test to determine whether the null hypothesis is supported. The p-value obtained from the Student's T test is the probability of obtaining a test statistic, rejecting the null hypothesis, when the p-value is less than a predetermined level. When the p-value is less than this predetermined level, the expression difference for each miRNA and/or mRNA is triaged to be statistically significant. Thus, the triaged biomarkers having statistical significance are associated with a diseased state. In one refinement, the p-value is equal to or less than p=0.05. In another refinement, the p-value is equal to or less than p=0.01. Still further in another refinement, the p-value is equal to or less than p=0.03. Still even further in another refinement, the p-value is equal to or less than p=0.005.

### EXPERIMENTAL

The following examples are provided in order to demonstrate and further illustrate certain preferred embodiments and aspects of the present invention and are not to be construed as limiting the scope thereof.

### Example 1

### MicroRNA and Target Gene Signature in Advanced Neuroblastoma

Expression levels of mRNA depend on various factors including DNA copy number, DNA methylation, histone acetylation, active transcription factors, splicing factors, and regulating miRNAs. Here we analyse mRNA expression in a miRNA-centric manner, comparing it with miRNA expression data to increase the specificity of advanced neuroblastoma signatures. Unlike other miRNA-mRNA correlation studies, which look for any negatively- or positively correlated pairs with a certain statistical significance, our method investigates the consistency of expression value changes within each miRNA's targets. This is based on our hypothesis that if a miRNA's function is important and causal to progress neuroblastoma and the expression of the miRNA is up (down)-regulated in the advanced stage, its targets will be preferentially down (up)-regulated in the advanced neuroblastoma. Therefore, we will define a miRNA and its targets as important to the advanced neuroblastoma if such cases are found. Note that this method proceeds without bias of previous knowledge and simply searches for consistent expression data across different datasets (mRNA and miRNA expression), thus allowing novel findings.

The Vandesompele group, which has recently identified correlations between MYCN-induced miRNAs with poor outcome in MYCN-activated tumors (Mestdagh et al., 2010b), provided us matching mRNA and miRNA expression patterns with an annotation file. We also downloaded the mRNA data, disease state, and MYCN amplification status from the Gene Express Omnibus (Series accession number GSE21713). The disease states followed the International Neuroblastoma Staging System's four stages. Stages 1 and 2 indicate the cancer is still localized, while stages 3 and 4 indicate metastasis of the original tumor. To understand global genetic differences in advanced neuroblastoma, we defined two groups for comparison: stage 4 as one group and stage 1 and 2 as another, regardless of MYCN amplification status. Stage 3 data were excluded in order to obtain maximal contrast between the non-metastatic stages and metastatic stage 4. By comparing miRNA and mRNA expression of these two groups, we expected to obtain a more specific advanced neuroblastoma signature, which might not be apparent through mRNA or miRNA analysis alone.

### Example 2

### mRNA Expression Analysis and Differently Expressed mRNAs in Advanced Neuroblastoma

In total, there are 30 primary tumor sample data from 14 stage 4 subjects and 16 stage 1 and 2 subjects. Briefly, the Vandesomepele group derived the expression data as follows: after each sample expression dataset was obtained using GeneChip Human Exon 1.0 ST Arrays (Affymetrix), all exon data were combined to transcript clusters (hgl8/core exons), to obtain expression information per gene after normalization according to the RMA-sketch algorithm using Affymetrix Power Tools. We used these RMA normalized data calculated by the Vandesomepele group to obtain differentially expressed genes between the two groups. Student t-tests were performed using Microsoft Excel functions and mRNA lists with p-values less than 0.05 were prepared as up- and down-regulated mRNAs using HUGO Gene Nomenclature Committe (HGNC) gene symbol annotation. We ignored transcripts without gene symbol annotation or empty data points among subject samples. If more than two probe sets corresponded to one gene symbol, we chose the probe set with lower p-values. Among a total of 372 up-regulated and 689 down-regulated mRNAs with gene symbols having p<0.05, the top 50 genes with the greatest fold changes are shown in Table 1.

TABLE 1
Top 50 genes with the greatest fold changes among differently
expressed mRNAs in advanced neuroblastoma (p < 0.05).

| Down- | | Log2 | Up- | | Log2 |
|---|---|---|---|---|---|
| Regulated | | (Fold | Regulated | | (Fold |
| Gene | p-value | Change) | Gene | p-value | Change) |
| ALCAM | 0.012519 | −1.1404 | ACTA2 | 0.017706 | 0.84008 |
| APBA1 | 0.000975 | −1.00703 | BIRC5 | 0.007473 | 0.88797 |
| ATP2B4 | 0.008721 | −1.02324 | CCNA2 | 0.009386 | 0.79357 |
| CADM3 | 0.002402 | −1.24665 | CDC45L | 0.007403 | 0.75931 |
| CDH6 | 0.049003 | −1.01205 | CDCA5 | 0.008474 | 0.77314 |
| DOC2B | 0.013732 | −1.02395 | CMBL | 0.004642 | 0.72441 |
| DRD2 | 0.001234 | −1.08816 | DDX1 | 0.046072 | 1.22142 |
| ECEL1 | 0.003074 | −1.10837 | E2F3 | 0.000912 | 0.74161 |
| EPB41L3 | 0.008477 | −1.37384 | FN1 | 0.008058 | 1.03848 |
| HIST1H1A | 0.016193 | −1.34667 | FOXM1 | 0.008946 | 0.9346 |
| HS6ST3 | 0.000384 | −1.10939 | GJC1 | 0.002895 | 0.82971 |
| LRRTM4 | 0.040988 | −1.31354 | HIST1H2BM | 0.037272 | 0.85818 |
| NRCAM | 0.031584 | −1.04967 | HMGB2 | 0.006813 | 0.883 |
| NTRK1 | 0.001072 | −1.79752 | MAD2L1 | 0.012955 | 0.92231 |
| PGM2L1 | 0.010537 | −1.14954 | MYBL2 | 0.019576 | 0.82346 |
| PLXNC1 | 0.000939 | −1.08949 | MYCN | 0.019589 | 1.23078 |
| PMP22 | 0.003898 | −1.62031 | NAG | 0.020134 | 1.26792 |
| PRKCA | 0.011104 | −1.04588 | ODC1 | 0.001326 | 1.3045 |
| PRPH | 0.003777 | −1.41955 | PAICS | 0.000609 | 0.74121 |
| PTN | 0.00069 | −1.19001 | PHGDH | 0.026183 | 0.88181 |
| RAB3C | 0.025466 | −1.00737 | RRM2 | 0.00773 | 1.46016 |
| REEP1 | 0.002232 | −1.00576 | SLC16A1 | 0.000721 | 0.93447 |
| SCG2 | 0.009003 | −1.21159 | TYMS | 0.016238 | 0.95479 |
| SCN9A | 0.0207 | −1.02774 | VCAN | 0.019634 | 0.99934 |
| SYN3 | 0.006658 | −1.04893 | | | |
| TMEM176A | 0.006741 | −1.11877 | | | |

### Example 3

### Mapping Differentiated mRNAs to Regulating miRNAs

For all mRNAs identified as differentially regulated, their regulating miRNAs were predicted using the miBridge miRNA target prediction method (v.1). We then calculated the hypergeometric distribution function of miRNA targets to test whether overall targets are either up- or down-regulated. Table 2 shows the predicted regulating miRNAs (within the miRNA list in the array measured) with targets enriched in either up- or down-regulated mRNAs with p<0.05. Within the mir-17-92 cluster, hsa-miR-18* targets are enriched in the down-regulated mRNAs, supporting our hypothesis and miRNA target predictions (though miR-92a is not included in this p<0.05 list, inclusion of genes with less than 15 empty values in subject samples yields enriched miR-92a targets in the down-regulated mRNAs with p=0.048).

TABLE 2
Predicted miRNAs as potential regulators of advanced
neuroblastoma (hypergeometric distribution p < 0.05).

| | | Number of | | |
|---|---|---|---|---|
| | Number of | targets in | | |
| | targets in up- | down- | | |
| regulating | regulated | regulated | | Predicted |
| miRNA | genes | genes | p-value | as |
| hsa-miR-9 | 0 | 7 | 0.048179 | Up |
| hsa-miR-18a* | 3 | 17 | 0.031065 | Up |
| hsa-miR-136 | 0 | 8 | 0.031175 | Up |
| hsa-miR-152 | 0 | 7 | 0.048179 | Up |
| hsa-miR-185 | 4 | 23 | 0.01224 | Up |
| hsa-miR-205 | 1 | 12 | 0.025063 | Up |
| hsa-miR-214 | 9 | 32 | 0.026804 | Up |
| hsa-miR-221 | 2 | 17 | 0.013123 | up |
| hsa-miR-324-3p | 8 | 30 | 0.025304 | up |
| hsa-miR-326 | 6 | 36 | 0.001494 | up |
| hsa-miR-328 | 2 | 19 | 0.006699 | up |
| hsa-miR-346 | 5 | 23 | 0.024396 | up |
| hsa-miR-500 | 0 | 7 | 0.048179 | up |
| hsa-miR-610 | 4 | 18 | 0.045656 | up |
| hsa-miR-650 | 4 | 24 | 0.009198 | up |
| hsa-miR-489 | 13 | 10 | 0.017781 | down |

### Example 4

### miRNA Expression Analysis

The miRNA expression data were obtained from the same subject samples analyzed in the mRNA expression: total 14 stage 4 subjects and 16 stage 1 and 2 subjects. We used the RMA normalized data with 312 mature miRNA annotations as provided by the Vandesomepele group. Student t-test was performed using Microsoft Excel functions and miRNA lists of p<0.05 are shown in Table 3.

TABLE 3
Differentially expressed miRNAs in the advanced neuroblastoma (student t-test p < 0.05)

| up-regulated | | Log₂ (fold | down-regulated | | Log₂ (fold |
|---|---|---|---|---|---|
| miRNA | p-value | change) | miRNA | p-value | change) |
| hsa-miR-9 | 0.037541 | 1.173707 | hsa-miR-15a | 0.044916 | −0.49254 |
| hsa-miR-9* | 0.018107 | 1.235894 | hsa-miR-24 | 0.030286 | −0.44341 |
| hsa-miR-18a | 0.02853 | 0.570961 | hsa-miR-26a | 0.007846 | −0.53493 |
| hsa-miR-18a* | 0.001933 | 0.58714 | hsa-miR-26b | 0.011224 | −0.44942 |
| hsa-miR-19a | 0.038493 | 0.588543 | hsa-miR-30a-3p | 0.023514 | −0.68173 |
| hsa-mir-92 | 0.006916 | 0.782683 | hsa-miR-30b | 0.00698 | −0.50628 |
| hsa-miR-105 | 0.036113 | 1.681312 | hsa-miR-30e-3p | 0.023054 | −0.77332 |
| hsa-miR-320 | 0.023005 | 0.362074 | hsa-miR-95 | 0.012503 | −0.91388 |
| hsa-miR-375 | 0.03906 | 1.901862 | hsa-miR-103 | 0.0395 | −0.35021 |
| hsa-miR-517a | 0.045293 | 1.423221 | hsa-miR-125b | 0.00271 | −0.89665 |
| hsa-miR-520g | 0.024751 | 1.757529 | hsa-miR-128a | 0.016545 | −0.73381 |
| hsa-miR-526b* | 0.049281 | 0.601139 | hsa-miR-137 | 0.000167 | −1.78508 |
| hsa-miR-645 | 0.014262 | 0.758181 | hsa-miR-140 | 0.010729 | −0.3409 |
| | | | hsa-miR-148b | 0.009921 | −0.56818 |
| | | | hsa.miR-149 | 0.002849 | −1.13549 |
| | | | hsa-miR-190 | 0.048211 | −1.23233 |
| | | | hsa-miR-204 | 0.008341 | −2.21371 |
| | | | hsa-miR-215 | 0.005819 | −1.68041 |
| | | | hsa-miR-216 | 0.042686 | −1.38497 |
| | | | hsa-miR-218 | 0.045402 | −0.86703 |
| | | | hsa-miR-324-3p | 0.034135 | −0.52073 |
| | | | hsa-miR-324-5p | 0.028667 | −0.74833 |
| | | | hsa-miR-326 | 0.048664 | −0.71389 |
| | | | hsa-miR-330 | 0.010354 | −0.98399 |
| | | | hsa-miR-331 | 0.004691 | −0.73239 |
| | | | hsa-miR-340 | 0.010086 | −0.77428 |
| | | | hsa-miR-488 | 0.01028 | −1.08969 |
| | | | hsa-miR-491 | 0.042597 | −0.68084 |
| | | | hsa-miR-628 | 0.000553 | −1.00498 |

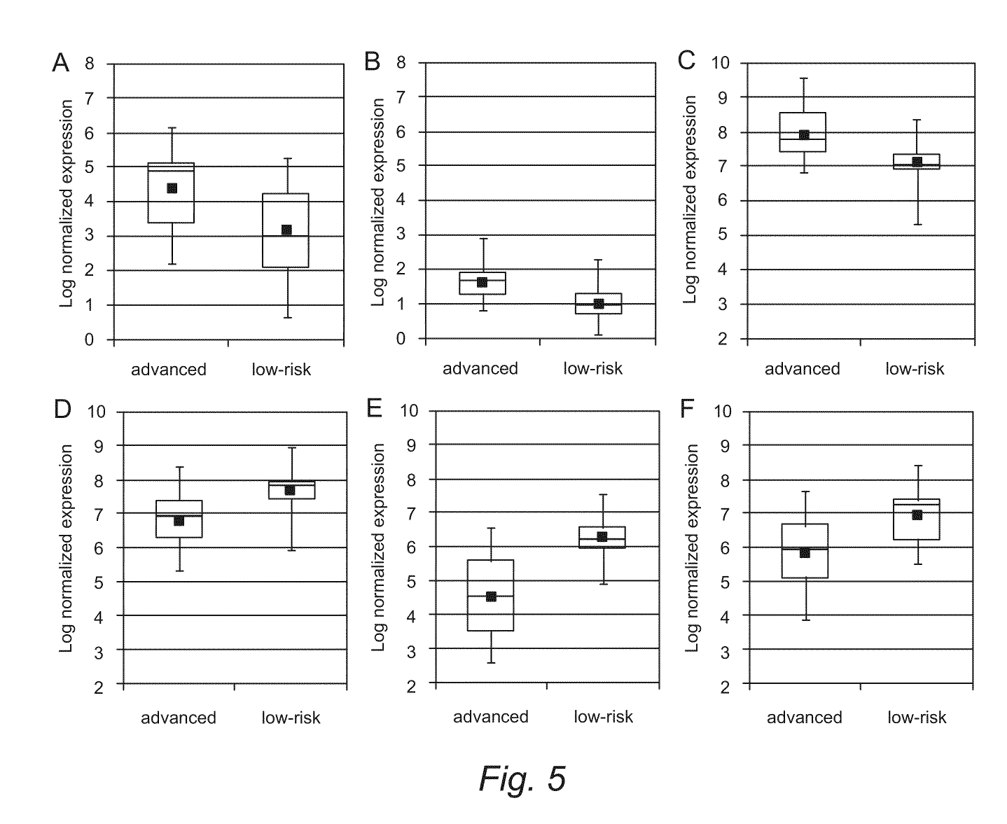

We found that two miRNAs predicted as up-regulated with hypergeometric distribution p<0.05, miR-9 and miR-18a*, are actually up-regulated in microarray experiments with student t-test p<0.05. Since miR-18a* is a minor strand (less present than miR-18a) within the mir-17-92 cluster, we conclude that miR-9 is the most consistent miRNA in terms of its expression and its targets' expression in our analysis. FIG. 5 shows the box plots of miR-9 expressions in advanced and low-risk stage subject samples together with five other miRNAs whose expression values are most significantly changed in up- and down-regulated miRNAs.

### Example 5

### Correlation of mRNA and miRNA Expression Data

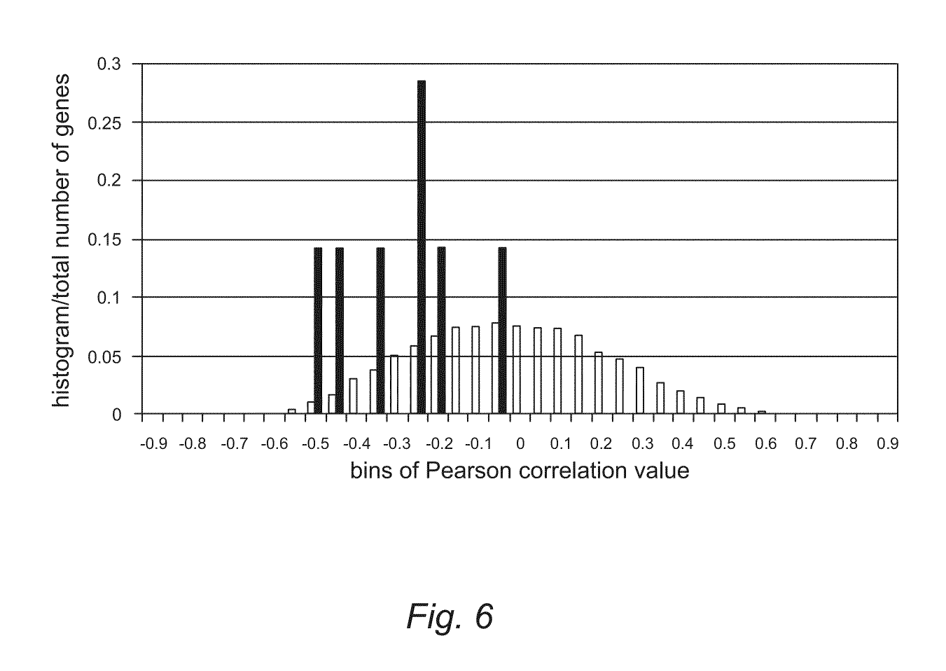

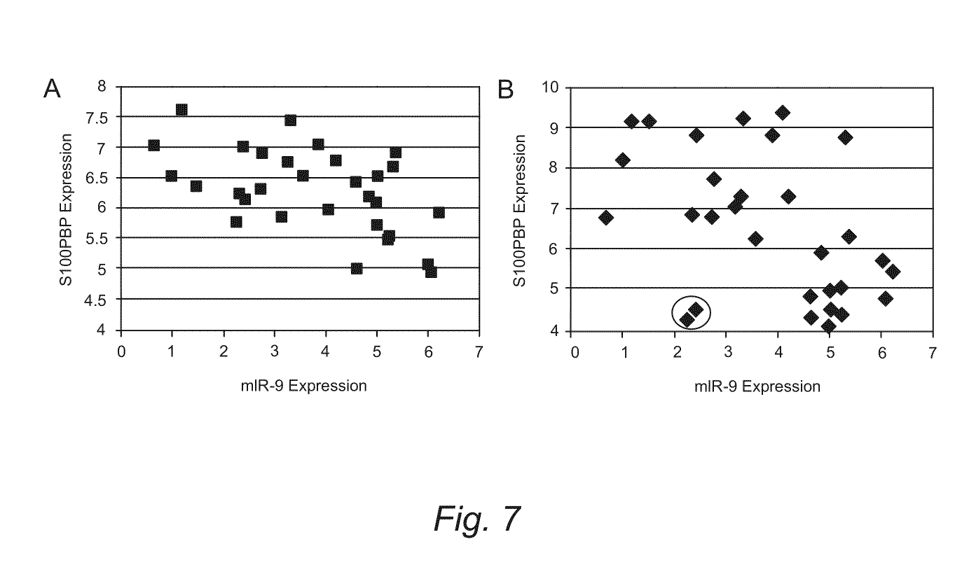

We found miR-9 to have the most consistent signal in terms of miRNA and mRNA expression level changes in advanced neuroblastoma. Note that miRNA expression pattern alone does not justify further in-depth analyses of miR-9 since other miRNAs have higher fold changes or lower p-value. Our approach thus provides a new way to prioritize important miRNAs. Interestingly, miR-9's seven predicted targets genes are found only in the down-regulated genes. However, this does not mean that expressions of miR-9 and its target genes are negatively correlated across entire samples. FIG. 6 shows the Pearson product moment correlation coefficient of miR-9 and its seven target genes across the 30 subject samples. For context, correlations between miR-9 and all mRNA expression are also shown. The correlations of miR-9 and target genes stand out from all other correlations and six of the seven targets are negatively correlated with miR-9. The six negatively correlated predicted targets are AGPAT4, BTBD9, GABBR1, KCNK10, LRRTM4, and S100PBP. Among them, LRRTM4 is in Table 1, containing the top 50 greatest fold change genes. FIG. 7 shows the scatter plots of the two most negatively correlated miR-9 target genes.

Though miR-9 can target multiple genes at the same time, the expression of each target gene varies from person to person, so that its function on each target gene might vary among subjects. In our analysis, six genes are predicted as functional targets in the neuroblastoma, potentially responsible for disease progression. As an example, some outliers shown in the circle in FIG. 7B are no longer outliers in the S100PBP case in FIG. 7A. Therefore, rather than one miRNA or one miRNA and its target, collective miR-9 targets and miR-9 might allow more accurate prediction of whether a neuroblastoma will progress to advanced stage.

### Example 6

### miR-9 Signature in Short- and Long-Lived Subjects

Scaruffi, et al. published research on non-coding RNA expressions with regards to outcome in high-risk neuroblastoma (Scaruffi et al., 2009). Among 31 high-risk, stage 4 neuroblastoma samples, miRNA expressions came from 17 short-term survivors (dead within 36 months from diagnosis) and 14 long-term survivors (alive with an overall survival time >36 months) were deposited in GEO (Series accession number GSE16444). We downloaded the data, which were log₂-transformed and quantile normalized, after obtaining the raw data using miRNA microarray System protocol v. 1.5 (Agilent Tenchnologies). Since they removed probes with less than 50% of detected slops across all arrays, many low signals were not present. We used the downloaded data to identify differential miRNAs between short- and long-term survivor groups. Significance was calculated with a student's t-test (unpaired, two-tailed, unequal variance); miRNAs up- and down-regulated in short-term survivors with p-values less than 0.05 are shown in Table 4.

TABLE 4
Differentially expressed miRNAs in short-term survivors compared with
long-term survivors. All subjects had advanced neuroblastoma.

| up- | | | down- | | |
|---|---|---|---|---|---|
| regulated | | Log₂ (fold | regulated | | Log₂ (fold |
| miRNA | p-value | change) | miRNA | p-value | change) |
| hsa-miR-9 | 0.0377472 | 0.68625181 | hsa-miR-22 | 0.040065 | −0.990610191 |
| hsa-miR-210 | 0.0284823 | 1.147684424 | hsa-miR-139-3p | 0.0478034 | −0.803202819 |
| hsa-miR-425 | 0.0127439 | 0.62221312 | hsa-miR-181c* | 0.0497988 | −1.015817709 |
| hsa-miR-758 | 0.0080157 | 1.01630988 | hsa-miR-302a | 0.0338064 | −0.915468934 |
| hsa-miR-885-5p | 0.0117879 | 1.125425787 | hsa-miR-502-3p | 0.0481067 | −0.69939469 |
| hsa-miR-885-3p | 0.0118758 | 0.669348934 | hsa-miR-886-3p | 0.0424204 | −1.691328238 |
| hsa-miR-877 | 0.0276158 | 0.571214174 | | | |
| hsa-miR-936 | 0.0388439 | 0.656436824 | | | |

Due to the distinct difference between diseased and healthy status, the overall number of differentially expressed miRNAs with a certain p-value cutoff may be much greater than that within the disease group. Within the disease group, though neuroblastoma has diverse outcomes, differentiating stages within the neuroblastoma is more difficult than differentiating disease from normal. To increase differentiating power, we did not include stage 3 neuroblastoma data in our section 2 analysis. Here, we further differentiate a narrower range within stage 4. Therefore, it is not surprising that fewer miRNAs were differentiated here than between advanced and low-risk neuroblastoma. Also, it is common for different research groups to produce differing lists of significantly changed genes.

However, if underlying causes exist for advanced neuroblastoma which further leads to short-term survival, we might identify them from a persistent signal in various contexts. We find miR-9, the only miRNA common between Tables 3 and 4, to be such a consistent signal. We want to emphasize that miR-9 must be evaluated in connection with target gene expressions. It was found to be the most consistent miRNA in terms of down-regulating target mRNAs when induced. Moreover, its expression differs between short- and long-term survivors. When it comes to identifying miRNA signature from expression data, it is therefore crucial to compare a miRNA's target expression patterns with its own expression. While many studies focus on global correlations between miRNA and mRNA expressions without a clear regulating matrix, our method of assessing consistency clearly helps pinpoint the signature miRNA in the disease progression.

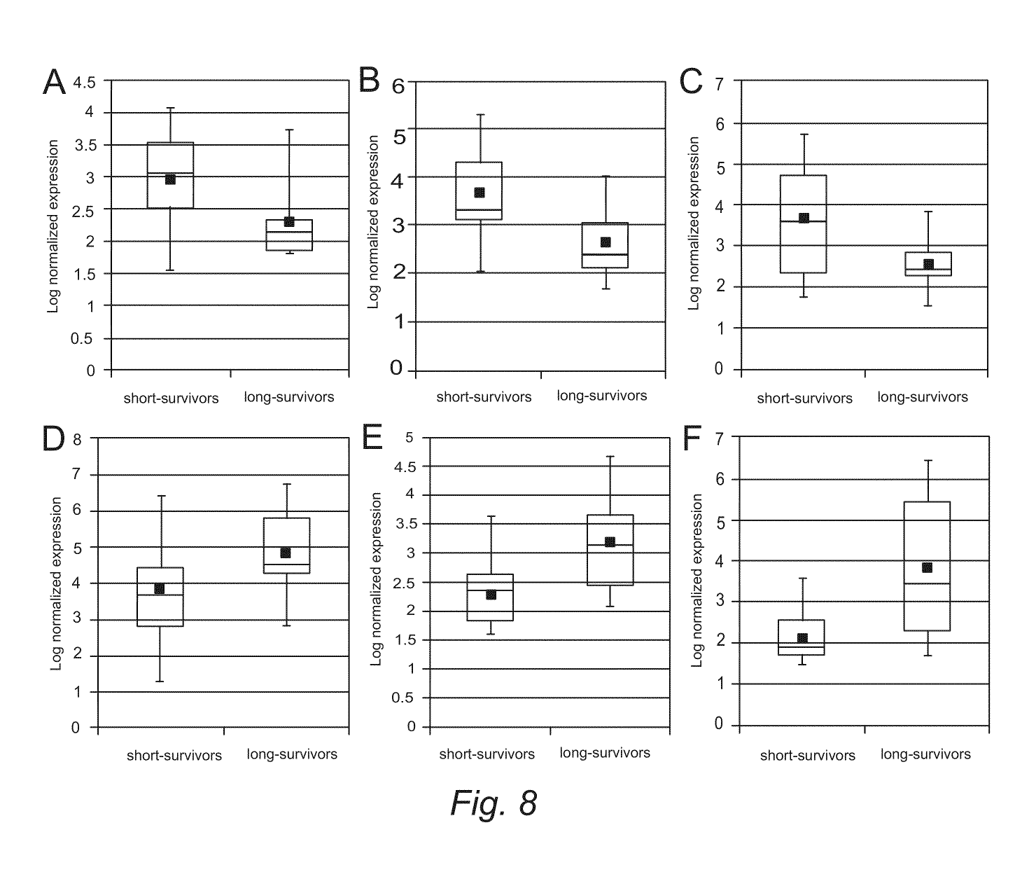

FIG. 8 shows the box plot of miR-9 and five other miRNA expressions (chosen in order of smaller p-values) of samples from short- and long-term survivors in Table 4. Though the p-value of miR-9 is not as low as others, its differentiating power is close to that of miR-758. In FIG. 5, the miR-9 level itself may not be powerful enough to differentiate advanced stage from low-risk neuroblastoma. Here, the data suggest that the broader range of miR-9 expression in advanced stage is due to its differentiating power between two sub-groups of advanced stage subjects (absolute number of normalized data in each dataset is not meaningful). In terms of early prognostic power, miR-9 and its targets hold high promise. Further investigation using larger samples is needed.

### Example 7

### miR-9 Functional Model in Advanced Neuroblastoma

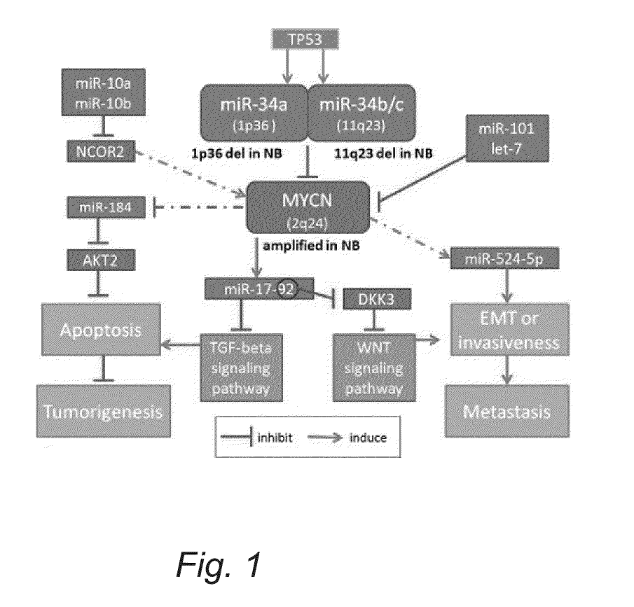

Recently, Ma et al. reported that miR-9 directly targets E-cadherin (CDH1), leading to increased cell motility and invasiveness in breast cancer (Ma et al., 2010). Though in a different cell type, since the function of CDH1 in solid tumors is the same, we expect miR-9 function to be similar in the neuroblastoma. Moreover, as a MYCN-activated miRNA, miR-9 fits well within the neuroblastoma miRNA networks (FIG. 1) as a function of developing advanced metastasized neuroblastoma. Our identification of miR-9 and its targets thus makes sense functionally as an early prognostic marker for developing high mortality neuroblastoma. FIG. 1 includes miR-9 and CDH1 as a functional model of developing advanced stage neuroblastoma.

We have used our new method to analyze mRNA and miRNA expression data to identify signature miRNA and target genes in stage 4 neuroblastoma. To obtain maximal contrast between the non-metastatic stages and metastatic stage 4, we excluded stage 3 data and combined stages 1 and 2 as low-risk. When we compared advanced and low-risk stage neuroblastoma, miR-9 related expressions had the most consistent data between mRNA and miRNA expression. We also confirmed that six out of the seven predicted targets were negatively correlated with miR-9 expression across entire samples. Furthermore, miR-9 expression was significantly up-regulated in samples from short-term survivors compared with those from long-term survivors within the same advanced-stage neuroblastoma group. In addition to these data-driven analyses, note that miR-9 has been identified as inducing metastasis in breast cancer by targeting E-cadherin. Therefore, our claim of expressions of miR-9 and its targets as a signature of advancing neuroblastoma fits well with previous studies. Further investigation with a larger number of samples is needed.

### Example 8

### mRNA and miRNA Markers Associated with Parkinson's Disease

We have identified a new miRNA target class, referred to herein as miBridge, in which the 5′- and 3′-end of a miRNA can simultaneously interact with the 3′-UTR (untranslated region) and 5′-UTR of a single gene [9], in contrast to other existing target prediction methods, which consider mostly interaction of the 5′-end of a miRNA with the 3′-UTR of a target gene. Utilizing miBridge for target prediction, we analyzed publicly available gene expression data of substantia nigra (SN) pars compacta neurons, SN brain region (focused gene sets), dorsal motor nucleus of the vagus (DMNV) brainstem, inferior olivary nucleus (ION) brainstem, whole blood, and B lymphoblast cell lines (BLCL) from a total of 80 PD patients and 83 controls, including 33 other neurodegenerative disease patient controls. Without having miRNA profiles available, we predicted differently expressed miRNAs between PD and controls based on target gene enrichment (hypergeometric distribution function p<0.05) within the differentially expressed mRNAs (two sided t-test p<0.05) for each of these 6 different sample data sets.

While many differentially predicted miRNAs are tissue specific, such as miR-133b (predicted only in the SNpc neuron sample data, in accordance with previous findings [10]), we found six miRNAs consistently predicted throughout the investigated sample data. Among them, miR-1184 can target LRRK2; miR-221, miR-760 and miR-1207-5p can target PARK2; and miR-1207-5p and miR-221 can target SNCA. Note that neither LRRK2, PARK2, nor SNCA mRNA levels were significantly different between PD and control for all the sample data, though all these genes are clearly associated with PD. It is possible that these miRNAs, whose targets are enriched in the differentially expressed gene lists and also include PD-associated genes, may mark the PD condition, while LRRK2 and SNCA mRNAs are not sensitive enough. Since these miRNA predictions are from both brain and blood samples, the miRNAs can be used as blood biomarkers with therapeutic potential if the miRNA level changes are found to be critical to PD pathology.

Recently, miRNA expression profiling of peripheral blood mononuclear cells of the Parkinson's disease patients and controls have been publicly available. We analyzed the data and found that miR-221 was significantly downregulated (p=0.0018, two-tail student t-test), confirming that miR-221 and its targets can serve as a PD blood biomarker (miR-1184 and miR-1207-5p were not available). Note that the research group (Sofia Oliveira group at Instituto de Medicina Molecular in Lisboa, Portugal) that generated the PD miRNA did not find miR-221 as important because it was under their criteria for follow up study. On the other hand, they identified SNCA pathway as important from the miRNA profiling, while we predicted miR-221 targeting SNCA.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method comprising:
a) identifying candidate miRNA-mRNA complexes in which an mRNA sequence from a set of mRNA sequences stably hybridizes to a miRNA from a set of miRNA sequences, each mRNA sequence having an upstream region that is upstream of a translation start site and a downstream region that is downstream of a translation stop site, each miRNA having a 5′ miRNA section and a 3′ miRNA section, the candidate miRNA-mRNA complexes having stably hybridizing sub-regions of the downstream region to portions of the 5′ miRNA section and stably hybridizing sub-regions of the upstream region to portions of the 3′ miRNA section;
b) identifying candidate mRNA sequences as mRNA sequences that form candidate microRNA-mRNA complexes and/or candidate miRNA sequences as miRNA sequences that form candidate microRNA-mRNA complexes;
c) optionally determining differences between expression levels between candidate mRNA sequences in subjects having a disease and subjects not having the disease for each candidate mRNA sequence and/or expression levels between candidate miRNA sequences in subjects having a disease and subjects not having the disease for each candidate miRNA sequence;
d) identifying candidate RNA disease markers as candidate mRNA sequences and/or miRNA sequences in which the expression levels in subjects having a disease are different from subjects not having the disease;
e) obtaining a biological sample from a test subject;
f) determining the expression levels of each candidate RNA disease markers from the biological sample; and
g) comparing the expression level of each candidate RNA disease marker to a control such that deviation of expression levels of at least one candidate RNA disease marker from the control indicates presence of the disease.

2. The method of claim 1 further comprising repeating steps e), f), and g) to monitor progression of the diseased state in the subject such that continued deviation from the control indicates progression of the diseased state.

3. The method of claim 1 wherein the candidate disease markers are selected such that candidate miRNA sequences predominately up regulates or predominately down regulates a group of mRNA sequences.

4. The method of claim 1 wherein deviation of expression levels of at least six candidate mRNA sequences indicates progression of diseased state in the subject.

5. The method of claim 1 wherein the biological sample is blood, plasma, serum, CSF, urine, feces, saliva, cell free plasma, tissue, skin, hair, or tumor.

6. The method of claim 1 wherein candidate RNA disease markers are candidate mRNA sequences in which the difference between the expression level between subjects having a disease and subjects not having the disease is greater than 20 percent or candidate miRNA sequences in which the difference between the expression level between subjects having a disease and subjects not having the disease is greater than 20 percent.

7. The method of claim 1 further comprising increasing an amount of a therapeutic agent administered to the subject if progression of the diseased state is determined.

8. The method of claim 7 further comprising the disease is neuroblastoma.

9. The method of claim 8 wherein the RNA biomarkers comprise mRNA sequences selected from the group consisting of AGPAT4 having NCBI number NM_{—}020133 (SEQ ID No: 1), BTBD9 having NCBI number NM_{—}001172418 (SEQ ID NO: 2), BTBD9 having NCBI number NM_{—}152733 (SEQ ID NO: 3), BTBD9 having NCBI number NM_{—}001099272 (SEQ ID NO: 4), BTBD9 having NCBI number NM_{—}052893 (SEQ ID NO: 5), GABBR1 having NCBI number NM_{—}001470 (SEQ ID NO: 6), GABBR1 having NCBI number NM_{—}021904 (SEQ ID NO: 7), GABBR1 having NCBI number NM_{—}021903 (SEQ ID NO: 8), KCNK10 having NCBI number NM_{—}021161 (SEQ ID NO: 9), KCNK10 having NCBI number NM_{—}138318 (SEQ ID NO: 10), KCNK10 having NCBI number NM_{—}138317 (SEQ ID NO: 11), LRRTM4 having NCBI number NM_{—}024993 (SEQ ID NO: 12), LRRTM4 having NCBI number NM_{—}001134745 (SEQ ID NO: 13), S100PBP having NCBI number NM_{—}022753 (SEQ ID NO: 14), S100PBP having NCBI number NM_{—}001256121 (SEQ ID NO: 15), and combinations thereof.

10. The method of claim 8 wherein the RNA biomarkers comprise miRNA sequences selected from the group consisting of hsa-miR-9 (SEQ ID NO: 16), hsa-miR-18a* (SEQ ID NO: 17), hsa-miR-136 (SEQ ID NO: 18), hsa-miR-152 (SEQ ID NO: 19), hsa-miR-185 (SEQ ID NO: 20), hsa-miR-205 (SEQ ID NO: 21), hsa-miR-214 (SEQ ID NO: 22), hsa-miR-221 (SEQ ID NO: 23), hsa-miR-324-3p (SEQ ID NO: 24), hsa-miR-326 (SEQ ID NO: 25), hsa-miR-328 (SEQ ID NO: 26), hsa-miR-346 (SEQ ID NO: 27), hsa-miR-489 (SEQ ID NO: 28), hsa-miR-500a (SEQ ID NO: 29), hsa-miR-610 (SEQ ID NO: 30), hsa-miR-650 (SEQ ID NO: 31), and combinations thereof.

11. The method of claim 8 wherein the therapeutic agent is an anti-cancer drug.

12. The method of claim 7 wherein the disease state is Parkinson's disease.

13. The method of claim 12 wherein the RNA biomarkers comprise mRNA sequences selected from the group consisting of AMMECR1 having NCBI number NM_{—}001025580 (SEQ ID NO: 32), AMMECR1 having NCBI number NM_{—}001171689 (SEQ ID NO: 33), AMMECR1 having NCBI number NM_{—}015365 (SEQ ID NO: 34), CASZ1 having NCBI number NM_{—}001079843 (SEQ ID NO: 35), CASZ1 having NCBI number NM_{—}017766 (SEQ ID NO: 36), CCNG2 having NCBI number NM_{—}004354 (SEQ ID NO: 37), FBXO41 having NCBI number NM_{—}001080410 (SEQ ID NO: 38), FOXP1 having NCBI number NM_{—}001244813 (SEQ ID NO: 39), FOXP1 having NCBI number NM_{—}001244814 (SEQ ID NO: 40), FOXP1 having NCBI number NM_{—}001244815 (SEQ ID NO: 41), FOXP1 having NCBI number NM_{—}001012505 (SEQ ID NO: 42), FOXP1 having NCBI number NM_{—}001244808 (SEQ ID NO: 43), FOXP1 having NCBI number NM_{—}001244810 (SEQ ID NO: 44), FOXP1 having NCBI number NM_{—}001244812 (SEQ ID NO: 45), FOXP1 having NCBI number NM_{—}001244816 (SEQ ID NO: 46), FOXP1 having NCBI number NM_{—}032682 (SEQ ID NO: 47), JRK having NCBI number NM_{—}001077527 (SEQ ID NO: 48), JRK having NCBI number NM_{—}003724 (SEQ ID NO: 49), MAPT having NCBI number NM_{—}001123066 (SEQ ID NO: 50), MAPT having NCBI number NM_{—}001123067 (SEQ ID NO: 51), MAPT having NCBI number NM_{—}001203251 (SEQ ID NO: 52), MAPT having NCBI number NM_{—}001203252 (SEQ ID NO: 53), MAPT having NCBI number NM_{—}005910 (SEQ ID NO: 54), MAPT having NCBI number NM_{—}016834 (SEQ ID NO: 55), MAPT having NCBI number NM_{—}016835 (SEQ ID NO: 56), MAPT having NCBI number NM_{—}016841 (SEQ ID NO: 57), NFYC having NCBI number NM_{—}001142587 (SEQ ID NO: 58), NFYC having NCBI number NM_{—}001142588 (SEQ ID NO: 59), NFYC having NCBI number NM_{—}001142589 (SEQ ID NO: 60), NFYC having NCBI number NM_{—}001142590 (SEQ ID NO: 61), NFYC having NCBI number NM_{—}014223 (SEQ ID NO: 62), QKI having NCBI number NM_{—}006775 (SEQ ID NO: 63), QKI having NCBI number NM_{—}206853 (SEQ ID NO: 64), QKI having NCBI number NM_{—}206854 (SEQ ID NO: 65), QKI having NCBI number NM_{—}206855 (SEQ ID NO: 66), RAB1A having NCBI number NM_{—}004161 (SEQ ID NO: 67), RAB1A having NCBI number NM_{—}015543 (SEQ ID NO: 68), RAPGEF3 having NCBI number NM_{—}001098531 (SEQ ID NO: 69), RAPGEF3 having NCBI number NM_{—}001098532 (SEQ ID NO: 70), RAPGEF3 having NCBI number NM_{—}006105 (SEQ ID NO: 71), STAT2 having NCBI number NM_{—}005419 (SEQ ID NO: 72), STAT2 having NCBI number NM_{—}198332 (SEQ ID NO: 73), VASH1 having NCBI number NM_{—}014909 (SEQ ID NO: 74), and combinations thereof.

14. The method of claim 12 wherein the RNA biomarkers comprise miRNA sequences selected from the group consisting hsa-miR-1184 (SEQ ID NO: 75), hsa-miR-221 (SEQ ID NO: 23), hsa-miR-1207-5p (SEQ ID NO: 76), hsa-miR-760 (SEQ ID NO: 77), and combinations thereof.

15. A method comprising:
a) obtaining a biological sample from a subject; and
b) determining expression levels of candidate RNA disease markers from the biological sample, the candidate RNA disease markers selected from the group consisting of AGPAT4 having NCBI number NM_{—}020133 (SEQ ID No: 1), BTBD9 having NCBI number NM_{—}001172418 (SEQ ID NO: 2), BTBD9 having NCBI number NM_{—}152733 (SEQ ID NO: 3), BTBD9 having NCBI number NM_{—}001099272 (SEQ ID NO: 4), BTBD9 having NCBI number NM_{—}052893 (SEQ ID NO: 5), GABBR1 having NCBI number NM_{—}001470 (SEQ ID NO: 6), GABBR1 having NCBI number NM_{—}021904 (SEQ ID NO: 7), GABBR1 having NCBI number NM_{—}021903 (SEQ ID NO: 8), KCNK10 having NCBI number NM_{—}021161 (SEQ ID NO: 9), KCNK10 having NCBI number NM_{—}138318 (SEQ ID NO: 10), KCNK10 having NCBI number NM_{—}138317 (SEQ ID NO: 11), LRRTM4 having NCBI number NM_{—}024993 (SEQ ID NO: 12), LRRTM4 having NCBI number NM_{—}001134745 (SEQ ID NO: 13), S100PBP having NCBI number NM_{—}022753 (SEQ ID NO: 14), S100PBP having NCBI number NM_{—}001256121 (SEQ ID NO: 15), hsa-miR-9 (SEQ ID NO: 16), hsa-miR-18a* (SEQ ID NO: 17), hsa-miR-136 (SEQ ID NO: 18), hsa-miR-152(SEQ ID NO: 19), hsa-miR-185(SEQ ID NO: 20), hsa-miR-205(SEQ ID NO: 21), hsa-miR-214(SEQ ID NO: 22), hsa-miR-221(SEQ ID NO: 23), hsa-miR-324-3p (SEQ ID NO: 24), hsa-miR-326 (SEQ ID NO: 25), hsa-miR-328(SEQ ID NO: 26), hsa-miR-346 (SEQ ID NO: 27), hsa-miR-489(SEQ ID NO: 28), hsa-miR-500a (SEQ ID NO: 29), hsa-miR-610 (SEQ ID NO: 30), hsa-miR-650 (SEQ ID NO: 31), and combinations thereof; and
c) comparing the expression level of each candidate RNA disease marker to a control such that deviation of expression levels of at least one candidate RNA disease marker from the control indicates presence of neuroblastoma.

16. The method of claim 15 further comprising repeating steps a), b), and c) to monitor progression of neuroblastoma in the subject such that continued deviation from the control indicates progression of neuroblastoma.

17. The method of claim 15 wherein deviation of expression levels of at least two candidate mRNA sequences and/or one miRNA sequences indicates progression of neuroblastoma.

18. A method comprising:
a) obtaining a biological sample from a subject; and
b) determining expression levels of candidate RNA disease markers from the biological sample, the candidate RNA disease markers selected from the group consisting AMMECR1 having NCBI number NM_{—}001025580 (SEQ ID NO: 32), AMMECR1 having NCBI number NM_{—}001171689 (SEQ ID NO: 33), AMMECR1 having NCBI number NM_{—}015365 (SEQ ID NO: 34), CASZ1 having NCBI number NM_{—}001079843 (SEQ ID NO: 35), CASZ1 having NCBI number NM_{—}017766 (SEQ ID NO: 36), CCNG2 having NCBI number NM_{—}004354 (SEQ ID NO: 37), FBXO41 having NCBI number NM_{—}001080410 (SEQ ID NO: 38), FOXP1 having NCBI number NM_{—}001244813 (SEQ ID NO: 39), FOXP1 having NCBI number NM_{—}001244814 (SEQ ID NO: 40), FOXP1 having NCBI number NM_{—}001244815 (SEQ ID NO: 41), FOXP1 having NCBI number NM_{—}001012505 (SEQ ID NO: 42), FOXP1 having NCBI number NM_{—}001244808 (SEQ ID NO: 43), FOXP1 having NCBI number NM_{—}001244810 (SEQ ID NO: 44), FOXP1 having NCBI number NM_{—}001244812 (SEQ ID NO: 45), FOXP1 having NCBI number NM_{—}001244816 (SEQ ID NO: 46), FOXP1 having NCBI number NM_{—}032682 (SEQ ID NO: 47), JRK having NCBI number NM_{—}001077527 (SEQ ID NO: 48), JRK having NCBI number NM_{—}003724 (SEQ ID NO: 49), MAPT having NCBI number NM_{—}001123066 (SEQ ID NO: 50), MAPT having NCBI number NM_{—}001123067 (SEQ ID NO: 51), MAPT having NCBI number NM_{—}001203251 (SEQ ID NO: 52), MAPT having NCBI number NM_{—}001203252 (SEQ ID NO: 53), MAPT having NCBI number NM_{—}005910 (SEQ ID NO: 54), MAPT having NCBI number NM_{—}016834 (SEQ ID NO: 55), MAPT having NCBI number NM_{—}016835 (SEQ ID NO: 56), MAPT having NCBI number NM_{—}016841 (SEQ ID NO: 57), NFYC having NCBI number NM_{—}001142587 (SEQ ID NO: 58), NFYC having NCBI number NM_{—}001142588 (SEQ ID NO: 59), NFYC having NCBI number NM_{—}001142589 (SEQ ID NO: 60), NFYC having NCBI number NM_{—}001142590 (SEQ ID NO: 61), NFYC having NCBI number NM_{—}014223 (SEQ ID NO: 62), QKI having NCBI number NM_{—}006775 (SEQ ID NO: 63), QKI having NCBI number NM_{—}206853 (SEQ ID NO: 64), QKI having NCBI number NM_{—}206854 (SEQ ID NO: 65), QKI having NCBI number NM_{—}206855 (SEQ ID NO: 66), RAB1A having NCBI number NM_{—}004161 (SEQ ID NO: 67), RAB1A having NCBI number NM_{—}015543 (SEQ ID NO: 68), RAPGEF3 having NCBI number NM_{—}001098531 (SEQ ID NO: 69), RAPGEF3 having NCBI number NM_{—}001098532 (SEQ ID NO: 70), RAPGEF3 having NCBI number NM_{—}006105 (SEQ ID NO: 71), STAT2 having NCBI number NM_{—}005419 (SEQ ID NO: 72), STAT2 having NCBI number NM_{—}198332 (SEQ ID NO: 73), VASH1 having NCBI number NM_{—}014909 (SEQ ID NO: 74), hsa-miR-1184 (SEQ ID NO: 75), hsa-miR-221 (SEQ ID NO: 23), hsa-miR-1207-5p (SEQ ID NO: 76), hsa-miR-760 (SEQ ID NO: 77), and combinations thereof; and
c) comparing the expression level of each candidate RNA disease marker to a control such that deviation of expression levels of at least one candidate RNA disease marker from the control indicates presence of Parkinson's disease.

19. The method of claim 18 further comprising repeating steps a), b), and c) to monitor progression of Parkinson's disease in the subject such that continued deviation from the control indicates progression of Parkinson's disease.

20. The method of claim 18 wherein deviation of expression levels of at least two candidate mRNA sequences and/or one miRNA sequences indicates progression of Parkinson's disease.


SEQUENCE LISTING (77 sequences)

SEQ ID NO: 1 - RNA, 7889 nt, Homo sapiens
auggguggagucucgcuuuuucuuuccaguguuggcugacuuacagcucuuauaaacuaguggcaauuucugaacccagccggcuccaucucagcuucugguuucuaaguccaugugccaaaggcugccaggaaggagacgccuuccugaguccuggaucuuucuuccuucuggaaaucuuugacuguggguaguuauuuauuucugaauaagagcguccacgcaucauggaccucgcgggacugcugaagucucaguuccugugccaccuggucuucugcuacgucuuuauugccucagggcuaaucaucaacaccauucagcucuucacucuccuccucuggcccauuaacaagcagcucuuccggaagaucaacugcagacuguccuauugcaucucaagccagcuggugaugcugcuggagugguggucgggcacggaaugcaccaucuucacggacccgcgcgccuaccucaaguaugggaaggaaaaugccaucgugguucucaaccacaaguuugaaauugacuuucuguguggcuggagccuguccgaacgcuuugggcuguuagggggcuccaagguccuggccaagaaagagcuggccuaugucccaauuaucggcuggaugugguacuucaccgagauggucuucuguucgcgcaagugggagcaggaucgcaagacgguugccaccaguuugcagcaccuccgggacuaccccgagaaguauuuuuuccugauucacugugagggcacacgguucacggagaagaagcaugagaucagcaugcagguggcccgggccaaggggcugccucgccucaagcaucaccuguugccacgaaccaagggcuucgccaucaccgugaggagcuugagaaauguaguuucagcuguauaugacuguacacucaauuucagaaauaaugaaaauccaacacugcugggaguccuaaacggaaagaaauaccaugcagauuuguauguuaggaggaucccacuggaagacaucccugaagacgaugacgagugcucggccuggcugcacaagcucuaccaggagaaggaugccuuucaggaggaguacuacaggacgggcaccuucccagagacgcccauggugcccccccggcggcccuggacccucgugaacuggcuguuuugggccucgcuggugcucuacccuuucuuccaguuccuggucagcaugaucaggagcgggucuucccugacgcuggccagcuucauccucgucuucuuuguggccuccgugggaguucgauggaugauuggugugacggaaauugacaagggcucugccuacggcaacucugacagcaagcagaaacugaaugacugacucagggaggugucaccauccgaagggaaccuuggggaacugguggccucugcauauccuccuuagugggacacggugacaaaggcugggugagccccugcugggcacggcggaagucacgaccucuccagccagggagucuggucucaaggccggauggggaggaagauguuuuguaaucuuuuuuuccccaugugcuuuagugggcuuugguuuucuuuuugugcgagugugugugagaauggcuguguggugagugugaacuuuguucugugaucauagaaaggguauuuuaggcugcaggggagggcagggcuggggaccgaaggggacaaguuccccuuucauccuuuggugcugaguuuucuguaacccuugguugccagagauaaagugaaaagugcuuuaggugagaugacuaaauuaugccuccaagaaaaaaaaauuaaagugcuuuucugggucaucugugguguauguugacauggaccgcugccccuccauccugcccuugcccguggcuuugguguuucagauccuacucacgggaggcagcugccgcggaggugugggcagcugagaggggugggcaggccagcacagcuccuggcaggagacacagguccaggagcccgugagcauuugugagagcagagauggcaagcacgugcguggagaccaacgaagcgugucccuggcacacagcaagggagaguuucgcucggucucugcagugcagucuccuggcguaagucuugaagauggacccaacucccugaagaaauggaucuugaaauugaacacaaacaucaugaacguacagccugggcauucugcagugauuugugagaugagcuugcaucgacuguuccucucagcagugacagccaaagucaccccugauaaaaugcaguuucacuuuauaaaaugccucgccccuucccagcauguagagcuugucugcagcccuaaaggagccagguccugcucugaugagggucaaaucagagccgacaucccauguacaaaaaacccccaggcuucugccuuccuuccgggcuccugucugcucccauuuuacuccacggaauaguucugcccuguagcaucuccagcggccuuugcagacugcugucagccaacaggugccaaaaaugcaaaagaagaagaaacuaaaagcagugcuauuuuugcagaacaaaugcuagcuauuaaaucaucuuaggccaagacaccaauggcacuuggaccgauagguauuuggagcaugaguuaggaaggauaugucuuucuccaggugcgcagcugaaaguugcugagaaaagcugccuguguugcugugguaacaugagaaggaaugaacagcucuauacaaaaacuggccacacauuauaaggccuauucuaugcaguuauguuuuuggcuauuugcucauuuagucauuguuuaucaaccaaacuagguaagccaaggaguuacaagacagcucuagagaagcucccucugcuuacaggacaauaaugcauuguuuguuuguugauacaaaugaauuauuugaauccacagaagugaaaaagggaaaaaauccuggcugacuugugggaagagcguacugcagagccuagggccugaguuaacucgcuggcggcccugcaggcaggcuugaugggagcgcccagcacaucugcccacccugcacagggcaugaagcaccgugugcagagaucugugggcaaaagcccaggacgugaccuuaggcaagagggguccuuggagaaguggaggugugcaugguguguagccuugcgugucugugagcacgcccuaguggaccugcgccuggauuaucgggccagucucucggagggguccgugaugcugaguuacgccauucaccugaacucugcagcucaucggcuguaggauuugggugagacacuuuuccuugcugacucauuuccccaucugcaaaaugggggugacgauaucuaccucuuggggugugucaaggguaaaagagcacacuuuaagaauccuaaagcacuaagccaaggaugaaagacuagaaagcugccucuauguugcuacucucaaaaaugcucagaaauuuucuugucguacuaaaaauguguuauuuuucucauuuucauucuuccauucaacacagaaguaaucuaaguaaguucuaaaaacauagauccgggggcccacagacauucaauuuucuucugaauuggcucugguaucacacaacacacaagcaagcaaacagccugccugcuagggccuuggguuccaggcuggaggaaugcaggcagaaaucucuuaggaaauaacuaagugagcgccaaguuuugaauugccuuagagccgaaagagaauguggcgugcuggggagagaucuggaccucuuucucaucagucauuugaccuguccacgaagaacaauuuuguuaugcagccugggaaccugguuuccuuacacccugcgcccuuaaugacacagacaaccacggguacuccuggacggaagcucaucacgccuccucaaauaccuucacccauguucaggucacacggaggucaccaucuccucagcacacaacaacugcuuugguucccaggguuauuuuuucuacgaguugaaaacccacggagccuaagccaucuuuuuguccccuaaggagagccccauagacuaagagcuacacuuccugucugucuuucccaccuuuccaucucauucuagcuuaaagcuuaucacgcuacugagagccuguucuguaguucaagguggcagagaaaaauacgaaccuugagugaaaucuggagagcagugcagggucauccccagacgugacgggccauauccuaggcgacuccugcagcuuauagaagcagggaauuuaauggacuucuuccaggaaagccagcccacggggacucuuaaaaagacaccaaccauuuggaagaugcagcuugaggucugggggacuggacugauucugccaccauauagugaacacgauuaaucgggaggaagaaguaaggcacucugugaguuagucuaccacccuauugacuuugacagagagaaaaccaacuuuuuucauaugaucacacaguaguacuggaauaucucgacugcaauauccucuaaacaaaugcaugcaaagcuuuguaaaagcagcugaugauuaaaaaaauuuuaaaacauuaggguuuuucuaauauauccauaugugggacagugauuuaaaugucauuaauuguccagccacaacuuguaacagugaugugaaagacacaaaaaaggccccacuggaguggaggaguggucuggcugguccccaagcaaaacagaaugggcuucgcauaggcuuuagaacuuauucauaaauauugaccaaagugugaagcaagguauccagugaagauucucuagggucucuacuucugcucaagagauuuaauccagcuuucucuaaagcagugguccagacuugccacauacuagguuaaagauaaagugagaaaaacaaauacuuugguuugcacgucucuugaggggcccuaggcuguauggaggaauauuuagaggaauauuugugagcugccauuuguguuuaaaagaacaugaaagcaagacgggcccuaacccgccugugucuguagcugaccaggaggccacugcuuccuucccccagagcuucuaggagagccccccaaaaugcaacucuccaagguccuguggaguaguccccucauaaugccuccaccacaccugugucucacgaugccuuaacugacguagcuccuaagugauguucacagagaaggaaauacuuaggcaagcccauguguaacaugcucacagaugugcuggcuuugccaaugcuuucggacuccaccaugcuuucugauucuucugguccuagucacgccuuggcacggggcagaagcggccgagcuaugcugacaggugcugcugagagccaacagccauucagccgagaaggcguuucuaggugagggcaugagagcuggcauuccaggcugcugagugcggcaucugcaggugcaccacauugcuccacgguguguucagaagagaccagcaucuugaaucucaggcucugggcucauucuuugccucugccaagggaccauucaggggcuucaagccagcugcugaaaaucccccuuuucuggauuuuuccccaucuauauaaggauaugguggcuguguggacaagaaauugguuucaucucagaagggugcuucccuacaaagaucuucagaaauuuaauugaaaacaauuuuuugaacuuuuuuuguuuggccaggaugaaugaagcugaagucacauccugaaugugcaugccagacaaaggguaagcaagcagucuuuacaaaugccaaucccucugcacuccaaaaccggagcccguaaugaaggcccagguuccagucaucacaggaucccccaagccagcacagugccugaugccucccuaggcauuagcacaucccagcacucuaauucacauccucagcaucccugccuccugcauccagggcuuagguugaguguuuccagagugucaaguacauuuaaacacaaaccaaauacuccaggguuuugaccugucacaauggcuuuugaaaacaacagacguacaauuugagagaaggacucgauggacuucuuaggcugaggacuaaggacauguugcuguuccucuugguccaggacauucaauaccauucacagcauucaucaccaacugacauaguaucgaucacgacauucaacuguuugaaauuacugcuauuuuggucaaaaacuaagaaaucagaaauugcaccuacauagugugucuccucauuggagucccaucucucugagcacagggaugggcuccugagcucacagcccugcccugcccccccagcagggccucacccucauggcccucagugaauagugacuacaugaagggggcacuuacuagaggccugaguaauggaauuucaauuagcuucugcuucccuuuucuaaaaaagaaccaccuuucuuaagaauaauuaggccaaaauaaacuugaguaauuuguacagucaauauuuugaaguuucggaguuaccagugaaauagcaaagcuguucuccaggaaaggacauccagguucccacccaggccuuccuuccucagcuccugcagccucauccuaguuuaauaugaagguugugaauggguuaaaaagcaguucuuaagcaccuacuuagaucuuuucaguaucuggaaauagcacccagggcuguguuaguaaagcuuuucacuggguaagagucauacaauuuuuacauuuaauuuguuuuuuuaaaacaaggcagucuuguuuuacauauuggaaugacuagauauuuuuugacauuucuggcaccucugguucugcugaauuuuaagggaggugagugguauguacacaaaguuaagauugcugcauaauaauuccuuugcauuuccaaaauaaauuacacaaaugucacuuuuccuuuucuauuccauaguacuaguuuuagccagaucuuugccaaacauucuaggacauucagaaaagucauccauggaauuuuggaaugccauucucucuguaucuuugcagaucaugaggaaggaaaguugaugaacuaguacgggauggcucagacuuguaaagugucuaagucacaacgcugcagagccagcuuccggguagcaauggcauggccucagcugugccaaggcuacaacuggagcuuccuuacucgcaucugaggcaaacacagcaaggcuggcccaggccgcccacagucaacagauguucacgggagauggaucagccccucuuuugcuuguucuaucaaggaaagagaucuaaccaugggcaaggguggaacacacccgguucaggucagggaaugaguugcucuaagugaacagugaauucuuacuucucagccuguaucaauuguacccaggguucaugauuacugugacccacuaagggaugaucuuugaggaaacagaccuggagggagguaauuuaaggagggaaagcgaguggguuuuacagacugcuuuguggcgacauucaggaacagaccauucaauggaugacuuuucaguacauaaaagcaagcaguaauggccaggagagagcaaagaugcccuaagaaugacaucagacugaccaaaugugccuuuucaaauaaguuaugagaacgaaugaauuggaauuagagcaagguauuggacagaaccaugcuagucuuugagagaacguguggauuggguggauguuuggaccgaaugaaugguaacuuuuuaaugugaaacagagaacugauauaugggcugucaugucacccuaggguacugucgcucugucauguguagcaaucuuuccaaauuuuuguuggugggcugaacaaagauaaaacuaauaaaguauguguggauaccacuagaaaaaaaaaaa

SEQ ID NO: 2 - RNA, 8639 nt, Homo sapiens
agauaaggaauuguauaggaggaaacuucauaguaaacauugaaccucugugcuugucuccuuguugaaguuggggguucauguuuauuguuccaagggagccucaucaaaaccacuuuuaaagcauugagaauccaaauaaauaccauggaucauacaggauuggggauaagucuugaggucuccccgagaaggagaaguucuuuaauuugguaauuuuaggguaaugguuggaugcucucuggauuaauggaaaccuucagggguacuuuuaggcagaguauaacccagugaagagcaaauccuauuugaggguuucuucauguguuucuucccugaugugcagagcauuauuauaugguggaaugcgagagucucagccugaagcagaaauuccucuccaagacaccacugcagaagcauucacaaugcuacucaaauauaucuacacugggcgggcaacgcugacagaugagaaggaggaggugcugcuggacuuuuugagccuggcucauaaauauggauuuccagagcuagaggauucuaccucugaguaucucugcaccauacuuaacauucagaaugucugcaugacuuuugauguugccagucucuacucacuucccaaguuaacuuguaugugcugcauguuuauggauaggaaugcucaggaaguccucucaagugaagguuuccucucccuuucuaagacagcacuuuuaaacaucguguuaagagacucauuugcagcucccgaaaaagauauuuuccuagccuuauuaaacugguguaagcacaauucaaaggagaaucaugcugaaaucaugcaggcugugcguuuaccucucaugagccucacagagcuucugaauguugugaggccuucaggacugcugucuccugaugccauccuggaugccauuaaagugcgaucugagagccgggauauggaccucaauuauagaggcaugcucauaccagaagaaaacauugcaacuaugaaguauggagcccaaguuguaaagggggagcugaaaucagccuuauuagauggugauacucaaaauuaugauuuggaucauggauuuucaaggcacccaauugaugaugacugccguuccggcaucgagauuaagcuaggucagccauccauuaucaaucacauacggauacucuugugggaccgagauagccggucuuacucauacuucauugaagugucaauggaugaacuugauugggucagagugauagaucauucacaauaucugugucguucuuggcagaaauuauauuuuccagcccgugucugcaguggagauggagucucacuauggugcccacucuggucucgaacuccugagcucaagcaauccucccuccuuggccuuccaaagugcagguauauucgaauuguugggacucacaacacagugaacaagauuuuucacauuguggcuuuugaauguauguuuacaaacaaaaccuucacucuugagaaggggcugauaguucccauggagaauguugcaacaauugcugauugugccagugugauugaaggagucagucggagccgaaaugccuugcugaauggggacacuaagaauuaugacugggauucuggcuacacaugucaccagcuaggaaguggugcgauugugguucaguuggcacaaccguacaugauugggucaauacgguuacuacuuugggauugugaugaucgaagcuauagcuacuacguugagguuucuaccaaccagcaacaguggaccaugguugcugacagaacuaaagucuccugcaaguccuggcagucaguaacuuuugaaaggcagccugccuccuucauccguaucguugggacacacaacacagcaaaugagguguuccacuguguccacuuugaguguccagagcagcagagcagccagaaggaggaaaauagugaggaaucggggacaggggacaccagccuggccggucagcagcucgacucccaugcgcugcgggcgccuaguggcagcucacuacccuccagcccaggcuccaacucacgcucccccaaccggcagcaccaauaaaggaggcagcgggccuggugugacuuggugggcucgggcaacggcaggaaacggucuccucccugagcaggggucucuguugacugcccccaccucugccccuuuccagggaggagccgaccuagcugcaaaagcagacaccgaacagguuuucucccaaggacagaaaggggcugcuuuguucuucucaauuuauccaaaucaggcaggucucaagggggagaaaaugggucuucaaucuucaucaaguccaccauuaacacccuaccucucuaaucuagcccaggcgcagggaacaggaggaagucauagacaggccuguguuggaauacaaaaauggcacgcaaacacucaggcggaggagaaagccauugggccuuugcuaccuggaggcacccuuuaggaaugggccuccauagaguguucauucugcuggaauacauugcccuggcaacuaaaugauuuuuuauuuguuuuaguuauucuccacucagugaaucauagguguuccggaucuauuugaaaugcucaaacagcaaaucacuaguuuuaaucccuuuugaugcuauaauuuuccuuccuugguuuugaacgguucagcuugcggggacagagacccaucgugcaugucuccuaaaugagacacggaugcagcucauccuggcacuugggccacagaggaagugccacaaaugcacacaagcaaaucaaccuguggacacuggaguuaauagucacucguguggcugaguuacucaccugucauuccugacauggauccagccuuuaagcuugggaggcugacaaggaaauaguuacuaugcccgcugcuaauucacuguuccgaaguccauguagauaaacauauaccgaguucuggcguggccaaaccugccggggacugccguggcuuagcugccccuccaguuguguuuaucgaagcauuggagaggcagccaccugcccaucagcgucaggaacgcagaacugggacuccgugcuucccccacaccacaguacucaacuggggacugaaaaauagcaguuccaagaagcuuuucuccuaauccucaucaaaaggaccucauuugacgaugguggcuugauguggcuccgucuccuacuuagugauuuccauuccagugcugccugcccuggguaacugcgcacaacauugccugccccuccgcuguccccagagcuccggccucugucgccaucagcaucagccugggagucuccccaggacggcauggcacgugagcagcacugcaguccuugggucuucggcgccaaccugcaacccugaucaaaggagggugagggcgugggaguuuccuuucaggugugucaaagacauaucagcugcgcucuagaaagagccaacgggucagccgcuuccuccuaguuuaacuugauuuugagugauuuguccauauggaaauaguaggguagcaucaaacugcgugauuaaauuggaagacugucuuuccgugugaacacaggauggcagacaucagagggcuaccggggcaggguggacacguuccugaggagaagccagggucugcagcucuccauccuccccgguagcuccagcugcagggcugagcugcuuccuauccccucagcucugaucucugccacuucccuguugugugggagaguggaacacuccugggaugccaggcauuucauaaugggaggaggcuccuguaaggaacggugggcgcccgcucacaguggggggcccuccagcaugguugcugacucugucagcccccagccccggguugccaauuagccagagagcuccugcacacacagcacuuccuuccccaguguguuuucuacccagauggggacaaaggagccagacagucauaucaagggcagaguucaaacauuuugaaaugagccauucacaugcuucuuuuuaaacaugggcaucaccugcccagaggcccuuggccugaagcagcaugcugcugaccuugggaagagagggugggcuggugggggugcuggaggagcugcccggcccugcaucugcaacccuccaguccccugcccacagagcacgcagagcccuccugugcuccgggaacgagauagcagcuuggcagcucggccucgcaucugaaaaggaauacgcugcaaagccucggcccuagacccacaucugcagcucccuggccuguggcgguucuuuccuucggggcaugcccuuccagaucucugucccuccugucuucuagcacuuuaaagaugccccuuaucaucagauaaagagaaguauggggacgaugggguggcacuaacaguagaugucauuggugaggacugauggcaaagucucauucccucacagccgcgcugccagcucgccucccccucgcuuugcugggagccugcccuggacuccaaaaggacacggguucaccuccuuucccaggcuggagggguacugcccuucccugaggggccuucugacuggggcaggaacucuuuucguguguccagcugggccuggaggccaagucaaggugugucacaccacacaccagggcaaggaccucaucuccagggaacagggacaagugaguggcuaccagaaaguccccgggcagccuggaguuccccagagccuggcugguccuacagagcacuucuccccugggccaccaggaccaaagcgaccuaaacacuugaagcuaaaagcaagugcugaugaugggaugggcuggcacguggcugggagggcuccugggcaccacagagcccucagcccagcagagaguggaggcucaguccguggggagcggggguugugaaggagacauggccaagccccuggucggggagauaagcucccccaugcccaggccagccccaggggcaacaagccaacauggagagagguggcaggugaggcugacaggugggugccuguggccugggcccaugagggcaaugguccucaguaaccaauggaaaggacacaagggcgccauggucaccaccacccaggcaaagaaaugggagguggccagcacccccacccuucacuuuucauuauuauuauuauuauuauuauuauuauuuuuuuuuuuuuuuuuuuuuuuuuuuuuuuuuuuugagaguagaaaggcaacuucauuggagcuuuuggggaaaaaagccuucaaagucaccuucuggcagcucuggggagggucauuugggguggacugagaguguagggcagguagggugugugcugggcagcagcuguguccaugcagccagcacccagcagggcagucucacaggagccguugugagcacuuuggacuugaagcagcuaugaaccaccccccgcccccaccgucucucccucucacacacacacacacacacacacacacacacacacacacacacacacacccguuuuaccauuuucucagugcuuuuucugcuuucauuccuuugauuuuaguuagaagcacaagccaugauggaugugggacucuucucaauugcucuuuaaauugucacuuuuuaaauccuauuaaaugaagugucgauuccugacaaauacauuaaaaguguuuuauuccuagaagaguugggaaaagaaauuauuuucagcaagaguaggauguuuguuaaacugauuccuuggcaaaaaagagaaaaaaagcuuuugaauugugcaaaaaauugcccaguauuaugcugucgccccaguugucaaacugcugugcagauggcuccagcccagucaacuucaccuucuuuuuaauuuagaagauaacaaaauuguaaucacuuauccuuuccagagccaagggggaaaaggaagguauaaucuacaauaaaaagcgagcguucuguguacugaggccacuuggugauaaagagauggagcgcuccccucacagacuucaauuaagaaccucccuuuggacagggaagaaaggugucaaagaggaaaagaaaauuaaauuugcuuccuuccaggaguuuuuccucauuagugcuugcuugucgguguuauuauuuuaaucuuaccuucuauguggugaccagcuccuccgagcgaugcccaggucgggcacggcccgggcagggcaggucugcagcgaugccguggcagaggugggcauacuccauuuguuuuggcagcugcagccauugauucugcauauuuuuccugacaacagccccggcaggaguucaguuaggaauuuaaagugcaguucaugguucugugccaccguggcuuuuauuauuauaauauuaaauuagaaguuguccuagugccugguguuugcucagaguuuccagaagagagggaagggcaagguuuaaauggcaugcaggacaacuggaaugcccccaucucucucgcugacacggauccagucauaccuggggcuggacgggauuuggaggcccuggucauuugcagagugaauggugaagggcgcggaaaagguuugcuuggaggaaagaccgguugcagaggcgaggcggagggaggaggggcggaggcagcaggucuuuguuugugguaggucucuggcuuccaucagggaggaggaaagaggcugugcccuuccuggcucuuggcugcaccacugaggacgcuccgagggacagcgugcucacccauccuuugcacagugcuggccccaagccccacggccuuccagcuaggauuuucugcugggcucaugcagaggcaggggacaggugcauggaagagccgccccacccgacacaccauuguuugaaaaucacuguucucuuuacucacuuaaaaaaguguacagggaacaccuguuccuggcauaaugcuccaaccucgcggaaggggccaggugcccuucaucuggcucuggcugcuuccgaccugggcccacgucaucguucacguccucugugaccaccacuggucacggugcucccuggcccagccuccaacccacccagcacccuggcaucucccaggccagucugcugcacccagcgagcuuccagucagaagccaggcugaacggcccuccugccccaucagcuucgugucuucuuuuuuuuaaagaacugaaauaguccccaagaggccucauggccugaagacucacaaucauccaccuguaauuuaugauaaaugucugggagcauuuaccauuugcguccgugaguauuuauagcccugaaugggcgggggggaggggggguggaggaggcccugcagccaggagcuacacaccuguccccacuaguguccccugguugacagagcccccucagccuccccaaggcugucacugcggcugugacagcugaggagugccgccuuugaaagccaguggacagucgcuccacuagggggagaggcccuggcccuggcgcagaggaggcguugcgaggcgggacgggggcuggaggggcugagcagccuucagggcagggacugggcccugggucacuggagacguugauauuaguccgucugcugccaaauugcuccccaccacaugagccccagggguuuaugucccaggaaggcgagggugcccaucugagcggaauugggaggggacggcaccagcucaucucccucagggccuuugccuccuggugcugcccugguggcugcuccugcaccacagccccugauggcugcugcuaguccugaguugcuggguuuacccccagcccacacuucccaccugggccugagggugcggccagugcccuaguccuagccacuacaggagucauucugagaccugcuggaggccauggggucuucccaggccccucaaucagcugcuuccagggucagcagggcagggugcugccaguaagguccucagggagcacagcccggccgcccaggcugggggauacuggggcagagcuuccaggucuguggggccugaucucuccccaaggcucuccaggccuuggugcgcccuccacagugaccuucagagaggcugcacccccucagaagaacagugagaaaucucuccaucacacgcucccugguccuuaugucccugaggccacccuuccccaccccccagugccuggagaagcgugagacucuggaggggcgccaggaggccagggguccucagggcuaggccuggagcucggcccaagagcugcuuuugcgaagccugucuugaauccggauucaccagagaacaagagccucccagccuuuggcguuucugggccuguaaagauguguguaccucccaggccacucugaugcaagggcagggaccaugccaggccuggguugggaauggcucugugacuccagaagcuccgucuaaaacuccaaagaugcccaaaaggcugugcugcuauguggaauguguauuauuugugagcacgaugcggcuucuuccucauuuugcagagcaaccuaagcgggcagauguacaaaccguguguucgaaaccccugaguccaugugugugaaaaugcagguuuucucuuagaaauaaaguggugacuugugcuguaaaaaaaaaaaaaaaaaaa

SEQ ID NO: 3 - RNA, 8504 nt, Homo sapiens
agauaaggaauuguauaggaggaaacuucauaguaaacauugaaccucugugcuugucuccuuguugaaguuggggguucauguuuauuguuccaagggagccucaucaaaaccacuuuuaaagcauugagaauccaaauaaauaccauggaucauacaggauuggggauaagucuugaggucuccccgagaaggagaaguucuuuaauuugguaauuuuaggguaaugguuggaugcucucuggauuaauggaaaccuucagggguacuuuuaggcagaguauaacccagugaagagagcauuauuauaugguggaaugcgagagucucagccugaagcagaaauuccucuccaagacaccacugcagaagcauucacaaugcuacucaaauauaucuacacugggcgggcaacgcugacagaugagaaggaggaggugcugcuggacuuuuugagccuggcucauaaauauggauuuccagagcuagaggauucuaccucugaguaucucugcaccauacuuaacauucagaaugucugcaugacuuuugauguugccagucucuacucacuucccaaguuaacuuguaugugcugcauguuuauggauaggaaugcucaggaaguccucucaagugaagguuuccucucccuuucuaagacagcacuuuuaaacaucguguuaagagacucauuugcagcucccgaaaaagauauuuuccuagccuuauuaaacugguguaagcacaauucaaaggagaaucaugcugaaaucaugcaggcugugcguuuaccucucaugagccucacagagcuucugaauguugugaggccuucaggacugcugucuccugaugccauccuggaugccauuaaagugcgaucugagagccgggauauggaccucaauuauagaggcaugcucauaccagaagaaaacauugcaacuaugaaguauggagcccaaguuguaaagggggagcugaaaucagccuuauuagauggugauacucaaaauuaugauuuggaucauggauuuucaaggcacccaauugaugaugacugccguuccggcaucgagauuaagcuaggucagccauccauuaucaaucacauacggauacucuugugggaccgagauagccggucuuacucauacuucauugaagugucaauggaugaacuugauugggucagagugauagaucauucacaauaucugugucguucuuggcagaaauuauauuuuccagcccgugucugcagguauauucgaauuguugggacucacaacacagugaacaagauuuuucacauuguggcuuuugaauguauguuuacaaacaaaaccuucacucuugagaaggggcugauaguucccauggagaauguugcaacaauugcugauugugccagugugauugaaggagucagucggagccgaaaugccuugcugaauggggacacuaagaauuaugacugggauucuggcuacacaugucaccagcuaggaaguggugcgauugugguucaguuggcacaaccguacaugauugggucaauacgguuacuacuuugggauugugaugaucgaagcuauagcuacuacguugagguuucuaccaaccagcaacaguggaccaugguugcugacagaacuaaagucuccugcaaguccuggcagucaguaacuuuugaaaggcagccugccuccuucauccguaucguugggacacacaacacagcaaaugagguguuccacuguguccacuuugaguguccagagcagcagagcagccagaaggaggaaaauagugaggaaucggggacaggggacaccagccuggccggucagcagcucgacucccaugcgcugcgggcgccuaguggcagcucacuacccuccagcccaggcuccaacucacgcucccccaaccggcagcaccaauaaaggaggcagcgggccuggugugacuuggugggcucgggcaacggcaggaaacggucuccucccugagcaggggucucuguugacugcccccaccucugccccuuuccagggaggagccgaccuagcugcaaaagcagacaccgaacagguuuucucccaaggacagaaaggggcugcuuuguucuucucaauuuauccaaaucaggcaggucucaagggggagaaaaugggucuucaaucuucaucaaguccaccauuaacacccuaccucucuaaucuagcccaggcgcagggaacaggaggaagucauagacaggccuguguuggaauacaaaaauggcacgcaaacacucaggcggaggagaaagccauugggccuuugcuaccuggaggcacccuuuaggaaugggccuccauagaguguucauucugcuggaauacauugcccuggcaacuaaaugauuuuuuauuuguuuuaguuauucuccacucagugaaucauagguguuccggaucuauuugaaaugcucaaacagcaaaucacuaguuuuaaucccuuuugaugcuauaauuuuccuuccuugguuuugaacgguucagcuugcggggacagagacccaucgugcaugucuccuaaaugagacacggaugcagcucauccuggcacuugggccacagaggaagugccacaaaugcacacaagcaaaucaaccuguggacacuggaguuaauagucacucguguggcugaguuacucaccugucauuccugacauggauccagccuuuaagcuugggaggcugacaaggaaauaguuacuaugcccgcugcuaauucacuguuccgaaguccauguagauaaacauauaccgaguucuggcguggccaaaccugccggggacugccguggcuuagcugccccuccaguuguguuuaucgaagcauuggagaggcagccaccugcccaucagcgucaggaacgcagaacugggacuccgugcuucccccacaccacaguacucaacuggggacugaaaaauagcaguuccaagaagcuuuucuccuaauccucaucaaaaggaccucauuugacgaugguggcuugauguggcuccgucuccuacuuagugauuuccauuccagugcugccugcccuggguaacugcgcacaacauugccugccccuccgcuguccccagagcuccggccucugucgccaucagcaucagccugggagucuccccaggacggcauggcacgugagcagcacugcaguccuugggucuucggcgccaaccugcaacccugaucaaaggagggugagggcgugggaguuuccuuucaggugugucaaagacauaucagcugcgcucuagaaagagccaacgggucagccgcuuccuccuaguuuaacuugauuuugagugauuuguccauauggaaauaguaggguagcaucaaacugcgugauuaaauuggaagacugucuuuccgugugaacacaggauggcagacaucagagggcuaccggggcaggguggacacguuccugaggagaagccagggucugcagcucuccauccuccccgguagcuccagcugcagggcugagcugcuuccuauccccucagcucugaucucugccacuucccuguugugugggagaguggaacacuccugggaugccaggcauuucauaaugggaggaggcuccuguaaggaacggugggcgcccgcucacaguggggggcccuccagcaugguugcugacucugucagcccccagccccggguugccaauuagccagagagcuccugcacacacagcacuuccuuccccaguguguuuucuacccagauggggacaaaggagccagacagucauaucaagggcagaguucaaacauuuugaaaugagccauucacaugcuucuuuuuaaacaugggcaucaccugcccagaggcccuuggccugaagcagcaugcugcugaccuugggaagagagggugggcuggugggggugcuggaggagcugcccggcccugcaucugcaacccuccaguccccugcccacagagcacgcagagcccuccugugcuccgggaacgagauagcagcuuggcagcucggccucgcaucugaaaaggaauacgcugcaaagccucggcccuagacccacaucugcagcucccuggccuguggcgguucuuuccuucggggcaugcccuuccagaucucugucccuccugucuucuagcacuuuaaagaugccccuuaucaucagauaaagagaaguauggggacgaugggguggcacuaacaguagaugucauuggugaggacugauggcaaagucucauucccucacagccgcgcugccagcucgccucccccucgcuuugcugggagccugcccuggacuccaaaaggacacggguucaccuccuuucccaggcuggagggguacugcccuucccugaggggccuucugacuggggcaggaacucuuuucguguguccagcugggccuggaggccaagucaaggugugucacaccacacaccagggcaaggaccucaucuccagggaacagggacaagugaguggcuaccagaaaguccccgggcagccuggaguuccccagagccuggcugguccuacagagcacuucuccccugggccaccaggaccaaagcgaccuaaacacuugaagcuaaaagcaagugcugaugaugggaugggcuggcacguggcugggagggcuccugggcaccacagagcccucagcccagcagagaguggaggcucaguccguggggagcggggguugugaaggagacauggccaagccccuggucggggagauaagcucccccaugcccaggccagccccaggggcaacaagccaacauggagagagguggcaggugaggcugacaggugggugccuguggccugggcccaugagggcaaugguccucaguaaccaauggaaaggacacaagggcgccauggucaccaccacccaggcaaagaaaugggagguggccagcacccccacccuucacuuuucauuauuauuauuauuauuauuauuauuauuuuuuuuuuuuuuuuuuuuuuuuuuuuuuuuuuuugagaguagaaaggcaacuucauuggagcuuuuggggaaaaaagccuucaaagucaccuucuggcagcucuggggagggucauuugggguggacugagaguguagggcagguagggugugugcugggcagcagcuguguccaugcagccagcacccagcagggcagucucacaggagccguugugagcacuuuggacuugaagcagcuaugaaccaccccccgcccccaccgucucucccucucacacacacacacacacacacacacacacacacacacacacacacacacccguuuuaccauuuucucagugcuuuuucugcuuucauuccuuugauuuuaguuagaagcacaagccaugauggaugugggacucuucucaauugcucuuuaaauugucacuuuuuaaauccuauuaaaugaagugucgauuccugacaaauacauuaaaaguguuuuauuccuagaagaguugggaaaagaaauuauuuucagcaagaguaggauguuuguuaaacugauuccuuggcaaaaaagagaaaaaaagcuuuugaauugugcaaaaaauugcccaguauuaugcugucgccccaguugucaaacugcugugcagauggcuccagcccagucaacuucaccuucuuuuuaauuuagaagauaacaaaauuguaaucacuuauccuuuccagagccaagggggaaaaggaagguauaaucuacaauaaaaagcgagcguucuguguacugaggccacuuggugauaaagagauggagcgcuccccucacagacuucaauuaagaaccucccuuuggacagggaagaaaggugucaaagaggaaaagaaaauuaaauuugcuuccuuccaggaguuuuuccucauuagugcuugcuugucgguguuauuauuuuaaucuuaccuucuauguggugaccagcuccuccgagcgaugcccaggucgggcacggcccgggcagggcaggucugcagcgaugccguggcagaggugggcauacuccauuuguuuuggcagcugcagccauugauucugcauauuuuuccugacaacagccccggcaggaguucaguuaggaauuuaaagugcaguucaugguucugugccaccguggcuuuuauuauuauaauauuaaauuagaaguuguccuagugccugguguuugcucagaguuuccagaagagagggaagggcaagguuuaaauggcaugcaggacaacuggaaugcccccaucucucucgcugacacggauccagucauaccuggggcuggacgggauuuggaggcccuggucauuugcagagugaauggugaagggcgcggaaaagguuugcuuggaggaaagaccgguugcagaggcgaggcggagggaggaggggcggaggcagcaggucuuuguuugugguaggucucuggcuuccaucagggaggaggaaagaggcugugcccuuccuggcucuuggcugcaccacugaggacgcuccgagggacagcgugcucacccauccuuugcacagugcuggccccaagccccacggccuuccagcuaggauuuucugcugggcucaugcagaggcaggggacaggugcauggaagagccgccccacccgacacaccauuguuugaaaaucacuguucucuuuacucacuuaaaaaaguguacagggaacaccuguuccuggcauaaugcuccaaccucgcggaaggggccaggugcccuucaucuggcucuggcugcuuccgaccugggcccacgucaucguucacguccucugugaccaccacuggucacggugcucccuggcccagccuccaacccacccagcacccuggcaucucccaggccagucugcugcacccagcgagcuuccagucagaagccaggcugaacggcccuccugccccaucagcuucgugucuucuuuuuuuuaaagaacugaaauaguccccaagaggccucauggccugaagacucacaaucauccaccuguaauuuaugauaaaugucugggagcauuuaccauuugcguccgugaguauuuauagcccugaaugggcgggggggaggggggguggaggaggcccugcagccaggagcuacacaccuguccccacuaguguccccugguugacagagcccccucagccuccccaaggcugucacugcggcugugacagcugaggagugccgccuuugaaagccaguggacagucgcuccacuagggggagaggcccuggcccuggcgcagaggaggcguugcgaggcgggacgggggcuggaggggcugagcagccuucagggcagggacugggcccugggucacuggagacguugauauuaguccgucugcugccaaauugcuccccaccacaugagccccagggguuuaugucccaggaaggcgagggugcccaucugagcggaauugggaggggacggcaccagcucaucucccucagggccuuugccuccuggugcugcccugguggcugcuccugcaccacagccccugauggcugcugcuaguccugaguugcuggguuuacccccagcccacacuucccaccugggccugagggugcggccagugcccuaguccuagccacuacaggagucauucugagaccugcuggaggccauggggucuucccaggccccucaaucagcugcuuccagggucagcagggcagggugcugccaguaagguccucagggagcacagcccggccgcccaggcugggggauacuggggcagagcuuccaggucuguggggccugaucucuccccaaggcucuccaggccuuggugcgcccuccacagugaccuucagagaggcugcacccccucagaagaacagugagaaaucucuccaucacacgcucccugguccuuaugucccugaggccacccuuccccaccccccagugccuggagaagcgugagacucuggaggggcgccaggaggccagggguccucagggcuaggccuggagcucggcccaagagcugcuuuugcgaagccugucuugaauccggauucaccagagaacaagagccucccagccuuuggcguuucugggccuguaaagauguguguaccucccaggccacucugaugcaagggcagggaccaugccaggccuggguugggaauggcucugugacuccagaagcuccgucuaaaacuccaaagaugcccaaaaggcugugcugcuauguggaauguguauuauuugugagcacgaugcggcuucuuccucauuuugcagagcaaccuaagcgggcagauguacaaaccguguguucgaaaccccugaguccaugugugugaaaaugcagguuuucucuuagaaauaaaguggugacuugugcuguaaaaaaaaaaaaaaaaaaa

SEQ ID NO: 4 - RNA, 8767 nt, Homo sapiens
ggagccaggugccggcagggcagccacacccuggggcccccuguucuggaggggcagagggucgggccgaugcgaaaaagccgcggaagcugccuuaggcgacuuucugggccugcaucgccggacccagcagcccgcugucgaucgguugcuaagugacgccagcgcgcaguggcgcgugcgcggccacggcggagugcgugaaagcggacgcgcgccgcagcagccggggcggcggcgaggacggcggccacggaagcggccaagggggcgguggcgcagagaggggugcggagcgggcggaagaggcgucugugucgcagcuuggucucagaggagcaucgcugggaacaacgacuaucgucuauuccacaagaugaguaacagccacccucuucgccccuuuacugcagugggggaaauugaucaugugcacauuuugucugaacauauuggugccuuguugauuggggaagaauauggcgacgucacauucgugguggaaaagaaacguuuuccugcccacaggguaauuuuagcagccaggugccaauauuuucgagcauuauuauaugguggaaugcgagagucucagccugaagcagaaauuccucuccaagacaccacugcagaagcauucacaaugcuacucaaauauaucuacacugggcgggcaacgcugacagaugagaaggaggaggugcugcuggacuuuuugagccuggcucauaaauauggauuuccagagcuagaggauucuaccucugaguaucucugcaccauacuuaacauucagaaugucugcaugacuuuugauguugccagucucuacucacuucccaaguuaacuuguaugugcugcauguuuauggauaggaaugcucaggaaguccucucaagugaagguuuccucucccuuucuaagacagcacuuuuaaacaucguguuaagagacucauuugcagcucccgaaaaagauauuuuccuagccuuauuaaacugguguaagcacaauucaaaggagaaucaugcugaaaucaugcaggcugugcguuuaccucucaugagccucacagagcuucugaauguugugaggccuucaggacugcugucuccugaugccauccuggaugccauuaaagugcgaucugagagccgggauauggaccucaauuauagaggcaugcucauaccagaagaaaacauugcaacuaugaaguauggagcccaaguuguaaagggggagcugaaaucagccuuauuagauggugauacucaaaauuaugauuuggaucauggauuuucaaggcacccaauugaugaugacugccguuccggcaucgagauuaagcuaggucagccauccauuaucaaucacauacggauacucuugugggaccgagauagccggucuuacucauacuucauugaagugucaauggaugaacuugauugggucagagugauagaucauucacaauaucugugucguucuuggcagaaauuauauuuuccagcccgugucugcagguauauucgaauuguugggacucacaacacagugaacaagauuuuucacauuguggcuuuugaauguauguuuacaaacaaaaccuucacucuugagaaggggcugauaguucccauggagaauguugcaacaauugcugauugugccagugugauugaaggagucagucggagccgaaaugccuugcugaauggggacacuaagaauuaugacugggauucuggcuacacaugucaccagcuaggaaguggugcgauugugguucaguuggcacaaccguacaugauugggucaauacgguuacuacuuugggauugugaugaucgaagcuauagcuacuacguugagguuucuaccaaccagcaacaguggaccaugguugcugacagaacuaaagucuccugcaaguccuggcagucaguaacuuuugaaaggcagccugccuccuucauccguaucguugggacacacaacacagcaaaugagguguuccacuguguccacuuugaguguccagagcagcagagcagccagaaggaggaaaauagugaggaaucggggacaggggacaccagccuggccggucagcagcucgacucccaugcgcugcgggcgccuaguggcagcucacuacccuccagcccaggcuccaacucacgcucccccaaccggcagcaccaauaaaggaggcagcgggccuggugugacuuggugggcucgggcaacggcaggaaacggucuccucccugagcaggggucucuguugacugcccccaccucugccccuuuccagggaggagccgaccuagcugcaaaagcagacaccgaacagguuuucucccaaggacagaaaggggcugcuuuguucuucucaauuuauccaaaucaggcaggucucaagggggagaaaaugggucuucaaucuucaucaaguccaccauuaacacccuaccucucuaaucuagcccaggcgcagggaacaggaggaagucauagacaggccuguguuggaauacaaaaauggcacgcaaacacucaggcggaggagaaagccauugggccuuugcuaccuggaggcacccuuuaggaaugggccuccauagaguguucauucugcuggaauacauugcccuggcaacuaaaugauuuuuuauuuguuuuaguuauucuccacucagugaaucauagguguuccggaucuauuugaaaugcucaaacagcaaaucacuaguuuuaaucccuuuugaugcuauaauuuuccuuccuugguuuugaacgguucagcuugcggggacagagacccaucgugcaugucuccuaaaugagacacggaugcagcucauccuggcacuugggccacagaggaagugccacaaaugcacacaagcaaaucaaccuguggacacuggaguuaauagucacucguguggcugaguuacucaccugucauuccugacauggauccagccuuuaagcuugggaggcugacaaggaaauaguuacuaugcccgcugcuaauucacuguuccgaaguccauguagauaaacauauaccgaguucuggcguggccaaaccugccggggacugccguggcuuagcugccccuccaguuguguuuaucgaagcauuggagaggcagccaccugcccaucagcgucaggaacgcagaacugggacuccgugcuucccccacaccacaguacucaacuggggacugaaaaauagcaguuccaagaagcuuuucuccuaauccucaucaaaaggaccucauuugacgaugguggcuugauguggcuccgucuccuacuuagugauuuccauuccagugcugccugcccuggguaacugcgcacaacauugccugccccuccgcuguccccagagcuccggccucugucgccaucagcaucagccugggagucuccccaggacggcauggcacgugagcagcacugcaguccuugggucuucggcgccaaccugcaacccugaucaaaggagggugagggcgugggaguuuccuuucaggugugucaaagacauaucagcugcgcucuagaaagagccaacgggucagccgcuuccuccuaguuuaacuugauuuugagugauuuguccauauggaaauaguaggguagcaucaaacugcgugauuaaauuggaagacugucuuuccgugugaacacaggauggcagacaucagagggcuaccggggcaggguggacacguuccugaggagaagccagggucugcagcucuccauccuccccgguagcuccagcugcagggcugagcugcuuccuauccccucagcucugaucucugccacuucccuguugugugggagaguggaacacuccugggaugccaggcauuucauaaugggaggaggcuccuguaaggaacggugggcgcccgcucacaguggggggcccuccagcaugguugcugacucugucagcccccagccccggguugccaauuagccagagagcuccugcacacacagcacuuccuuccccaguguguuuucuacccagauggggacaaaggagccagacagucauaucaagggcagaguucaaacauuuugaaaugagccauucacaugcuucuuuuuaaacaugggcaucaccugcccagaggcccuuggccugaagcagcaugcugcugaccuugggaagagagggugggcuggugggggugcuggaggagcugcccggcccugcaucugcaacccuccaguccccugcccacagagcacgcagagcccuccugugcuccgggaacgagauagcagcuuggcagcucggccucgcaucugaaaaggaauacgcugcaaagccucggcccuagacccacaucugcagcucccuggccuguggcgguucuuuccuucggggcaugcccuuccagaucucugucccuccugucuucuagcacuuuaaagaugccccuuaucaucagauaaagagaaguauggggacgaugggguggcacuaacaguagaugucauuggugaggacugauggcaaagucucauucccucacagccgcgcugccagcucgccucccccucgcuuugcugggagccugcccuggacuccaaaaggacacggguucaccuccuuucccaggcuggagggguacugcccuucccugaggggccuucugacuggggcaggaacucuuuucguguguccagcugggccuggaggccaagucaaggugugucacaccacacaccagggcaaggaccucaucuccagggaacagggacaagugaguggcuaccagaaaguccccgggcagccuggaguuccccagagccuggcugguccuacagagcacuucuccccugggccaccaggaccaaagcgaccuaaacacuugaagcuaaaagcaagugcugaugaugggaugggcuggcacguggcugggagggcuccugggcaccacagagcccucagcccagcagagaguggaggcucaguccguggggagcggggguugugaaggagacauggccaagccccuggucggggagauaagcucccccaugcccaggccagccccaggggcaacaagccaacauggagagagguggcaggugaggcugacaggugggugccuguggccugggcccaugagggcaaugguccucaguaaccaauggaaaggacacaagggcgccauggucaccaccacccaggcaaagaaaugggagguggccagcacccccacccuucacuuuucauuauuauuauuauuauuauuauuauuauuuuuuuuuuuuuuuuuuuuuuuuuuuuuuuuuuuugagaguagaaaggcaacuucauuggagcuuuuggggaaaaaagccuucaaagucaccuucuggcagcucuggggagggucauuugggguggacugagaguguagggcagguagggugugugcugggcagcagcuguguccaugcagccagcacccagcagggcagucucacaggagccguugugagcacuuuggacuugaagcagcuaugaaccaccccccgcccccaccgucucucccucucacacacacacacacacacacacacacacacacacacacacacacacacccguuuuaccauuuucucagugcuuuuucugcuuucauuccuuugauuuuaguuagaagcacaagccaugauggaugugggacucuucucaauugcucuuuaaauugucacuuuuuaaauccuauuaaaugaagugucgauuccugacaaauacauuaaaaguguuuuauuccuagaagaguugggaaaagaaauuauuuucagcaagaguaggauguuuguuaaacugauuccuuggcaaaaaagagaaaaaaagcuuuugaauugugcaaaaaauugcccaguauuaugcugucgccccaguugucaaacugcugugcagauggcuccagcccagucaacuucaccuucuuuuuaauuuagaagauaacaaaauuguaaucacuuauccuuuccagagccaagggggaaaaggaagguauaaucuacaauaaaaagcgagcguucuguguacugaggccacuuggugauaaagagauggagcgcuccccucacagacuucaauuaagaaccucccuuuggacagggaagaaaggugucaaagaggaaaagaaaauuaaauuugcuuccuuccaggaguuuuuccucauuagugcuugcuugucgguguuauuauuuuaaucuuaccuucuauguggugaccagcuccuccgagcgaugcccaggucgggcacggcccgggcagggcaggucugcagcgaugccguggcagaggugggcauacuccauuuguuuuggcagcugcagccauugauucugcauauuuuuccugacaacagccccggcaggaguucaguuaggaauuuaaagugcaguucaugguucugugccaccguggcuuuuauuauuauaauauuaaauuagaaguuguccuagugccugguguuugcucagaguuuccagaagagagggaagggcaagguuuaaauggcaugcaggacaacuggaaugcccccaucucucucgcugacacggauccagucauaccuggggcuggacgggauuuggaggcccuggucauuugcagagugaauggugaagggcgcggaaaagguuugcuuggaggaaagaccgguugcagaggcgaggcggagggaggaggggcggaggcagcaggucuuuguuugugguaggucucuggcuuccaucagggaggaggaaagaggcugugcccuuccuggcucuuggcugcaccacugaggacgcuccgagggacagcgugcucacccauccuuugcacagugcuggccccaagccccacggccuuccagcuaggauuuucugcugggcucaugcagaggcaggggacaggugcauggaagagccgccccacccgacacaccauuguuugaaaaucacuguucucuuuacucacuuaaaaaaguguacagggaacaccuguuccuggcauaaugcuccaaccucgcggaaggggccaggugcccuucaucuggcucuggcugcuuccgaccugggcccacgucaucguucacguccucugugaccaccacuggucacggugcucccuggcccagccuccaacccacccagcacccuggcaucucccaggccagucugcugcacccagcgagcuuccagucagaagccaggcugaacggcccuccugccccaucagcuucgugucuucuuuuuuuuaaagaacugaaauaguccccaagaggccucauggccugaagacucacaaucauccaccuguaauuuaugauaaaugucugggagcauuuaccauuugcguccgugaguauuuauagcccugaaugggcgggggggaggggggguggaggaggcccugcagccaggagcuacacaccuguccccacuaguguccccugguugacagagcccccucagccuccccaaggcugucacugcggcugugacagcugaggagugccgccuuugaaagccaguggacagucgcuccacuagggggagaggcccuggcccuggcgcagaggaggcguugcgaggcgggacgggggcuggaggggcugagcagccuucagggcagggacugggcccugggucacuggagacguugauauuaguccgucugcugccaaauugcuccccaccacaugagccccagggguuuaugucccaggaaggcgagggugcccaucugagcggaauugggaggggacggcaccagcucaucucccucagggccuuugccuccuggugcugcccugguggcugcuccugcaccacagccccugauggcugcugcuaguccugaguugcuggguuuacccccagcccacacuucccaccugggccugagggugcggccagugcccuaguccuagccacuacaggagucauucugagaccugcuggaggccauggggucuucccaggccccucaaucagcugcuuccagggucagcagggcagggugcugccaguaagguccucagggagcacagcccggccgcccaggcugggggauacuggggcagagcuuccaggucuguggggccugaucucuccccaaggcucuccaggccuuggugcgcccuccacagugaccuucagagaggcugcacccccucagaagaacagugagaaaucucuccaucacacgcucccugguccuuaugucccugaggccacccuuccccaccccccagugccuggagaagcgugagacucuggaggggcgccaggaggccagggguccucagggcuaggccuggagcucggcccaagagcugcuuuugcgaagccugucuugaauccggauucaccagagaacaagagccucccagccuuuggcguuucugggccuguaaagauguguguaccucccaggccacucugaugcaagggcagggaccaugccaggccuggguugggaauggcucugugacuccagaagcuccgucuaaaacuccaaagaugcccaaaaggcugugcugcuauguggaauguguauuauuugugagcacgaugcggcuucuuccucauuuugcagagcaaccuaagcgggcagauguacaaaccguguguucgaaaccccugaguccaugugugugaaaaugcagguuuucucuuagaaauaaaguggugacuugugcuguaaaaaaaaaaaaaaaaaaa

SEQ ID NO: 5 - RNA, 8967 nt, Homo sapiens
ggagccaggugccggcagggcagccacacccuggggcccccuguucuggaggggcagagggucgggccgaugcgaaaaagccgcggaagcugccuuaggcgacuuucugggccugcaucgccggacccagcagcccgcugucgaucgguugcuaagugacgccagcgcgcaguggcgcgugcgcggccacggcggagugcgugaaagcggacgcgcgccgcagcagccggggcggcggcgaggacggcggccacggaagcggccaagggggcgguggcgcagagaggggugcggagcgggcggaagaggcgucugugucgcagcuuggucucagaggagcaucgcugggcucgucucaauuuggcccugcuguauuucaagugcucgguagccacauguggcaaguggccaucacuguggccauugcagcuccaaagggacaggagaguugauggaaauuagacacugcauucuacagccucaugagcccaagagagcaaaccuugguuugcugaaaaucagcagauaaggcaauugaaagggaagaagaacaacgacuaucgucuauuccacaagaugaguaacagccacccucuucgccccuuuacugcagugggggaaauugaucaugugcacauuuugucugaacauauuggugccuuguugauuggggaagaauauggcgacgucacauucgugguggaaaagaaacguuuuccugcccacaggguaauuuuagcagccaggugccaauauuuucgagcauuauuauaugguggaaugcgagagucucagccugaagcagaaauuccucuccaagacaccacugcagaagcauucacaaugcuacucaaauauaucuacacugggcgggcaacgcugacagaugagaaggaggaggugcugcuggacuuuuugagccuggcucauaaauauggauuuccagagcuagaggauucuaccucugaguaucucugcaccauacuuaacauucagaaugucugcaugacuuuugauguugccagucucuacucacuucccaaguuaacuuguaugugcugcauguuuauggauaggaaugcucaggaaguccucucaagugaagguuuccucucccuuucuaagacagcacuuuuaaacaucguguuaagagacucauuugcagcucccgaaaaagauauuuuccuagccuuauuaaacugguguaagcacaauucaaaggagaaucaugcugaaaucaugcaggcugugcguuuaccucucaugagccucacagagcuucugaauguugugaggccuucaggacugcugucuccugaugccauccuggaugccauuaaagugcgaucugagagccgggauauggaccucaauuauagaggcaugcucauaccagaagaaaacauugcaacuaugaaguauggagcccaaguuguaaagggggagcugaaaucagccuuauuagauggugauacucaaaauuaugauuuggaucauggauuuucaaggcacccaauugaugaugacugccguuccggcaucgagauuaagcuaggucagccauccauuaucaaucacauacggauacucuugugggaccgagauagccggucuuacucauacuucauugaagugucaauggaugaacuugauugggucagagugauagaucauucacaauaucugugucguucuuggcagaaauuauauuuuccagcccgugucugcagguauauucgaauuguugggacucacaacacagugaacaagauuuuucacauuguggcuuuugaauguauguuuacaaacaaaaccuucacucuugagaaggggcugauaguucccauggagaauguugcaacaauugcugauugugccagugugauugaaggagucagucggagccgaaaugccuugcugaauggggacacuaagaauuaugacugggauucuggcuacacaugucaccagcuaggaaguggugcgauugugguucaguuggcacaaccguacaugauugggucaauacgguuacuacuuugggauugugaugaucgaagcuauagcuacuacguugagguuucuaccaaccagcaacaguggaccaugguugcugacagaacuaaagucuccugcaaguccuggcagucaguaacuuuugaaaggcagccugccuccuucauccguaucguugggacacacaacacagcaaaugagguguuccacuguguccacuuugaguguccagagcagcagagcagccagaaggaggaaaauagugaggaaucggggacaggggacaccagccuggccggucagcagcucgacucccaugcgcugcgggcgccuaguggcagcucacuacccuccagcccaggcuccaacucacgcucccccaaccggcagcaccaauaaaggaggcagcgggccuggugugacuuggugggcucgggcaacggcaggaaacggucuccucccugagcaggggucucuguugacugcccccaccucugccccuuuccagggaggagccgaccuagcugcaaaagcagacaccgaacagguuuucucccaaggacagaaaggggcugcuuuguucuucucaauuuauccaaaucaggcaggucucaagggggagaaaaugggucuucaaucuucaucaaguccaccauuaacacccuaccucucuaaucuagcccaggcgcagggaacaggaggaagucauagacaggccuguguuggaauacaaaaauggcacgcaaacacucaggcggaggagaaagccauugggccuuugcuaccuggaggcacccuuuaggaaugggccuccauagaguguucauucugcuggaauacauugcccuggcaacuaaaugauuuuuuauuuguuuuaguuauucuccacucagugaaucauagguguuccggaucuauuugaaaugcucaaacagcaaaucacuaguuuuaaucccuuuugaugcuauaauuuuccuuccuugguuuugaacgguucagcuugcggggacagagacccaucgugcaugucuccuaaaugagacacggaugcagcucauccuggcacuugggccacagaggaagugccacaaaugcacacaagcaaaucaaccuguggacacuggaguuaauagucacucguguggcugaguuacucaccugucauuccugacauggauccagccuuuaagcuugggaggcugacaaggaaauaguuacuaugcccgcugcuaauucacuguuccgaaguccauguagauaaacauauaccgaguucuggcguggccaaaccugccggggacugccguggcuuagcugccccuccaguuguguuuaucgaagcauuggagaggcagccaccugcccaucagcgucaggaacgcagaacugggacuccgugcuucccccacaccacaguacucaacuggggacugaaaaauagcaguuccaagaagcuuuucuccuaauccucaucaaaaggaccucauuugacgaugguggcuugauguggcuccgucuccuacuuagugauuuccauuccagugcugccugcccuggguaacugcgcacaacauugccugccccuccgcuguccccagagcuccggccucugucgccaucagcaucagccugggagucuccccaggacggcauggcacgugagcagcacugcaguccuugggucuucggcgccaaccugcaacccugaucaaaggagggugagggcgugggaguuuccuuucaggugugucaaagacauaucagcugcgcucuagaaagagccaacgggucagccgcuuccuccuaguuuaacuugauuuugagugauuuguccauauggaaauaguaggguagcaucaaacugcgugauuaaauuggaagacugucuuuccgugugaacacaggauggcagacaucagagggcuaccggggcaggguggacacguuccugaggagaagccagggucugcagcucuccauccuccccgguagcuccagcugcagggcugagcugcuuccuauccccucagcucugaucucugccacuucccuguugugugggagaguggaacacuccugggaugccaggcauuucauaaugggaggaggcuccuguaaggaacggugggcgcccgcucacaguggggggcccuccagcaugguugcugacucugucagcccccagccccggguugccaauuagccagagagcuccugcacacacagcacuuccuuccccaguguguuuucuacccagauggggacaaaggagccagacagucauaucaagggcagaguucaaacauuuugaaaugagccauucacaugcuucuuuuuaaacaugggcaucaccugcccagaggcccuuggccugaagcagcaugcugcugaccuugggaagagagggugggcuggugggggugcuggaggagcugcccggcccugcaucugcaacccuccaguccccugcccacagagcacgcagagcccuccugugcuccgggaacgagauagcagcuuggcagcucggccucgcaucugaaaaggaauacgcugcaaagccucggcccuagacccacaucugcagcucccuggccuguggcgguucuuuccuucggggcaugcccuuccagaucucugucccuccugucuucuagcacuuuaaagaugccccuuaucaucagauaaagagaaguauggggacgaugggguggcacuaacaguagaugucauuggugaggacugauggcaaagucucauucccucacagccgcgcugccagcucgccucccccucgcuuugcugggagccugcccuggacuccaaaaggacacggguucaccuccuuucccaggcuggagggguacugcccuucccugaggggccuucugacuggggcaggaacucuuuucguguguccagcugggccuggaggccaagucaaggugugucacaccacacaccagggcaaggaccucaucuccagggaacagggacaagugaguggcuaccagaaaguccccgggcagccuggaguuccccagagccuggcugguccuacagagcacuucuccccugggccaccaggaccaaagcgaccuaaacacuugaagcuaaaagcaagugcugaugaugggaugggcuggcacguggcugggagggcuccugggcaccacagagcccucagcccagcagagaguggaggcucaguccguggggagcggggguugugaaggagacauggccaagccccuggucggggagauaagcucccccaugcccaggccagccccaggggcaacaagccaacauggagagagguggcaggugaggcugacaggugggugccuguggccugggcccaugagggcaaugguccucaguaaccaauggaaaggacacaagggcgccauggucaccaccacccaggcaaagaaaugggagguggccagcacccccacccuucacuuuucauuauuauuauuauuauuauuauuauuauuuuuuuuuuuuuuuuuuuuuuuuuuuuuuuuuuuugagaguagaaaggcaacuucauuggagcuuuuggggaaaaaagccuucaaagucaccuucuggcagcucuggggagggucauuugggguggacugagaguguagggcagguagggugugugcugggcagcagcuguguccaugcagccagcacccagcagggcagucucacaggagccguugugagcacuuuggacuugaagcagcuaugaaccaccccccgcccccaccgucucucccucucacacacacacacacacacacacacacacacacacacacacacacacacccguuuuaccauuuucucagugcuuuuucugcuuucauuccuuugauuuuaguuagaagcacaagccaugauggaugugggacucuucucaauugcucuuuaaauugucacuuuuuaaauccuauuaaaugaagugucgauuccugacaaauacauuaaaaguguuuuauuccuagaagaguugggaaaagaaauuauuuucagcaagaguaggauguuuguuaaacugauuccuuggcaaaaaagagaaaaaaagcuuuugaauugugcaaaaaauugcccaguauuaugcugucgccccaguugucaaacugcugugcagauggcuccagcccagucaacuucaccuucuuuuuaauuuagaagauaacaaaauuguaaucacuuauccuuuccagagccaagggggaaaaggaagguauaaucuacaauaaaaagcgagcguucuguguacugaggccacuuggugauaaagagauggagcgcuccccucacagacuucaauuaagaaccucccuuuggacagggaagaaaggugucaaagaggaaaagaaaauuaaauuugcuuccuuccaggaguuuuuccucauuagugcuugcuugucgguguuauuauuuuaaucuuaccuucuauguggugaccagcuccuccgagcgaugcccaggucgggcacggcccgggcagggcaggucugcagcgaugccguggcagaggugggcauacuccauuuguuuuggcagcugcagccauugauucugcauauuuuuccugacaacagccccggcaggaguucaguuaggaauuuaaagugcaguucaugguucugugccaccguggcuuuuauuauuauaauauuaaauuagaaguuguccuagugccugguguuugcucagaguuuccagaagagagggaagggcaagguuuaaauggcaugcaggacaacuggaaugcccccaucucucucgcugacacggauccagucauaccuggggcuggacgggauuuggaggcccuggucauuugcagagugaauggugaagggcgcggaaaagguuugcuuggaggaaagaccgguugcagaggcgaggcggagggaggaggggcggaggcagcaggucuuuguuugugguaggucucuggcuuccaucagggaggaggaaagaggcugugcccuuccuggcucuuggcugcaccacugaggacgcuccgagggacagcgugcucacccauccuuugcacagugcuggccccaagccccacggccuuccagcuaggauuuucugcugggcucaugcagaggcaggggacaggugcauggaagagccgccccacccgacacaccauuguuugaaaaucacuguucucuuuacucacuuaaaaaaguguacagggaacaccuguuccuggcauaaugcuccaaccucgcggaaggggccaggugcccuucaucuggcucuggcugcuuccgaccugggcccacgucaucguucacguccucugugaccaccacuggucacggugcucccuggcccagccuccaacccacccagcacccuggcaucucccaggccagucugcugcacccagcgagcuuccagucagaagccaggcugaacggcccuccugccccaucagcuucgugucuucuuuuuuuuaaagaacugaaauaguccccaagaggccucauggccugaagacucacaaucauccaccuguaauuuaugauaaaugucugggagcauuuaccauuugcguccgugaguauuuauagcccugaaugggcgggggggaggggggguggaggaggcccugcagccaggagcuacacaccuguccccacuaguguccccugguugacagagcccccucagccuccccaaggcugucacugcggcugugacagcugaggagugccgccuuugaaagccaguggacagucgcuccacuagggggagaggcccuggcccuggcgcagaggaggcguugcgaggcgggacgggggcuggaggggcugagcagccuucagggcagggacugggcccugggucacuggagacguugauauuaguccgucugcugccaaauugcuccccaccacaugagccccagggguuuaugucccaggaaggcgagggugcccaucugagcggaauugggaggggacggcaccagcucaucucccucagggccuuugccuccuggugcugcccugguggcugcuccugcaccacagccccugauggcugcugcuaguccugaguugcuggguuuacccccagcccacacuucccaccugggccugagggugcggccagugcccuaguccuagccacuacaggagucauucugagaccugcuggaggccauggggucuucccaggccccucaaucagcugcuuccagggucagcagggcagggugcugccaguaagguccucagggagcacagcccggccgcccaggcugggggauacuggggcagagcuuccaggucuguggggccugaucucuccccaaggcucuccaggccuuggugcgcccuccacagugaccuucagagaggcugcacccccucagaagaacagugagaaaucucuccaucacacgcucccugguccuuaugucccugaggccacccuuccccaccccccagugccuggagaagcgugagacucuggaggggcgccaggaggccagggguccucagggcuaggccuggagcucggcccaagagcugcuuuugcgaagccugucuugaauccggauucaccagagaacaagagccucccagccuuuggcguuucugggccuguaaagauguguguaccucccaggccacucugaugcaagggcagggaccaugccaggccuggguugggaauggcucugugacuccagaagcuccgucuaaaacuccaaagaugcccaaaaggcugugcugcuauguggaauguguauuauuugugagcacgaugcggcuucuuccucauuuugcagagcaaccuaagcgggcagauguacaaaccguguguucgaaaccccugaguccaugugugugaaaaugcagguuuucucuuagaaauaaaguggugacuugugcuguaaaaaaaaaaaaaaaaaaa

SEQ ID NO: 6 - RNA, 4547 nt, Homo sapiens
cccucucuuccccccgcccugccuucccuugcacccuccuucuucccuccgcccgggagcucucccugguccccggcgccgccuccuucccucccggcuccccgcuccccgcucccguggcugccgccgccccggggaagaagagacaggggugggguuugggggaagcgagagaggaggggagagacccuggccaggcuggagccuggauucgaggggaggagggacgggaggaggagaaagguggaggagaagggaggggggagcggggaggagcggccgggccuggggccuugaggcccggggagagccggggagccgggcccgcgcgccgagauguugcugcugcugcuacuggcgccacucuuccuccgccccccgggcgcgggcggggcgcagacccccaacgccaccucagaagguugccagaucauacacccgcccugggaagggggcaucagguaccggggccugacucgggaccaggugaaggcuaucaacuuccugccaguggacuaugagauugaguaugugugccggggggagcgcgaggugguggggcccaagguccgcaagugccuggccaacggcuccuggacagauauggacacacccagccgcuguguccgaaucugcuccaagucuuauuugacccuggaaaaugggaagguuuuccugacggguggggaccucccagcucuggacggagcccggguggauuuccggugugaccccgacuuccaucuggugggcagcucccggagcaucuguagucagggccaguggagcacccccaagccccacugccaggugaaucgaacgccacacucagaacggcgcgcaguguacaucggggcacuguuucccaugagcgggggcuggccagggggccaggccugccagcccgcgguggagauggcgcuggaggacgugaauagccgcagggacauccugccggacuaugagcucaagcucauccaccacgacagcaagugugauccaggccaagccaccaaguaccuauaugagcugcucuacaacgacccuaucaagaucauccuuaugccuggcugcagcucugucuccacgcugguggcugaggcugcuaggauguggaaccucauugugcuuuccuauggcuccagcucaccagcccugucaaaccggcagcguuuccccacuuucuuccgaacgcacccaucagccacacuccacaacccuacccgcgugaaacucuuugaaaaguggggcuggaagaagauugcuaccauccagcagaccacugaggucuucacuucgacucuggacgaccuggaggaacgagugaaggaggcuggaauugagauuacuuuccgccagaguuucuucucagauccagcugugcccgucaaaaaccugaagcgccaggaugcccgaaucaucgugggacuuuucuaugagacugaagcccggaaaguuuuuugugagguguacaaggagcgucucuuugggaagaaguacgucugguuccucauugggugguaugcugacaauugguucaagaucuacgacccuucuaucaacugcacaguggaugagaugacugaggcgguggagggccacaucacaacugagauugucaugcugaauccugccaauacccgcagcauuuccaacaugacaucccaggaauuuguggagaaacuaaccaagcgacugaaaagacacccugaggagacaggaggcuuccaggaggcaccgcuggccuaugaugccaucugggccuuggcacuggcccugaacaagacaucuggaggaggcggccguucuggugugcgccuggaggacuucaacuacaacaaccagaccauuaccgaccaaaucuaccgggcaaugaacucuucguccuuugagggugucucuggccaugugguguuugaugccagcggcucucggauggcauggacgcuuaucgagcagcuucaggguggcagcuacaagaagauuggcuacuaugacagcaccaaggaugaucuuuccugguccaaaacagauaaauggauuggagggucccccccagcugaccagacccuggucaucaagacauuccgcuuccugucacagaaacucuuuaucuccgucucaguucucuccagccugggcauuguccuagcuguugucugucuguccuuuaacaucuacaacucacauguccguuauauccagaacucacagcccaaccugaacaaccugacugcugugggcugcucacuggcuuuagcugcugucuucccccuggggcucgaugguuaccacauugggaggaaccaguuuccuuucgucugccaggcccgccucuggcuccugggccugggcuuuagucugggcuacgguuccauguucaccaagauuugguggguccacacggucuucacaaagaaggaagaaaagaaggaguggaggaagacucuggaacccuggaagcuguaugccacagugggccugcuggugggcauggauguccucacucucgccaucuggcagaucguggacccucugcaccggaccauugagacauuugccaaggaggaaccuaaggaagauauugacgucucuauucugccccagcuggagcauugcagcuccaggaagaugaauacauggcuuggcauuuucuaugguuacaaggggcugcugcugcugcugggaaucuuccuugcuuaugagaccaagaguguguccacugagaagaucaaugaucaccgggcugugggcauggcuaucuacaauguggcaguccugugccucaucacugcuccugucaccaugauucuguccagccagcaggaugcagccuuugccuuugccucucuugccauaguuuucuccuccuauaucacucuuguugugcucuuugugcccaagaugcgcaggcugaucacccgaggggaauggcagucggaggcgcaggacaccaugaagacagggucaucgaccaacaacaacgaggaggagaagucccggcuguuggagaaggagaaccgugaacuggaaaagaucauugcugagaaagaggagcgugucucugaacugcgccaucaacuccagucucggcagcagcuccgcucccggcgccacccaccgacacccccagaacccucugggggccugcccaggggacccccugagccccccgaccggcuuagcugugaugggagucgagugcauuugcuuuauaagugaggguagggugagggaggacaggccaguagggggagggaaagggagaggggaagggcaggggacucaggaagcaggggguccccauccccagcugggaagaacaugcuauccaaucucaucucuuguaaauacaugucccccugugaguucugggcugauuugggucucucauaccucugggaaacagaccuuuuucucucuuacugcuucauguaauuuuguaucaccucuucacaauuuaguucguaccuggcuugaagcugcucacugcucacacgcugccuccucagcagccucacugcaucuuucucuucccaugcaacacccucuucuaguuaccacggcaaccccugcagcuccucugccuuugugcucuguuccuguccagcaggggucucccaacaagugcucuuuccaccccaaaggggccucuccuuuucuccacugucauaaucucuuuccaucuuacuugcccuucuauacuuucucacauguggcucccccugaauuuugcuuccuuugggagcucauucuuuucgccaaggcucacaugcuccuugccucugcucugugcacucacgcucagcacacaugcauccuccccucuccugcgugugcccacugaacaugcucauguguacacacgcuuuucccguaugcuuucuucauguucagucacaugugcucucgggugcccugcauucacagcuacgugugccccucucauggucaugggucugcccuugagcguguuuggguaggcaugugcaauuugucuagcaugcugagucaugucuuuccuauuugcacacguccauguuuauccauguacuuucccuguguacccuccauguaccuuguguacuuucuucccuuaaaucaugguauucuucugacagagccauauguacccuacccugcacauuguuaugcacuuuuccccaauucauguuugguggggccauccacacccucuccuugucacagaaucuccauuucugcucagauuccccccaucuccauugcauucauguacuacccucagucuacacucacaaucaucuucucccaagacugcucccuuuuguuuuguguuuuuuugaggggaauuaaggaaaaauaagugggggcagguuuggagagcugcuuccaguggauaguugaugagaauccugaccaaaggaaggcacccuugacuguugggauagacagauggaccuauggggugggagguggugucccuuucacacuguggugucucuuggggaaggaucuccccgaaucucaauaaaccagugaacagugugacucggcaaaaaaaaaaa

SEQ ID NO: 7 - RNA, 4361 nt, Homo sapiens
cccucucuuccccccgcccugccuucccuugcacccuccuucuucccuccgcccgggagcucucccugguccccggcgccgccuccuucccucccggcuccccgcuccccgcucccguggcugccgccgccccggggaagaagagacaggggugggguuugggggaagcgagagaggaggggagagacccuggccaggcuggagccuggauucgaggggaggagggacgggaggaggagaaagguggaggagaagggaggggggagcggggaggagcggccgggccuggggccuugaggcccggggagagccggggagccgggcccgcgcgccgagauguugcugcugcugcuacuggcgccacucuuccuccgccccccgggcgcgggcggggcgcagacccccaacgccaccucagaagguugccagaucauacacccgcccugggaagggggcaucagguaccggggccugacucgggaccaggugaaggcuaucaacuuccugccaguggacuaugagauugaguaugugugccggggggagcgcgaggugguggggcccaagguccgcaagugccuggccaacggcuccuggacagauauggacacacccagccgcugugugaaucgaacgccacacucagaacggcgcgcaguguacaucggggcacuguuucccaugagcgggggcuggccagggggccaggccugccagcccgcgguggagauggcgcuggaggacgugaauagccgcagggacauccugccggacuaugagcucaagcucauccaccacgacagcaagugugauccaggccaagccaccaaguaccuauaugagcugcucuacaacgacccuaucaagaucauccuuaugccuggcugcagcucugucuccacgcugguggcugaggcugcuaggauguggaaccucauugugcuuuccuauggcuccagcucaccagcccugucaaaccggcagcguuuccccacuuucuuccgaacgcacccaucagccacacuccacaacccuacccgcgugaaacucuuugaaaaguggggcuggaagaagauugcuaccauccagcagaccacugaggucuucacuucgacucuggacgaccuggaggaacgagugaaggaggcuggaauugagauuacuuuccgccagaguuucuucucagauccagcugugcccgucaaaaaccugaagcgccaggaugcccgaaucaucgugggacuuuucuaugagacugaagcccggaaaguuuuuugugagguguacaaggagcgucucuuugggaagaaguacgucugguuccucauugggugguaugcugacaauugguucaagaucuacgacccuucuaucaacugcacaguggaugagaugacugaggcgguggagggccacaucacaacugagauugucaugcugaauccugccaauacccgcagcauuuccaacaugacaucccaggaauuuguggagaaacuaaccaagcgacugaaaagacacccugaggagacaggaggcuuccaggaggcaccgcuggccuaugaugccaucugggccuuggcacuggcccugaacaagacaucuggaggaggcggccguucuggugugcgccuggaggacuucaacuacaacaaccagaccauuaccgaccaaaucuaccgggcaaugaacucuucguccuuugagggugucucuggccaugugguguuugaugccagcggcucucggauggcauggacgcuuaucgagcagcuucaggguggcagcuacaagaagauuggcuacuaugacagcaccaaggaugaucuuuccugguccaaaacagauaaauggauuggagggucccccccagcugaccagacccuggucaucaagacauuccgcuuccugucacagaaacucuuuaucuccgucucaguucucuccagccugggcauuguccuagcuguugucugucuguccuuuaacaucuacaacucacauguccguuauauccagaacucacagcccaaccugaacaaccugacugcugugggcugcucacuggcuuuagcugcugucuucccccuggggcucgaugguuaccacauugggaggaaccaguuuccuuucgucugccaggcccgccucuggcuccugggccugggcuuuagucugggcuacgguuccauguucaccaagauuugguggguccacacggucuucacaaagaaggaagaaaagaaggaguggaggaagacucuggaacccuggaagcuguaugccacagugggccugcuggugggcauggauguccucacucucgccaucuggcagaucguggacccucugcaccggaccauugagacauuugccaaggaggaaccuaaggaagauauugacgucucuauucugccccagcuggagcauugcagcuccaggaagaugaauacauggcuuggcauuuucuaugguuacaaggggcugcugcugcugcugggaaucuuccuugcuuaugagaccaagaguguguccacugagaagaucaaugaucaccgggcugugggcauggcuaucuacaauguggcaguccugugccucaucacugcuccugucaccaugauucuguccagccagcaggaugcagccuuugccuuugccucucuugccauaguuuucuccuccuauaucacucuuguugugcucuuugugcccaagaugcgcaggcugaucacccgaggggaauggcagucggaggcgcaggacaccaugaagacagggucaucgaccaacaacaacgaggaggagaagucccggcuguuggagaaggagaaccgugaacuggaaaagaucauugcugagaaagaggagcgugucucugaacugcgccaucaacuccagucucggcagcagcuccgcucccggcgccacccaccgacacccccagaacccucugggggccugcccaggggacccccugagccccccgaccggcuuagcugugaugggagucgagugcauuugcuuuauaagugaggguagggugagggaggacaggccaguagggggagggaaagggagaggggaagggcaggggacucaggaagcaggggguccccauccccagcugggaagaacaugcuauccaaucucaucucuuguaaauacaugucccccugugaguucugggcugauuugggucucucauaccucugggaaacagaccuuuuucucucuuacugcuucauguaauuuuguaucaccucuucacaauuuaguucguaccuggcuugaagcugcucacugcucacacgcugccuccucagcagccucacugcaucuuucucuucccaugcaacacccucuucuaguuaccacggcaaccccugcagcuccucugccuuugugcucuguuccuguccagcaggggucucccaacaagugcucuuuccaccccaaaggggccucuccuuuucuccacugucauaaucucuuuccaucuuacuugcccuucuauacuuucucacauguggcucccccugaauuuugcuuccuuugggagcucauucuuuucgccaaggcucacaugcuccuugccucugcucugugcacucacgcucagcacacaugcauccuccccucuccugcgugugcccacugaacaugcucauguguacacacgcuuuucccguaugcuuucuucauguucagucacaugugcucucgggugcccugcauucacagcuacgugugccccucucauggucaugggucugcccuugagcguguuuggguaggcaugugcaauuugucuagcaugcugagucaugucuuuccuauuugcacacguccauguuuauccauguacuuucccuguguacccuccauguaccuuguguacuuucuucccuuaaaucaugguauucuucugacagagccauauguacccuacccugcacauuguuaugcacuuuuccccaauucauguuugguggggccauccacacccucuccuugucacagaaucuccauuucugcucagauuccccccaucuccauugcauucauguacuacccucagucuacacucacaaucaucuucucccaagacugcucccuuuuguuuuguguuuuuuugaggggaauuaaggaaaaauaagugggggcagguuuggagagcugcuuccaguggauaguugaugagaauccugaccaaaggaaggcacccuugacuguugggauagacagauggaccuauggggugggagguggugucccuuucacacuguggugucucuuggggaaggaucuccccgaaucucaauaaaccagugaacagugugacucggcaaaaaaaaaaa

SEQ ID NO: 8 - RNA, 4297 nt, Homo sapiens
cguuccuuuccuccucgagguugcauccccccuccccuccccgccccuccgacugucgcucccaccucggcgcucgcuucccuccccgcccccuuccugccuccccagcucccgcccgcccccccaccccccgcugccgcgcgccgcccgugacgucagagcccccucccagccccacaucucccuccugcuccuccuccuccccuccgucggucagucaguccgcgaggagaguccgcgguggcggcgacgguggcgagagccgcgggggccguaggaagccaaccuucccugcuucuccggggcccucgcccccuccuccccacaaaaucagggauggaggcgccuccccggcacccucuuagcagcccuccccaggaaaaguguccccccugagcuccuaacgcuccccaacagcuaccccugccccccacgccauggggcccggggccccuuuugcccgggugggguggccacugccgcuucugguugugauggcggcagggguggcuccggugugggccucccacuccccccaucucccgcggccucacucgcggguccccccgcaccccuccucagaacggcgcgcaguguacaucggggcacuguuucccaugagcgggggcuggccagggggccaggccugccagcccgcgguggagauggcgcuggaggacgugaauagccgcagggacauccugccggacuaugagcucaagcucauccaccacgacagcaagugugauccaggccaagccaccaaguaccuauaugagcugcucuacaacgacccuaucaagaucauccuuaugccuggcugcagcucugucuccacgcugguggcugaggcugcuaggauguggaaccucauugugcuuuccuauggcuccagcucaccagcccugucaaaccggcagcguuuccccacuuucuuccgaacgcacccaucagccacacuccacaacccuacccgcgugaaacucuuugaaaaguggggcuggaagaagauugcuaccauccagcagaccacugaggucuucacuucgacucuggacgaccuggaggaacgagugaaggaggcuggaauugagauuacuuuccgccagaguuucuucucagauccagcugugcccgucaaaaaccugaagcgccaggaugcccgaaucaucgugggacuuuucuaugagacugaagcccggaaaguuuuuugugagguguacaaggagcgucucuuugggaagaaguacgucugguuccucauugggugguaugcugacaauugguucaagaucuacgacccuucuaucaacugcacaguggaugagaugacugaggcgguggagggccacaucacaacugagauugucaugcugaauccugccaauacccgcagcauuuccaacaugacaucccaggaauuuguggagaaacuaaccaagcgacugaaaagacacccugaggagacaggaggcuuccaggaggcaccgcuggccuaugaugccaucugggccuuggcacuggcccugaacaagacaucuggaggaggcggccguucuggugugcgccuggaggacuucaacuacaacaaccagaccauuaccgaccaaaucuaccgggcaaugaacucuucguccuuugagggugucucuggccaugugguguuugaugccagcggcucucggauggcauggacgcuuaucgagcagcuucaggguggcagcuacaagaagauuggcuacuaugacagcaccaaggaugaucuuuccugguccaaaacagauaaauggauuggagggucccccccagcugaccagacccuggucaucaagacauuccgcuuccugucacagaaacucuuuaucuccgucucaguucucuccagccugggcauuguccuagcuguugucugucuguccuuuaacaucuacaacucacauguccguuauauccagaacucacagcccaaccugaacaaccugacugcugugggcugcucacuggcuuuagcugcugucuucccccuggggcucgaugguuaccacauugggaggaaccaguuuccuuucgucugccaggcccgccucuggcuccugggccugggcuuuagucugggcuacgguuccauguucaccaagauuugguggguccacacggucuucacaaagaaggaagaaaagaaggaguggaggaagacucuggaacccuggaagcuguaugccacagugggccugcuggugggcauggauguccucacucucgccaucuggcagaucguggacccucugcaccggaccauugagacauuugccaaggaggaaccuaaggaagauauugacgucucuauucugccccagcuggagcauugcagcuccaggaagaugaauacauggcuuggcauuuucuaugguuacaaggggcugcugcugcugcugggaaucuuccuugcuuaugagaccaagaguguguccacugagaagaucaaugaucaccgggcugugggcauggcuaucuacaauguggcaguccugugccucaucacugcuccugucaccaugauucuguccagccagcaggaugcagccuuugccuuugccucucuugccauaguuuucuccuccuauaucacucuuguugugcucuuugugcccaagaugcgcaggcugaucacccgaggggaauggcagucggaggcgcaggacaccaugaagacagggucaucgaccaacaacaacgaggaggagaagucccggcuguuggagaaggagaaccgugaacuggaaaagaucauugcugagaaagaggagcgugucucugaacugcgccaucaacuccagucucggcagcagcuccgcucccggcgccacccaccgacacccccagaacccucugggggccugcccaggggacccccugagccccccgaccggcuuagcugugaugggagucgagugcauuugcuuuauaagugaggguagggugagggaggacaggccaguagggggagggaaagggagaggggaagggcaggggacucaggaagcaggggguccccauccccagcugggaagaacaugcuauccaaucucaucucuuguaaauacaugucccccugugaguucugggcugauuugggucucucauaccucugggaaacagaccuuuuucucucuuacugcuucauguaauuuuguaucaccucuucacaauuuaguucguaccuggcuugaagcugcucacugcucacacgcugccuccucagcagccucacugcaucuuucucuucccaugcaacacccucuucuaguuaccacggcaaccccugcagcuccucugccuuugugcucuguuccuguccagcaggggucucccaacaagugcucuuuccaccccaaaggggccucuccuuuucuccacugucauaaucucuuuccaucuuacuugcccuucuauacuuucucacauguggcucccccugaauuuugcuuccuuugggagcucauucuuuucgccaaggcucacaugcuccuugccucugcucugugcacucacgcucagcacacaugcauccuccccucuccugcgugugcccacugaacaugcucauguguacacacgcuuuucccguaugcuuucuucauguucagucacaugugcucucgggugcccugcauucacagcuacgugugccccucucauggucaugggucugcccuugagcguguuuggguaggcaugugcaauuugucuagcaugcugagucaugucuuuccuauuugcacacguccauguuuauccauguacuuucccuguguacccuccauguaccuuguguacuuucuucccuuaaaucaugguauucuucugacagagccauauguacccuacccugcacauuguuaugcacuuuuccccaauucauguuugguggggccauccacacccucuccuugucacagaaucuccauuucugcucagauuccccccaucuccauugcauucauguacuacccucagucuacacucacaaucaucuucucccaagacugcucccuuuuguuuuguguuuuuuugaggggaauuaaggaaaaauaagugggggcagguuuggagagcugcuuccaguggauaguugaugagaauccugaccaaaggaaggcacccuugacuguugggauagacagauggaccuauggggugggagguggugucccuuucacacuguggugucucuuggggaaggaucuccccgaaucucaauaaaccagugaacagugugacucggcaaaaaaaaaaa

SEQ ID NO: 9 - RNA, 7518 nt, Homo sapiens
acgcggcaggcaucgucuccuccggucucacagcuaccccgggcuggagcguggggaggggguaagggagccagccaacucggggaaacuucguugccagagaugacugggguuuucgggcuuucccagccuucccaugccgggccacacggcauucauuaaagugguggggcaacuauuggugcgcuccagccgggcagguuuugagcugcagagacccgugcccgguagcagcgcuuugccaccgagugcacccaggacgcaguggccgccagcccgggucuccugcugggggccggcgagugcagggagacuuugcgccacgauguuuuguuuuccugccgcaggaacgcuaggcagucucuuuccaccagacuuaacccgccugaagcaauagccauggaaguugcggcggcacagagggagacagaaccgcuuggagcggcaucuuucuuggauguuuuuucucuacacagacuucuuucuuuccuugguggccguucccgcagcagcaccggugugccagcccaagagcgccacuaacgggcaacccccggcuccggcuccgacuccaacuccgcgccuguccauuuccucccgagccacagugguagccaggauggaaggcaccucccaagggggcuugcagaccgucaugaaguggaagacggugguugccaucuuugugguugugguggucuaccuugucacuggcggucuugucuuccgggcauuggagcagcccuuugagagcagccagaagaauaccaucgccuuggagaaggcggaauuccugcgggaucaugucugugugagcccccaggagcuggagacguugauccagcaugcucuugaugcugacaaugcgggagucaguccaauaggaaacucuuccaacaacagcagccacugggaccucggcagugccuuuuucuuugcuggaacugucauuacgaccauaggguaugggaauauugcuccgagcacugaaggaggcaaaaucuuuuguauuuuauaugccaucuuuggaauuccacucuuugguuucuuauuggcuggaauuggagaccaacuuggaaccaucuuugggaaaagcauugcaagaguggagaaggucuuucgaaaaaagcaagugagucagaccaagauccgggucaucucaaccauccuguucaucuuggccggcugcauuguguuugugacgaucccugcugucaucuuuaaguacaucgagggcuggacggccuuggaguccauuuacuuugugguggucacucugaccacggugggcuuuggugauuuuguggcagggggaaacgcuggcaucaauuaucgggagugguauaagccccuagugugguuuuggauccuuguuggccuugccuacuuugcagcuguccucaguaugaucggagauuggcuacggguucuguccaaaaagacaaaagaagaggugggugaaaucaaggcccaugcggcagaguggaaggccaaugucacggcugaguuccgggagacacggcgaaggcucagcguggagauccacgauaagcugcagcgggcggccaccauccgcagcauggagcgccggcggcugggccuggaccagcgggcccacucacuggacaugcugucccccgagaagcgcucugucuuugcugcccuggacaccggccgcuucaaggccucaucccaggagagcaucaacaaccggcccaacaaccugcgccugaaggggccggagcagcugaacaagcaugggcagggugcguccgaggacaacaucaucaacaaguucggguccaccuccagacucaccaagaggaaaaacaaggaccucaaaaagaccuugcccgaggacguucagaaaaucuacaagaccuuccggaauuacucccuggacgaggagaagaaagaggaggagacggaaaagauguguaacucagacaacuccagcacagccaugcugacggacuguauccagcagcacgcugaguuggagaacggaaugauacccacggacaccaaagaccgggagccggagaacaacucauuacuugaagacagaaacuaaaugugaaggacauuggucuuggacugagcguuguguguguguguguguguguuuuuaauauucacacugagacaugugccuuaaacagacuuuuuaguccaaaauuacauagcauugaagaauauauuucacugugccauaaacaacugaaagcuugcucugccaaaaggaaucagagaacaagaacuucauuucagauagcaaacgcaggacacaccaagaguguccgugcacguagccgguucuggccguacauguuaagggcauuucaguggcagugcuguaccccugggcagugcuaccugggcacacacguagacaagggcagcuauuccuuagaccagccuccugaaagaaacaggugugucuuuuuaguggagucguaguaauaugugcacacacagaaggggaccugauugggugggagcugguuauguguaacuagcguuggaguugacauuuuggcaugugcucugagcuugaauuuugauaccaaccauucagugcaucauaccuagucuuucuaugcuccaaaugaaugucuguggggaccugagagcaccuggaauuuguuggaagcagaucagagcacacguacgaaaaggugcaauugcuuuucucaugacaaaagaaaaaaaauaaucacaaacacaucacacuguggauaucaccuuaaccaaugacagaagccugcugaguuuggucuuucucacagggguagguaguccacaugaacuggcgagcaguggugaaaacaugacaucaacugcugcgugguuaaaggccaaucaugcauuuuccucagugggugcaauggugacaauaaacugauuuggaaauguccauguacuuugguuauaacuuguuuugauaagagggguagaaauaccaaaacaauugccuugcaugaugccaguggcuugcuguucugucuagcugauccuacauuuuuacaaaaguagacauccugcauuucugagacacacugaggaggaccauggcaucuuuccaucucagggcuuuuugucccuugggugucuuaggguagacauagugauaagauccaaucucgcuaucuccaaacaaaaagaaaauguaccccuuguacaguaagcuugaaauguuuuuauugcuugcagcguaagugccauuaauuccauuuaacaaugauacuuagaaauauuuaaaauauuucauuuuacuuuuauguuugggcauuccuuuauucugaaaaagucaccacugcuccuugcuuguuucuacaucucacaucgguuacucacuaaaacuggaacacccuagacugauuguucccaggcaaacgauaagcuaacacaucauaauuaugauuggucugugaggcuagaacaucccgcagaggaaagucggggaauggcuagucauagguugcaugaugauuuuaaauggguuuguaucagaaaggggaaaauacauacuccuguguaugccucagucuuuaucauaauuggcugguucacagcuagcucuuugggccacacgugaaagcauuuggaccuggcaacaccaggcaguugcugguuauggguguucuaggcucacugcuaccuuuuacagaguucacgggauuuuacguaacuaacccgcaguguggauuuaccaaaagaucacagcacuagcggagacuuccagaaacugucucaugcuuucugaccuccagggaaaguuguacccacgacauccucaaccacugguaccauggaagaccugucccugcccauuucucaaaaaugcacagcucuucccugccuggagcacaugaagucccaucucccuguccccagaccuuuaggcucuguugaaguucuggcauugcuuaugagacccuuccuguccaaggaaggucuaagucaacuccuccauuggacuggagaugaggcucacuggugacauccugugucgacucaguaauaaauagucacuugggaggcgauguuuaugcagguguggauguugaguuuuacuaugggaaucauuccagccugacaaaauguguuuuaaggaauagguagauaacugggacuuccucuguuauucuaacaagucgaagcuuccuaauuaaacuguccauggaucucacuuauuugguuuccacucuguguuaucugguacguucuuuguagcaguguguuaaaauuccaggcccuacggccaaaucuggcagaggcuucccucucauuuauccagugccuucugugggccaggcacaggauuaggcacuuuaugaacgucagcucauugaaacuucacgacacuaugugggaaguauuugcaaacccauuauauacacaaggacacagaggcucagggaagcuauguuauuuauccaauggcacacagcaagaaagugguagagcugguauucaaagucugaccaaagucugcuuucaacuucacuacagugucugugucaugcauccuugugauuguguguguuuguguguaugugugucuuucuagcuucauggacacagcuuacagauguggggagcagauaugguggaaucuccaccaccaagagggcacaaggucuuuguguaaacauggcucaaaggguugccccugcagacaccuacuguacauuuauuugguuuuggaaauuuuguauguggcacccuuuaaaaaaugcccuuugaaagcacucuuuugcacuuuacuugcuaacuuuguagaaacucugcauacagcaggaauaaaauaguucaaagcacuaagcugcauacucuaccaaauggaacaggugcauguguugguaugugcauagaugcuuccccaaaugagucaaaucagucacacagagggaucaaacauaaccuugggcugggggugggaaaauuuucuacauaacccauucccugagacauuuggccaagaaugugaugaacaaaaucaaagaagauccucuauggugauugaucgauuaaauaugugugcaaaguguuuagaaaccuaugaaauacucucgcaaagaugcugagagagaauaagagguuggauuccucuucauauaaacuaauuuuggaggaggccaguugguuugaaguuacuugaauguuaccuuuuuuagauggggccaaauggcauguagaauacacgugauaggucaaagcugcuacacauucuauacaugcaucagcacagcccccccuuuccaaucugcacucccauuccagcauaaaccuaggagaaauguuucgauuucacacaaagaaagagcacacguccaccaucuucagugggggcugucuuuugcuucacuggcaagcaggcacugaauuuuucuugcaugacaaaucuggagguuuacuggugagagagccaaugggcauuuuuuccuggaaagaguacagcuccauacccaguccuaacccaacagugauauuuaucacuuuggggcagggcuguauagagugugugugugugugugugugugugugugugugugugugugugugugugcggguuggggugguguugggccaucucuggccuguuacuaagguaacuaggacuauuuguguuccagcagucauagccugugauugugggugcaucaguucucugccuagaucucuuguuaccuugucugcacaucaagggagggaguugagcacagauacuugucaagggccauuguaguugugcaguucucuaaugaaacacucccuaguccaugaguucacaaaauuuauuaagauuaaauuauaaguuggauuugugaauaaugacuaauuaauugucuugcccauuuuagguuaaggugagagcuuagucucuugcccuuugggauuugucuuuugggggauuaauggagaccagauguacuugggagacugguguccaaauucggaucaugcccuguguaggcucucucuauccucccuuauagcucuuuaguguacugucaccgggagggcucaugcugugagggcauuuuuugcaugguguuaagacuaguuaaagaaauuuuaagcuguuguuauuugcagucaauuguaguacuucauguaucaugaauucaaguacuaugaucagacagacaucucucucucucucacacacacacacacacacacacacgcacacauacacacacacacacacaccugaggaaauggcugcuuuggguucuauaaggaccauuccauguuuaaaguccuaguugagcugaaugcuaagaaccugcccccuugccucccucugagaugauaucauuuccuggcuucgucaaugcugccugucuauuugcaugcuggguucugaggacuagugagaaggugaccagaguuuggguggggcugguuuuuacccacuggauuuggugagaauaugaagcauccaguguguaccaggguuucugaaccacgggaaaggcguaggaaaacaaacauucagagccccuguaaaacgagaaaggaaaaaccagccaguguugcauuccacaucucugcuuguugcauuuugcuaauaugggguuauucuuucucacuguuaggaugcaauugugugcaaagacaguggcugagugaacaguaagagcuggcuaguaauggccuuaaaaagaaaaaggguaacucucugaaacaaagaucacuuuaguguggcauuguggaugcuguuaauucugcauagggaaacuuuggaacagcaugcuaauuacauggcuguaagcaaagcccuguccucugucucugcaccauaccuucauuggacuucaccaacccauccauacuccauguaaaccucaguucucucaugccugcccuaagucaguugacaucagugcaguggcauugaggagaaaugagaggugucucugauuuuacugaaaguuauuaucauuuucacaggugccugagauuugguaucuacuuuguguucuugauucuuaggugaaaaaucugaaauaguucccugugcauuaaaauaaauuauuuugagaggacuccugcuccgucgauucagcagaccuaugcugcagaagguaacugcggaagcucucuuuugcugucagggcucugagcuugaagggagaaggugcaguggugccuagaagugauaugcaaaccaccucacaugccagccucuggccuccuucccaucccagagucacagacaggggacccagugacaaugaugauaaauccauguguggagguguuuuacuuauuuuucuuuccguaggauuucauggugcuuuaaaaaaaaaggcauuuuacagaaaauaauguggggggagggagauuucauaauguucuuagggaaaguacaaaacaaauuugcuugugacauuucaauaagcugugcugcuauugucuuuauuugaugauguaauuuuuuuuucaaugauggagaaaaauugcaacaaagaccuucuggaagauccaaaaaaaaaaaaaaaaaaa

SEQ ID NO: 10 - RNA, 7198 nt, Homo sapiens
gcaaggcauggagccugcacuuuacucugaauguugguggcgccuucagagcauuguuagcaaggguccaugugaucacacugacaccugagagagugacacaccauguacauggcugguauauggaggauggauuuaagggggacaggacugaaggcugucgcagugauucaguggccguucccgcagcagcaccggugugccagcccaagagcgccacuaacgggcaacccccggcuccggcuccgacuccaacuccgcgccuguccauuuccucccgagccacagugguagccaggauggaaggcaccucccaagggggcuugcagaccgucaugaaguggaagacggugguugccaucuuugugguugugguggucuaccuugucacuggcggucuugucuuccgggcauuggagcagcccuuugagagcagccagaagaauaccaucgccuuggagaaggcggaauuccugcgggaucaugucugugugagcccccaggagcuggagacguugauccagcaugcucuugaugcugacaaugcgggagucaguccaauaggaaacucuuccaacaacagcagccacugggaccucggcagugccuuuuucuuugcuggaacugucauuacgaccauaggguaugggaauauugcuccgagcacugaaggaggcaaaaucuuuuguauuuuauaugccaucuuuggaauuccacucuuugguuucuuauuggcuggaauuggagaccaacuuggaaccaucuuugggaaaagcauugcaagaguggagaaggucuuucgaaaaaagcaagugagucagaccaagauccgggucaucucaaccauccuguucaucuuggccggcugcauuguguuugugacgaucccugcugucaucuuuaaguacaucgagggcuggacggccuuggaguccauuuacuuugugguggucacucugaccacggugggcuuuggugauuuuguggcagggggaaacgcuggcaucaauuaucgggagugguauaagccccuagugugguuuuggauccuuguuggccuugccuacuuugcagcuguccucaguaugaucggagauuggcuacggguucuguccaaaaagacaaaagaagaggugggugaaaucaaggcccaugcggcagaguggaaggccaaugucacggcugaguuccgggagacacggcgaaggcucagcguggagauccacgauaagcugcagcgggcggccaccauccgcagcauggagcgccggcggcugggccuggaccagcgggcccacucacuggacaugcugucccccgagaagcgcucugucuuugcugcccuggacaccggccgcuucaaggccucaucccaggagagcaucaacaaccggcccaacaaccugcgccugaaggggccggagcagcugaacaagcaugggcagggugcguccgaggacaacaucaucaacaaguucggguccaccuccagacucaccaagaggaaaaacaaggaccucaaaaagaccuugcccgaggacguucagaaaaucuacaagaccuuccggaauuacucccuggacgaggagaagaaagaggaggagacggaaaagauguguaacucagacaacuccagcacagccaugcugacggacuguauccagcagcacgcugaguuggagaacggaaugauacccacggacaccaaagaccgggagccggagaacaacucauuacuugaagacagaaacuaaaugugaaggacauuggucuuggacugagcguuguguguguguguguguguguuuuuaauauucacacugagacaugugccuuaaacagacuuuuuaguccaaaauuacauagcauugaagaauauauuucacugugccauaaacaacugaaagcuugcucugccaaaaggaaucagagaacaagaacuucauuucagauagcaaacgcaggacacaccaagaguguccgugcacguagccgguucuggccguacauguuaagggcauuucaguggcagugcuguaccccugggcagugcuaccugggcacacacguagacaagggcagcuauuccuuagaccagccuccugaaagaaacaggugugucuuuuuaguggagucguaguaauaugugcacacacagaaggggaccugauugggugggagcugguuauguguaacuagcguuggaguugacauuuuggcaugugcucugagcuugaauuuugauaccaaccauucagugcaucauaccuagucuuucuaugcuccaaaugaaugucuguggggaccugagagcaccuggaauuuguuggaagcagaucagagcacacguacgaaaaggugcaauugcuuuucucaugacaaaagaaaaaaaauaaucacaaacacaucacacuguggauaucaccuuaaccaaugacagaagccugcugaguuuggucuuucucacagggguagguaguccacaugaacuggcgagcaguggugaaaacaugacaucaacugcugcgugguuaaaggccaaucaugcauuuuccucagugggugcaauggugacaauaaacugauuuggaaauguccauguacuuugguuauaacuuguuuugauaagagggguagaaauaccaaaacaauugccuugcaugaugccaguggcuugcuguucugucuagcugauccuacauuuuuacaaaaguagacauccugcauuucugagacacacugaggaggaccauggcaucuuuccaucucagggcuuuuugucccuugggugucuuaggguagacauagugauaagauccaaucucgcuaucuccaaacaaaaagaaaauguaccccuuguacaguaagcuugaaauguuuuuauugcuugcagcguaagugccauuaauuccauuuaacaaugauacuuagaaauauuuaaaauauuucauuuuacuuuuauguuugggcauuccuuuauucugaaaaagucaccacugcuccuugcuuguuucuacaucucacaucgguuacucacuaaaacuggaacacccuagacugauuguucccaggcaaacgauaagcuaacacaucauaauuaugauuggucugugaggcuagaacaucccgcagaggaaagucggggaauggcuagucauagguugcaugaugauuuuaaauggguuuguaucagaaaggggaaaauacauacuccuguguaugccucagucuuuaucauaauuggcugguucacagcuagcucuuugggccacacgugaaagcauuuggaccuggcaacaccaggcaguugcugguuauggguguucuaggcucacugcuaccuuuuacagaguucacgggauuuuacguaacuaacccgcaguguggauuuaccaaaagaucacagcacuagcggagacuuccagaaacugucucaugcuuucugaccuccagggaaaguuguacccacgacauccucaaccacugguaccauggaagaccugucccugcccauuucucaaaaaugcacagcucuucccugccuggagcacaugaagucccaucucccuguccccagaccuuuaggcucuguugaaguucuggcauugcuuaugagacccuuccuguccaaggaaggucuaagucaacuccuccauuggacuggagaugaggcucacuggugacauccugugucgacucaguaauaaauagucacuugggaggcgauguuuaugcagguguggauguugaguuuuacuaugggaaucauuccagccugacaaaauguguuuuaaggaauagguagauaacugggacuuccucuguuauucuaacaagucgaagcuuccuaauuaaacuguccauggaucucacuuauuugguuuccacucuguguuaucugguacguucuuuguagcaguguguuaaaauuccaggcccuacggccaaaucuggcagaggcuucccucucauuuauccagugccuucugugggccaggcacaggauuaggcacuuuaugaacgucagcucauugaaacuucacgacacuaugugggaaguauuugcaaacccauuauauacacaaggacacagaggcucagggaagcuauguuauuuauccaauggcacacagcaagaaagugguagagcugguauucaaagucugaccaaagucugcuuucaacuucacuacagugucugugucaugcauccuugugauuguguguguuuguguguaugugugucuuucuagcuucauggacacagcuuacagauguggggagcagauaugguggaaucuccaccaccaagagggcacaaggucuuuguguaaacauggcucaaaggguugccccugcagacaccuacuguacauuuauuugguuuuggaaauuuuguauguggcacccuuuaaaaaaugcccuuugaaagcacucuuuugcacuuuacuugcuaacuuuguagaaacucugcauacagcaggaauaaaauaguucaaagcacuaagcugcauacucuaccaaauggaacaggugcauguguugguaugugcauagaugcuuccccaaaugagucaaaucagucacacagagggaucaaacauaaccuugggcugggggugggaaaauuuucuacauaacccauucccugagacauuuggccaagaaugugaugaacaaaaucaaagaagauccucuauggugauugaucgauuaaauaugugugcaaaguguuuagaaaccuaugaaauacucucgcaaagaugcugagagagaauaagagguuggauuccucuucauauaaacuaauuuuggaggaggccaguugguuugaaguuacuugaauguuaccuuuuuuagauggggccaaauggcauguagaauacacgugauaggucaaagcugcuacacauucuauacaugcaucagcacagcccccccuuuccaaucugcacucccauuccagcauaaaccuaggagaaauguuucgauuucacacaaagaaagagcacacguccaccaucuucagugggggcugucuuuugcuucacuggcaagcaggcacugaauuuuucuugcaugacaaaucuggagguuuacuggugagagagccaaugggcauuuuuuccuggaaagaguacagcuccauacccaguccuaacccaacagugauauuuaucacuuuggggcagggcuguauagagugugugugugugugugugugugugugugugugugugugugugugugugcggguuggggugguguugggccaucucuggccuguuacuaagguaacuaggacuauuuguguuccagcagucauagccugugauugugggugcaucaguucucugccuagaucucuuguuaccuugucugcacaucaagggagggaguugagcacagauacuugucaagggccauuguaguugugcaguucucuaaugaaacacucccuaguccaugaguucacaaaauuuauuaagauuaaauuauaaguuggauuugugaauaaugacuaauuaauugucuugcccauuuuagguuaaggugagagcuuagucucuugcccuuugggauuugucuuuugggggauuaauggagaccagauguacuugggagacugguguccaaauucggaucaugcccuguguaggcucucucuauccucccuuauagcucuuuaguguacugucaccgggagggcucaugcugugagggcauuuuuugcaugguguuaagacuaguuaaagaaauuuuaagcuguuguuauuugcagucaauuguaguacuucauguaucaugaauucaaguacuaugaucagacagacaucucucucucucucacacacacacacacacacacacacgcacacauacacacacacacacacaccugaggaaauggcugcuuuggguucuauaaggaccauuccauguuuaaaguccuaguugagcugaaugcuaagaaccugcccccuugccucccucugagaugauaucauuuccuggcuucgucaaugcugccugucuauuugcaugcuggguucugaggacuagugagaaggugaccagaguuuggguggggcugguuuuuacccacuggauuuggugagaauaugaagcauccaguguguaccaggguuucugaaccacgggaaaggcguaggaaaacaaacauucagagccccuguaaaacgagaaaggaaaaaccagccaguguugcauuccacaucucugcuuguugcauuuugcuaauaugggguuauucuuucucacuguuaggaugcaauugugugcaaagacaguggcugagugaacaguaagagcuggcuaguaauggccuuaaaaagaaaaaggguaacucucugaaacaaagaucacuuuaguguggcauuguggaugcuguuaauucugcauagggaaacuuuggaacagcaugcuaauuacauggcuguaagcaaagcccuguccucugucucugcaccauaccuucauuggacuucaccaacccauccauacuccauguaaaccucaguucucucaugccugcccuaagucaguugacaucagugcaguggcauugaggagaaaugagaggugucucugauuuuacugaaaguuauuaucauuuucacaggugccugagauuugguaucuacuuuguguucuugauucuuaggugaaaaaucugaaauaguucccugugcauuaaaauaaauuauuuugagaggacuccugcuccgucgauucagcagaccuaugcugcagaagguaacugcggaagcucucuuuugcugucagggcucugagcuugaagggagaaggugcaguggugccuagaagugauaugcaaaccaccucacaugccagccucuggccuccuucccaucccagagucacagacaggggacccagugacaaugaugauaaauccauguguggagguguuuuacuuauuuuucuuuccguaggauuucauggugcuuuaaaaaaaaaggcauuuuacagaaaauaauguggggggagggagauuucauaauguucuuagggaaaguacaaaacaaauuugcuugugacauuucaauaagcugugcugcuauugucuuuauuugaugauguaauuuuuuuuucaaugauggagaaaaauugcaacaaagaccuucuggaagauccaaaaaaaaaaaaaaaaaaa

SEQ ID NO: 11 - RNA, 7527 nt, Homo sapiens
gugugcgggugugugugugcgggagugugugcgcacacgccuucgggggcuggagugacacugcagugggcuucuccguagccccagccuucaggcugcgcagccccuaaccugccggcaccgcuuuugggaagcagcuuggcucuuccaucuccccaagccuuccucuccauccuuucauccaccccucgccgaucuuacuuuuucuuuuacccacggggccgcggccggccacccccuggccggugcaaacacccaagcccucuccaguucucccccaagcuaauauuuucccaccugucuuuuucugggguuccuccacgagccaguccaaggcucccccauccucggaaauuguuuuguuggacugcuaaaccgaggcguguaaagcuugaggacuuuauuauuauuuggguucuuuucauuucuuccccuucugggcaacgaagcaaugaaauuuccaaucgagacgccaagaaaacaggugaacugggauccuaaaguggccguucccgcagcagcaccggugugccagcccaagagcgccacuaacgggcaacccccggcuccggcuccgacuccaacuccgcgccuguccauuuccucccgagccacagugguagccaggauggaaggcaccucccaagggggcuugcagaccgucaugaaguggaagacggugguugccaucuuugugguugugguggucuaccuugucacuggcggucuugucuuccgggcauuggagcagcccuuugagagcagccagaagaauaccaucgccuuggagaaggcggaauuccugcgggaucaugucugugugagcccccaggagcuggagacguugauccagcaugcucuugaugcugacaaugcgggagucaguccaauaggaaacucuuccaacaacagcagccacugggaccucggcagugccuuuuucuuugcuggaacugucauuacgaccauaggguaugggaauauugcuccgagcacugaaggaggcaaaaucuuuuguauuuuauaugccaucuuuggaauuccacucuuugguuucuuauuggcuggaauuggagaccaacuuggaaccaucuuugggaaaagcauugcaagaguggagaaggucuuucgaaaaaagcaagugagucagaccaagauccgggucaucucaaccauccuguucaucuuggccggcugcauuguguuugugacgaucccugcugucaucuuuaaguacaucgagggcuggacggccuuggaguccauuuacuuugugguggucacucugaccacggugggcuuuggugauuuuguggcagggggaaacgcuggcaucaauuaucgggagugguauaagccccuagugugguuuuggauccuuguuggccuugccuacuuugcagcuguccucaguaugaucggagauuggcuacggguucuguccaaaaagacaaaagaagaggugggugaaaucaaggcccaugcggcagaguggaaggccaaugucacggcugaguuccgggagacacggcgaaggcucagcguggagauccacgauaagcugcagcgggcggccaccauccgcagcauggagcgccggcggcugggccuggaccagcgggcccacucacuggacaugcugucccccgagaagcgcucugucuuugcugcccuggacaccggccgcuucaaggccucaucccaggagagcaucaacaaccggcccaacaaccugcgccugaaggggccggagcagcugaacaagcaugggcagggugcguccgaggacaacaucaucaacaaguucggguccaccuccagacucaccaagaggaaaaacaaggaccucaaaaagaccuugcccgaggacguucagaaaaucuacaagaccuuccggaauuacucccuggacgaggagaagaaagaggaggagacggaaaagauguguaacucagacaacuccagcacagccaugcugacggacuguauccagcagcacgcugaguuggagaacggaaugauacccacggacaccaaagaccgggagccggagaacaacucauuacuugaagacagaaacuaaaugugaaggacauuggucuuggacugagcguuguguguguguguguguguguuuuuaauauucacacugagacaugugccuuaaacagacuuuuuaguccaaaauuacauagcauugaagaauauauuucacugugccauaaacaacugaaagcuugcucugccaaaaggaaucagagaacaagaacuucauuucagauagcaaacgcaggacacaccaagaguguccgugcacguagccgguucuggccguacauguuaagggcauuucaguggcagugcuguaccccugggcagugcuaccugggcacacacguagacaagggcagcuauuccuuagaccagccuccugaaagaaacaggugugucuuuuuaguggagucguaguaauaugugcacacacagaaggggaccugauugggugggagcugguuauguguaacuagcguuggaguugacauuuuggcaugugcucugagcuugaauuuugauaccaaccauucagugcaucauaccuagucuuucuaugcuccaaaugaaugucuguggggaccugagagcaccuggaauuuguuggaagcagaucagagcacacguacgaaaaggugcaauugcuuuucucaugacaaaagaaaaaaaauaaucacaaacacaucacacuguggauaucaccuuaaccaaugacagaagccugcugaguuuggucuuucucacagggguagguaguccacaugaacuggcgagcaguggugaaaacaugacaucaacugcugcgugguuaaaggccaaucaugcauuuuccucagugggugcaauggugacaauaaacugauuuggaaauguccauguacuuugguuauaacuuguuuugauaagagggguagaaauaccaaaacaauugccuugcaugaugccaguggcuugcuguucugucuagcugauccuacauuuuuacaaaaguagacauccugcauuucugagacacacugaggaggaccauggcaucuuuccaucucagggcuuuuugucccuugggugucuuaggguagacauagugauaagauccaaucucgcuaucuccaaacaaaaagaaaauguaccccuuguacaguaagcuugaaauguuuuuauugcuugcagcguaagugccauuaauuccauuuaacaaugauacuuagaaauauuuaaaauauuucauuuuacuuuuauguuugggcauuccuuuauucugaaaaagucaccacugcuccuugcuuguuucuacaucucacaucgguuacucacuaaaacuggaacacccuagacugauuguucccaggcaaacgauaagcuaacacaucauaauuaugauuggucugugaggcuagaacaucccgcagaggaaagucggggaauggcuagucauagguugcaugaugauuuuaaauggguuuguaucagaaaggggaaaauacauacuccuguguaugccucagucuuuaucauaauuggcugguucacagcuagcucuuugggccacacgugaaagcauuuggaccuggcaacaccaggcaguugcugguuauggguguucuaggcucacugcuaccuuuuacagaguucacgggauuuuacguaacuaacccgcaguguggauuuaccaaaagaucacagcacuagcggagacuuccagaaacugucucaugcuuucugaccuccagggaaaguuguacccacgacauccucaaccacugguaccauggaagaccugucccugcccauuucucaaaaaugcacagcucuucccugccuggagcacaugaagucccaucucccuguccccagaccuuuaggcucuguugaaguucuggcauugcuuaugagacccuuccuguccaaggaaggucuaagucaacuccuccauuggacuggagaugaggcucacuggugacauccugugucgacucaguaauaaauagucacuugggaggcgauguuuaugcagguguggauguugaguuuuacuaugggaaucauuccagccugacaaaauguguuuuaaggaauagguagauaacugggacuuccucuguuauucuaacaagucgaagcuuccuaauuaaacuguccauggaucucacuuauuugguuuccacucuguguuaucugguacguucuuuguagcaguguguuaaaauuccaggcccuacggccaaaucuggcagaggcuucccucucauuuauccagugccuucugugggccaggcacaggauuaggcacuuuaugaacgucagcucauugaaacuucacgacacuaugugggaaguauuugcaaacccauuauauacacaaggacacagaggcucagggaagcuauguuauuuauccaauggcacacagcaagaaagugguagagcugguauucaaagucugaccaaagucugcuuucaacuucacuacagugucugugucaugcauccuugugauuguguguguuuguguguaugugugucuuucuagcuucauggacacagcuuacagauguggggagcagauaugguggaaucuccaccaccaagagggcacaaggucuuuguguaaacauggcucaaaggguugccccugcagacaccuacuguacauuuauuugguuuuggaaauuuuguauguggcacccuuuaaaaaaugcccuuugaaagcacucuuuugcacuuuacuugcuaacuuuguagaaacucugcauacagcaggaauaaaauaguucaaagcacuaagcugcauacucuaccaaauggaacaggugcauguguugguaugugcauagaugcuuccccaaaugagucaaaucagucacacagagggaucaaacauaaccuugggcugggggugggaaaauuuucuacauaacccauucccugagacauuuggccaagaaugugaugaacaaaaucaaagaagauccucuauggugauugaucgauuaaauaugugugcaaaguguuuagaaaccuaugaaauacucucgcaaagaugcugagagagaauaagagguuggauuccucuucauauaaacuaauuuuggaggaggccaguugguuugaaguuacuugaauguuaccuuuuuuagauggggccaaauggcauguagaauacacgugauaggucaaagcugcuacacauucuauacaugcaucagcacagcccccccuuuccaaucugcacucccauuccagcauaaaccuaggagaaauguuucgauuucacacaaagaaagagcacacguccaccaucuucagugggggcugucuuuugcuucacuggcaagcaggcacugaauuuuucuugcaugacaaaucuggagguuuacuggugagagagccaaugggcauuuuuuccuggaaagaguacagcuccauacccaguccuaacccaacagugauauuuaucacuuuggggcagggcuguauagagugugugugugugugugugugugugugugugugugugugugugugugugcggguuggggugguguugggccaucucuggccuguuacuaagguaacuaggacuauuuguguuccagcagucauagccugugauugugggugcaucaguucucugccuagaucucuuguuaccuugucugcacaucaagggagggaguugagcacagauacuugucaagggccauuguaguugugcaguucucuaaugaaacacucccuaguccaugaguucacaaaauuuauuaagauuaaauuauaaguuggauuugugaauaaugacuaauuaauugucuugcccauuuuagguuaaggugagagcuuagucucuugcccuuugggauuugucuuuugggggauuaauggagaccagauguacuugggagacugguguccaaauucggaucaugcccuguguaggcucucucuauccucccuuauagcucuuuaguguacugucaccgggagggcucaugcugugagggcauuuuuugcaugguguuaagacuaguuaaagaaauuuuaagcuguuguuauuugcagucaauuguaguacuucauguaucaugaauucaaguacuaugaucagacagacaucucucucucucucacacacacacacacacacacacacgcacacauacacacacacacacacaccugaggaaauggcugcuuuggguucuauaaggaccauuccauguuuaaaguccuaguugagcugaaugcuaagaaccugcccccuugccucccucugagaugauaucauuuccuggcuucgucaaugcugccugucuauuugcaugcuggguucugaggacuagugagaaggugaccagaguuuggguggggcugguuuuuacccacuggauuuggugagaauaugaagcauccaguguguaccaggguuucugaaccacgggaaaggcguaggaaaacaaacauucagagccccuguaaaacgagaaaggaaaaaccagccaguguugcauuccacaucucugcuuguugcauuuugcuaauaugggguuauucuuucucacuguuaggaugcaauugugugcaaagacaguggcugagugaacaguaagagcuggcuaguaauggccuuaaaaagaaaaaggguaacucucugaaacaaagaucacuuuaguguggcauuguggaugcuguuaauucugcauagggaaacuuuggaacagcaugcuaauuacauggcuguaagcaaagcccuguccucugucucugcaccauaccuucauuggacuucaccaacccauccauacuccauguaaaccucaguucucucaugccugcccuaagucaguugacaucagugcaguggcauugaggagaaaugagaggugucucugauuuuacugaaaguuauuaucauuuucacaggugccugagauuugguaucuacuuuguguucuugauucuuaggugaaaaaucugaaauaguucccugugcauuaaaauaaauuauuuugagaggacuccugcuccgucgauucagcagaccuaugcugcagaagguaacugcggaagcucucuuuugcugucagggcucugagcuugaagggagaaggugcaguggugccuagaagugauaugcaaaccaccucacaugccagccucuggccuccuucccaucccagagucacagacaggggacccagugacaaugaugauaaauccauguguggagguguuuuacuuauuuuucuuuccguaggauuucauggugcuuuaaaaaaaaaggcauuuuacagaaaauaauguggggggagggagauuucauaauguucuuagggaaaguacaaaacaaauuugcuugugacauuucaauaagcugugcugcuauugucuuuauuugaugauguaauuuuuuuuucaaugauggagaaaaauugcaacaaagaccuucuggaagauccaaaaaaaaaaaaaaaaaaa

SEQ ID NO: 12 - RNA, 3623 nt, Homo sapiens
aguguaucacaaacucaagcauuagcaccaacaagcucugagcaucaucagucucuggaaagccuucugaauuagacaagggcugccucccagcacagcuacaaaacacuuuaaaccugaccagcuaaauggauaaaccuagccugcauagcuuuuaaacuggggucucauacagcacaggaggccuacuugcuucaagaacugaaaauccagaggaugaauugcuuuaucugggaauggcaaaagccagcacaauaaggaaugccaguuuguauggggcuacuagcucacaugcgggaucagaauggugugaaugacagccgcacugugucaugaaggugguggugguuuccgcacaagagaccaaauaagaagaaagcugagagaggggggaaacguuuuuggaugacaaaggauggguuuccauuuaauuacgcagcugaaaggcaugaguguggugcuggugcuacuuccuacacugcugcuuguuaugcucacgggugcucagagagcuugcccaaagaacugcagaugugauggcaaaauuguguacugugagucucaugcuuucgcagauaucccugagaacauuucuggagggucacaaggcuuaucauuaagguucaacagcauucagaagcucaaauccaaucaguuugccggccuuaaccagcuuauauggcuuuaucuugaccauaauuacauuagcucaguggaugaagaugcauuucaagggauccguagacugaaagaauuaauucuaagcuccaacaaaauuacuuaucugcacaauaaaacauuucacccaguucccaaucuccgcaaucuggaccucuccuacaauaagcuucagacauugcaaucugaacaauuuaaaggccuucggaaacucaucauuuugcacuugagaucuaacucacuaaagacugugcccauaagaguuuuucaagacugucggaaucuugauuuuuuggauuuggguuacaaucgucuucgaagcuugucccgaaaugcauuugcuggccucuugaaguuaaaggagcuccaccuggagcacaaccaguuuuccaagaucaacuuugcucauuuuccacgucucuucaaccuccgcucaauuuacuuacaauggaacaggauucgcuccauuagccaagguuugacauggacuuggaguuccuuacacaacuuggauuuaucagggaaugacauccaaggaauugagccgggcacauuuaaaugccuccccaauuuacaaaaauugaauuuggauuccaacaagcucaccaauaucucacaggaaacugucaaugcguggauaucauuaauauccaucacauugucuggaaauaugugggaaugcagucggagcauuuguccuuuauuuuauuggcuuaagaauuucaaaggaaauaaggaaagcaccaugauaugugcgggaccuaagcacauccagggugaaaagguuagugaugcaguggaaacauauaauaucuguucugaaguccagguggucaacacagaaagaucacaccuggugccccaaacuccccagaaaccucugauuaucccuagaccuaccaucuucaaaccugacgucacccaauccaccuuugaaacaccaagcccuuccccaggguuucagauuccuggcgcagagcaagaguaugagcauguuucauuucacaaaauuauugccgggaguguggcucucuuucucucaguggccaugauccucuuggugaucuaugugucuuggaaacgcuacccagccagcaugaaacaacuccagcaacacucucuuaugaagaggcggcggaaaaaggccagagagucugaaagacaaaugaauuccccuuuacaggaguauuauguggacuacaagccuacaaacucugagaccauggauauaucgguuaauggaucugggcccugcacauauaccaucucuggcuccagggaaugugagguaugaaccaugauccuccuaaaagcauuucuacugcggggaaggagagguaaauguuugaagcccuagaggugucucuaaucacuagaaagauuaaugacccuuuugcuuuuggguuuugcucagugugaaagguuacuuaauuaaauuacaaccaccaggaaauugacugcuuuuuuuuuuuuuuuaaaugguugaaacuugaaggaaguucauucaaggauaaguuggaauaaagcacuauguuaaaacaucugcuuuuuaacaauuuguauacagggguuggacuuaaaaacacacauacaaacaaaacucuuuucauucugaaauuucugucugguucuugguguuugacuguuguaauggaguaaagaagagggccagcuuaauuuaaaaauaaagugaguuuaccaaaauuccagaagguaaugaagauuuaaaccaaagagcaauuuucccaaggguugauuuuguuguaaauuuuuaguuuuaaugaaagcaugcaacagggaucucacacccauguuacuugccauuuaguuauuauaccagcauagagaaugucagaggccuacuguguaauuguaucaggcuccuaaggcuucuuucuuuuuuuuuuuuuguuuccuucuuuccuacuuucuuucacuccuuccuuuccuuccuuucucuuccuucuuuuuuuguuuguuuguuuguuuuuuaauuacugggauauagauccgauuucaaagguuuuuaugcacuggcuauuuguguuuaaucagaaugaaacuucaauuccaugcuuuggcuuugucccugguaacuaaaaucaggucacaauuuuguggaugauuuagcaauaauaaaauggcagucauaacagggacuguuugucaguauugcugucauccccaaaagaaaugauauguauucuuguuaaacuuauuaaaaaauaagugauacaaauauuuauaaauaaaaggagagagguuugaguucuggguauccucccuuucuguaacagccucaaauaaagguguaccuuccauguuauauuauuuuuguuuuuauaagaauaaauuuggacccgguuucugauuauuuaauuuaaaagaucacuuacaggaucauguuagugagcagacaaauagucacaaauuucaaacuugucaauaaguaaugcuggauguaaauuguucuuuucuugguaauguugaucauuaugugcaaagacugacuacuaaggccuuguugcuggaagaaacuagugaagagagguuaaauucuggcaauauaauguuuuaguaucugauuuuagaugaucacucuuucaaaagacaguuuuaaugaguaauuaaagauguugcccuuucuaaugcuaguuaacaguuggaaaauauaauugcucuuugauagcauauacuccaauuuuuccauaguccuaacuacaaaugcuaaugaaaauguuuucagacaaagcacugaauuugaaauaaucucuaacucaguacaauuuggacuauugaacuguucuucaaagaauucauuuucuucuuuuuuguguaucauugaaaauaagagaaguaaucccagauaauaaauuccuaaaaggaaacuguauaagcaauugcccuuaaaaauuaaaaagaauaaaagauuuucuauaaaaguc

SEQ ID NO: 13 - RNA, 3167 nt, Homo sapiens
aguguaucacaaacucaagcauuagcaccaacaagcucugagcaucaucagucucuggaaagccuucugaauuagacaagggcugccucccagcacagcuacaaaacacuuuaaaccugaccagcuaaauggauaaaccuagccugcauagcuuuuaaacuggggucucauacagcacaggaggccuacuugcuucaagaacugaaaauccagaggaugaauugcuuuaucugggaauggcaaaagccagcacaauaaggaaugccaguuuguauggggcuacuagcucacaugcgggaucagaauggugugaaugacagccgcacugugucaugaaggugguggugguuuccgcacaagagaccaaauaagaagaaagcugagagaggggggaaacguuuuuggaugacaaaggauggguuuccauuuaauuacgcagcugaaaggcaugaguguggugcuggugcuacuuccuacacugcugcuuguuaugcucacgggugcucagagagcuugcccaaagaacugcagaugugauggcaaaauuguguacugugagucucaugcuuucgcagauaucccugagaacauuucuggagggucacaaggcuuaucauuaagguucaacagcauucagaagcucaaauccaaucaguuugccggccuuaaccagcuuauauggcuuuaucuugaccauaauuacauuagcucaguggaugaagaugcauuucaagggauccguagacugaaagaauuaauucuaagcuccaacaaaauuacuuaucugcacaauaaaacauuucacccaguucccaaucuccgcaaucuggaccucuccuacaauaagcuucagacauugcaaucugaacaauuuaaaggccuucggaaacucaucauuuugcacuugagaucuaacucacuaaagacugugcccauaagaguuuuucaagacugucggaaucuugauuuuuuggauuuggguuacaaucgucuucgaagcuugucccgaaaugcauuugcuggccucuugaaguuaaaggagcuccaccuggagcacaaccaguuuuccaagaucaacuuugcucauuuuccacgucucuucaaccuccgcucaauuuacuuacaauggaacaggauucgcuccauuagccaagguuugacauggacuuggaguuccuuacacaacuuggauuuaucagggaaugacauccaaggaauugagccgggcacauuuaaaugccuccccaauuuacaaaaauugaauuuggauuccaacaagcucaccaauaucucacaggaaacugucaaugcguggauaucauuaauauccaucacauugucuggaaauaugugggaaugcagucggagcauuuguccuuuauuuuauuggcuuaagaauuucaaaggaaauaaggaaagcaccaugauaugugcgggaccuaagcacauccagggugaaaagguuagugaugcaguggaaacauauaauaucuguucugaaguccagguggucaacacagaaagaucacaccuggugccccaaacuccccagaaaccucugauuaucccuagaccuaccaucuucaaaccugacgucacccaauccaccuuugaaacaccaagcccuuccccaggguuucagauuccuggcgcagagcaagaguaugagcauguuucauuucacaaaauuauugccgggaguguggcucucuuucucucaguggccaugauccucuuggugaucuaugugucuuggaaacgcuacccagccagcaugaaacaacuccagcaacacucucuuaugaagaggcggcggaaaaaggccagagagucugaaagacaaaugaauuccccuuuacaggaguauuauguggacuacaagccuacaaacucugagaccauggauauaucgguuaauggaucugggcccugcacauauaccaucucuggcuccagggaaugugagaugccacaccacaugaagcccuugccauauuacagcuaugaccagccugugaucggguacugccaggcccaccagccacuccaugucaccaagggcuaugagacagugucuccagagcaggacgaaagccccggccuggagcugggccgagaccacagcuucaucgccaccaucgccaggucggcagcaccggccaucuaccuagagagaauugcaaacuaacgcugaagccaacuccucacuggggagcuccaugggggggagggagggccuucaucuuaaaggagaauggguguccacaaucgcgcaaucgagcaagcucaucguuccuguuaaaacauuuauggcauagagaaaagaaaaagagaaaaaaaaacugcuugaagagggcagcgaaugcaccugcagcauuaccgggaggacugcagaacuuucuuugcuuuaaaacauaaaacaaacaaaaaugcucccggauguaaauaguaagagugucaaguucuuauaaaaauagauuucacccuagcuacauauagguuuguuuguauauuugauuuuucuacacugcaugguuccugggcuggagaaccacaggggagcaaauaaacaggggugggugggaagcuagggaaacaaccguuuuccucuuuagguguguguaauucucacagauuuuauuuaaauugcuuucauuuauuuuuucuuuaaaaaugcuguuuguucuuguuuuguuuguuuuaauuuuuuucuuuccuggucaaguuucuaauacaaaacaugagcauuaaagcuuauguuguaccaaaagaaaaugugcuuuaccacggaccacggagaaaguugaccacaaaguuugacaaaacuacuggaaaacccaauaagucugggugaaugacccuucaugacaaaugccauauaugguacaugauagaaggaauuuuguuuugucccauuggcagcccucacuguguaggaugccugagagagccaugaagaucuccgaaugagaagaccagcagaaucauucucauguggacuaaggcuauaagagagauaugaccaugggcucaaugaaugaaacugccaagcugacaaauuucuaaacacaagauaauguguaacauugcuuuuuaaauuaauuaucaauuagcuuuuguguaguuuuuaucaauaaaagaaaaaauauugaaaaauaaaaaaaaaa

SEQ ID NO: 14 - RNA, 4370 nt, Homo sapiens
acaacccuccuagaggcgggacgguuuccugccaaucacugcugguuugcccucccccugguguuggcccggcugacucgggguaggggaggucggggagggggcgauaaaauggcgcagggggcggagugaggcgcagucguucgcccaggcuuuggcccggcuuccggaggaaggugggagucaaucauuuugacaagucuccugaaaggaacagcuagcaggaacugaaaccuuuuuccauuuggucucguggcaaaggcagagauugcuccagcagcuccacacaaaaugaugugcucacgggugcccucugaacagucuucugguaccucucucuugccuaaagacggugccccauuuucuugggauuccuuggaugaggauggauuggaugacuccuugcuggagcugucagagggagaagaagaugauggugauguaaauuacacagaggaagagauugaugcacuguugaaggaagaugacccaucauaugagcagucuucuggggaagaugauggugggcauguugagaagggagaaagagggagucaaauucuacuugauacuccccgagagaaaaauucaucguacagccugggaccaguagcugagacuccugaccucuucaaacuaccucagcuaaguacaucaaguggucauggaccagcucauacuaaaccauuaaacagacgcucuguacuagaaaagaaucuuauaaaaguaacuguugcaccauuuaauccaacaguuugugaugcucugcuugauaaggacgagacugauucguccaaagauacugaaaaacucucuucccuuggagaagagaugagagaagauggucuuagcccaaaugaaagcaaacuuuguacugaaucugaagggaucagccccaauaacucugccuggaaugggccccagcucucuucuucaaacaauaacuuucaacagacugucucugauaaaaauaugccugacagugagaacccuacgucuguauucucucggaucucagaccauucagagacuccuaauauggaguuauccugcagaaauggugguucacacaagucaaguugugaaaugagaucucugguuguuuccaccucaucaaacaaacaggauguucuuaacaaggauucugggaagaugaaaggccaugagagaagacuaggcaaagucauuccuguucuacaaacuaagaccaggacuaauguuccgacguuuucacagucaaaucuagaacagcagaagcagcuuuaucucaggagugucauugcucauauagaagacccagaggacacuaaccaagguaucucgggggagcuuugugccuugauggaucaaguucaucauaugcagcacucaaaauggcagcauccuucggaccucaccacgcgaaacuacgcccgccgacagaaacaucugcaaagauacagucugacucaguggguugacaggaacaugcgaagccaccaucgguuccagcgucucccagacuucucguacaguuaauuugugucaucccaucagcaaugaaggucccuauccaggguccugcuuggagcagcauuucauguucuuuugcuguuuugugcuuugccgauuuuggauuuuauuuuucacaaaauuuuuauuuaaaaaacucgucaccuuuuggaaaugcccauugccgacuugaauuuuuuuguaugaagucccuccugauuuugugugugugugucuguguuuaagcaagcguucgguugguauaguuuuuuuuuguuuuuuuaauuuaaauugaagguagcugccuccugaaagccagcauuaagccagaacacccagguucaagcaaaagacccaccucucugcagaggcaaagucuacuuucugguaccucaaagaaaucauuguucaaucuuccauaaggaagagauucuuuaccaggcugugagccaguguuagauaacuugugaauggauauaaguuacuuuuaacaaccccucuuacuuuuuuauuugaauccucugaauaccugucaguauuuuaaaguuggcaauccaggacauuauaaguaggauggagcaggagaagaauccuauugaaaggacaaauuaaaauaguaaauccucuccucucccuucuguagguacuagcugccuugauuuuuuuuugugggugggggggauuuuuucauuccuuauugaacuuauuuugcacaauaguguuuacaaaucuuauacaucuuacuuugacagaagacuuauaauaccuuguggucuugagacugguugucuccuuugccuuucuucaaggauuuuccuuuuucuuuuuaccccccagugaccacucugcucuuuaggauucuaguggcaguauucuuguaaccuguuagacguggauauaccucuucaggauggccuccuugcugucuuuauugaagagcugcccacccagagucuugacucuagggcaggcuagugcuauuguggugucaguugauauuuaguuucauuugcugaaccccaaauuugaguguuaguuagggagccuccauucugugggagaguguggaaaggcacucuuuuuucuccauauuaguuaacaagaggugucuuuaggcccuggauguuaccaucucauuuggucaagccccugauaccuaguuucacauggaugcuaacguggauguaauauaguugugacagccuaugcacaaaagcauuaccugaucuuaagguuugguauucuaaccaaaguugacagacugugcgucugucuuaauggaugucuuaggaguugguuauucucucuuuuuuuuuugagacggaguuucgcuguuguugcccaggcuggagugcaauggcguaucuccacucaccacaaccuccgccuccuggguucaagggauucuccugucuuagccuccugaguagcugugauuacaggcaugcgccaccaugccuggcuaauuucauauuuuuuaguagagacaggauuucuccauguuggucaggcuggucuugaacuccugaccucaggugaucugcccaccucggccuccuaaagugcugggauuacaggcaugagccacugcaccuggccgguuauucucucuuuacagauagcuauagacaucauuuuaggaaguguugcagucuggcauuugugcuauuguucauucucugugaaggcuguucauaguugcuauagccuguguuuaguuuugugauuucaucaaucccaucuuucugugugaguaaugcauucuaaacauccuaccccacuuuagaaacggacguggggaacgcuuggucauuuaagccaacaauaaauuuaggugaaugucccuaaguguuuacuguuuuuauccagucaaggauuugcuuuuccuugaacauuuguuuuaaauucuggggccaaaaugcaaaggagaaguucuauucaaaggcaguaguugaaaucuauuauuuuaguuagccuacuuggcauuuacuacaucggucacuucuccaggcugcccuaaauuagguugauggagugagacaugccaaacauccaccuuugggaccauagcauaguuaaaauuaaauguaguuggaauagcuagcauugcagcuacaguagggaacuguagucuaguucccuacagaaaacccaaggagugaagggacaggauuuugccuaggcaaaaaucuaagacucgugcccuccugguacaugggguuuuaagacugaauguguaauaggagcacugccuuugccaaaucaaaugagugacagguuaacuagaaaaugugacaaucacauuuccucuuagcucaaauaauucuguuuuuccaaagcuuuagcagcuuaauuaaaucuguuggacugggggaggagagagcuguucucuagugguuaacaugguauucuuuaagaagaaaaaacaaagccaaagaaaacucauuaucuggcauguucgccuuaaagaugguacuggguagaaucuggaguuuucaucucuuuucaaagcugcauaucucuuauauuugguauuggccucuaagucuaauauugcaguuggaauucuugcuguauuauuuuuuaagcaaguguuaggugcauuuaacugcuuucuucauccaugacgacauucccaccaugggggucuugacaaagcagaguaaaaauaugcuguuuacauuguuuacuuacaaguaaggagccugaaauaaccuguaguuucgaaugcaggcccugauuuacuggcguugucaguuucaauuaugaaacugaaguuuggugccuccucuuuaucauguuuuuucccuuguagcaguuguguuuaaugucauuaaaaagaaauaaaaguucuuugucagugacauaaaaaaaaaaaa

SEQ ID NO: 15 - RNA, 4356 nt, Homo sapiens
gaugagacgggaaaagguggagccagugggcacggugggcggugcggagggcggggcugaggcgccugagcggggcacaacccuccuagaggcgggacgguuuccugccaaucacugcugguuugcccucccccugguguuggcccggcugacucggggaaggugggagucaaucauuuugacaagucuccugaaaggaacagcuagcaggaacugaaaccuuuuuccauuuggucucguggcaaaggcagagauugcuccagcagcuccacacaaaaugaugugcucacgggugcccucugaacagucuucugguaccucucucuugccuaaagacggugccccauuuucuugggauuccuuggaugaggauggauuggaugacuccuugcuggagcugucagagggagaagaagaugauggugauguaaauuacacagaggaagagauugaugcacuguugaaggaagaugacccaucauaugagcagucuucuggggaagaugauggugggcauguugagaagggagaaagagggagucaaauucuacuugauacuccccgagagaaaaauucaucguacagccugggaccaguagcugagacuccugaccucuucaaacuaccucagcuaaguacaucaaguggucauggaccagcucauacuaaaccauuaaacagacgcucuguacuagaaaagaaucuuauaaaaguaacuguugcaccauuuaauccaacaguuugugaugcucugcuugauaaggacgagacugauucguccaaagauacugaaaaacucucuucccuuggagaagagaugagagaagauggucuuagcccaaaugaaagcaaacuuuguacugaaucugaagggaucagccccaauaacucugccuggaaugggccccagcucucuucuucaaacaauaacuuucaacagacugucucugauaaaaauaugccugacagugagaacccuacgucuguauucucucggaucucagaccauucagagacuccuaauauggaguuauccugcagaaauggugguucacacaagucaaguugugaaaugagaucucugguuguuuccaccucaucaaacaaacaggauguucuuaacaaggauucugggaagaugaaaggccaugagagaagacuaggcaaagucauuccuguucuacaaacuaagaccaggacuaauguuccgacguuuucacagucaaaucuagaacagcagaagcagcuuuaucucaggagugucauugcucauauagaagacccagaggacacuaaccaagguaucucgggggagcuuugugccuugauggaucaaguucaucauaugcagcacucaaaauggcagcauccuucggaccucaccacgcgaaacuacgcccgccgacagaaacaucugcaaagauacagucugacucaguggguugacaggaacaugcgaagccaccaucgguuccagcgucucccagacuucucguacaguuaauuugugucaucccaucagcaaugaaggucccuauccaggguccugcuuggagcagcauuucauguucuuuugcuguuuugugcuuugccgauuuuggauuuuauuuuucacaaaauuuuuauuuaaaaaacucgucaccuuuuggaaaugcccauugccgacuugaauuuuuuuguaugaagucccuccugauuuugugugugugugucuguguuuaagcaagcguucgguugguauaguuuuuuuuuguuuuuuuaauuuaaauugaagguagcugccuccugaaagccagcauuaagccagaacacccagguucaagcaaaagacccaccucucugcagaggcaaagucuacuuucugguaccucaaagaaaucauuguucaaucuuccauaaggaagagauucuuuaccaggcugugagccaguguuagauaacuugugaauggauauaaguuacuuuuaacaaccccucuuacuuuuuuauuugaauccucugaauaccugucaguauuuuaaaguuggcaauccaggacauuauaaguaggauggagcaggagaagaauccuauugaaaggacaaauuaaaauaguaaauccucuccucucccuucuguagguacuagcugccuugauuuuuuuuugugggugggggggauuuuuucauuccuuauugaacuuauuuugcacaauaguguuuacaaaucuuauacaucuuacuuugacagaagacuuauaauaccuuguggucuugagacugguugucuccuuugccuuucuucaaggauuuuccuuuuucuuuuuaccccccagugaccacucugcucuuuaggauucuaguggcaguauucuuguaaccuguuagacguggauauaccucuucaggauggccuccuugcugucuuuauugaagagcugcccacccagagucuugacucuagggcaggcuagugcuauuguggugucaguugauauuuaguuucauuugcugaaccccaaauuugaguguuaguuagggagccuccauucugugggagaguguggaaaggcacucuuuuuucuccauauuaguuaacaagaggugucuuuaggcccuggauguuaccaucucauuuggucaagccccugauaccuaguuucacauggaugcuaacguggauguaauauaguugugacagccuaugcacaaaagcauuaccugaucuuaagguuugguauucuaaccaaaguugacagacugugcgucugucuuaauggaugucuuaggaguugguuauucucucuuuuuuuuuugagacggaguuucgcuguuguugcccaggcuggagugcaauggcguaucuccacucaccacaaccuccgccuccuggguucaagggauucuccugucuuagccuccugaguagcugugauuacaggcaugcgccaccaugccuggcuaauuucauauuuuuuaguagagacaggauuucuccauguuggucaggcuggucuugaacuccugaccucaggugaucugcccaccucggccuccuaaagugcugggauuacaggcaugagccacugcaccuggccgguuauucucucuuuacagauagcuauagacaucauuuuaggaaguguugcagucuggcauuugugcuauuguucauucucugugaaggcuguucauaguugcuauagccuguguuuaguuuugugauuucaucaaucccaucuuucugugugaguaaugcauucuaaacauccuaccccacuuuagaaacggacguggggaacgcuuggucauuuaagccaacaauaaauuuaggugaaugucccuaaguguuuacuguuuuuauccagucaaggauuugcuuuuccuugaacauuuguuuuaaauucuggggccaaaaugcaaaggagaaguucuauucaaaggcaguaguugaaaucuauuauuuuaguuagccuacuuggcauuuacuacaucggucacuucuccaggcugcccuaaauuagguugauggagugagacaugccaaacauccaccuuugggaccauagcauaguuaaaauuaaauguaguuggaauagcuagcauugcagcuacaguagggaacuguagucuaguucccuacagaaaacccaaggagugaagggacaggauuuugccuaggcaaaaaucuaagacucgugcccuccugguacaugggguuuuaagacugaauguguaauaggagcacugccuuugccaaaucaaaugagugacagguuaacuagaaaaugugacaaucacauuuccucuuagcucaaauaauucuguuuuuccaaagcuuuagcagcuuaauuaaaucuguuggacugggggaggagagagcuguucucuagugguuaacaugguauucuuuaagaagaaaaaacaaagccaaagaaaacucauuaucuggcauguucgccuuaaagaugguacuggguagaaucuggaguuuucaucucuuuucaaagcugcauaucucuuauauuugguauuggccucuaagucuaauauugcaguuggaauucuugcuguauuauuuuuuaagcaaguguuaggugcauuuaacugcuuucuucauccaugacgacauucccaccaugggggucuugacaaagcagaguaaaaauaugcuguuuacauuguuuacuuacaaguaaggagccugaaauaaccuguaguuucgaaugcaggcccugauuuacuggcguugucaguuucaauuaugaaacugaaguuuggugccuccucuuuaucauguuuuuucccuuguagcaguuguguuuaaugucauuaaaaagaaauaaaaguucuuugucagugacauaaaaaaaaaaaa

SEQ ID NO: 16 - RNA, 23 nt, Homo sapiens
ucuuugguuaucuagcuguauga

SEQ ID NO: 17 - RNA, 23 nt, Homo sapiens
acugcccuaagugcuccuucugg

SEQ ID NO: 18 - RNA, 23 nt, Homo sapiens
acuccauuuguuuugaugaugga

SEQ ID NO: 19 - RNA, 21 nt, Homo sapiens
ucagugcaugacagaacuugg

SEQ ID NO: 20 - RNA, 22 nt, Homo sapiens
uggagagaaaggcaguuccuga

SEQ ID NO: 21 - RNA, 22 nt, Homo sapiens
uccuucauuccaccggagucug

SEQ ID NO: 22 - RNA, 22 nt, Homo sapiens
acagcaggcacagacaggcagu

SEQ ID NO: 23 - RNA, 23 nt, Homo sapiens
agcuacauugucugcuggguuuc

SEQ ID NO: 24 - RNA, 20 nt, Homo sapiens
acugccccaggugcugcugg

SEQ ID NO: 25 - RNA, 20 nt, Homo sapiens
ccucugggcccuuccuccag

SEQ ID NO: 26 - RNA, 22 nt, Homo sapiens
cuggcccucucugcccuuccgu

SEQ ID NO: 27 - RNA, 23 nt, Homo sapiens
ugucugcccgcaugccugccucu

SEQ ID NO: 28 - RNA, 22 nt, Homo sapiens
gugacaucacauauacggcagc

SEQ ID NO: 29 - RNA, 23 nt, Homo sapiens
uaauccuugcuaccugggugaga

SEQ ID NO: 30 - RNA, 21 nt, Homo sapiens
ugagcuaaaugugugcuggga

SEQ ID NO: 31 - RNA, 21 nt, Homo sapiens
aggaggcagcgcucucaggac

SEQ ID NO: 32 - RNA, 5320 nt, Homo sapiens
ugagccgcagcgugcucgcccccgccgccagcucgccucacuuaugggucggaaacgcugcgugggggagacuguuccaagauggcggcggguugcugcggggugaagaagcagaaacuguccaguucgccccccucuggcucggguggcggugguggcgccuccuccuccucccacugcagcggagagagccagugccgagcuggggagcugggacuaggaggcgccgguacgcggcucaacgggcugggaggucuaaccggaggagguagcggcagcggcuguacccucucucccccccagggcugcggcggcggcggcggggggaucgcccugucgccaccuccgagcugcggaguggggacccuacuuucuaccccggccgccgccaccucuuccucacccuccucaucguccgccgccucguccucaucgccgggcucccggaagaugguggugucagcagagaugugcugcuuuugcuucgaugugcucuacugucaccuguauggauaccagcagccccggaccccccgauucaccaacgagcccuaugcccuuaaagauagccguuuucccccaaugacaagggaugagcugccacggcuuuucugcucagugucucugcucacuaacuuugaagaugucugugauuauuuggacugggagguggguguacauggcauuagaauagaauucaucaaugaaaaaggaucaaaacgcaccgccaccuaccuaccggagguugcaaaggagcaaggaugggaccauauacagaccauagacuccuuauugaggaaaggaggauacaaagcuccgauuacuaaugaauucaggaaaaccauaaaacugaccagguaucguagugaaaagaugacccugagcuaugcugaauaccuugcucaucgccagcaucaucauuuccaaaauggcauugggcauccccuuccgccauacaaccauuauuccugacacugagccgcacaaccagucacugggccucucugcagaccucuucccaggagacccuacaccuucuuggucuagcuaucucuuuuacuguaccauuuuaugaugauaguuuccguugccauggugaagcuucgacaucgucaacuaagaucaucaugguaacggguagaaaaauggcauuuguuaagauccuguuuuauuauuuuagaucguugauuuuuuuuuugcagcauauauaauuuuggguuuuuuuuuccuuauaauauuauguagaauuuugcuuugcugucucagucagguuucuguuuuucuguccuccuugccuuucuucccguuuugcuuccuguuuuccuucuuccuucccguuuuccuucuuccuucccugccucccuuccuuugacuguggauggaagaaagugugcaguuuuuagggauuuuacuuagguucgucuuuaguuuuccucaguaagauaguuguuuuuugauaccugaguuugggauuaauucauaucaaauucagguauuuguauauuacucuugauuuuugucugaaauucacuuugcuaugacagccuaguaguugggucuucacuccuuaaguauauguuuuucccauggugaaaauacaugaacuuuaguucuaguagugagcauucaaagguccgugauagggcuugucacagagagagagaaaucauuuaucccuauugugcugguuaucauagaagagacugccucaccacuuuauagaaccaauuugcaaacuaaaacacuguuuggagaagguacaaacuaccuuucaaaaaggcuacaggaaacauuuuaugagcagcaaauauuaggaagagagcagauacagaauuuagugccuuugagaagaauauaauuaaguggaauuaagagggguuagaaaaaaggcaauuuagaaaaauauucuuguaaaacuuuauuguuucuauaugugaaacagaagauuagacaaauuccuuacucuuaaguauuuuuuuaagcugaaaaaaaauuauuuugagcaaauaaccaauaagagacauuugucuauuuuauauuaauuuaaauuuguuagcucuagguuucaauaagucaugauuggcuagaauccauuuuagcuaccaucuaaagauaugguaaagauaauuuaguaaaacaaaaguucauauuugccaugauuucagauauguuugucauggggacaauauuagaguuuuagagauacuauaaaauagcuugaauuucuuucugcacuacauacuuuugauagaaacacauuuacuacauugauaagauguuaugucacaccugugaaaauggaguuggcauucagauaugcuauaguuagagcccagcucaucugcaaguauagucuucccaugaugcauacaaagauaacuuacugaugucgauaggagguccauacauauauccccaggaaaaaugacagauuauucuggaggaagaugaccuucauuuuaugguuaugugugugugucugugugucuuuggaaaaauauauauaauuuuuucaaauaggaagccaacauaucaagugaugaauuaaaguaugcugcagaauauauauucuaaaacuacaaaaaagucacugaauaucaaaaugauacagcuuauacauauaguuacugugacaagugacagacugcuaguucagaauucaaaaauccuuuccuaguuugugagauaaugggcuaaauuccuucugccugccacuggggcaaagcaaauugcuuuaguuuuugaugagaguucuuagaaguuuguugguauuccuucauccacagcauccauuguugaaauaaccauuuucaguugugaugccuuaacuaagaagccaauuguuagccugaaaugcaaucuugguagccaguuucaaugaagcuagagauuagucagaaaaaguuagcuguugggcuuuagaaagggauuuugaguccugucauuucuacuugggagcauuuuggagcagauuagucuuucaguauaaaaacaaguggcuaccugauggaaacuuuucuuacccuuauagggaaacugagcacaagcugaaugauauugucugcugcaaaaaaaaacaaacaaaaaaaaaacaaaacaaaaaacaaaaaaaaggaaaaaaagguaaauaaaacaagcaaacugaagagagacaaaauagaccaauauuaucgugaaauccaucuuauccaucaucucaucgguucaacaggcuccucguccugggugagcuuaugguuagaaguuuuuuuagcaaagauuuauuacauacauagauguguguguguacauauacuuacacauacauacauacauauauguguguuuauuuaacggugcuaaucaaucuugagcaggucacgugacuggucaugcggacucuaaaaucauaucagauuuuuuaaaauccuugauauaugcagauguuauacagauuuucuuagcaguguaauaacguuaaacugaagaaauaaccauccugcaggcuucaaaggacgaggucagcaauauuucccaacauggcuugggggaaaaggauaguuuugucuccuuuuguauucaaguaaugcaaagugcauuuuugucucuaaguagcuuuugacuaaaagacuuuguaguguaaaggguguuauacaaacuguaauggugugcuucaaacaaaugcuagacccuuucaccuuugucguauauacaaagcuguuaauuaugugguaugaauuaacuuugaacauguugaaauauguagcaugucuuuaacucagacgaagauucugauugcacagguuguguucuagccaguugugauuuauuuguucagaaacacaaaugugaauugcacucuuaucaucagaauauuguaugaguucagugaucuuuaaacagcucagaaguaauacuugaacuucauuugaguagcacaaaguuuacauagccccuuuuaaauguuaauucguuccauuuaaguuuuccauuuucagaugcaacuggguauuuuuccucuuggaaauaguacguacuuugcuuugguauguagcaaaaggcuuccauuucuggaaggguuuucuuguucuuaaacaaggucagccagguaccagauucuaaaagaacaaauugucccccaccccucaaguggccuucugacugcacgagaaugugcuacuacccucuguagucuccccuaacacgagaggucagugaaauugauggaggaagugcaaggcuugcuuccccucacccuuaaagcuuaaagcaguuguaagauuuucaguccagcacauacagucccagugcccagcacaccggaaguuuguauacuggccucuuaucuugucuucaaaugugcaggaaauuugaaacugucaucuggaauagguuccaucucuuauguguguuagaucaaaccaaagaagcccccagccaugcccaaaugcugcuucaaaagccugccuuccaguccuuacaaaaaccuugcaggauuaaauguguuaacuuuuuuauuuuuguacaguuucuaagugauuuuugcuagugauguuaaaaugaauuaaguuuauuugaggggugugagcaccuccuccauuuaaauaaacuggugacuuuccuuuuauuuuuuaaaaguggaaacccguugugugccucucgauuuaaggguuucugaugacauuauucuuaagaccagcauugauccuuuauauacaaauauauguuuuccuuguuuuuuauugcuauuucagaagaaaaugaaccuuuuauuuugcucuacccaguaacugcgcugguacauagacgcacugagcaaacaaacuuuuguaaccaccucugacuacuuuuguauaucaaaguugauuacuuuugaaauauuuggaucuaguuucuaaguuauuuuaguguuuuauauguuccccaaaauuacuuugaaucugucaccagcauaaugaguucuuggugauacaauggugagacuuguaaugagcaaaaagcuaccuggaguagcuccucuuuuccuucuuucuuaguaaccuguuuuuugauuaccguaagacaucacagauuaccugaaaccccuuccaguauaacuaggcccuccuuccuuccacauuuaauugcuuaauaguuugaagaaacuauuggaaaugggcauuuuauaugacauguauggcuuuacaauauuaaauaugagcaaaaagaaaaaauaugagaagguuuaugugggucuguaagguauggcuguggaaagauauuguaguaguuuugacacuauuguuauuaccuuuaaaaucauuuacccaauaaaauguuaaaaccgaaaaaaaaaaaaaaa

SEQ ID NO: 33 - RNA, 5618 nt, Homo sapiens
cucuuucucugggcuaaucguuuagaggacagucgcuccugccaaccuaaucccaugagccugacuaccuugccgggccacaggcucaaagaagcaguguggcugccagcacccucacgguaaggggcacggaggcaccaggcagucuggcaggaugagagacauccuggggguggucugaguuuggaugugggcugagaagacaaagcagggggcuuucgggggacaagauuuuccaaacaucuuauucuuaggaucaucacuuccuaaucugaugcaacugacucggcaggcauucgggagccuguaagcuccauuuuccuguuccaaauucuucugcaccucaccaugaguccucaccuugacccuaaucaacuugggauucuccucaaaguacuuagaaaggaaaaggaagacggugacuacccugacaugauggccacccacccgagcuccagguaugaagccugcuccucuggaauuacucuagggcugcggcggcggcggcggggggaucgcccugucgccaccuccgagcugcggaguggggacccuacuuucuaccccggccgccgccaccucuuccucacccuccucaucguccgccgccucguccucaucgccgggcucccggaagaugguggugucagcagagaugugcugcuuuugcuucgaugugcucuacugucaccuguauggauaccagcagccccggaccccccgauucaccaacgagcccuacccacuguuuguaacauggaagauuggucgagacaaaagauuacguggaugcauagguacuuuuucugccaugaauuugcauucaggacucagggaguacacacuuaccagugcccuuaaagauagccguuuucccccaaugacaagggaugagcugccacggcuuuucugcucagugucucugcucacuaacuuugaagaugucugugauuauuuggacugggagguggguguacauggcauuagaauagaauucaucaaugaaaaaggaucaaaacgcaccgccaccuaccuaccggagguugcaaaggagcaaggaugggaccauauacagaccauagacuccuuauugaggaaaggaggauacaaagcuccgauuacuaaugaauucaggaaaaccauaaaacugaccagguaucguagugaaaagaugacccugagcuaugcugaauaccuugcucaucgccagcaucaucauuuccaaaauggcauugggcauccccuuccgccauacaaccauuauuccugacacugagccgcacaaccagucacugggccucucugcagaccucuucccaggagacccuacaccuucuuggucuagcuaucucuuuuacuguaccauuuuaugaugauaguuuccguugccauggugaagcuucgacaucgucaacuaagaucaucaugguaacggguagaaaaauggcauuuguuaagauccuguuuuauuauuuuagaucguugauuuuuuuuuugcagcauauauaauuuuggguuuuuuuuuccuuauaauauuauguagaauuuugcuuugcugucucagucagguuucuguuuuucuguccuccuugccuuucuucccguuuugcuuccuguuuuccuucuuccuucccguuuuccuucuuccuucccugccucccuuccuuugacuguggauggaagaaagugugcaguuuuuagggauuuuacuuagguucgucuuuaguuuuccucaguaagauaguuguuuuuugauaccugaguuugggauuaauucauaucaaauucagguauuuguauauuacucuugauuuuugucugaaauucacuuugcuaugacagccuaguaguugggucuucacuccuuaaguauauguuuuucccauggugaaaauacaugaacuuuaguucuaguagugagcauucaaagguccgugauagggcuugucacagagagagagaaaucauuuaucccuauugugcugguuaucauagaagagacugccucaccacuuuauagaaccaauuugcaaacuaaaacacuguuuggagaagguacaaacuaccuuucaaaaaggcuacaggaaacauuuuaugagcagcaaauauuaggaagagagcagauacagaauuuagugccuuugagaagaauauaauuaaguggaauuaagagggguuagaaaaaaggcaauuuagaaaaauauucuuguaaaacuuuauuguuucuauaugugaaacagaagauuagacaaauuccuuacucuuaaguauuuuuuuaagcugaaaaaaaauuauuuugagcaaauaaccaauaagagacauuugucuauuuuauauuaauuuaaauuuguuagcucuagguuucaauaagucaugauuggcuagaauccauuuuagcuaccaucuaaagauaugguaaagauaauuuaguaaaacaaaaguucauauuugccaugauuucagauauguuugucauggggacaauauuagaguuuuagagauacuauaaaauagcuugaauuucuuucugcacuacauacuuuugauagaaacacauuuacuacauugauaagauguuaugucacaccugugaaaauggaguuggcauucagauaugcuauaguuagagcccagcucaucugcaaguauagucuucccaugaugcauacaaagauaacuuacugaugucgauaggagguccauacauauauccccaggaaaaaugacagauuauucuggaggaagaugaccuucauuuuaugguuaugugugugugucugugugucuuuggaaaaauauauauaauuuuuucaaauaggaagccaacauaucaagugaugaauuaaaguaugcugcagaauauauauucuaaaacuacaaaaaagucacugaauaucaaaaugauacagcuuauacauauaguuacugugacaagugacagacugcuaguucagaauucaaaaauccuuuccuaguuugugagauaaugggcuaaauuccuucugccugccacuggggcaaagcaaauugcuuuaguuuuugaugagaguucuuagaaguuuguugguauuccuucauccacagcauccauuguugaaauaaccauuuucaguugugaugccuuaacuaagaagccaauuguuagccugaaaugcaaucuugguagccaguuucaaugaagcuagagauuagucagaaaaaguuagcuguugggcuuuagaaagggauuuugaguccugucauuucuacuugggagcauuuuggagcagauuagucuuucaguauaaaaacaaguggcuaccugauggaaacuuuucuuacccuuauagggaaacugagcacaagcugaaugauauugucugcugcaaaaaaaaacaaacaaaaaaaaaacaaaacaaaaaacaaaaaaaaggaaaaaaagguaaauaaaacaagcaaacugaagagagacaaaauagaccaauauuaucgugaaauccaucuuauccaucaucucaucgguucaacaggcuccucguccugggugagcuuaugguuagaaguuuuuuuagcaaagauuuauuacauacauagauguguguguguacauauacuuacacauacauacauacauauauguguguuuauuuaacggugcuaaucaaucuugagcaggucacgugacuggucaugcggacucuaaaaucauaucagauuuuuuaaaauccuugauauaugcagauguuauacagauuuucuuagcaguguaauaacguuaaacugaagaaauaaccauccugcaggcuucaaaggacgaggucagcaauauuucccaacauggcuugggggaaaaggauaguuuugucuccuuuuguauucaaguaaugcaaagugcauuuuugucucuaaguagcuuuugacuaaaagacuuuguaguguaaaggguguuauacaaacuguaauggugugcuucaaacaaaugcuagacccuuucaccuuugucguauauacaaagcuguuaauuaugugguaugaauuaacuuugaacauguugaaauauguagcaugucuuuaacucagacgaagauucugauugcacagguuguguucuagccaguugugauuuauuuguucagaaacacaaaugugaauugcacucuuaucaucagaauauuguaugaguucagugaucuuuaaacagcucagaaguaauacuugaacuucauuugaguagcacaaaguuuacauagccccuuuuaaauguuaauucguuccauuuaaguuuuccauuuucagaugcaacuggguauuuuuccucuuggaaauaguacguacuuugcuuugguauguagcaaaaggcuuccauuucuggaaggguuuucuuguucuuaaacaaggucagccagguaccagauucuaaaagaacaaauugucccccaccccucaaguggccuucugacugcacgagaaugugcuacuacccucuguagucuccccuaacacgagaggucagugaaauugauggaggaagugcaaggcuugcuuccccucacccuuaaagcuuaaagcaguuguaagauuuucaguccagcacauacagucccagugcccagcacaccggaaguuuguauacuggccucuuaucuugucuucaaaugugcaggaaauuugaaacugucaucuggaauagguuccaucucuuauguguguuagaucaaaccaaagaagcccccagccaugcccaaaugcugcuucaaaagccugccuuccaguccuuacaaaaaccuugcaggauuaaauguguuaacuuuuuuauuuuuguacaguuucuaagugauuuuugcuagugauguuaaaaugaauuaaguuuauuugaggggugugagcaccuccuccauuuaaauaaacuggugacuuuccuuuuauuuuuuaaaaguggaaacccguugugugccucucgauuuaaggguuucugaugacauuauucuuaagaccagcauugauccuuuauauacaaauauauguuuuccuuguuuuuuauugcuauuucagaagaaaaugaaccuuuuauuuugcucuacccaguaacugcgcugguacauagacgcacugagcaaacaaacuuuuguaaccaccucugacuacuuuuguauaucaaaguugauuacuuuugaaauauuuggaucuaguuucuaaguuauuuuaguguuuuauauguuccccaaaauuacuuugaaucugucaccagcauaaugaguucuuggugauacaauggugagacuuguaaugagcaaaaagcuaccuggaguagcuccucuuuuccuucuuucuuaguaaccuguuuuuugauuaccguaagacaucacagauuaccugaaaccccuuccaguauaacuaggcccuccuuccuuccacauuuaauugcuuaauaguuugaagaaacuauuggaaaugggcauuuuauaugacauguauggcuuuacaauauuaaauaugagcaaaaagaaaaaauaugagaagguuuaugugggucuguaagguauggcuguggaaagauauuguaguaguuuugacacuauuguuauuaccuuuaaaaucauuuacccaauaaaauguuaaaaccgaaaaaaaaaaaaaaa

SEQ ID NO: 34 - RNA, 5431 nt, Homo sapiens
ugagccgcagcgugcucgcccccgccgccagcucgccucacuuaugggucggaaacgcugcgugggggagacuguuccaagauggcggcggguugcugcggggugaagaagcagaaacuguccaguucgccccccucuggcucggguggcggugguggcgccuccuccuccucccacugcagcggagagagccagugccgagcuggggagcugggacuaggaggcgccgguacgcggcucaacgggcugggaggucuaaccggaggagguagcggcagcggcuguacccucucucccccccagggcugcggcggcggcggcggggggaucgcccugucgccaccuccgagcugcggaguggggacccuacuuucuaccccggccgccgccaccucuuccucacccuccucaucguccgccgccucguccucaucgccgggcucccggaagaugguggugucagcagagaugugcugcuuuugcuucgaugugcucuacugucaccuguauggauaccagcagccccggaccccccgauucaccaacgagcccuacccacuguuuguaacauggaagauuggucgagacaaaagauuacguggaugcauagguacuuuuucugccaugaauuugcauucaggacucagggaguacacacuuaccagugcccuuaaagauagccguuuucccccaaugacaagggaugagcugccacggcuuuucugcucagugucucugcucacuaacuuugaagaugucugugauuauuuggacugggagguggguguacauggcauuagaauagaauucaucaaugaaaaaggaucaaaacgcaccgccaccuaccuaccggagguugcaaaggagcaaggaugggaccauauacagaccauagacuccuuauugaggaaaggaggauacaaagcuccgauuacuaaugaauucaggaaaaccauaaaacugaccagguaucguagugaaaagaugacccugagcuaugcugaauaccuugcucaucgccagcaucaucauuuccaaaauggcauugggcauccccuuccgccauacaaccauuauuccugacacugagccgcacaaccagucacugggccucucugcagaccucuucccaggagacccuacaccuucuuggucuagcuaucucuuuuacuguaccauuuuaugaugauaguuuccguugccauggugaagcuucgacaucgucaacuaagaucaucaugguaacggguagaaaaauggcauuuguuaagauccuguuuuauuauuuuagaucguugauuuuuuuuuugcagcauauauaauuuuggguuuuuuuuuccuuauaauauuauguagaauuuugcuuugcugucucagucagguuucuguuuuucuguccuccuugccuuucuucccguuuugcuuccuguuuuccuucuuccuucccguuuuccuucuuccuucccugccucccuuccuuugacuguggauggaagaaagugugcaguuuuuagggauuuuacuuagguucgucuuuaguuuuccucaguaagauaguuguuuuuugauaccugaguuugggauuaauucauaucaaauucagguauuuguauauuacucuugauuuuugucugaaauucacuuugcuaugacagccuaguaguugggucuucacuccuuaaguauauguuuuucccauggugaaaauacaugaacuuuaguucuaguagugagcauucaaagguccgugauagggcuugucacagagagagagaaaucauuuaucccuauugugcugguuaucauagaagagacugccucaccacuuuauagaaccaauuugcaaacuaaaacacuguuuggagaagguacaaacuaccuuucaaaaaggcuacaggaaacauuuuaugagcagcaaauauuaggaagagagcagauacagaauuuagugccuuugagaagaauauaauuaaguggaauuaagagggguuagaaaaaaggcaauuuagaaaaauauucuuguaaaacuuuauuguuucuauaugugaaacagaagauuagacaaauuccuuacucuuaaguauuuuuuuaagcugaaaaaaaauuauuuugagcaaauaaccaauaagagacauuugucuauuuuauauuaauuuaaauuuguuagcucuagguuucaauaagucaugauuggcuagaauccauuuuagcuaccaucuaaagauaugguaaagauaauuuaguaaaacaaaaguucauauuugccaugauuucagauauguuugucauggggacaauauuagaguuuuagagauacuauaaaauagcuugaauuucuuucugcacuacauacuuuugauagaaacacauuuacuacauugauaagauguuaugucacaccugugaaaauggaguuggcauucagauaugcuauaguuagagcccagcucaucugcaaguauagucuucccaugaugcauacaaagauaacuuacugaugucgauaggagguccauacauauauccccaggaaaaaugacagauuauucuggaggaagaugaccuucauuuuaugguuaugugugugugucugugugucuuuggaaaaauauauauaauuuuuucaaauaggaagccaacauaucaagugaugaauuaaaguaugcugcagaauauauauucuaaaacuacaaaaaagucacugaauaucaaaaugauacagcuuauacauauaguuacugugacaagugacagacugcuaguucagaauucaaaaauccuuuccuaguuugugagauaaugggcuaaauuccuucugccugccacuggggcaaagcaaauugcuuuaguuuuugaugagaguucuuagaaguuuguugguauuccuucauccacagcauccauuguugaaauaaccauuuucaguugugaugccuuaacuaagaagccaauuguuagccugaaaugcaaucuugguagccaguuucaaugaagcuagagauuagucagaaaaaguuagcuguugggcuuuagaaagggauuuugaguccugucauuucuacuugggagcauuuuggagcagauuagucuuucaguauaaaaacaaguggcuaccugauggaaacuuuucuuacccuuauagggaaacugagcacaagcugaaugauauugucugcugcaaaaaaaaacaaacaaaaaaaaaacaaaacaaaaaacaaaaaaaaggaaaaaaagguaaauaaaacaagcaaacugaagagagacaaaauagaccaauauuaucgugaaauccaucuuauccaucaucucaucgguucaacaggcuccucguccugggugagcuuaugguuagaaguuuuuuuagcaaagauuuauuacauacauagauguguguguguacauauacuuacacauacauacauacauauauguguguuuauuuaacggugcuaaucaaucuugagcaggucacgugacuggucaugcggacucuaaaaucauaucagauuuuuuaaaauccuugauauaugcagauguuauacagauuuucuuagcaguguaauaacguuaaacugaagaaauaaccauccugcaggcuucaaaggacgaggucagcaauauuucccaacauggcuugggggaaaaggauaguuuugucuccuuuuguauucaaguaaugcaaagugcauuuuugucucuaaguagcuuuugacuaaaagacuuuguaguguaaaggguguuauacaaacuguaauggugugcuucaaacaaaugcuagacccuuucaccuuugucguauauacaaagcuguuaauuaugugguaugaauuaacuuugaacauguugaaauauguagcaugucuuuaacucagacgaagauucugauugcacagguuguguucuagccaguugugauuuauuuguucagaaacacaaaugugaauugcacucuuaucaucagaauauuguaugaguucagugaucuuuaaacagcucagaaguaauacuugaacuucauuugaguagcacaaaguuuacauagccccuuuuaaauguuaauucguuccauuuaaguuuuccauuuucagaugcaacuggguauuuuuccucuuggaaauaguacguacuuugcuuugguauguagcaaaaggcuuccauuucuggaaggguuuucuuguucuuaaacaaggucagccagguaccagauucuaaaagaacaaauugucccccaccccucaaguggccuucugacugcacgagaaugugcuacuacccucuguagucuccccuaacacgagaggucagugaaauugauggaggaagugcaaggcuugcuuccccucacccuuaaagcuuaaagcaguuguaagauuuucaguccagcacauacagucccagugcccagcacaccggaaguuuguauacuggccucuuaucuugucuucaaaugugcaggaaauuugaaacugucaucuggaauagguuccaucucuuauguguguuagaucaaaccaaagaagcccccagccaugcccaaaugcugcuucaaaagccugccuuccaguccuuacaaaaaccuugcaggauuaaauguguuaacuuuuuuauuuuuguacaguuucuaagugauuuuugcuagugauguuaaaaugaauuaaguuuauuugaggggugugagcaccuccuccauuuaaauaaacuggugacuuuccuuuuauuuuuuaaaaguggaaacccguugugugccucucgauuuaaggguuucugaugacauuauucuuaagaccagcauugauccuuuauauacaaauauauguuuuccuuguuuuuuauugcuauuucagaagaaaaugaaccuuuuauuuugcucuacccaguaacugcgcugguacauagacgcacugagcaaacaaacuuuuguaaccaccucugacuacuuuuguauaucaaaguugauuacuuuugaaauauuuggaucuaguuucuaaguuauuuuaguguuuuauauguuccccaaaauuacuuugaaucugucaccagcauaaugaguucuuggugauacaauggugagacuuguaaugagcaaaaagcuaccuggaguagcuccucuuuuccuucuuucuuaguaaccuguuuuuugauuaccguaagacaucacagauuaccugaaaccccuuccaguauaacuaggcccuccuuccuuccacauuuaauugcuuaauaguuugaagaaacuauuggaaaugggcauuuuauaugacauguauggcuuuacaauauuaaauaugagcaaaaagaaaaaauaugagaagguuuaugugggucuguaagguauggcuguggaaagauauuguaguaguuuugacacuauuguuauuaccuuuaaaaucauuuacccaauaaaauguuaaaaccgaaaaaaaaaaaaaaa

SEQ ID NO: 35 - RNA, 7964 nt, Homo sapiens
uccgggauccggcaacuuugggcagcgcaugcgcgcccgcggcgcuccaucccaaacacacacacauuuuucccccggacuuggaaagucacccaaagccgccccaagugcaggcaaacagaccuugcugacugaaggcaaagacuccuccaucccaccugcccucccagcugccacggccaucgaccccuccuuuacaaggggcuccugcaacuccccagcagcacguguuggaauggacuucggacccuggccucuugggaugcaaaggaugaagugacacccccagcuacauccgaggagguucuaggaccugcuacgagaguuugggaccaaggagaagagaauggaucuuggaacagcugagggcacccggugcacggacccgccugcaggcaagcccgccauggcgcccaaacgcaaggguggccugaagcugaacgccaucugcgccaagcugagccgccagguggugguggagaagcgagcugacgccggcucccacacggagggcagcccaucgcagccccgggaccaagagcgcaguggcccugagucuggggcagcccgggccccccgcagcgaggaagacaagagacgggcagugaucgagaagugggugaacggggaguacagcgaggagccggcacccacacccguguuggggcggauugcccgcgagggccuggagcugccucccgagggugucuacauggugcagccccaggggugcagcgaugaggaagaccacgcggaggagcccuccaaggacggcggugcccuggaggagaaggauucggacggggcagccuccaaggaggacagcggccccagcaccaggcaggcuucaggagaggccuccucgcugcgggacuacgcggccuccaccaugaccgaguuccucggcauguuuggcuaugaugaccagaacacgcgggacgagcuggccaggaagaucagcuuugagaagcugcacgcgggcuccaccccggaggcagccaccuccuccaugcugcccaccuccgaggauacccucagcaagcgggcgcgguucucuaaguaugaggaguacauccgcaagcucaaggcuggcgagcagcucuccuggccggcccccagcaccaagaccgaggagcgggugggcaaggagguggugggcacccugcccggccugcggcugcccagcagcacggcccaccuggagaccaaggccaccauccugccccugccgucgcacagcaguguccagaugcagaaccugguagcccgggccuccaaguacgacuucuucauccaaaaacugaagaccggcgagaaucugcggccccagaacgggagcaccuacaagaagccauccaaguacgaccuggagaaugucaaguaccugcaccucuucaaacccggggagggcagccccgacaugggcggggccaucgccuucaagacaggcaagguggggcgcccuuccaaguacgacguccggggcauccagaagccaggccccgccaagguuccgcccacccccagccuggcucccgcaccccucgccagcgugcccagugcccccagcgcccccgggccagggccagagccuccugccucccuguccuucaacacucccgaguaccugaagucaaccuucuccaaaacagacuccaucaccacggggaccgucuccacugucaagaacggacugcccacagauaaaccagccgucacugaagauguaaacauuuaccagaaauauauugccagguucucgggcagccagcacuguggccacauccacugugccuaccaguaccgcgagcacuaccacugccuugacccugaguguaacuaccagagguucacgaguaagcaggacgugauccgccacuacaacaugcacaagaagcgcgacaacucccugcagcacggcuucaugcguuucagcccgcuggacgacugcagcgucuacuaccacggcugccaccucaaugggaagagcacccacuaucacugcaugcaggugggcuguaacaagguguacacgagcacgucugacgugaugacccacgagaacuuccacaagaagaauacccagcucauuaacgacggcuuccagcgcuuccgagccaccgaagacuguggcacagccgacugccaguucuacggacagaagaccacgcacuuccacugcaggcgccccggcugcacauucacuuucaagaacaagugugacaucgagaagcacaagagcuaccacaucaaggacgaugccuacgccaaggacggcuucaagaaguucuacaaguacgaggagugcaaguacgagggcugcguguacagcaaggcuaccaaccacuuccacugcauccgcgccggcugcggcuucaccuucaccuccaccagccagaugaccucucacaagcgcaagcaugagcgccggcacauccgcuccucgggcgcgcuggggcugccgcccucgcugcugggcgccaaggacacggagcacgaggaguccagcaacgacgaccuuguugacuucuccgcccugagcagcaagaacuccagccugagcgccuccccuaccagccagcaguccucugcgucccuggcugccgccacugccgccaccgaggcugggcccagugccaccaaaccucccaacagcaagaucucggggcugcugccccagggccugccuggcucaaucccccuggcccuggcccucuccaacucgggccugcccacccccacgcccuacuuccccauacuggcuggccgugggagcaccucccugccugugggcacccccagccuccugggugccgugucgucugggucagcagccucagccaccccugacacacccacgcuggucgccucgggagcuggagacucagcccccguggcugccgccucugucccggcaccacccgccuccaucauggagaggaucucugcaagcaagggccucaucucgcccaugauggccaggcuggcugcagcugcccucaagcccucugccaccuuugacccaggaagcgggcagcaggucaccccagccagguuccccccggcccaagugaagccggaacccggugagagcaccggcgccccaggcccccacgaagccucccaggaccgcagucuagaccugacugugaaggagcccagcaacgaaucaaauggccacgcagucccggcaaauucaucucuuuuauccucgcuuaugaauaagaugucucagggcaacccuggccugggcagccugcugaacaucaaggcggaagcggaggggagccccgcugcggagcccucgcccuuccuaggcaaggccgugaaggcgcugguucaggagaaguuggcagagcccuggaagguguaccugcgcagguuugguacaaaggacuucugugacggccagugugacuuccuccacaaggcccacuuccacugcgugguggaggaaugcggcgcgcucuucagcaccuuggacggggccaucaagcacgcaaacuuccacuuccggacagagggaggagcagcaaaaggaaacacagaggcugccuuuccggccucggccgccgagaccaaaccucccauggcccccucguccccuccggucccuccugucaccacggccacgguguccucucuggaggggcccgcucccagcccggccuccgugcccuccacccccacccugcucgccuggaagcagcuggcuuccaccauaccccagaugccucagaucccagcgucagugccucaccugcccgccucgcccuuggcaacgacuucucuagagaacgccaagccccaggucaaacccggauuccuccaguuccaggagaacgauccuugccucgccacggacugcaaguacgccaacaaguuccacuuccacugucucuuugggaacugcaaguacgucugcaaaacgucuggcaaggccgaaucccacugccuggaccacaucaaccccaacaacaaccuggugaacgugcgagaccaguuugcauacuacucucugcagugucucugucccaaccagcacugcgaguuccgaaugcgugggcacuaccacugccuccgcaccggcugcuauuuugugaccaacaucaccaccaagcuccccuggcacaucaagaagcaugagaaggcggagcggcgggcagccaauggcuucaaauacuucaccaagcgcgaggaguguggcaggcuagguugcaaguacaaccaggugaacagccacuuccacugcauccgggagggcugccaguucuccuuccuccucaagcaccagaugaccucccacgcgcggaagcacaugcggaggaugcuggggaagaacuucgaccgcgugccccccucccagggccccccaggccugauggaugcugagacagaugagugcauggacuacacuggcugcagcccaggcgccauguccucugagucauccaccauggaccggagcugcuccagcacccccguggguaacgagagcaccgcggcagggaacaccaucucuaugccgacagccucgggggccaaaaagcgcuucuggaucaucgaggacaugucgcccuucggcaagcggcggaagacggcguccucccggaagaugcuggacgagggcaugaugcuggagggcuuccggcgcuucgaccuuuacgaggacugcaaggacgcagcuugucaguucucgcucaaggucacccacuaccacugcacgcgcgagaacugcggcuacaaguucugcgggcgcacgcacauguacaagcacgcgcagcaccacgaccgcguggacaaccuggugcuggacgacuucaagcgcuucaaggccucacucagcugccacuucgccgacugccccuucucgggcaccagcacgcacuuccacugccugcgcugccgcuuccgcugcaccgacagcaccaaggucacggcgcaucgcaagcaccacggcaaacaggacgugaucagcgccgcgggcuucugccaguucagcuccagcgccgacugcgccgugcccgacugcaaguacaagcucaagugcucgcacuuccacugcaccuucccgggcugccgccacacgguggugggcaugucgcagauggacucgcacaagcgcaagcacgagaagcaggagcgcggcgagcccgcggcagagggccccgcgcccgggccucccaucagucuggacggcucccugucgcugggcgccgagccgggcucgcugcucuuccugcagucggcggccgccggccugggccuggcgcugggcgacgcgggcgaccccggcccgcccgacgccgccgcccccgggccgcgcgagggcgccgccgccgccgccgccgcagcuggggaguccucgcaggaggacgaggaggaggagcuggagcugccggaggaggaggccgaagacgacgaggacgaggacgacgacgaggacgacgacgacgaggacgacgacgaggacgacgacgacgaggaccugcgcaccgacucggaggagucgcugcccgaggcggcggcggaggcggcgggcgcgggcgcgcggaccccggccuuggcggcgcuggcggcccugggcgcccccggccccgcgcccacugcagccuccucgcccuagcggcgcccgcggguggcccuggcggcggccucggccucggccucgggagcggcgccgccccgcgcgaagcggccccugggcagagccccgcgccccgccggugcuuccggaccccgaguccgucucaggacagaggcggggaccccucuggugcuccgcggccgaggacacaggcggcggcgguggcccugcucuagggaggccgggucucuucagggaaacagaaggugcucuugucccgggguagggaaccuaggagcaggggucccgggcgaaugcggcgcagcucccggccgcugccccggcgccccuccccgcggccgauucggccccgcgaccauugcucaggacugggacgggcgcugggggccaccgggacccccaccgcgcgccaccagggcgggguggcuccuggagccgcuggcggggccucccucaugcagcuaggauccuccccccugcccagggggagacgggcagaaggcagucagcacgggugacacuacagggaccguggaggaagccucacgggaguagcagccgcggagcuccagcucagccccaccccagggcccggcugcucccccaggccugggucgggucuguuuggcgacucccuggccccagccuccgccccagggaaaccgaggcugcguccaaaacccagaguagcccgcugggggccggggcggggucucggccacccugguggugcaaacaggaaggaugcgccugguuuuggcuuuaauguucaaaauggaaaaaaauacaagaaaguuguacaguauuuuuccuguaaagguuauuauucuacauagaacaaaaaggaagaaagcaggcguggugagagugcagacccgccugggcccggccugagguuggaggcucagucccguuuauccugugggauuugguccaggguuuuuguagaggccuuaacuguuugaggcugucugucuucacaauagcacugaugggagcugcgugauuccaacucuuaacuugaauuuuguagagacaagaaaaaaggacuauuacuguugauccaaguuuguaguuaauuuaacauuugaguucucucuuggugaugcguguggcuuuauagggcuuuuuuuuuuuuccaguuuguuuuauuacuuuguuugggaugcuucuucgcuggugcagucuacccagauuggguucccuuugcaaaacaucccccuuccuggagaugaugaugccaucgaagcccgggccagggccugaccuggcaggcacacaccuggccaguggcucugagguccccggggaccgaguccccugcgagggcaggcugcccgaggccacaccugcccaccuucccuacuccaggccaccagcuggugucagauugggccaccgggagagaacaggccuaggagggaauggaaggcuggcggggaggcugucauccagaagccuccuccccagaggucuccaggaggaaccuggcucaacuggggggcagugguuuacuuuucccuuuuuucuuccuuucuuuuguccucucagcugcccacagccagaaagccaggaccacccgaacuggacagggagcagggagggaggaguggucgcacggagcguaccugauucaacaggaaaucaguuauuuucuaaaacagccccuaccagagccuccugaacucugugggcaccacgugcugcugugagcaucugcccagacagugacccgccacaggccccgggcgucuguggaaggaguuuuguaaaggaaaaaaaaacacacacacacaaacauuuuuucaauguagcaaaaucaaacuuaaaaaaaaaaaagaagaaaagaagaagaugccgacagugcggagucuagccuuuuguaaacuucauauugcacacuaggacuauaagccauugcuagcucauuuugaauuuuaacguguaauuuuuguuuuauuuucuuucuguggggaaacaaugcuugauccaccaaugcucuuuuuaauguuuuauaacuauguauguguauauauauaaauaucaaauaauauguaugcacauauguguguguauauaucuauauguauauacauauauagauauauagagauauauagagagguuuugagacaaaaaaaauacagaacgugaaacccgagcugaacagcgugugcucugauuacuugaaucuggucuccucggcaccugguuauuaagaacugaauauuuuuccacuugaauuuagugcuauuagaaauuuaauauauguguuuuauuuauuugauuuuuuuuuuuuuuuuugcaugacugaugcaagguuugacauuuucacucaauaaaaacuggaaaaaaa

SEQ ID NO: 36 - RNA, 4438 nt, Homo sapiens
uccgggauccggcaacuuugggcagcgcaugcgcgcccgcggcgcuccaucccaaacacacacacauuuuucccccggacuuggaaagucacccaaagccgccccaagugcaggcaaacagaccuugcugacugaaggcaaagacuccuccaucccaccugcccucccagcugccacggccaucgaccccuccuuuacaaggggcuccugcaacuccccagcagcacguguuggaauggacuucggacccuggccucuugggaugcaaaggaugaagugacacccccagcuacauccgaggagguucuaggaccugcuacgagaguuugggaccaaggagaagagaauggaucuuggaacagcugagggcacccggugcacggacccgccugcaggcaagcccgccauggcgcccaaacgcaaggguggccugaagcugaacgccaucugcgccaagcugagccgccagguggugguggagaagcgagcugacgccggcucccacacggagggcagcccaucgcagccccgggaccaagagcgcaguggcccugagucuggggcagcccgggccccccgcagcgaggaagacaagagacgggcagugaucgagaagugggugaacggggaguacagcgaggagccggcacccacacccguguuggggcggauugcccgcgagggccuggagcugccucccgagggugucuacauggugcagccccaggggugcagcgaugaggaagaccacgcggaggagcccuccaaggacggcggugcccuggaggagaaggauucggacggggcagccuccaaggaggacagcggccccagcaccaggcaggcuucaggagaggccuccucgcugcgggacuacgcggccuccaccaugaccgaguuccucggcauguuuggcuaugaugaccagaacacgcgggacgagcuggccaggaagaucagcuuugagaagcugcacgcgggcuccaccccggaggcagccaccuccuccaugcugcccaccuccgaggauacccucagcaagcgggcgcgguucucuaaguaugaggaguacauccgcaagcucaaggcuggcgagcagcucuccuggccggcccccagcaccaagaccgaggagcgggugggcaaggagguggugggcacccugcccggccugcggcugcccagcagcacggcccaccuggagaccaaggccaccauccugccccugccgucgcacagcaguguccagaugcagaaccugguagcccgggccuccaaguacgacuucuucauccaaaaacugaagaccggcgagaaucugcggccccagaacgggagcaccuacaagaagccauccaaguacgaccuggagaaugucaaguaccugcaccucuucaaacccggggagggcagccccgacaugggcggggccaucgccuucaagacaggcaagguggggcgcccuuccaaguacgacguccggggcauccagaagccaggccccgccaagguuccgcccacccccagccuggcucccgcaccccucgccagcgugcccagugcccccagcgcccccgggccagggccagagccuccugccucccuguccuucaacacucccgaguaccugaagucaaccuucuccaaaacagacuccaucaccacggggaccgucuccacugucaagaacggacugcccacagauaaaccagccgucacugaagauguaaacauuuaccagaaauauauugccagguucucgggcagccagcacuguggccacauccacugugccuaccaguaccgcgagcacuaccacugccuugacccugaguguaacuaccagagguucacgaguaagcaggacgugauccgccacuacaacaugcacaagaagcgcgacaacucccugcagcacggcuucaugcguuucagcccgcuggacgacugcagcgucuacuaccacggcugccaccucaaugggaagagcacccacuaucacugcaugcaggugggcuguaacaagguguacacgagcacgucugacgugaugacccacgagaacuuccacaagaagaauacccagcucauuaacgacggcuuccagcgcuuccgagccaccgaagacuguggcacagccgacugccaguucuacggacagaagaccacgcacuuccacugcaggcgccccggcugcacauucacuuucaagaacaagugugacaucgagaagcacaagagcuaccacaucaaggacgaugccuacgccaaggacggcuucaagaaguucuacaaguacgaggagugcaaguacgagggcugcguguacagcaaggcuaccaaccacuuccacugcauccgcgccggcugcggcuucaccuucaccuccaccagccagaugaccucucacaagcgcaagcaugagcgccggcacauccgcuccucgggcgcgcuggggcugccgcccucgcugcugggcgccaaggacacggagcacgaggaguccagcaacgacgaccuuguugacuucuccgcccugagcagcaagaacuccagccugagcgccuccccuaccagccagcaguccucugcgucccuggcugccgccacugccgccaccgaggcugggcccagugccaccaaaccucccaacagcaagaucucggggcugcugccccagggccugccuggcucaaucccccuggcccuggcccucuccaacucgggccugcccacccccacgcccuacuuccccauacuggcuggccgugggagcaccucccugccugugggcacccccagccuccugggugccgugucgucugggucagcagccucagccaccccugacacacccacgcuggucgccucgggagcuggagacucagcccccguggcugccgccucugucccggcaccacccgccuccaucauggagaggaucucugcaagcaagggccucaucucgcccaugauggccaggcuggcugcagcugcccucaagcccucugccaccuuugacccaggaagcgggcagcaggucaccccagccagguuccccccggcccaagugaagccggaacccggugagagcaccggcgccccaggcccccacgaagccucccaggaccgcagucuagaccugacugugaaggagcccagcaacgaaucaaauggccacgcagucccggcaaauucaucucuuuuauccucgcuuaugaauaagaugucucagggcaacccuggccugggcagccugcugaacaucaaggcggaagcggaggggagccccgcugcggagcccucgcccuuccuaggcaaggccgugaaggcgcugguucaggagaaguuggcagagcccuggaagguguaccugcgcagguuugguacaaaggacuucugugacggccagugugacuuccuccacaaggcccacuuccacugcgugguggaggaaugcggcgcgcucuucagcaccuuggacggggccaucaagcacgcaaacuuccacuuccggacagagggaggagcagcaaaaggaaacacagaggcugccuuuccggccucggccgccgagaccaaaccucccauggcccccucguccccuccggucccuccugucaccacggccacgguguccucucuggaggggcccgcucccagcccggccuccgugcccuccacccccacccugcucgccuggaagcagcuggcuuccaccauaccccagaugccucagaucccagcgucagugccucaccugcccgccucgcccuuggcaacgacuucucuagagaacgccaagccccaggucaaacccggauuccuccaguuccaggagaagugagucccucgaugagccgggagucccgcguuccccucgcgucucgggaguaggugcuagcaagggcgcuaggaggcccuguuccucacugcggauggugcugcuguccccagccucucuggggcauggccaucgggugauguccuucuagccaaagaugcugcugcuccuaccucacugccugucccagagcaggccagcccgcgugggccgaugguggcggcaguggcuacugcuccugcagggcauguggugauccugccagggccaggugggguggacugggcguggugguccucagaggacaacucccagccugacaaggagggcugcgucucccuccgagccuccguauuggccuccucuguggcucacacccauggcugaaucucugcagggcacgugaagucacggguaggggccaggccccuccaggccgucacuggccugcacaguggucugagcucuuggguggaagggacccuccucacuggauggugguggcuccuugcagggagaacggugacuuuguauugucaugugugcggcuuccuguucucaauaaaaguaauaaauuggauuauuuauaucgcc

SEQ ID NO: 37 - RNA, 5489 nt, Homo sapiens
gaaacucuuaacaaaaacaaggggcucggggagguuuccgcugaggcggcgggggugcggcggugggcuggucuuccgcggccggcguugcgccgcggcggagggugggcgcgcggggagcgggauggagccggggcugugaggccgaggcggcggugccugggaggaagggucggaugccggaccgggggcaccgcugaggcgguggguccccgaccugcgagacagguuuggaagcccccgcugcgcccaguccgugcggaccgcgaggccgcgggcggguggaggcgcgucuccggcacgaugaaggauuugggggcagagcacuuggcaggucaugaagggguccaacuucucggguuguugaacgucuaccuggaacaagaagagagauuccaaccucgagaaaaagggcugaguuugauugaggcuaccccggagaaugauaacacuuuguguccaggauugagaaaugccaaaguugaagauuuaaggaguuuagccaacuuuuuuggaucuugcacugaaacuuuuguccuggcugucaauauuuuggacagguucuuggcucuuaugaaggugaaaccuaaacauuugucuugcauuggagucuguucuuuuuugcuggcugcuagaauaguugaagaagacugcaauauuccauccacucaugaugugauccggauuagucaguguaaauguacugcuucugacauaaaacggauggaaaaaauaauuucagaaaaauugcacuaugaauuggaagcuacuacugccuuaaacuuuuugcacuuauaccauacuauuauacuuugucauacuucagaaaggaaagaaauacugagccuugauaaacuagaagcucagcugaaagcuugcaacugccgacucaucuuuucaaaagcaaaaccaucuguauuagccuugugccuucucaauuuggaaguggaaacuuugaaaucuguugaauuacuggaaauucucuugcuaguuaaaaaacauuccaagauuaaugacacugaguucuucuacuggagagaguugguuucuaaaugccuagccgaguauucuucuccugaauguugcaaaccagaucuuaagaaguugguuuggaucguuucaaggcgcacagcccagaaccuccacaacagcuacuauaguguuccugagcugccaacgauaccugagggggguuguuuugaugaaagugaaagugaggacucuugugaagauaugaguuguggagaggagagucucagcagcucuccucccagugaucaagagugcaccuucuuuuucaacuucaaaguggcacaaacacugugcuuuccaucuuagaaaucugauuguucugucagaauuuauauuuacagguuucaaagcaauaaaugggggaauagguaguuuccugguuuagcccccaucuagucaggaauuaauauacuggaauaccuaccuucuauuuguuauucagaucagaucuggccuauuuucauauuuauccuaagccaucaaaugggguagugccucuuaaaccauuaacaguacuuuagacauuggcacuuuauuuuucucguagaucuuuagcuacuuuggggaggagggaaggugcugauaccuucaauuuguuacuuuucaagauuuuuaaaaauaacuaguguagcuuaucuuaaacauuuuauaaaaccuucagaugucuuuaagcagauuggaaguaugcaagugcuuccuuagcagggacaguggauaauccuuaaugguuuaucauagauuucacccuccccccuucucagaagagugaguaugcucuuaaaugucaaacacauuuuuguuguuuuguuuuuuaaaugaucagugucuauuugaugugaugcagaucuuauaaauuugggaauuauaauauugacauuucugugauuuuuauauauguaaugucuuaauugagauuucuguuaaggcagaaauaauuaggcuagggcucuuaguuuucauuccuauugcccaaguauugucaaacuaugguauuauuuuaauguuacuuuaaaaauccauaaucugcuaguuuugcauguacuuauaugaaaacagugcaguaaguugaaaacucaguaucuauggaauugauaaauguugaucugguguaguauauuuuaucgcauuuucuuauauuaaaaaaugucugcaugauuacauuuuauuuccuuuguaauuuacauuucagaauaguguauugcuauaugggugccaagauugaauaugaagaacccgaguguuuguaguauuauaguuuuaagcaaaucuguguggugauacagccauaagaauggggcuuauauaaacucuguacauguaagauuuuguacagagaauuuuuaacuuuauaaauuguauaugaacauguaaaucuuuuaaaauguacauaaaauacuguauuuuuuuaccuugugugugauagucuagucauugcauguaaauauaauuuauuauguauucuguaguauaaaucauacauugaugacuuacauuuuuacugguaagucaacauccguuggauguuuucugaaguggcucuuuuugaagugauaauagauuguaauucaaaauaaaauuauuaaugaauucuccuuguuugggaucacaucuuaauuuuuaaucuguuaaaaguucuugauguauuuuaaugagaagacuuuaggugaggcuacagugauuccagagugagccuucuaacuggcuagcagaaguucucuagguuuggcaucugugccuuggagauacugaaagagaaucugucauuugacaauugaccucuuugugggauggacucauuaaguaugcucucagagacugguauauuaccagaaugccuauuaauuuucagugagaggcaacagguauuaaguagaacagaaugcucagguuggcagauuagaacgaucuuucaggagacaaagcaaguuuuaaucaguuguuugguuaauaaguaugggguguucgcugugauagggccccgccagcuucuggcucuuguggaccucaaaaguaucaggugguuuugcaaguggugguccuuuccccugccccaccccaauagguuccccaucugucuaguuugauuuuuguagaccuuuguuuucucuaguuagaaaaucagguacacugaauaugguuuucauguaacaccucuucucuggagauagggguauguuuuccuacccuucuaguggagaauccuacuugaggaugaccuuuccucucuuacuaaauaauauuaguaaauagugggcaauauauucugcuuucagauuuugauuuguugagauguaaaaguuguuuggggcuuaccaaaucucaagacucucuuuagcuccugcaggauuguauugcuuuucuuacuggauauuuuuccuggguaagcaucuuuguggcuucaucucuucccccugugguuuucaguguauuuagucgagaccucucugcugagcuugcaaccuguuuauucacauggccugccaugccacuuggagguuucugauuacucccaaaccugcugguucuuuaugucuuucucagcgaauaauuccaucuauucauguuggaaacuuaggugauaugcucaucuccuuuugccuguuuauggaggucaccagccucuaucauuuguaugauuucguuuacacuguuuauaucucucuguccccccuuuuucugccauuggcaugguuuagaccuguacucuuuaucagcagagguacuguaauauauuugugaucccucagcuuccaggcuuacuccuggucucugccuuccuaucuacauauccuuuuaaaauaaaauuuuaacuaucuccugaaaaauuguugaguaggucacgcacaaucaggagaaaaaucuauucaugacauacaagucucugucuaaucugaacacugcaccugucucuggccuuuuuuucuugucauuuccuagaccuuaaaaaauguguauugagaaagaacucuguuagcuauacagaagaugaacugggcaauauagaguagcagcauggagaccagucugacugaacuaaggcaguggaaguguggaugaggaagagaggugaaaauugagaagcgcuauccuuucucuuugggcauuauuaggaggcucacagacaaguccaggagccugguuauacccuccugugccauucaaccagguggcuuucccaugacugugaugaauaaaauugagaagccccugcccuuuucagagcagagggugaggagaaagcuaccauuuuguccucauccuuacccccguugacuuggcgagagauuugaccuuucagguuuugauccugucauuuucuaggauguggugcacgcacuuugcuguugcgcauggugaaguauugugccuagguccugggucuucaucuguuuggcucugcuacuguuuccuccucccaggaagugugguuagacaaauaauguguuuuaauuaccugucacacucaggauuaauacauacucagguuaacuguagagaggcauuggcuucagaacacuccucgugacaauuuuaaccauuuucuuugucuagagucugccuuuuucuuuuuuacaauuucuuuuauuucaacacuagguuucaauaugguguuccugcuaccucccaccucccuccucccucaucacacaugcaaauugucagcuuauugagacaacccacuuagauucauauauggacaaggacaagguauuuugcauuuguuacuggaauucaguuuuccuaacuauuuacuaccagaaauggucaauaacuuacuuuguguuuagcaaaucaaauugugugauagauaguuucccaguaugauggccagucagucuuuccaucccugugccuacaugcugcucuucccguccacaaguggagucuguuucucuugaguuuuggcuggccuuaugaauggcuuugcuuacugaagugcagcagaagaaauuuaguauauguccaagccuaggcuuuaagagacuggcagcuuuccuuuuauccuuuuuggaagcuagccaccaugcugcaaagaagcucagcuggauuacugaaagaugagaggccauguggagagagacucuugaggaugagagauuaucuuggauguuccagccuuaagcucccagcugaauguggguguauccucagcuacaccacagaaaacagaggaacuacucagucgaucccaaucaacccacagacucacuagaaauaacaaauuauuguuuuaagccacgagguuuugggggaggguuguuaaacaguaauagauaagugagacagauugcuuguuauuuauggucaaauggugauuaucucuggugagauuacaggugauguuuuuuuuaaguuaugccuaucuguaguuuccuuuuuuuccuaaaauugauuugaauuauuaguguauuaacagaauaaagaaugaacuuuaaaacacaaaaaaaaaaaaaaaa

SEQ ID NO: 38 - RNA, 6939 nt, Homo sapiens
auggccucgcuggaccugccguaccgcugcccccgcugcggggagcacaagcgcuuccggagccugucgucgcugcgcgcgcaccuggaguacagccacaccuacgagacgcucuacauccucuccaagaccaacagcaucugcgacggcgccgccgccgccgcggccgccgccgccgcugccucgggguucccgcuggcucccgagcccgccgcccugcuggccgugcccggcgcccggcgagaggucuucgagagcacuuccuuccagggcaaggagcaggcggccgggccgucgcccgcggcgccgcaccugcugcaccaccaccaucaccacgcuccccucgcccacuuccccggcgaccuggugcccgcuagccugcccugugaggaguuggccgagccgggccuugugcccgccgcagcagcgcgcuaugcgcugcgcgagaucgagaucccgcugggggagcuguucgcccgcaaguccguggcguccucggcgugcucgacgccgccgccuggccccggccccggcccuugccccgggccugccuccgcuucgcccgcgucccccucacccgcugauguggccuacgaagagggccuggcgcgccucaagauccgcgcgcuggagaagcuggagguggaccggcggcuggagcggcugagcgaggagguggagcagaagaucgcgggccaggugggccggcugcaggccgagcuggagcgcaaggcggccgaacuggagacugcgcggcaggagagugcgaggcucgggcgcgagaaggaggagcuggaggagcgcgcgucugagcucucccgccagguggacgugagcguagagcugcuggccucacucaagcaggaccuggugcacaaggaacaggagcugagccgcaagcagcaggagguggugcagaucgaccaguuccugaaggagacggcggcgcgggaggccagcgccaagcugcggcugcagcaguucauugaggagcuccuugagcgggcugaccgugccgagcggcagcugcaggucaucagcagcagcuguggcagcacgcccagcgccagccugggccguggaggugggggcgguggugcuggacccaaugcccggggcccaggcagaaugcgagaacaccacgugggcccggccgugccuaacacauaugcagugucacggcauggcuccucuccgagcacaggggccuccagccgugugccagccgcaucccagagcucaggcugcuaugacagugacagucuggagcugcccaggccagaggagggggccccugaggacaguggcccugggggcuugggcacacgggcccaggcugccaacgggggcucagagcggucccagcccccucgcagcucaggccugcggcgccaggccauccagaacuggcagcgcagaccccgccgacacagcacugagggggaagagggugaugucuccgacguuggcucccgaaccacugagucagaggcugagggcccguuggaugcgccccgccccgggccugcuauggcugggccauugagcagcugccggcucucagcccgccccgagggaggcagugggcggggucggcgagcagagagggucagccccucacgcuccaaugaggucaucagcccagagauccugaagaugcgagcugcccucuucugcaucuucaccuaccuggacacgcgcacacugcugcaugcugccgaggucugccgggacuggcgcuucguggcccgccaccccgcagucuggacaagggugcugcuugagaaugcccgugucugcuccaaguuccuggcaaugcuggcucaguggugcacccaggcccacucucugacgcugcagaacuugaagccccggcagcggggaaagaaggagagcaaggaggaguaugcccggagcacccggggcugccuggaagcugggcuggagucccugcugaaggcagcuggggggaaccugcugauccugcgcaucucccacuguccaaacauccucaccgaccgcucgcucuggcuggccagcugcuacugccgugcccugcaggcugucacguacaggagugccacagaccccgugggccaugaggucauuugggcccugggcgcaggcugcagagagaucgucucccuccaaguggcaccacuucaccccugccagcagcccacacgcuucaguaaccgcugccugcagaugauuggucgcuguuggccccaccugcgggcccugggggucgggggugccggcuguggggugcagggccuggcaucacucgcgagaaacugcaugcggcugcagguccuggagcuugaccacgugucagagaucacccaggagguggcagcagaggucugccgggaaggccugaagggacuggagaugcuggugcucacggcgacucccgucaccccuaaggcccuacugcacuucaacagcaucugccggaaccucaaguccauugugguccagauugggauugcggauuauuucaaagagcccagcagcccugaggcccagaagcuguuugaggacauggugacaaaacuccaggcucugcgacggaggcccggcuucucuaagauucugcacaucaagguggaaggcggcugcuaacccggguagggggcggcagggccccugccagccccacaccagggcacucucuuuggaccucagagggacccugguuuggacuagaccuuuggaggccgaguguuaucccuggcuucuggagggggacugucaagucuccuguccuccuccuggagcagcagagcaacaggccugacccagggcacugccuccccaguacaggggcuuggacagaagcugcccuccgacccccacccuaccccggcuggaguagccucuggcacagccagugaggagcugucaccaccagcgccuggugucaucaccuggaggaucugcaauaaccacccaguggcuccucagcuguucuggcuggccucuccuuccugaggcccagccuccuggucaggagcaucuggggccccaagccaaugggggcuccacaaggcagcucagacuuggcaaggagggcucuucuccucaaccuugcugcagccuucugggggcaccccuucagacagccugcccaggcuguggauccacauuuccuggggguaccacagccagaccuaggggccugggcacguggucagccaaaagcugggggcagcaguacagugggguaguggggguggguuuggaaaggaaacagucacccagaacuucuccccaggaugagaccacccuuccaaggugggggauugccagggggagaaaacuuauuuauugcuguaagacaggaccccuccucccaaccucauaccccaccgcacaccagagcuaaauucaaagcugaaaggcgcacguuucuauaccuacauucauuccugagggacccuccagagggucaaggucccagccccaggcagcccugucacagugagaaguaguuccuguccuuaaggaauuuccuucuaauccaggugcuugggcaggaacccgauggccuucgggucaccaaggcugucugggagggaggcacagggccgcccucugugcugaggccguggaggaagccaggaggaggguggcuugcuuugcuuccuugucuaauuagcuugcuugaagauguggccuuggcagggagccagacccauggggccaaggaagaggaagagcauccucaauagacucacucccccuuccuuggucuccacgggccccguggacugagggcugcauuggggucuucugccuaggggaagugcuggaccugagcuggagccacuuggcuuagaagccacaggauucacuuuucacuggccuuugcaguccccaaaggaucaggucucagaaccaaggcuccaaaggcugaggucuccccaguuccuccucucagaacucccacaguagcucagaggccggggguccugccaacuuucauuuggaaaguucuuucgaacaucuaaacuagaucuaucuuaggguuucuuucucuccuagauaggaucagcucccagcccuagccauuaggcugcugguccuggcgggggaugggguccccucguuacccaguccuucccagggacccaacuuccuaacacaaccuggcuuggacaugaagacccucccccagguuaccuuguaaagaguccuccagagcugggaucccaugggcgcagcagcacacccagcucccauggcgucacucccuagcucugucccagcuuuugcuaucauugcugacuuuuccuccuguggcucauucugucccugcccuuugaaaaccuaaaauaccaagggugucaugcuggcaacucccugcccaguccugcacaaagccuuggcuguguguggcaccccuugccuccuaccccagagcagcuggcuccauuggcuucucccugcaccagcccuguccucaggggucaggaaaaagcagcacagcuuucuuuccucuccuccagaggccuggaagggagguggagguccaguaagggccuggcugccuuggauuucuugguccugccuugccaacugcacccuguagcuccugcucccugugaccccagaaccagaggugcugccuucccugucuccuagacaaagcacaaagggaugcccugcuuggcuugagccugcccaacugaaggauuuucucugccccagggaccuuccaucccugaauacaaggcucuaggcaacuucucucugggugguacacacuagaaugccuggcauuagcccuagaaaggagguugggguguauggguagugagcuagggugggagaaagguggugcugaaaggacagaugcuaguuguaguuucacucacucauucauucauuagugcaacaguacugagcaccaccugcacuagaggcagaggggugaacaagauacccuucugccuggggggacguccacuucccauggguuuggcuauuuccaggaaagccccucaguccuccacccuguucuggcugugugugaaggaugugugugagcaggcccaauccuuugcagcaagaaugagaggucagaguauuccauugcacacgcacccuggggcugacagacuugugcccccuagccuucaugcaugcccaagcacuggcagcuuugcagccccugccccaccagccccuugacgcucuucuuuuguucucuccucggggaugagcucugcugcugaguagggagcuuuugcuugcugggaggcucuaugcauggauuuuuuuggugaccauacagcuagggcugaggaugggaacagggacagagggccuggcuaucccuagaagcacuucauccaucuuuacccacccaaacgggaucccuucacaucucauacccaguaagaugcaagaaaggaauaucugagagcaagcagcccugcuccaggggccccagguauguguagaggcccaguggggguggccacuugguguuucuaccacccccugccauccagucuggccccaguaccuaccugggagguugguguacuuggcuuaaguacuucaugcuuuauucaggcugcuuccccacagcaccggcaggaaaugaaggugcacuuauaugcaucccugcaggaauaaagaguggguggccugcccagcccagcaccacagccuuuccccagccaggagagaccaccuaaggaucaaggcagcuccuguuuucuugguucugugacacucgagucugagccagccccucaggaauugccucaaaagagaaaaacaaaaaaaaguccuccuucccaaggccugcuacuccaagguuuggcuccaucccuugccuuuggguccugccuauuuccccacuccuggucucuuaucuuuggggccaccaguggggagucacccgggccccaaucccucuaaggcgcuaaguugaaggaggccuucccagagugacuauuggugccaaagucccaguuccuguuggacuugggguaaaaacaggagauggugaguggguguaaggcccaaaugcccagagaaguuaacucgaacccaugggaccugucccagccugucagucccugaugaguguaacuuccuuccccugggggccuggcccuucucuccaacccaguggccaugcuuucucacccagccuugugcccggccugcauuucuguauauauugcuguguauuguguguauguauguauuccuggacaaguguguucaucugcagcccuugccugaggauaagguuuaggauuggguaaagaucagaauaccagggccagcuaaggcaacgacucccuccccaaacccuugggaccucagccagucccaaggcugcccugacaaucaggcaggcuccccaccgugaggccaagccuccucugccacugccagcauggcccaagggaggcuuggccuugggcuugccagccucagcucugcccugacaagggucuuguauccagggcagaggccugaggugacccaggcuugcuuuguggcugaugccagcaggcuugguucuagugggcaccacuggugggcaaccuccauaacuggcccuuaggcccuaccuuccuacacagcuaggcuauaaugggccugagugagaggguagcuuccccagccccaagcacaggcagagggguggagagcaauuuuugguuuuauuuuuguuucugaaguggugccuguaccuccagcccccagggggccuucccuggccacacuucucugccccacccaggcaucgccaucccagcacuuugcuccaugucacccguaagaugcccuuugcugaauguaccugaguguauguauuuaaaaggacucacaugggcaucagagaauuuauggcucuguauccaauaaaaaagauggugaaacuggucuaucugccaaaaaaaaaaa

SEQ ID NO: 39 - RNA, 6394 nt, Homo sapiens
caguccgccugcacgccgaggccgccggaccccacgcccggcccgcgcgggcgccccgagcccggcuccgcgcgccuggcggcuacauggagagugucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 40 - RNA, 6899 nt, Homo sapiens
agacaauaagaaacaaacgguacaacuccacaaaaggcuucccggaaaauaagugucaaauaaauccuacggaacucucgcuucugccacugagcucuguauaugcaaaccuguuuuuaugccaucgcuuccccaggcugagcugcguuuguuuggaaauguaagccacugccuuuucuuuuuuucuuuuuuccuuuuuuuuuuuuccccccucacuucaucugagggcucuggauuaauuccuugagacauaucuuugcauacauaucucaugaguuauguuuccuggagccuuuuacuaggaacauuaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaggcacuucagguggcaagacagcuccuucuucagcagcaacagcagcagcaaguuaguggauuaaaaucucccaagaggaaugacaaacaaccagcucuucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 41 - RNA, 6840 nt, Homo sapiens
agggcuuggucuagccagggcgcacugcgugugcugugucgcugcuugcagaccuggagcgguuuagauauguguggcauuaauagauuucuuuaagugcuaaaugcaacuuugaaaauuaccugcaagcuccaacugaucuuucaacuuugaaagugaagaauuacuugaggaauucuugugguuucuuugucauuuagugagagcccagccagcuacuguggaggcaaaccucccugagguugccuacaaagggcaauguuccaguguguuuucucuucgagugugcuucagccgcacaguacuuccuguuuauucaaacaucuuuuuuaccacucggcaacccccgcuucgcagaaacagccagaacccaucuacagcaagaagacggaaauccaaaggcagacaguacgggcucccuucgccaaacucuucauuuucucugcacuucagguggcaagacagcuccuucuucagcagcaacagcagcagcaaguuaguggauuaaaaucucccaagaggaaugacaaacaaccagcucuucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 42 - RNA, 937 nt, Homo sapiens
ggggggugggcgccagcgccccggcgaacggcaaagagggagccgcucccgcucggggggccgcuggagugcccagcgggaacccgaaaguuuguaagaggaagagagcgcgcggcgagcgagcgagcgggccgggggcagcggcagcggcgccggggaccauggugcugccggcgccuccuccgcgggcgugaaggcggcgcuccuacucccuccccggacuccgcggugucccagaagcuuuuguugacaauuccaguuuccgaacaaaacauuucggcaauggugagggcuucgaucccuucucugauuugcugucagccaugaacggauggaugugaugccugcuagccaaaaggcuucccucuguguguugcaguccuguggcauuaugcaugcccccucccagugaccccaggcuuuuuauggcugugagacacguuaaaauuucagggguaagacgugaccuuuugaggugacuauaacugaagauugcuuuacagaagccaaaaaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaguggcaucucauaaaccaucagcccucuaggagucccagcaguuggcuuaagagacuaauuucaagcccuugggaguuggaaguccugcagguccccuuguggggagcaguugcugagacgaagaugaguggaccugugugucagccuaacccuuccccauuuugaauaaaauuauucuuuggagaaaaaaaaaaaaaaaaaaaaaaaaaaaaaaaaaaaaaaaaaaaaaaa

SEQ ID NO: 43 - RNA, 7202 nt, Homo sapiens
auuuauuguguggcgguugcacgcuguacucgcggcggcccggcccucgcccgggagugcgugugugcgcgcgugugugcgcgagugagugugugcgcggggugcgcgcgcgggcgggagugggcgcgcgcggggaaaggcccgaaaacaccuuaguuugcaccgagagaccauuugcagcggaacccgaaaguuuguaagaggaagagagcgcgcggcgagcgagcgagcgggccgggggcagcggcagcggcgccggggaccauggugcugccggcgccuccuccgcgggcgugaaggcggcgcuccuacucccuccccggacuccgcggugucccagaagcuuuuguugacaauuccaguuuccgaacaaaacauuucggcaauggugagggcuucgaucccuucucugauuugcugucagccaugaacggauggaugugaugccugcuagccaaaaggcuucccucuguguguugcaguccuguggcauuaugcaugcccccucccagugaccccaggcuuuuuauggcugugagacacguuaaaauuucagggguaagacgugaccuuuugaggugacuauaacugaagauugcuuuacagaagccaaaaaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaggcacuucagguggcaagacagcuccuucuucagcagcaacagcagcagcaaguuaguggauuaaaaucucccaagaggaaugacaaacaaccagcucuucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 44 - RNA, 7150 nt, Homo sapiens
ggggggugggcgccagcgccccggcgaacggcaaagagggagccgcucccgcucggggggccgcuggagugcccagcgggaacccgaaaguuuguaagaggaagagagcgcgcggcgagcgagcgagcgggccgggggcagcggcagcggcgccggggaccauggugcugccggcgccuccuccgcgggcgugaaggcggcgcuccuacucccuccccggacuccgcggugucccagaagcuuuuguugacaauuccaguuuccgaacaaaacauuucggcaauggugagggcuucgaucccuucucugauuugcugucagccaugaacggauggaugugaugccugcuagccaaaaggcuucccucuguguguugcaguccuguggcauuaugcaugcccccucccagugaccccaggcuuuuuauggcugugagacacguuaaaauuucagggguaagacgugaccuuuugaggugacuauaacugaagauugcuuuacagaagccaaaaaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaggcacuucagguggcaagacagcuccuucuucagcagcaacagcagcagcaaguuaguggauuaaaaucucccaagaggaaugacaaacaaccagcucuucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagggugccauucgcaccaaccucucacugcauaagugcuuuaucagaguagaggaugaguuuggguccuuuuggacaguugaugaugaagaguuuaaacguggccgccauauucaacgaggccguccucgcaaauacugccccgaugaaaacuuugacgagcuugucgcacauaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 45 - RNA, 6502 nt, Homo sapiens
agucuagagacuuagaagcauuuauuaacagugaggcugcugaagguuuuuaauaacaaaauuacaaauacucuaucuaauugguaagacgugaccuuuugaggugacuauaacugaagauugcuuuacagaagccaaaaaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaggcacuucagguggcaagacagcuccuucuucagcagcaacagcagcagcaaguuaguggauuaaaaucucccaagaggaaugacaaacaaccagcucuucagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 46 - RNA, 7007 nt, Homo sapiens
aucacagcuuggugcuggggcucagcauggccagguggcuaaccagggcuuggugucaacuacauuugggaugaugacacuuggaggacacagagccacugugcagcaggagucgccacugcuaacagccagagugucccagaagcuuuuguugacaauuccaguuuccgaacaaaacauuucggcaauggugagggcuucgaucccuucucugauuugcugucagccaugaacggauggaugugaugccugcuagccaaaaggcuucccucuguguguugcaguccuguggcauuaugcaugcccccucccagugaccccaggcuuuuuauggcugugagacacguuaaaauuucagggguaagacgugaccuuuugaggugacuauaacugaagauugcuuuacagaagccaaaaaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaggcacuucagguggcaagacagcuccuucuucagcagcaacagcagcagcaaguuaguggauuaaaaucucccaagaggaaugacaaacaaccagcucuucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 47 - RNA, 7102 nt, Homo sapiens
ggggggugggcgccagcgccccggcgaacggcaaagagggagccgcucccgcucggggggccgcuggagugcccagcgggaacccgaaaguuuguaagaggaagagagcgcgcggcgagcgagcgagcgggccgggggcagcggcagcggcgccggggaccauggugcugccggcgccuccuccgcgggcgugaaggcggcgcuccuacucccuccccggacuccgcggugucccagaagcuuuuguugacaauuccaguuuccgaacaaaacauuucggcaauggugagggcuucgaucccuucucugauuugcugucagccaugaacggauggaugugaugccugcuagccaaaaggcuucccucuguguguugcaguccuguggcauuaugcaugcccccucccagugaccccaggcuuuuuauggcugugagacacguuaaaauuucagggguaagacgugaccuuuugaggugacuauaacugaagauugcuuuacagaagccaaaaaagguuuuugagucaugaugcaagaaucugggacugagacaaaaaguaacgguucagccauccagaaugggucgggcggcagcaaccacuuacuagagugcggcggucuucgggaggggcgguccaacggagagacgccggccguggacaucggggcagcugaccucgcccacgcccagcagcagcagcaacaggcacuucagguggcaagacagcuccuucuucagcagcaacagcagcagcaaguuaguggauuaaaaucucccaagaggaaugacaaacaaccagcucuucagguucccgugucaguggcuaugaugacaccucaaguuaucacuccccagcaaaugcagcagauccuccagcaacaagugcugagcccucagcagcuccagguucuccuccagcagcagcaggcccucaugcuucaacagcagcagcuucaagaguuuuauaaaaaacaacaggaacaguugcagcuucaacuuuuacaacaacaacaugcuggaaaacagccuaaagagcaacagcagguggcuacccagcaguuggcuuuucagcagcagcuuuuacagaugcagcaguuacagcagcagcaccuccugucuuugcagcgccaaggccuucugacaauucagcccgggcagccugcccuuccccuucaaccucuugcucaaggcaugauuccaacagaacugcagcagcucuggaaagaagugacaagugcucauacugcagaagaaaccacaggcaacaaucacagcaguuuggaucugaccacgacaugugucuccuccucugcaccuuccaagaccuccuuaauaaugaacccacaugccucuaccaauggacagcucucaguccacacucccaaaagggaaaguuugucccaugaggagcacccccauagccauccucucuauggacaugguguaugcaaguggccaggcugugaagcagugugcgaagauuuccaaucauuucuaaaacaucucaacagugagcaugcgcuggacgauagaaguacagcccaauguagaguacaaaugcagguuguacagcaguuagagcuacagcuugcaaaagacaaagaacgccugcaagccaugaugacccaccugcaugugaagucuacagaacccaaagccgccccucagcccuugaaucugguaucaagugucacucucuccaaguccgcaucggaggcuucuccacagagcuuaccucauacuccaacgaccccaaccgccccccugacucccgucacccaaggccccucugucaucacaaccaccagcaugcacacggugggacccauccgcaggcgguacucagacaaauacaacgugcccauuucgucagcagauauugcgcagaaccaagaauuuuauaagaacgcagaaguuagaccaccauuuacauaugcaucuuuaauuaggcaggccauucucgaaucuccagaaaagcagcuaacacuaaaugagaucuauaacugguucacacgaauguuugcuuacuuccgacgcaacgcggccacguggaagaaugcagugcgucauaaucuuagucuucacaaguguuuugugcgaguagaaaacguuaaaggggcaguauggacaguggaugaaguagaauuccaaaaacgaaggccacaaaagaucagugguaacccuucccuuauuaaaaacaugcagagcagccacgccuacugcacaccucucaaugcagcuuuacaggcuucaauggcugagaauaguauaccucuauacacuaccgcuuccaugggaaaucccacucugggcaacuuagccagcgcaauacgggaagagcugaacggggcaauggagcauaccaacagcaacgagagugacagcaguccaggcagaucuccuaugcaagccgugcauccuguacacgucaaagaagagccccucgauccagaggaagcugaagggccccuguccuuagugacaacagccaaccacaguccagauuuugaccaugacagagauuacgaagaugaaccaguaaacgaggacauggagugacuaucggggcgggccaaccccgagaaugaagauuggaaaaaggaaaaaaaaaaaaacacgucaaaaguuagcagugaaauuguucuccauuuguuguacagucuggaggauuuucacuacguuuugacaacucugaaauguguuaacucuuagugccaucaagaaccccauuugggaguauuuuugauuuuucuacuuuuuguugaaaaaaggaauuuguacucugugcauuggauggacuuguuugguacuugggauuuuccucucuuaaccgucaacaucaguguuguaaauuugcuaaacugauucacuuuuagcagcagacuuugaacugcaguccugccaacguuggacacugaggacgcccgacagagcuugugcaccuaagcugcagaccaagccuuugcccagaauuuaaggauuccaauggacgaccuauuugcacaguacugcauguugauuaucacugccuuuacuccuuuuuuuuuuuuuuuuuuuuuuuuuuuuuugcuuccaguugggauggggaaggccuuuguguguguauuggggggagggguuaaaaaauaauuaucccaaacuuuuuaauguauugcuuuuuuuuuuuuuuuuuuuccuucuacuauaccauuuuaaguucugaccucaggccuccauuugggccgauggccucuuggaggcuuaaaguuuucuguaccuugugaugaauguuaauagguguuuuuauuauacaaagcugaaugucauuucucguuuguagcuuucugucacucauuccaucuuccuucagacaucaccacguuucucuaaagucagaaaacauuccguuuuggucuuuuucaaaaaggucccaaaugcugcacucuacacaugaaggcccucucacacagacgugacguccugccagaaagagaaugaaugacagaaaaaaaaaagagagacaaacucuaggaacaaugccgauucauuccacgcagcaguauugggggugguucgggggagggguguuucggauuuucuuuuuuucuuuucuuuucuuuuuuuuuuuuugcagcaaccauuaauaaaugccaccacauucuaccagcacaaggaaacauaggcagcacugaaaaaaaaaaaaaagcucauauuaauuagacugacaauauggccuuggaaggcucucccuuguggaaccaaguugccaugggccuugggugcucugcgauaacggguguggguugguuuuguuugcaaaauggccaaaaaaaaaaaccggcuuccccgagcagcugcccugaaaguagggguggcggcggcggcgcugaguuuauacauuaguucagaccuacuugguggcauuaaacuguuugaaugcaaauucgauuucagauugaacuuguuaagggaguuaacgagggcugaguucagcaaaugcuaaaguguuaauuucaaauaugcaaauuugguacugcaguuuguuaugcaauauuauaucaccaacccaguaucacaaaaacucauagaagauaucauguaggcccugggcuuugggggggucccaaacaugguaugcagaaaugugaugguuacaggucaguacaaccucaguccuuagaaccccuccacacuucagcucugcacccacuuuccugucauuuauuuauauaggacuguaguuuuuuuuaguucgagagccuuucgaagcuuaauuuauauucuuucuuuguaccuuuuuucuaaaauuaccaaagauauuacacaaagguaaauuauguucucuguuuuaugcuuuaucugaugaagccaaauauccucuuauuguugaucaaaggaggcaaaagaauuuagaggcaaaugacaagcgauaggcuauugcaaccugagaaagagaacugcuccuucaucguaaauuuagaagaccaaguagauaauggaaccaaaguuguuacuuuuuucuaguaguuauuuuuccuuuuucuuuuuguguaccucuacagagaccaaaacucauucucuuaaagagauuuuauggggcuacugcagauaaaaauaggacacaauauuaaaggagcuacagaaggaagggagucccaucucaaaaaaaaaaugaauguaugccacugcaauuagaguauccaauaaaggagacaguuuagagucaggacagaaaagcuuccauaauugaacuagauuacauaauaguauuucuagaaaaagagauauuuuuagauuguaugccacuuuuguuuaagaacugugcugugaucacuguauuaauuuugguuuaucuuggcauauauccuucaguuuguuuuuauuuuuaauuuuuccuuuuuuuccgauuaggcuuuggucagcauuuuucauuuaaagaaaaguaacacucccauccacucauaagcuugguacaaaaacuucucuggcaguuacuuuugaagcuucacucugcuuucuguauaaagggcagucuguggucacgcaagacuuuaaaaaaaaaaaaaaaaaaaaaaaaaaaaaaaaaaaaaaaaacuuuuccaggcagcuucaugaugugcaggcaguagccagacagggucaugggaagggggcccugugcuucuaaacugagugguugcugguuaguuugguauucaaaagaggauaaaaaucugguagauuaguucauucucagcauguguagcuagacaugaguaaagauaacagcaugagaaacuguuaguacgcauaccucaguucaaaccuuuagggaaugauuaaaauuuaaaaaaaaaacauuucacucaguugcacuuagucguaugucuugcaugcuuagucuaaagacuguagcaaaaaaaaaaaaaaaagaaaaauuagauuuuacauaucuuugcagguaucacagccuugcagaagaaccaacugaaaaaaaaauucucaggcuuuacagcaagcaaacuucacuaugauuuuuacaauucugauucuguauccccuggggguuaucccaguugcuucuuuaggaugggguuuauuacguuguacauauaucccgaugugucugugugaaucuuugucuuuuuugggggagggcagagggcgguucuuuuuuuagauauuguuccuaaaaaggaauaaaugcauacaccuguuugucaaaacaccuuugcuuuuugugcaacugcuuuauauuaacgauacuaaaaaaaaauagcuuuggaaaaaaaacuacuguauguaacggaauugcagaauaugcugcacauguauuuuauuuaguuauccuugcuuuaagaauauuggaugacauuuccugacaugugggagggagaaacucccuaacuuuuuuuuucugcuuuuaaacuguaacauaguugaagauuucuuuuuucuguucucauugauuggagcauuuuguacagguuuugugugugugugugugugugugugcgcgcgugcguguguguuaaucuguuuuuugauacauuccuaucccuuguguuuauccuaccacugccuuccuggcuaucuuaaacaaguucauacauuugaaaagaaaaaaaaauguuguuuaaaaaauguuuucuccugcugcaguaaauauuuugcaugaugaaauuccagggucacacuuuuccaaguuuaucagugaaguagugauuaacaauggggagugucaaaacuauugaacuuuuguauaaaaaaaaaaaaacuuuacaaggugccaagauguaaagaaaaucuguuacuuuuuuuuucucaaagaaaagcauacauuagggagguagucccgugugucagacaaaugcacugucaggaaugaggauccaaccuaccugucccuagaguccgucuuguaagaugaguuaggcugccccuuggaccagccacaaaauggaauaucaaggcuuauguacauacgugaagaguuaccaccaguccugccaccuuuggacagcucuaacaccauccccagcauccagucagaccuaguaaagaaaaccuuggauucuuaacccaagauaggcuguaaaucacuagcuuuuuuuuccucaugaaaaaaaauagaguuaaaaaauauuuccucucuuuuccauauuccagcugaacuccguuuccaaaggcacaaagaagagugugcuuauucagauuuugaaucuuuuugguaccuuuugguuaaugacauagccuccugaaauucuggaugucuucaaagucaguuuugcuucuuuauccugaaaaucagauuuacaaugcugaaggcauuucuugggcccaguguagcucacgcaaucucugcuacccauaagccuugaugaagaugauacaguccggacugugagcauggugcuucauguauaugugcugccaguaacaagaauuuuuuuguuuuguuuuguuuuguuuugauaaggcauaaaagaaacucauuccuugacaucaacuguaauuccaucauuccaugucugcggauacagacaauaaaaaaaauguuguguagucaguacuaauuacugacauuauaagcauucucaaaugcaauaaaaaugcugguuguucacgcugguaaaaaaaaaa

SEQ ID NO: 48 - RNA, 3578 nt, Homo sapiens
gaaccgaguggccggcacuuccggccggcggcggcugcuccgggugagcaggccccugaagugcugugccuggagauaccauuguggacccuggagaggcaccugcugcuuaugcgguaaaaccucuaccaccccaaaccuggucaguuugaggggagccgcugccauaguauccacagagaaggccggaguuccagccguguggagcagcagaggggagucugacagggucaccuccuacacagagagaauuggcagcugacugcacugccaggagccaaggccuagguguguggagccuggaagaccagggauaccugaggcaggaggugcagcguguggagugagaaacccgagagggacuggggccugugcucaggaucagcagagcaggcaggcagaguggagagggagagacggagccccagaaggggagcaggaggagagggaaguaguggcagcagcccaggccaggcguguguccugcaagugcuaggaccagccaccccuccccauggccuccaagccggcugccgggaagagcagaggggagaagcggaagaggguggugcugacacugaaggagaagauugacaucugcacgcgccuggagaagggcgagagccggaaggcacugaugcaggaguacaaugugggcauguccacccucuacgacaucagggcccacaaggcgcagcugcuccgguucuucgccagcucggacuccaacaaggcgcuggagcagcggcgcacgcugcacacgcccaagcuggagcaccuggaccgcguccuguacgagugguuccuggggaagcgcuccgagggcguccccgugucaggccccaugcucaucgagaaggccaaggacuucuacgagcagaugcagcucacugagcccugcguguucuccggaggguggcuuuggcgcuuuaaggccagacacggcauuaaaaagcuagaugcauccagugaaaagcagucagccgaccaccaggccgcggagcaguucugugcguuuuucaggagcuuggcugcugagcacgggcuguccgccgagcagguuuacaacgcugaugagaccggccuuuucuggcggugccugccaaaucccacuccggaaggcggggcugugccuggccccaagcagggcaaggaccggcugaccgugcugaugugugccaacgccacgggcucccacaggcucaagcccuuggccaucgggaagugcagcggucccagggcuuucaaaggcauccagcaccugcccgucgccuauaaggcccaggggaaugccuggguggacaaggagauuuuuuccgauugguuccaucauaucuuugugcccucggugagagagcacuucagaaccauagguuugccggaagacagcaaagccguucucuugcuggacagcucccgggcucacccgcaggaggccgagcugguguccaguaacguuuucaccaucuuccugccugccagcguggccucauuggugcagcccauggagcagggcauucggagagauuucaugaggaacuucauuaacccuccggucccccugcagggcccccaugcccgcuacaacaugaacgaugccauauucagcguggccugugccuggaacgcagucccuagccacgucuucaggcgggccuggaggaagcuguggccgucgguugcguuugccgaaggcuccuccucugaggaggaguuggaggcagagugcuucccagugaagccccacaacaaguccuuugcacacauccuggagcuugugaaggaaggcuccuccugcccgggccagcuucgccagcgccaggccgccagcugggggguagcgggaagggaggcagaagggggacggcccccugcugccacgucgccagcagagguuguguggaguucagaaaagacuccaaaagcugaccaggacggcggaggagauccuggugagggcgaggagguggccugggagcaggcggccguggccuuugacgcaguccugcgcuuugcggagcggcagccaugcuucagugcgcaggaaguggggcagcugcgggcgcugcgugccguguuccggagccagcagcaggagacugugggccuugaggaugugguagugaccucaccagaggaguuggcaauuccuaagugcugccuggaggccucuacagagacauaauguuggagaacuucugccacauggcagugcuggguaagaccccucuguccccaccagugcccuugagguuauggguguugguaggucuguggccuccauuuccaugacgaugguguuaaggacugcauguuuguguccccccccaaaguuuauauguugaaacccugacccccaggagggugguauuaggagguggggccuuugggaggugacuaggcuuaggugagaucgugaggguggggcucgccgaugagaucgaguccuuauaagaaaaggaaggaacuagagcgaggucacuuugugcugugugagcauacaagaaaacugccaucuucaagcugggugcaguggcucacgccuguaaucccagcacuggaaggccaaggcauuaggauugcaugaguccaggaguucgagaccagccuaggcaugauaagaccuuauugcuaaaaaaaaaaaaaggaaauaaaauagaaaauugccaucugcaaaugagaaagccuucagccgacaccagacugccagugccuuugauuuuggacuuucaguccccagaucugugaaaaauuaauuuauguuguuuaagacaccuagccugugguauuuuguuaucgcaauccaaacugacugagacaggcaguaaggagagaacagagguuccagccugcuucagguccaggcaguucugccauugccccuaacuccucagugccagucaugggccaggccccugggggacuuggaaugaguaaaacaugcaccuauuguuaagguucaccugguccagaaggggaggcagacccaccugcccaccaugacccggacagaggcgauaaauacggagcaccucauggccagggccuggaggauucccaccauacccuacccagcgccaauaacagagggacccuggaguuguguacacaagauagcaguucagugcuugcacacauacuaguugagaagggaaaucagggcuaaugaaacagcucaacugucuggcagagccaggcuccuucugccaugauacaguuuggauguuugucccuuucuaaucuuauauggaaauuugauccccaguguugaagguggggccuggugggagauguuccuguuaucgggauggauccuucaugaauagccuggugccaucuuugugguaacaagugaguucucacuugauuaguuucugugagaacuguuuguuaaaaagagccugguggcaccucccucccuucuuccuccuucucuugcuaugugaugcugcugcccuuugccuuccgucaugaguggaagcucccugaagcccccaccagaagcaaaugcuggcaccaugcugcuuguacagccugcagaacugugagccaaauaaaccucuuuucuuuauaaauua

SEQ ID NO: 49 - RNA, 9116 nt, Homo sapiens
gaaccgaguggccggcacuuccggccggcggcggcugcuccgggugagcaggccccugaagugcugugccuggagauaccauuguggacccuggagaggcaccugcugcuuaugcgguaaaaccucuaccaccccaaaccuggucaguuugaggggagccgcugccauaguauccacagagaaggccggaguuccagccguguggagcagcagaggggagucugacagggucaccuccuacacagagagaauuggcagcugacugcacugccaggagccaaggccuagguguguggagccuggaagaccagggauaccugaggcaggaggugcagcguguggagugagaaacccgagagggacuggggccugugcucaggaucagcagagcaggcaggcagaguggagagggagagacggagccccagaaggggagcaggaggagagggaaguaguggcagcagcccaggccaggcguguguccugcaagugcuaggaccagccaccccuccccauggccuccaagccggcugccgggaagagcagaggggagaagcggaagaggguggugcugacacugaaggagaagauugacaucugcacgcgccuggagaagggcgagagccggaaggcacugaugcaggaguacaaugugggcauguccacccucuacgacaucagggcccacaaggcgcagcugcuccgguucuucgccagcucggacuccaacaaggcgcuggagcagcggcgcacgcugcacacgcccaagcuggagcaccuggaccgcguccuguacgagugguuccuggggaagcgcuccgagggcguccccgugucaggccccaugcucaucgagaaggccaaggacuucuacgagcagaugcagcucacugagcccugcguguucuccggaggguggcuuuggcgcuuuaaggccagacacggcauuaaaaagcuagaugcauccagugaaaagcagucagccgaccaccaggccgcggagcaguucugugcguuuuucaggagcuuggcugcugagcacgggcuguccgccgagcagguuuacaacgcugaugagaccggccuuuucuggcggugccugccaaaucccacuccggaaggcggggcugugccuggccccaagcagggcaaggaccggcugaccgugcugaugugugccaacgccacgggcucccacaggcucaagcccuuggccaucgggaagugcagcggucccagggcuuucaaaggcauccagcaccugcccgucgccuauaaggcccaggggaaugccuggguggacaaggagauuuuuuccgauugguuccaucauaucuuugugcccucggugagagagcacuucagaaccauagguuugccggaagacagcaaagccguucucuugcuggacagcucccgggcucacccgcaggaggccgagcugguguccaguaacguuuucaccaucuuccugccugccagcguggccucauuggugcagcccauggagcagggcauucggagagauuucaugaggaacuucauuaacccuccggucccccugcagggcccccaugcccgcuacaacaugaacgaugccauauucagcguggccugugccuggaacgcagucccuagccacgucuucaggcgggccuggaggaagcuguggccgucgguugcguuugccgaaggcuccuccucugaggaggaguuggaggcagagugcuucccagugaagccccacaacaaguccuuugcacacauccuggagcuugugaaggaaggcuccuccugcccgggccagcuucgccagcgccaggccgccagcugggggguagcgggaagggaggcagaagggggacggcccccugcugccacgucgccagcagagguuguguggaguucagaaaagacuccaaaagcugaccaggacggcggaggagauccuggugagggcgaggagguggccugggagcaggcggccguggccuuugacgcaguccugcgcuuugcggagcggcagccaugcuucagugcgcaggaaguggggcagcugcgggcgcugcgugccguguuccggagccagcagcaggugaggaggcggcguggugcccucggggcuguggucaagguugaagcccuccaggagggcccugguggcuguggggccacagcucagucucccuugcccugcucauccacagcaggugacaacugauggcuucucugcccugcccuggccacuggcccuguuucuccccacacccuggaguggcaugguccugugccccgaccccaccugaggcaggagggcaugugcagacacucaagagcccuuccaggagugggucgcccacggguguggcucgggugcccaggacggucugugcccgagguuccugucaauacagguuuuauuuuaucacuugccgugucauccgaaagugaggaaauguuuuggaaggguccacccuagccuagaacaagccagagccgcacccuggcuggaaugggggccaggcugagccgaucuggucucguguucgcuggcugaucauugcaguaucagaggguggagaugucagucuguccacguggagagaaguugcccuccaggccgacaggaggccaugcccaccgccccuggacaggcuucgucucagaaggcucuaucugcugggcuggcggccauccccguguuggguggaccccgagcacgguugccugagguccgauggcccgagagcugggacucaguucuuggccugcuagcggcugaacaggccgcacaucucacuucaguuguggccucauucagcagaaugacucuggaaccauccucuguuacccgcagauccugucccaugggcucuggccccaagauguuggggggccccacggagaguugacuugguagaguuccuuucugggaagaaaguaggaguggcugaccaggcccugcucaucacccggauagaggacacggacccugugugguauuuuggcauuuuggcucagaguccaauguaccauguugcccagaauuucauacuuacggccuuuacaugaauacgucuugaucagacauucagagauuagucuuagguuuccacguaagucacugaaaacaguaaaacaggcugcuuagauuucuagcaguaugugugcucuuauccccagcuguuagcacguuaguaauacuuuuggggaggaguugucuguccuggucaguuuucacagaggcgguuguaagaggaugugguuauuuucuccaugucuguaguugcuuugcggcaucuccauuucacgggcaacacucacugccugcagcugagauuccagaacgcccuguguccacagugauuagccucgucuuucagcgagcauaaaauaggugcauuguccuuuucuuuucuugaggcagggucucacucuguugaccaggcuggagugaaguggugugaucccagcucacugcagccucgaccucccuggcuugagugauccucccgccccagccuccugaguagcugggacuagaggcacgcaccaccgcgccuaccuuuuuauuuuuuuuguagagacagggucucacuguguugcccaggcuggucuugaacucuuggccucaagcgauccuccuuccucauccuucuaaagugcugggauuacaggugugagccacagcacucggccuaaauagguuuuucuugcuauguuauuuucagugcuuugcccuugaauucuuuuccucucuuaaauucauaucauuuggccgagucagcacguaguuugcauuccagcgucugcuguugauuccacugaaaccucuguucucaugaaggagaggauuuggcucgcugugguuucucguacuuucacaucucuguagauacaggggcaaaagucauugcugacagagcccuaucuauguccuggaaguguguguuucuugcuuugaaugcaugugugaagauuauaaaagccaaauaguuuuuaaagaucucggugcugaacgacagucccaguucccauuugugucccauugucuuuguggugucacccgcacuguuugcuucauugucuggggaacuucugcacauccauguacuuugugccauuucugggcucacgugucacuccuaauagagccucuuaaccaagacugcuggauugcagcugcuugaggcaacacaggagguuagaacaugucccgagggcucugaaacaguaggacauccuggguggguggagcuggggagggcaugggcaggccagcuggccgagcuccuggcuggcucuccugaguccucuggacauggcuugucccugugagacuguagcucuucaggaccugagcucuucccguucuugaccugcucuugugcugacugugcugggcugauggguaggcgcuuguccccagugaguggcuguguccucagggugccccucuuccagaccaacugcagcaaggccuggagccugacucuaggugcagcccuuguggcuggaaucccuugugccacccaggggacucgggcccugcuccuccugcagccccuccucaccugggcucgcugcuggagugagguuuuuagggagcccagagcguacucaucucugagcugcugggcccagcugccuggugcauaccucuugccucgguccugucccccagaacgcuuuauggagacugccguguccuccgagcacuuuguuuucacuuccugacccgucucagaagcucaagugguugaauguuuuagccucagaaucuuuuuccccaagcccucucagguggagccccgcuguaccagacaccacagcacagcucugugaggcugugucucuucacucuguuucuggggcugcugaggccccugggaagcccuggagcuugccagagcagagcgagaaagucucugguccuguuuaaaugccuccgcguuuuaucugugagcaccaggcuggagccacucccugucuucucacagugccccugcaaguugcagccucaguuuuccaguggcucgugggguagcaggugcugcgucauaugguuugggaccucuguggcugcuuccagccugcuaauagaaccucuuuccaccacugccuucaucuuugggguggcaccccugagugcucaggccugagggcgcuccuguguccucacucgggacaggcagccuccacucugggagcucccaucccucgggaugcccaggagaggccauacuuucaggguggcauggugaauucagggguuaggauggguagguggucuuucagccccuucugcguugggguugguuuacagaacaccuguccugccugugccagccuggccuucugcgagcaccccaggcacgcuguccuggaaguugucugucuccugcauaggagggucuagagcagaccuugcccugggccugucugugcgugacuuccauaugcuucauucgugcucagccauggcugagcaugcgauggagagcaccuggaccgccccugacgucgcaggccacuguucuuauccagugggccaagugauugauugcuaguggugggguugauuggaagaaaggaagucgcagccccugcccacggcuuccccagcucgcugggguuggaggacugucaucaguuguccccaguuaaaguccauuuggaggagguucagggucccaguggcuggaugugcuuguggagggacacgguuugaggcagggguuugcagaagguguagacugcauccauucuugggaggcuguggcacagggcagugacaacuugggacaaaucaagacccguccacacugcaccucagggaccucaccucacgccccacauaaaugugcuuauuggggggccgggugugguggcuucugccugaaaucccaguacucuggaaggccacggcaggaggaucacuuuaguucaggaguuugaaaccagccugggcaacauagggagaccccaucucuacagaaaguaaaagaaaaugagccaggugugguggcaugugccuguaaccccagcuacucaggaggccgaggcgggaggauccccaagcccaggaguuugaggcugcagugaguuauggccucugcacuccagccugggugacagagugagagaccuaaucucuaaaaacaaaaaauaacaaaaugugcuuuggggucagguguggagaucagggucggggaggucaugagguagguaggggucaggauguggacagucaugggcugguggguguggcagccgugaggggugggugguggccugggaggcagggccugggcagggcuucugcacuucugcugcuuacccagcccccucuggguucgaggacucuccuugacaagggcugagcucucgggcacugcguuggacucagcugccuacugggaggcuggggagaauggaccugugagaccuuguuuguccucuucccugcccagcucuuguagaccccaggccuggcuguccgcagggccaggagcugcagccauggcagccaccagccucccacaugagccccagcucccagccccacacgcccacuccuuugcccauguggugucagcucuggccuccgcuuccacacauggacccucagaccuugcacaucuguuguuucccuugcagggcagugaugaugacaugaaaugagguggggucacuccuguuuagggaugagacuggggcccagggaggggcaggugugaaguugcugagggcagagggagacccagggcuaccugcuccuaaugaccagccucacguucuccacugcucacucccugccguucaccagcugcaugaccuugagcagugcacucaacuguaaacagaggugaaaacacuccuuuaccaacuguuuuaggggccacucuuguuugaggccuugaaccuccuuucaguuccguggugguccugagccucugaaucuggguauggccccugcuggguccuggucucuuaggcagcacgauggccucccugcucaaggcccugcacccaggggcagauugugggaggcccacuggccauggcugggcccuaucgcagagagccugucagcacggccauuuccacacccaggcuggcugggaagcaagcuacgaggugcucaaaggcccucuaugucugcagguagccccgucccacacaggcgaggaucguugggccuccuuaguagccaaaggaacugagguccagagcagggaauguguugcccagugucagcauggucugcucuggggucaggcgucuugcccugguccuaagccaccccaagccaaggcaugucccugggcccauuuccucucucgaggucuuggguucccuauuucugaugugugugaaaggaaggccugucuucuggggugaccaugaggcucagugggaggagcugccaggcuugagcugggacuuggcaccuggccagccuuuuuucuauuucauuguaccuugaugggcugucaguucugaagagaaaaaauacacuaggaauccauucuuccuccaccauucucccuggcagagggaagugacuacaccccuaugguguggacauacugacgcuccuccaugaagcagcaaaguucuguuguuucaggagacugugggccuugaggaugugguagugaccucaccagaggaguuggcaauuccuaagugcugccuggaggccucuacagagacauaauguuggagaacuucugccacauggcagugcuggguaagaccccucuguccccaccagugcccuugagguuauggguguugguaggucuguggccuccauuuccaugacgaugguguuaaggacugcauguuuguguccccccccaaaguuuauauguugaaacccugacccccaggagggugguauuaggagguggggccuuugggaggugacuaggcuuaggugagaucgugaggguggggcucgccgaugagaucgaguccuuauaagaaaaggaaggaacuagagcgaggucacuuugugcugugugagcauacaagaaaacugccaucuucaagcugggugcaguggcucacgccuguaaucccagcacuggaaggccaaggcauuaggauugcaugaguccaggaguucgagaccagccuaggcaugauaagaccuuauugcuaaaaaaaaaaaaaggaaauaaaauagaaaauugccaucugcaaaugagaaagccuucagccgacaccagacugccagugccuuugauuuuggacuuucaguccccagaucugugaaaaauuaauuuauguuguuuaagacaccuagccugugguauuuuguuaucgcaauccaaacugacugagacaggcaguaaggagagaacagagguuccagccugcuucagguccaggcaguucugccauugccccuaacuccucagugccagucaugggccaggccccugggggacuuggaaugaguaaaacaugcaccuauuguuaagguucaccugguccagaaggggaggcagacccaccugcccaccaugacccggacagaggcgauaaauacggagcaccucauggccagggccuggaggauucccaccauacccuacccagcgccaauaacagagggacccuggaguuguguacacaagauagcaguucagugcuugcacacauacuaguugagaagggaaaucagggcuaaugaaacagcucaacugucuggcagagccaggcuccuucugccaugauacaguuuggauguuugucccuuucuaaucuuauauggaaauuugauccccaguguugaagguggggccuggugggagauguuccuguuaucgggauggauccuucaugaauagccuggugccaucuuugugguaacaagugaguucucacuugauuaguuucugugagaacuguuuguuaaaaagagccugguggcaccucccucccuucuuccuccuucucuugcuaugugaugcugcugcccuuugccuuccgucaugaguggaagcucccugaagcccccaccagaagcaaaugcuggcaccaugcugcuuguacagccugcagaacugugagccaaauaaaccucuuuucuuuauaaauua

SEQ ID NO: 50 - RNA, 6816 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagaaucuccccugcagacccccacugaggacggaucugaggaaccgggcucugaaaccucugaugcuaagagcacuccaacagcggaagaugugacagcacccuuaguggaugagggagcucccggcaagcaggcugccgcgcagccccacacggagaucccagaaggaaccacagcugaagaagcaggcauuggagacacccccagccuggaagacgaagcugcuggucacgugacccaagagccugaaagugguaaggugguccaggaaggcuuccuccgagagccaggccccccaggucugagccaccagcucauguccggcaugccuggggcuccccuccugccugagggccccagagaggccacacgccaaccuucggggacaggaccugaggacacagagggcggccgccacgccccugagcugcucaagcaccagcuucuaggagaccugcaccaggaggggccgccgcugaagggggcagggggcaaagagaggccggggagcaaggaggagguggaugaagaccgcgacgucgaugaguccuccccccaagacuccccucccuccaaggccuccccagcccaagaugggcggccuccccagacagccgccagagaagccaccagcaucccaggcuucccagcggagggugccaucccccucccuguggauuuccucuccaaaguuuccacagagaucccagccucagagcccgacgggcccaguguagggcgggccaaagggcaggaugccccccuggaguucacguuucacguggaaaucacacccaacgugcagaaggagcaggcgcacucggaggagcauuugggaagggcugcauuuccaggggccccuggagaggggccagaggcccggggccccucuuugggagaggacacaaaagaggcugaccuuccagagcccucugaaaagcagccugcugcugcuccgcgggggaagcccgucagccgggucccucaacucaaagcucgcauggucaguaaaagcaaagacgggacuggaagcgaugacaaaaaagccaagacauccacacguuccucugcuaaaaccuugaaaaauaggccuugccuuagccccaaacaccccacuccugguagcucagacccucugauccaacccuccagcccugcugugugcccagagccaccuuccucuccuaaauacgucucuucugucacuucccgaacuggcaguucuggagcaaaggagaugaaacucaagggggcugaugguaaaacgaagaucgccacaccgcggggagcagccccuccaggccagaagggccaggccaacgccaccaggauuccagcaaaaaccccgcccgcuccaaagacaccacccagcucugcgacuaagcaaguccagagaagaccacccccugcagggcccagaucugagagaggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcagauaauuaauaagaagcuggaucuuagcaacguccaguccaaguguggcucaaaggauaauaucaaacacgucccgggaggcggcagugugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 51 - RNA, 5724 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagaaucuccccugcagacccccacugaggacggaucugaggaaccgggcucugaaaccucugaugcuaagagcacuccaacagcggaagcugaagaagcaggcauuggagacacccccagccuggaagacgaagcugcuggucacgugacccaagcucgcauggucaguaaaagcaaagacgggacuggaagcgaugacaaaaaagccaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcagauaauuaauaagaagcuggaucuuagcaacguccaguccaaguguggcucaaaggauaauaucaaacacgucccgggaggcggcagugugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 52 - RNA, 5631 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagaaucuccccugcagacccccacugaggacggaucugaggaaccgggcucugaaaccucugaugcuaagagcacuccaacagcggaagcugaagaagcaggcauuggagacacccccagccuggaagacgaagcugcuggucacgugacccaagcucgcauggucaguaaaagcaaagacgggacuggaagcgaugacaaaaaagccaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 53 - RNA, 5718 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagaaucuccccugcagacccccacugaggacggaucugaggaaccgggcucugaaaccucugaugcuaagagcacuccaacagcggaagaugugacagcacccuuaguggaugagggagcucccggcaagcaggcugccgcgcagccccacacggagaucccagaaggaaccacagcugaagaagcaggcauuggagacacccccagccuggaagacgaagcugcuggucacgugacccaagcucgcauggucaguaaaagcaaagacgggacuggaagcgaugacaaaaaagccaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 54 - RNA, 5811 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagaaucuccccugcagacccccacugaggacggaucugaggaaccgggcucugaaaccucugaugcuaagagcacuccaacagcggaagaugugacagcacccuuaguggaugagggagcucccggcaagcaggcugccgcgcagccccacacggagaucccagaaggaaccacagcugaagaagcaggcauuggagacacccccagccuggaagacgaagcugcuggucacgugacccaagcucgcauggucaguaaaagcaaagacgggacuggaagcgaugacaaaaaagccaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcagauaauuaauaagaagcuggaucuuagcaacguccaguccaaguguggcucaaaggauaauaucaaacacgucccgggaggcggcagugugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 55 - RNA, 5637 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagcugaagaagcaggcauuggagacacccccagccuggaagacgaagcugcuggucacgugacccaagcucgcauggucaguaaaagcaaagacgggacuggaagcgaugacaaaaaagccaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcagauaauuaauaagaagcuggaucuuagcaacguccaguccaaguguggcucaaaggauaauaucaaacacgucccgggaggcggcagugugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 56 - RNA, 6762 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagaaucuccccugcagacccccacugaggacggaucugaggaaccgggcucugaaaccucugaugcuaagagcacuccaacagcggaagaugugacagcacccuuaguggaugagggagcucccggcaagcaggcugccgcgcagccccacacggagaucccagaaggaaccacagcugaagaagcaggcauuggagacacccccagccuggaagacgaagcugcuggucacgugacccaagagccugaaagugguaaggugguccaggaaggcuuccuccgagagccaggccccccaggucugagccaccagcucauguccggcaugccuggggcuccccuccugccugagggccccagagaggccacacgccaaccuucggggacaggaccugaggacacagagggcggccgccacgccccugagcugcucaagcaccagcuucuaggagaccugcaccaggaggggccgccgcugaagggggcagggggcaaagagaggccggggagcaaggaggagguggaugaagaccgcgacgucgaugaguccuccccccaagacuccccucccuccaaggccuccccagcccaagaugggcggccuccccagacagccgccagagaagccaccagcaucccaggcuucccagcggagggugccaucccccucccuguggauuuccucuccaaaguuuccacagagaucccagccucagagcccgacgggcccaguguagggcgggccaaagggcaggaugccccccuggaguucacguuucacguggaaaucacacccaacgugcagaaggagcaggcgcacucggaggagcauuugggaagggcugcauuuccaggggccccuggagaggggccagaggcccggggccccucuuugggagaggacacaaaagaggcugaccuuccagagcccucugaaaagcagccugcugcugcuccgcgggggaagcccgucagccgggucccucaacucaaagcucgcauggucaguaaaagcaaagacgggacuggaagcgaugacaaaaaagccaagacauccacacguuccucugcuaaaaccuugaaaaauaggccuugccuuagccccaaacaccccacuccugguagcucagacccucugauccaacccuccagcccugcugugugcccagagccaccuuccucuccuaaauacgucucuucugucacuucccgaacuggcaguucuggagcaaaggagaugaaacucaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcagauaauuaauaagaagcuggaucuuagcaacguccaguccaaguguggcucaaaggauaauaucaaacacgucccgggaggcggcagugugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 57 - RNA, 5544 nt, Homo sapiens
ggacggccgagcggcagggcgcucgcgcgcgcccacuaguggccggaggagaaggcucccgcggaggccgcgcugcccgcccccuccccuggggaggcucgcguucccgcugcucgcgccugcgccgcccgccggccucaggaacgcgcccucuucgccggcgcgcgcccucgcagucaccgccacccaccagcuccggcaccaacagcagcgccgcugccaccgcccaccuucugccgccgccaccacagccaccuucuccuccuccgcuguccucucccguccucgccucugucgacuaucaggugaacuuugaaccaggauggcugagccccgccaggaguucgaagugauggaagaucacgcugggacguacggguugggggacaggaaagaucaggggggcuacaccaugcaccaagaccaagagggugacacggacgcuggccugaaagcugaagaagcaggcauuggagacacccccagccuggaagacgaagcugcuggucacgugacccaagcucgcauggucaguaaaagcaaagacgggacuggaagcgaugacaaaaaagccaagggggcugaugguaaaacgaagaucgccacaccgcggggagcagccccuccaggccagaagggccaggccaacgccaccaggauuccagcaaaaaccccgcccgcuccaaagacaccacccagcucuggugaaccuccaaaaucaggggaucgcagcggcuacagcagccccggcuccccaggcacucccggcagccgcucccgcaccccgucccuuccaaccccacccacccgggagcccaagaagguggcagugguccguacuccacccaagucgccgucuuccgccaagagccgccugcagacagcccccgugcccaugccagaccugaagaaugucaaguccaagaucggcuccacugagaaccugaagcaccagccgggaggcgggaaggugcaaauagucuacaaaccaguugaccugagcaaggugaccuccaaguguggcucauuaggcaacauccaucauaaaccaggagguggccagguggaaguaaaaucugagaagcuugacuucaaggacagaguccagucgaagauugggucccuggacaauaucacccacgucccuggcggaggaaauaaaaagauugaaacccacaagcugaccuuccgcgagaacgccaaagccaagacagaccacggggcggagaucguguacaagucgccaguggugucuggggacacgucuccacggcaucucagcaaugucuccuccaccggcagcaucgacaugguagacucgccccagcucgccacgcuagcugacgaggugucugccucccuggccaagcaggguuugugaucaggccccuggggcggucaauaauuguggagaggagagaaugagagaguguggaaaaaaaaagaauaaugacccggcccccgcccucugcccccagcugcuccucgcaguucgguuaauugguuaaucacuuaaccugcuuuugucacucggcuuuggcucgggacuucaaaaucagugaugggaguaagagcaaauuucaucuuuccaaauugaugggugggcuaguaauaaaauauuuaaaaaaaaacauucaaaaacauggccacauccaacauuuccucaggcaauuccuuuugauucuuuuuucuucccccuccauguagaagagggagaaggagaggcucugaaagcugcuucugggggauuucaagggacugggggugccaaccaccucuggcccuguugugggggugucacagaggcaguggcagcaacaaaggauuugaaacuugguguguucguggagccacaggcagacgaugucaaccuugugugagugugacggggguugggguggggcgggaggccacgggggaggccgaggcaggggcugggcagaggggagaggaagcacaagaagugggagugggagaggaagccacgugcuggagaguagacaucccccuccuugccgcugggagagccaaggccuaugccaccugcagcgucugagcggccgccuguccuugguggccgggggugggggccugcugugggucagugugccacccucugcagggcagccugugggagaagggacagcggguaaaaagagaaggcaagcuggcaggaggguggcacuucguggaugaccuccuuagaaaagacugaccuugaugucuugagagcgcuggccucuuccucccucccugcaggguagggggccugaguugaggggcuucccucugcuccacagaaacccuguuuuauugaguucugaagguuggaacugcugccaugauuuuggccacuuugcagaccugggacuuuagggcuaaccaguucucuuuguaaggacuugugccucuugggagacguccacccguuuccaagccugggccacuggcaucucuggagugugugggggucugggaggcaggucccgagcccccuguccuucccacggccacugcagucaccccgucugcgccgcugugcuguugucugccgugagagcccaaucacugccuauaccccucaucacacgucacaaugucccgaauucccagccucaccaccccuucucaguaaugacccugguugguugcaggagguaccuacuccauacugagggugaaauuaagggaaggcaaaguccaggcacaagagugggaccccagccucucacucucaguuccacucauccaacugggacccucaccacgaaucucaugaucugauucgguucccugucuccuccucccgucacagaugugagccagggcacugcucagcugugacccuagguguuucugccuuguugacauggagagagcccuuuccccugagaaggccuggccccuuccugugcugagcccacagcagcaggcugggugucuugguugucagugguggcaccaggauggaagggcaaggcacccagggcaggcccacagucccgcugucccccacuugcacccuagcuuguagcugccaaccucccagacagcccagcccgcugcucagcuccacaugcauaguaucagcccuccacacccgacaaaggggaacacacccccuuggaaaugguucuuuucccccagucccagcuggaagccaugcugucuguucugcuggagcagcugaacauauacauagauguugcccugcccuccccaucugcacccuguugaguuguaguuggauuugucuguuuaugcuuggauucaccagagugacuaugauagugaaaagaaaaaaaaaaaaaaaaaaggacgcauguaucuugaaaugcuuguaaagagguuucuaacccacccucacgaggugucucucacccccacacugggacucguguggccuguguggugccacccugcuggggccucccaaguuuugaaaggcuuuccucagcaccugggacccaacagagaccagcuucuagcagcuaaggaggccguucagcugugacgaaggccugaagcacaggauuaggacugaagcgaugauguccccuucccuacuuccccuuggggcucccugugucagggcacagacuaggucuuguggcuggucuggcuugcggcgcgaggaugguucucucuggucauagcccgaagucucauggcagucccaaaggaggcuuacaacuccugcaucacaagaaaaaggaagccacugccagcuggggggaucugcagcucccagaagcuccgugagccucagccaccccucagacuggguuccucuccaagcucgcccucuggaggggcagcgcagccucccaccaagggcccugcgaccacagcagggauugggaugaauugccuguccuggaucugcucuagaggcccaagcugccugccugaggaaggaugacuugacaagucaggagacacuguucccaaagccuugaccagagcaccucagcccgcugaccuugcacaaacuccaucugcugccaugagaaaagggaagccgccuuugcaaaacauugcugccuaaagaaacucagcagccucaggcccaauucugccacuucugguuuggguacaguuaaaggcaacccugagggacuuggcaguagaaauccagggccuccccuggggcuggcagcuucgugugcagcuagagcuuuaccugaaaggaagucucugggcccagaacucuccaccaagagccucccugccguucgcugagucccagcaauucuccuaaguugaagggaucugagaaggagaaggaaaugugggguagauuugguggugguuagagauaugccccccucauuacugccaacaguuucggcugcauuucuucacgcaccucgguuccucuuccugaaguucuugugcccugcucuucagcaccaugggccuucuuauacggaaggcucugggaucucccccuuguggggcaggcucuuggggccagccuaagaucaugguuuagggugaucagugcuggcagauaaauugaaaaggcacgcuggcuugugaucuuaaaugaggacaauccccccagggcugggcacuccuccccuccccucacuucucccaccugcagagccaguguccuugggugggcuagauaggauauacuguaugccggcuccuucaagcugcugacucacuuuaucaauaguuccauuuaaauugacuucaguggugagacuguauccuguuugcuauugcuuguugugcuauggggggaggggggaggaauguguaagauaguuaacaugggcaaagggagaucuuggggugcagcacuuaaacugccucguaacccuuuucaugauuucaaccacauuugcuagagggagggagcagccacggaguuagaggcccuugggguuucucuuuuccacugacaggcuuucccaggcagcuggcuaguucauucccuccccagccaggugcaggcguaggaauauggacaucugguugcuuuggccugcugcccucuuucagggguccuaagcccacaaucaugccucccuaagaccuuggcauccuucccucuaagccguuggcaccucugugccaccucucacacuggcuccagacacacagccugugcuuuuggagcugagaucacucgcuucacccuccucaucuuuguucuccaaguaaagccacgaggucggggcgagggcagaggugaucaccugcgugucccaucuacagaccugcagcuucauaaaacuucugauuucucuucagcuuugaaaaggguuacccugggcacuggccuagagccucaccuccuaauagacuuagccccaugaguuugccauguugagcaggacuauuucuggcacuugcaagucccaugauuucuucgguaauucugaggguggggggagggacaugaaaucaucuuagcuuagcuuucugucugugaaugucuauauaguguauuguguguuuuaacaaaugauuuacacugacuguugcuguaaaagugaauuuggaaauaaaguuauuacucugauuaaa

SEQ ID NO: 58 - RNA, 2102 nt, Homo sapiens
agaucucauuccgccucccucugucgucgccccuucccaauucucgcgagaccucaaggagcacagcuucugcgcaccgcacgauacugggaguccaggcgccaagggagggggaagggaaaaggggaaacggugcaaacggcguggccgccaucuugcuugugcccccgcuucgcgcgcgcuccguucuccgugacgcacacuucccccuccccuccgccgcgccugggccucugcauugcccgacuccguaggagcgcgggggcggcuccugcucuuccuggacuccugagcagaguugucgagauguccacagaaggaggauuuggugguacuagcagcagugaugcccagcaaagccuacagucguucuggccucgggucauggaagaaauccggaauuuaacagugaaagacuuccgagugcaggaacucccacuggcucguauuaagaagauuaugaaacuggaugaagaugugaagaugaucagugcagaagcgccuguacucuuugccaaggcagcccagauuuuuaucacagaguugacucuucgagccuggauucacacagaagauaacaagcgccggacucuacagagaaaugauaucgccauggcaauuacaaaauuugaucaguuugauuuucucaucgauauuguuccaagagaugaacugaaaccuccaaagcgucaggaggaggugcgccagucuguaacuccugccgagccaguccaguacuauuucacgcuggcucagcaacccaccgcuguccaaguccagggccagcagcaaggccagcagaccaccagcuccacgaccaccauccagccugggcagaucaucaucgcacagccucagcagggccagaccacaccugugacaaugcagguuggagaaggucagcaggugcagauuguccaggcucagccacagggucaagcccaacaggcccagaguggcacuggacagaccaugcaggugaugcagcagaucaucacuaacacaggagagauccagcagaucccggugcagcugaaugccggccagcugcaguauauccgcuuagcccagccuguaucaggcacucaaguugugcagggacagauccagacacuugccaccaaugcucaacagauuacacagacagagguccagcaaggacagcagcaguucagccaguucacagauggacagcucuaccagauccagcaagucaccaugccugcgggccaggaccucgcccagcccauguucauccagucagccaaccagcccuccgacgggcaggccccccaggugaccggcgacugagggccugagcuggcaaggccaaggacacccaacacaauuuuugccauacagccccaggcaaugggcacagccuuccuccccagaggacccggccgaccucagcgccuccugcaggcuaggacacuggugcacuacaccccaugccugggggccgagauucuccagcagaaagaugcaauauuuuuuguuuccuuuuuuuccauuuuuuucucuaaggaaucaauauuucaauauguugaguguguguccaaugcuaugaaauuaaaauauuaaauaacauauuuauggcauuuucuugaagagugugguugaagaaauauuucuccuuuuguuuuucuuuuuuuuuuguuuguuacugccacuucuuuuuaggagcaaaucuccccagggguguacgguauuucuugacucugggaacagcugcuacccccaagacuugccacguuguucugcccucagauggaauuaggugaauguguguagcugcuuuuucacucgugguccucuccccaucccuugcucugaccccagagcucuguguauuugcauccagaggccauggaaacauucuuugcauuuaagagacagauuuauucccuguggagaguggguggauucauugccacacucuuuucucccagggacccaggaaacuaggacuuuguguguuugcugcccaccucccuuuuauuuuuuaaaugcauuaaaaacugugcuagucuccuuugcauggacuucaagcugcaugaaaugcaauaaaucucauuuuagauaaaaaaaaaaaaaaaaaa

SEQ ID NO: 59 - RNA, 2162 nt, Homo sapiens
agaucucauuccgccucccucugucgucgccccuucccaauucucgcgagaccucaaggagcacagcuucugcgcaccgcacgauacugggaguccaggcgccaagggagggggaagggaaaaggggaaacggugcaaacggcguggccgccaucuugcuugugcccccgcuucgcgcgcgcuccguucuccgugacgcacacuucccccuccccuccgccgcgccugggccucugcauugcccgacuccguaggagcgcgggggcggcuccugcucuuccuggacuccugagcagaguugucgagauguccacagaaggaggauuuggugguacuagcagcagugaugcccagcaaagccuacagucguucuggccucgggucauggaagaaauccggaauuuaacagugaaagacuuccgagugcaggaacucccacuggcucguauuaagaagauuaugaaacuggaugaagaugugaagaugaucagugcagaagcgccuguacucuuugccaaggcagcccagauuuuuaucacagaguugacucuucgagccuggauucacacagaagauaacaagcgccggacucuacagagaaaugauaucgccauggcaauuacaaaauuugaucaguuugauuuucucaucgauauuguuccaagagaugaacugaaaccuccaaagcgucaggaggaggugcgccagucuguaacuccugccgagccaguccaguacuauuucacgcuggcucagcaacccaccgcuguccaaguccagggccagcagcaaggccagcagaccaccagcuccacgaccaccauccagccugggcagaucaucaucgcacagccucagcagggccagaccacaccugugacaaugcagguuggagaaggucagcaggugcagauuguccaggcucagccacagggucaagcccaacaggcccagaguggcacuggacagaccaugcaggugaugcagcagaucaucacuaacacaggagagauccagcagaucccggugcagcugaaugccggccagcugcaguauauccgcuuagcccagccuguaucaggcacucaaguugugcagggacagauccagacacuugccaccaaugcucaacaggggcaaagaaaugcaagucaggggaagccucgaaggugccugaaagaaaccuuacagauuacacagacagagguccagcaaggacagcagcaguucagccaguucacagauggacagcagcucuaccagauccagcaagucaccaugccugcgggccaggaccucgcccagcccauguucauccagucagccaaccagcccuccgacgggcaggccccccaggugaccggcgacugagggccugagcuggcaaggccaaggacacccaacacaauuuuugccauacagccccaggcaaugggcacagccuuccuccccagaggacccggccgaccucagcgccuccugcaggcuaggacacuggugcacuacaccccaugccugggggccgagauucuccagcagaaagaugcaauauuuuuuguuuccuuuuuuuccauuuuuuucucuaaggaaucaauauuucaauauguugaguguguguccaaugcuaugaaauuaaaauauuaaauaacauauuuauggcauuuucuugaagagugugguugaagaaauauuucuccuuuuguuuuucuuuuuuuuuuguuuguuacugccacuucuuuuuaggagcaaaucuccccagggguguacgguauuucuugacucugggaacagcugcuacccccaagacuugccacguuguucugcccucagauggaauuaggugaauguguguagcugcuuuuucacucgugguccucuccccaucccuugcucugaccccagagcucuguguauuugcauccagaggccauggaaacauucuuugcauuuaagagacagauuuauucccuguggagaguggguggauucauugccacacucuuuucucccagggacccaggaaacuaggacuuuguguguuugcugcccaccucccuuuuauuuuuuaaaugcauuaaaaacugugcuagucuccuuugcauggacuucaagcugcaugaaaugcaauaaaucucauuuuagauaaaaaaaaaaaaaaaaaa

SEQ ID NO: 60 - RNA, 1991 nt, Homo sapiens
agaucucauuccgccucccucugucgucgccccuucccaauucucgcgagaccucaaggagcacagcuucugcgcaccgcacgauacugggaguccaggcgccaagggagggggaagggaaaaggggaaacggugcaaacggcguggccgccaucuugcuugugcccccgcuucgcgcgcgcuccguucuccgugacgcacacuucccccuccccuccgccgcgccugggccucugcauugcccgacuccguaggagcgcgggggcggcuccugcucuuccuggacuccugagcagaguugucgagauguccacagaaggaggauuuggugguacuagcagcagugaugcccagcaaagccuacagucguucuggccucgggucauggaagaaauccggaauuuaacagugaaagacuuccgagugcaggaacucccacuggcucguauuaagaagauuaugaaacuggaugaagaugugaagagaaaugauaucgccauggcaauuacaaaauuugaucaguuugauuuucucaucgauauuguuccaagagaugaacugaaaccuccaaagcgucaggaggaggugcgccagucuguaacuccugccgagccaguccaguacuauuucacgcuggcucagcaacccaccgcuguccaaguccagggccagcagcaaggccagcagaccaccagcuccacgaccaccauccagccugggcagaucaucaucgcacagccucagcagggccagaccacaccugugacaaugcagguuggagaaggucagcaggugcagauuguccaggcucagccacagggucaagcccaacaggcccagaguggcacuggacagaccaugcaggugaugcagcagaucaucacuaacacaggagagauccagcagaucccggugcagcugaaugccggccagcugcaguauauccgcuuagcccagccuguaucaggcacucaaguugugcagggacagauccagacacuugccaccaaugcucaacagauuacacagacagagguccagcaaggacagcagcaguucagccaguucacagauggacagcagcucuaccagauccagcaagucaccaugccugcgggccaggaccucgcccagcccauguucauccagucagccaaccagcccuccgacgggcaggccccccaggugaccggcgacugagggccugagcuggcaaggccaaggacacccaacacaauuuuugccauacagccccaggcaaugggcacagccuuccuccccagaggacccggccgaccucagcgccuccugcaggcuaggacacuggugcacuacaccccaugccugggggccgagauucuccagcagaaagaugcaauauuuuuuguuuccuuuuuuuccauuuuuuucucuaaggaaucaauauuucaauauguugaguguguguccaaugcuaugaaauuaaaauauuaaauaacauauuuauggcauuuucuugaagagugugguugaagaaauauuucuccuuuuguuuuucuuuuuuuuuuguuuguuacugccacuucuuuuuaggagcaaaucuccccagggguguacgguauuucuugacucugggaacagcugcuacccccaagacuugccacguuguucugcccucagauggaauuaggugaauguguguagcugcuuuuucacucgugguccucuccccaucccuugcucugaccccagagcucuguguauuugcauccagaggccauggaaacauucuuugcauuuaagagacagauuuauucccuguggagaguggguggauucauugccacacucuuuucucccagggacccaggaaacuaggacuuuguguguuugcugcccaccucccuuuuauuuuuuaaaugcauuaaaaacugugcuagucuccuuugcauggacuucaagcugcaugaaaugcaauaaaucucauuuuagauaaaaaaaaaaaaaaaaaa

SEQ ID NO: 61 - RNA, 2380 nt, Homo sapiens
auguugugcucuugccuggcccgcugcugucgccgccgccgcgcucgugggguccgugugccucgucggcccggcccgcgggcuuucucccauuggcggaaggcgacggcgggggggcucgcucgugauuggucgguggcggggaggccgcggaccuggcaccuuauuggacgcgccagcuccggucuaggggacacgggcuuccaccauguuagccaggcuggucuucaacuccugaccucaggugauccacccaccucggccucccaaagugcugggauuauaggcgugagccaccgugcccagccaggcuaugcugggugucuuuuugagauuuaggaaccucucagaauaaaaucuggaagccuguuccauuugcauuaugagauuugcauuacaaugcaggcugaagugcaguagugcaaucauggcucauugcagucucccagcuccucggcucaagugauccuccugccccagccuccugaguagcgggagcuacagacacacaaacacaggauacugccagaaacugggcaagaggcuguuugugaaucucagaaggcacccggaucuugcucugcuguuccugcucaggcuggagugcaguggcgcaaucauggcucacugcaggcuugaccacccaggcucaagcaguccucccgccucagccucuugucgagauguccacagaaggaggauuuggugguacuagcagcagugaugcccagcaaagccuacagucguucuggccucgggucauggaagaaauccggaauuuaacagugaaagacuuccgagugcaggaacucccacuggcucguauuaagaagauuaugaaacuggaugaagaugugaagaugaucagugcagaagcgccuguacucuuugccaaggcagcccagauuuuuaucacagaguugacucuucgagccuggauucacacagaagauaacaagcgccggacucuacagagaaaugauaucgccauggcaauuacaaaauuugaucaguuugauuuucucaucgauauuguuccaagagaugaacugaaaccuccaaagcgucaggaggaggugcgccagucuguaacuccugccgagccaguccaguacuauuucacgcuggcucagcaacccaccgcuguccaaguccagggccagcagcaaggccagcagaccaccagcuccacgaccaccauccagccugggcagaucaucaucgcacagccucagcagggccagaccaugcaggugaugcagcagaucaucacuaacacaggagagauccagcagaucccggugcagcugaaugccggccagcugcaguauauccgcuuagcccagccuguaucaggcacucaaguugugcagggacagauccagacacuugccaccaaugcucaacagauuacacagacagagguccagcaaggacagcagcaguucagccaguucacagauggacagcagcucuaccagauccagcaagucaccaugccugcgggccaggaccucgcccagcccauguucauccagucagccaaccagcccuccgacgggcaggccccccaggugaccggcgacugagggccugagcuggcaaggccaaggacacccaacacaauuuuugccauacagccccaggcaaugggcacagccuuccuccccagaggacccggccgaccucagcgccuccugcaggcuaggacacuggugcacuacaccccaugccugggggccgagauucuccagcagaaagaugcaauauuuuuuguuuccuuuuuuuccauuuuuuucucuaaggaaucaauauuucaauauguugaguguguguccaaugcuaugaaauuaaaauauuaaauaacauauuuauggcauuuucuugaagagugugguugaagaaauauuucuccuuuuguuuuucuuuuuuuuuuguuuguuacugccacuucuuuuuaggagcaaaucuccccagggguguacgguauuucuugacucugggaacagcugcuacccccaagacuugccacguuguucugcccucagauggaauuaggugaauguguguagcugcuuuuucacucgugguccucuccccaucccuugcucugaccccagagcucuguguauuugcauccagaggccauggaaacauucuuugcauuuaagagacagauuuauucccuguggagaguggguggauucauugccacacucuuuucucccagggacccaggaaacuaggacuuuguguguuugcugcccaccucccuuuuauuuuuuaaaugcauuaaaaacugugcuagucuccuuugcauggacuucaagcugcaugaaaugcaauaaaucucauuuuagauaaaaaaaaaaaaaaaaaa

SEQ ID NO: 62 - RNA, 2105 nt, Homo sapiens
agaucucauuccgccucccucugucgucgccccuucccaauucucgcgagaccucaaggagcacagcuucugcgcaccgcacgauacugggaguccaggcgccaagggagggggaagggaaaaggggaaacggugcaaacggcguggccgccaucuugcuugugcccccgcuucgcgcgcgcuccguucuccgugacgcacacuucccccuccccuccgccgcgccugggccucugcauugcccgacuccguaggagcgcgggggcggcuccugcucuuccuggacuccugagcagaguugucgagauguccacagaaggaggauuuggugguacuagcagcagugaugcccagcaaagccuacagucguucuggccucgggucauggaagaaauccggaauuuaacagugaaagacuuccgagugcaggaacucccacuggcucguauuaagaagauuaugaaacuggaugaagaugugaagaugaucagugcagaagcgccuguacucuuugccaaggcagcccagauuuuuaucacagaguugacucuucgagccuggauucacacagaagauaacaagcgccggacucuacagagaaaugauaucgccauggcaauuacaaaauuugaucaguuugauuuucucaucgauauuguuccaagagaugaacugaaaccuccaaagcgucaggaggaggugcgccagucuguaacuccugccgagccaguccaguacuauuucacgcuggcucagcaacccaccgcuguccaaguccagggccagcagcaaggccagcagaccaccagcuccacgaccaccauccagccugggcagaucaucaucgcacagccucagcagggccagaccacaccugugacaaugcagguuggagaaggucagcaggugcagauuguccaggcucagccacagggucaagcccaacaggcccagaguggcacuggacagaccaugcaggugaugcagcagaucaucacuaacacaggagagauccagcagaucccggugcagcugaaugccggccagcugcaguauauccgcuuagcccagccuguaucaggcacucaaguugugcagggacagauccagacacuugccaccaaugcucaacagauuacacagacagagguccagcaaggacagcagcaguucagccaguucacagauggacagcagcucuaccagauccagcaagucaccaugccugcgggccaggaccucgcccagcccauguucauccagucagccaaccagcccuccgacgggcaggccccccaggugaccggcgacugagggccugagcuggcaaggccaaggacacccaacacaauuuuugccauacagccccaggcaaugggcacagccuuccuccccagaggacccggccgaccucagcgccuccugcaggcuaggacacuggugcacuacaccccaugccugggggccgagauucuccagcagaaagaugcaauauuuuuuguuuccuuuuuuuccauuuuuuucucuaaggaaucaauauuucaauauguugaguguguguccaaugcuaugaaauuaaaauauuaaauaacauauuuauggcauuuucuugaagagugugguugaagaaauauuucuccuuuuguuuuucuuuuuuuuuuguuuguuacugccacuucuuuuuaggagcaaaucuccccagggguguacgguauuucuugacucugggaacagcugcuacccccaagacuugccacguuguucugcccucagauggaauuaggugaauguguguagcugcuuuuucacucgugguccucuccccaucccuugcucugaccccagagcucuguguauuugcauccagaggccauggaaacauucuuugcauuuaagagacagauuuauucccuguggagaguggguggauucauugccacacucuuuucucccagggacccaggaaacuaggacuuuguguguuugcugcccaccucccuuuuauuuuuuaaaugcauuaaaaacugugcuagucuccuuugcauggacuucaagcugcaugaaaugcaauaaaucucauuuuagauaaaaaaaaaaaaaaaaaa

SEQ ID NO: 63 - RNA, 9479 nt, Homo sapiens
ugaacugaggcgaacuccgccgccaagugagugaggaggaggaggaggagaaggaggaggaggaagaggaggagcgcagucggagcgcggcggcagcggcagaggcgccgcggcggggaccagcccagagagaccccccgagcccgcggcacaggcggcggcggcgcugagcgggcucgaccagccgagcgagcggcggccccggcuccuccucguccggcgccagcagcccgcgccccgcgcccgcacucggccagccgagacccgccucccgcccgcccgccggccucgugagggacgcgccgagccgggcggccgagagcggcggcggcugggagcgcgucgggcccgggcggaaagugccugcggggggcgggcgagcgcgcggugccggccgccccggggcucggcgcgggagccagagcgggagccggcgcggagcgggacgccgggucccgagcggcccgcggccggggcucgcccccgccccucccuccucuccggcggcggcggcggcggcggcgggcggagugagcugcggagccuggaauauggucggggaaauggaaacgaaggagaagccgaagcccaccccagauuaccugaugcagcugaugaacgacaagaagcucaugagcagccugcccaacuucugcgggaucuucaaccaccucgagcggcugcuggacgaagaaauuagcagaguacggaaagacauguacaaugacacauuaaauggcaguacagagaaaaggagugcagaauugccugaugcugugggaccuauuguucaguuacaagagaaacuuuaugugccuguaaaagaauacccagauuuuaauuuuguugggagaauccuuggaccuagaggacuuacagccaaacaacuugaagcagaaaccggauguaaaaucaugguccgaggcaaaggcucaaugagggauaaaaaaaaggaggagcaaaauagaggcaagcccaauugggagcaucuaaaugaagauuuacauguacuaaucacuguggaagaugcucagaacagagcagaaaucaaauugaagagagcaguugaagaagugaagaaauuauugguaccugcagcagaaggagaagacagccugaagaagaugcagcugauggagcuugcgauucugaauggcaccuacagagaugccaacauuaaaucaccagcccuugccuuuucucuugcagcaacagcccaggcugcuccaaggaucauuacugggccugcgccgguucucccaccagcugcccugcguacuccuacgccagcuggcccuaccauaaugccuuugaucagacaaauacagaccgcugucaugccaaacggaacuccucacccaacugcugcaauaguuccuccagggcccgaagcugguuuaaucuauacacccuaugaguaccccuacacauuggcaccagcuacaucaauccuugaguauccuauugaaccuagugguguauuaggugcgguggcuacuaaaguucgaaggcacgauaugcguguccauccuuaccaaaggauugugaccgcagaccgagccgccaccggcaacuaaccuaugaccuucugaccucugaacucuucacccaaugaugaccugaccaugccugccugcugaucaguuaacugguaaucgccuuugcuugccugucgucagugcagcgagcugaggcacuuguccguucgucuuaccaucuaaccaaacaaaagacaaagaaauuguuguccuccaacucagcuuuuuuuuuuuuuuuuuccuguuugggugaaagugguucuagaaacugcacugaauaguaguaaagcaauaaggcccaauucaucccacagcacugaucaucuuuuaauaucccacccuaagcgaacgguaagaaggccucucuuaagaaggggagacagaugguccuuaacuacucaaugacagaggcaguuacugugagagacuucuaggaaucuuuuucuucucauagcgaagucaaagcucucucugaauguacugugugaugaugcaucaugcaugaaccuucggucagggauaucauuggugaagugauuucaaaaaguauucaaaauuugauaugcuguuuagucacuacagugcccucaaagggcagaaguugcagccuuuuuuauauugccugccaaaauuugaaguauuagaagaaagugugccaugagagaaaaacuuaaggaguuuugaaaaguaaugcaaauaacaaaacugcaacacuauuuuuaaaaagauaaauaucugaguuaaaauuacugaaucuuuauuuuacaccuaaaaaaauaugagaacaagguacaugcauuaugugucacauuacugggcaaacuguucaaguauuuuuuuuuaaaccucccuguauagaaaaaaaucauuaaggauguaaaagccaugcuugccuauuugcuguauacauguaaugaaauuguagauaaaguguagugcauugaaacaaaugaacaaaaaguagauacuuuuacuauacaagggugcuggugcagaaaaaaauauauauauuuuuggaaauguagcauuuuauacuuucaaguguuauaaaaaaaaagaaaaagaacaaagaaacccuuuauuucauuaaaugauuuuuucuggugguugucaagaaacaaaagaccaaaagagccuuuuugucuuucuuuuuuauuuuuuaaguauuggaauaagucuaaagaaaagaaagugccuuauuuuccuucauggucauucagucaagugucucauaaagaucagcuccucccucagaauacaaguuaaugcauguuacucagucgcccaguaugugaaagaagauaugagggagacaaaugaguugauaguuugaauuauaggauuuuugccacguuaccuugaugcaaauuugccaaaucugauacugugaaaagauuugauaacuuuucuaaugccugacuaguaaguuuuaaaacaucauucuuuaaaagaauaguuuaauacuuuugguuauuccaaucuucaauguuaacccaaaggauucugagauaaaaauuaugugucaucuaaacuucaguuuacagacaaaauuagagcuuuguuuuauuuggagaagggggaugcauucauuugauuuuguuuuuuguuuuuaauuaauugaauauaauaggauaccuagcaaauguuuucacaaaagugaaucuuugugauuuucauucuagaaaacaauguuuuaaaucacccaaauuuaaucacaugucuuuauaucauaccagauaacugguguaguguauccuuuuacuguuucaauaaccucugccucccccaccccuuaacuuuaaaagaaugugaaaaacuagcaucauagugcauauaaauacucaaccacauuucagcuuaagcuucuaauuucucaagaaguugaaacaguuaguuauguaguugaagcaauuugugugcaaagccuacaucuaaacacuugagugaauaaucauuaacugcucaguaccagacguggcaaaucucaagugacaguggacccccccccccgcccagcuuaucaacucguccccucuuccacaccuucccagcaucacagucagaggucagcaggaagcugagucauuaccuucuuggugaccucccccaccaucuugucucacuuggaaaccguugucaguuuauuuacuaugcaauagacauucauuguuuuguauccagcuagcuuugguuuaauaugccacugcuuugucuuucuguuacacauacaguuuugauuuauuuacaauauaugcugugccauuuugauuuuuauauuguuugguugguugaauaaauuugcgacacucaaacacuugaggugauaguuguuaaaaacaauaccaguauuugauccaguuucaucucaacuauguuauaccuggacauuuuagauguuuaucaaaggucuuuuauggggaagaaaguaaauguaugaaauaauaaaugugugacauuuauagggaauguuggcucugaacaaacuuuugaaaacuugguugacaaaauuuuggaucaacugaaaaaagcaugacuuuugaaaucucugaaugccuugguucucaguauuaucauucuuuauugaauuuauuucuuauuaaaauauguaguuuuuaagacuuuuuuucugacaguauuauguaauuuuuuagcguggguagaugggagugucgcuuguauguuaucguacagcugacauguauuuuugucuauucuuuauuaucuuaguuucaugcuauguauguaccauaaccaaccuauugccuaugagaaacauguaagauaauguauuuacagccauuguuacaaguuuauaauguauuuuucuaucuuguuuuauauguauguuauauaacauucaaaaagaauuuuuuucuugauugagaaaaggauacaaaaugcaaaauccacaauuuugauaacugaaaauugccaauuguuuugcaguacuuuauuauauugggugucuugucuuuuuugggcuuuuaguuagcuaaugaaugaauacauuagacacuuuuggguuuuaguugggauuuuacauagcuugcauuuuaauucuuugguucuuugcuguuucuauuaacccacagcauuauuuuaauaaauguuauaauaccaaccuacuaacucuggacuuugucuguuuauuaaccuuuauauguuuaauuaaaauaaacaaauaaagacaaaagaauguaaagucuugaguuacuggacuaacccugaagaacgugaccacagauaacauagugugacuuguuuuuauguuguuugucagcugacaguuuugcacacuacuguuaaaauggcuuugguuauucuggucuuuuagcuugggggccggggcggguguggguguaggugcaguagagaugggaacaaaguagccuuuguuugaggcauucuucucuugccacuugccuuucagcaucugcauuacuaauuccacaccuuauuuuuccuucuccaaaauaaucacugucugcucacagcaucgaguaaaaagauacauugucaugguauauaccuggagaaaccaguuugauucuuuguugaaccauuucuuuuaauuucuguaaauaaguaauuuuauggucacauuguaaagcagagaaacuguugauauuuuaccuuuauuuagauuuuuguuuuaacuugaccuuuuuccuuuggaaagcguugaacuuguuuagcuaacuacuccuauuaggggagcuuaacuuggguaaagucaaaggcccacuggcuguuucucaugguggccugaagccccgcugcuuuccagugaggaaaagaacccucagcacguuacugagcuuuccucagguauuuuuuucucugucaccuacuuuacaaauacagucagacuaaaacauuaaacaagaaguuuaagggggaauaacauugggauuugaaaaguuguaugugauuaauuucuugcaugcaaaucaauuuaagauuucuuaaauaucuucaugcuuuucuuggcuggaacuuggguaaauuaaccuguaauuaugaauuauuuaguuuaacucaaggugcauuuuuauuucacagauugauacuugcuuuuuucugugaaacaguucuccuagauaccaaaauccucaggaugaauuuuugcauuuguaaauaacaauuuaucuuuguaaauuacauuuuauuuauuuagaguucuaaaauugagggcugaaaugaaugacauguaauagaagacaaaacuguaccaauacgcuguuaaccaaucagacuguccagguauucucauuucauaaauauguauacacacauauaaauacacagaguaguguuagcugaugugagugauaacagcgaaucaccauugagauggucauuuggaucauguaaacaaaccgugucaugucucugaugggagcgguggguaguguacuguuagguggacaggucuuuuaacaugcuacuauuuguaaaaguuuccguugccauaacaguucauguaguagucugugaaaauucgaagcugucugucaagcugcuaauauacaaguugucaugguaccauguaacuuaaauugaugaauuucuaugcaauuuacuauucucugaagccuaugaaugcagccucaucucauuuucaguuaaggguacuuuauguauaauuaugucauugcugccuuuuauguaggaaauguaaaaaaguuuuaaaaggcgaaaauaacauuacagauuucauuuuuauacagguuacaauauuaaacaugaaacuaccaaauuccaagaauaguaaaauucuagagucaguugauugauugaaacuauuugacaaaacaguugaaagcugagcuuucagucaagaaguuggugaaauuaauguccuuaaacuuuuccuuuaggcaaaaugucagggaaaacuuaacaugaaaguaguuacucccauacgcaccagugcaguagcuccagguguaagaggucacgaaggcacccugcccauaaaaccaggaugugcauacguacacacaaucggugucugguuaugguuuucuaaacacuacauaaggaucugaauuuuuuagugugccaaaagcaaacgauagguuuaaaugaaauugcucacuaccaaaucaagacucuucauauauauacguguguguauauauauaugucauaacacuuucacgugugacagccuaagguuucuuuaguuuuaggaugggguggggugggagugguuuugcauguuugaccaggaugugaugccugucuuuauuccuugauaagaaguagcuguuacagcauuucaagcaaauuauuuuagguagaauagaacuuacugccaaaugauuauuuauuuagcaaaaauuauucuccaacuuuucgaauucacaauuuuucuaagugcauagaguaguuuuuuacauugucacgaauuccuuaauaccuuuauuuaaaauggaaaaaauauggacaauauuuuagaaauaugugcuacacaucuaauuuuaucuguaguuuaagugaucuaaauugagaugccuuugauaugaagagauuuaccaacauuauaugcacucgugccuucaauguggaaucaaacugguuaaccucagcacaguccucucguuucuguguuucugcugcacuaauugccagggggugggggagaaggcgaaugaauuaauucauaguagaaggaggcgauaggugcagcaaagggcagccacaguguugauuccacauuauaauguuguugccucuucuuggcaaaagacaccacauugugggaagaucucagcuuccaggguaaaaguuaauuuauaacuuaaaagugcuauuaaguuuuuauuaccaaauauaucuuuuaugguuuauauuguagugguauguaugaaacauuuaaaauuuuacuguggaaauuguguauauauauauauauagucgaaauagguguucacaggucacaugugaacggagaacugcaugaccguacaugaaaugcaauaaaccaacuggaaaaagugcaugugcuucauccucucaagccaacugcagcuggaaagugcugcuuauccuccacccccagaaaaugcauguaucaauaugagaauaaagaacgcacacuuucaauuuuauugaggcuuucaacacuauuuaaaagaaaauguaagaauuugacauucuggaguuauuauaacauuagaaaaugagcauaacauucacucugauuuuagccauuaagggagauuaguaaacagacugcuacaguguuccauaguuggacugugcauccaaaacauuuuuuuaucuuuaauaaaugguacaguuuuuauguaguuuucgaauguaagaagaaaggaaugcugaccaaaacuugauuucaucagcuucaugaaaaggacuagugucauuaaccuguugaacagaauugguuuauuaaaaaaaucauuuccaguagugugaaaccuuuacgagucuuuaacaucuaaauguuaugacuccuuguaccuuaaguuuuccagucuuucuuauuuauaucaucuccaaguaccucuggcuccuuuccucuugcucaccggaaccuuaguuuuccucaacagaaugcuuuguuaaaguagcccacaguugcaggauccauagcaccgucgugcagacuagcagcccaaagguguguuugguuuggcuuauacgguguuuugcuuuuuaaacuacuugccauaauuuaaaaguggcaacacuagacuuaaaaaaaaaaaagucugauugcccauauuagauuuuuuuuuuaauucuucacaaaaucugcucuuccugaaagaucaaagugucuagaaagcccaaacauguauucuuaacauaguaggcaccagcugaaacugaguaauuaaacgguccccugaagccaaguauucccugguuagucgccaccccaccacuccuuccugucucuagugucacacuugggcuguugauuuucuuacucuucucuugcuuuaauccuuccccccggcucucuggcuccuguggauaucugugcuuguuuccugguccaggauggugaucugacuuucaaaccagcuucucaaaaggggugacauaaaaucaguuuugauguuuuuccuccugaaaaaucagaugaauauuuuagucacuguuacucaugcacauuguguucuuauguuuacagaagugcuuaagugaauggaagcacuagacauuuggauuuccuuccuaacguaauuuuuaaugauuaccccuuuuauacaguaauuuguggucuuuagaaagcaguuaaacuaauugaccaucuaauaguuguacuauacauaugucuaaaauaauagucaugguaaguuuggcaucauaucuuccccaaaaaauguuuauuaaaauuagauuauuccaguuuaaugcuauuuugugaacuguauacccucugaaagcgcuuauuuuuacaugcuacacaacaguuccaauuuuaaggagugucuccuaaaauugggaugaaaaucuacuguagucuguuuuaaaguaugcuauacuauguucauugguuacuuaacuggauauuaauauaaagguuauuacaagaaaaaugaugaagagcaaaaggagaagaaaauauuugcaagugaauccacaauucuugcagaacuauuugaguugauacuaaagauuuuauguucacuccuuuaccuuagaacugucaagcuugggaaugaggaaagugccuuggcugugcuugaaaccugaauuuuagugcuucccuuauuacauucauuguuuucaaugauugauuuauaaaauuaagacauacugguaguacaaguugaaaguugguuugaauacauuuuaauuaaaugauaauauguuaauaugcuuuuguucauugcuuucucacugagguaaaacagcauuaaaaaguuguccagaauuuaaacugaaaaaaaaaaaaaaaaa

SEQ ID NO: 64 - RNA, 14152 nt, Homo sapiens
ugaacugaggcgaacuccgccgccaagugagugaggaggaggaggaggagaaggaggaggaggaagaggaggagcgcagucggagcgcggcggcagcggcagaggcgccgcggcggggaccagcccagagagaccccccgagcccgcggcacaggcggcggcggcgcugagcgggcucgaccagccgagcgagcggcggccccggcuccuccucguccggcgccagcagcccgcgccccgcgcccgcacucggccagccgagacccgccucccgcccgcccgccggccucgugagggacgcgccgagccgggcggccgagagcggcggcggcugggagcgcgucgggcccgggcggaaagugccugcggggggcgggcgagcgcgcggugccggccgccccggggcucggcgcgggagccagagcgggagccggcgcggagcgggacgccgggucccgagcggcccgcggccggggcucgcccccgccccucccuccucuccggcggcggcggcggcggcggcgggcggagugagcugcggagccuggaauauggucggggaaauggaaacgaaggagaagccgaagcccaccccagauuaccugaugcagcugaugaacgacaagaagcucaugagcagccugcccaacuucugcgggaucuucaaccaccucgagcggcugcuggacgaagaaauuagcagaguacggaaagacauguacaaugacacauuaaauggcaguacagagaaaaggagugcagaauugccugaugcugugggaccuauuguucaguuacaagagaaacuuuaugugccuguaaaagaauacccagauuuuaauuuuguugggagaauccuuggaccuagaggacuuacagccaaacaacuugaagcagaaaccggauguaaaaucaugguccgaggcaaaggcucaaugagggauaaaaaaaaggaggagcaaaauagaggcaagcccaauugggagcaucuaaaugaagauuuacauguacuaaucacuguggaagaugcucagaacagagcagaaaucaaauugaagagagcaguugaagaagugaagaaauuauugguaccugcagcagaaggagaagacagccugaagaagaugcagcugauggagcuugcgauucugaauggcaccuacagagaugccaacauuaaaucaccagcccuugccuuuucucuugcagcaacagcccaggcugcuccaaggaucauuacugggccugcgccgguucucccaccagcugcccugcguacuccuacgccagcuggcccuaccauaaugccuuugaucagacaaauacagaccgcugucaugccaaacggaacuccucacccaacugcugcaauaguuccuccagggcccgaagcugguuuaaucuauacacccuaugaguaccccuacacauuggcaccagcuacaucaauccuugaguauccuauugaaccuagugguguauuagguauggcuuucccaacgaaaggcuaagaauucaagaacggucuuaacugaacccucaucagaucugaauuuaacaaaugcuuagucucagcagccuccgggggaaaaaagcuuagccuagcagucagugacuuacuugcacuuuuugcacauagauauaaaguaaaauuauguuauuaauuugguuuagucuguaauauuacacaguaaugguaauuuauaaaggaguguauaguaguauacugacugcuaaguguacuauacuguuugcuuuacuaaucaccuaauucauugcaguucauacuuauugacucagaguuuaaaaccacaaucuguaggcuuuaagaauugcuuuuaaaccuuuuuaagugauaaacucuugaaguggucuuaagguuagcaaauaauugucuguauuaaacauggcguuauuuaaguaguccugcugcaagaaaggcacuucagugaauaugugacuaguaaagcuuaaauauguuuggaucuaauagagcucaugaaauacuguaacuuggggauuguaaaugaauggauacaauagguuaaggcccaagauguuugaaaugaaugcaauauuaauagaugcauauauacaugacauauugugguuaauuuuaaaacuacugugccuuaacguguuucuuaaacuuuuguaguaaaugaacauuugaaauccauuuugauaaaccugcuguuaauguuuuuuccccccuugugaauguuuucuaacuuugucuugguaauugcaauuuaacuaggugcgguggcuacuaaaguucgaaggcacgauaugcguguccauccuuaccaaaggauugugaccgcagaccgagguuaguuuaguucugcaguucuugucuauaagaaaugcguuggguguccauaaaauuugcaacaccacaccugauggaaaaauguaauugcuuaccugcacacaguuuuuuuuccuuuucuaaauuuguuugugaucauugacagauuuaaggguuggguuuuuuguuuugguuugguuuuuucucccuucauauuuuaacuuauuaaaucuucuauuauuauguacuaugauuuauuccuacuaaaacauaaauuagucaauuuggggagcuauuaaauuuugauguugccucaguuauuuucauuaauaacuuuugcuaaaauuuaccuuuuaaaaguucugaaauuugggguugguauugcaaaaugaauugaacggauggugcuuuaaugagagaaguaaaggaauauaugccuccuguguacuuuuggagaaaacugaagauuuaguuuccauuuuucuuuauaauaaaauauuugagccacuguauaaacaugagggugcacaugucaaguacuuagcuaaaaucaacaaagugaggacugugaagauaacuuucauguaguuggauuuuuuuauauaaauuagagcgucaauucccuuuuauucuuacauucucuugccaaaauggauccuuugauuuuugucauuugcugcuaaaauauauuaucugaagguaaacugucucuugaaguucuguggugguaugagauccagcugugugaauauaauuuuugacuaaccugugacuggaaaauaagucuucauuuuucuccuuuacaacuuuuauaauuuguguccuguauaugcccuuggacccuuuuauaaauuauuuuugcuuauugaggaaugguauuuucgguuuauugacauugaauuguuuucuaaguuuuuccaucacaagauaggaauaucaagcaugguaguaaaaaaggauuuuuuacaaguuucuauuauggcacauuuguuuccuacaaauuaaauugaucaaagucucaaauauuaguggaggaaaaagugcuggauuuguacuucaauuguacaucuucuaugaauuuaugaaggaccuaccugcuuuaugugauuaccuugaguacugaugauaaauacuagacacagcuguuguccacaucagaaauaacaugccuuucccaaagacauuguuaauauuuguuuagcauaauccauaaagaacguugaagacuuuuaaacuaauguauucaucacuaacagugauuguucagugaacaaaauagaccccagcuccguggucgucuaugaggaugguauuccuuguaaguguucuguaugagcacgucucauagggauggggaaguuuuuuggaaagucacuugaguuuuaaagaaacuggacaauuccaugaauaauuugagguuuuauucauauugcuaaugucuacaauuguucuaaagaguauuuuaaaguaguuuuuuucccccagcuaaucuuucccuauguaugcccccuuuuaggagauuucucaccauuuugcuuuaacacaauaauuauuggccuacuuaauacuccagugccuugagauaguuggacauauuuuaggaagauuuuauuguauuuggaaaauuacgacacauuccaugcauggaucuuuuguaguuacguguuuugauuaguccugucacuguuuuuauuuaaaaugcagugucagcaaaccgaaaugcucauuucuuuuuuaguaacagaguacucguauacuuaucacuguaugcacucauugggucuaagggagaugucugaaguccuucacuuaaauuauuuuaugauacuacuguuuucucuauuuuugagguuaaaauuauuuuaaaaaaauauuuugaagagaaguuuauuaguauaagcauucauuuaaugcaaaaaaacucagcccuuggacauauaaccauguaauucuauaugaacguuuagaguacuacaugguugaccaauaucuaguucuuuacuaaaaguaaacucaccucuuuauauauauuucaaaacugaauuugaagccuauuagagaccaacugaccuuuuaguuguaaugaacuguauggucuugcauggugacacaccuguguaggaggccaagacuuggcagaggauguagucuagguguccugguauauauaaauggucacucacuucguuuauauauuccacacucagaaaagaagucagaguuuuuucugaguaauacaaaaaccaucacuuacauaauuuguauaagaaacuuuaaacaguuuuaaucaaaacuaugaguaacaucaaaaaugcuuuaagauucuauauuuuggaauaccugugaaguaguaugacuacaucauguuaaauauuuauuuucuucuaugauuuuggagcagcaauuuuuauuuuaaauaaaauuccacuuuuaagaaauucagggaagauuuggucacauugaagauacaguauuuuuguaguauuuauaaacuguucuaaaugauagacuauagaaaacauuuuugucauaugaagguaaaucaguccauuauuuuggaucauuuaaacugaacauuacaccuucugggcuuugauuuaugaaguggcacagaaucuaacuuugcacugaauaucuuuucauucacaucuccucugccuuagucacaauggcaacaguacagaaaauucuaucaaaccucagaauauaguagaaauaauuaagcuguugaaugagucuuaaaaauuauacuacuguuaaguggaccaaguuuggugaagcagaaugugacaaagguugauuaaggaaggaacaacucaaggacauugggaaugauaacuuuuccacuugagaacuacuuuauguuuuacuguaauuuuuaaaguuuuuuuguccuuuuuguuauuuugcaaaagaaaauaguauuuacagguggcuucuuuuaaaauauaaaaauauaaagcaggaauguauaugaaaugucagauuuuauuguauuugcagaguauuagcuuugaaauugaauaaagaaagcuguuuguaguuuuaaaaugccuuauugguaucaauuagaaauuucuucuauuuuuugauguacuuagagcuuuuugaguguagaauuuuaaauggcaggauuuuacaguguuuacaugcaagugcauuuuauaaguguucuauauguguaaaauaguauuuucaacuggaaaguguuggcuagugcaaaaggccuggccauuuucugguucccaugauguugccuacacugcuagacuacaguuuaguaucgcuuuguaucaugaggccaagaaauuccauguuguuuguaaauagaauaauugaaaaagcaauaaacauuuauugaacaaaagaaaccuuguuuggcccagaguuuauguugaaccaaaugguucauagaauuuugaauuucuuuagauucauuucuguuaaucaucaguuauuaauaaucacauccaucauuaauaguugcuuuaaugcuugcaucuuagagaauacuaaugcuuaauagcagucagauacugauaacugcacugacuguuuaaaaauucuguauaccaaaccuauucauuguuguuuuucaauuuccuucauugacugcuacuaacuauuaguuguuaguuguauuuuauuucuauuuuuucuuguuaaccaguuuuuuuuuuuuuuuugugggagugggcucuucuucauuuguucaaaucaugauacgaaauugucaugauuuuaauuguauucuguagauggugucaaucaguuugucagaauuaaacaugcuuuuuuuuuuauuaguugugauuaguuuuucauguugucauuaagccgucacuagcugaaacuaaucucuucucuaucuuuucuuuucuuuuuuuuguuuuguuuuuuuuuguuucuaaccacccagccgccaccggcaacuaaccuaugaccuucugaccucugaacucuucacccaaugaugaccugaccaugccugccugcugaucaguuaacugguaaucgccuuugcuugccugucgucagugcagcgagcugaggcacuuguccguucgucuuaccaucuaaccaaacaaaagacaaagaaauuguuguccuccaacucagcuuuuuuuuuuuuuuuuuccuguuugggugaaagugguucuagaaacugcacugaauaguaguaaagcaauaaggcccaauucaucccacagcacugaucaucuuuuaauaucccacccuaagcgaacgguaagaaggccucucuuaagaaggggagacagaugguccuuaacuacucaaugacagaggcaguuacugugagagacuucuaggaaucuuuuucuucucauagcgaagucaaagcucucucugaauguacugugugaugaugcaucaugcaugaaccuucggucagggauaucauuggugaagugauuucaaaaaguauucaaaauuugauaugcuguuuagucacuacagugcccucaaagggcagaaguugcagccuuuuuuauauugccugccaaaauuugaaguauuagaagaaagugugccaugagagaaaaacuuaaggaguuuugaaaaguaaugcaaauaacaaaacugcaacacuauuuuuaaaaagauaaauaucugaguuaaaauuacugaaucuuuauuuuacaccuaaaaaaauaugagaacaagguacaugcauuaugugucacauuacugggcaaacuguucaaguauuuuuuuuuaaaccucccuguauagaaaaaaaucauuaaggauguaaaagccaugcuugccuauuugcuguauacauguaaugaaauuguagauaaaguguagugcauugaaacaaaugaacaaaaaguagauacuuuuacuauacaagggugcuggugcagaaaaaaauauauauauuuuuggaaauguagcauuuuauacuuucaaguguuauaaaaaaaaagaaaaagaacaaagaaacccuuuauuucauuaaaugauuuuuucuggugguugucaagaaacaaaagaccaaaagagccuuuuugucuuucuuuuuuauuuuuuaaguauuggaauaagucuaaagaaaagaaagugccuuauuuuccuucauggucauucagucaagugucucauaaagaucagcuccucccucagaauacaaguuaaugcauguuacucagucgcccaguaugugaaagaagauaugagggagacaaaugaguugauaguuugaauuauaggauuuuugccacguuaccuugaugcaaauuugccaaaucugauacugugaaaagauuugauaacuuuucuaaugccugacuaguaaguuuuaaaacaucauucuuuaaaagaauaguuuaauacuuuugguuauuccaaucuucaauguuaacccaaaggauucugagauaaaaauuaugugucaucuaaacuucaguuuacagacaaaauuagagcuuuguuuuauuuggagaagggggaugcauucauuugauuuuguuuuuuguuuuuaauuaauugaauauaauaggauaccuagcaaauguuuucacaaaagugaaucuuugugauuuucauucuagaaaacaauguuuuaaaucacccaaauuuaaucacaugucuuuauaucauaccagauaacugguguaguguauccuuuuacuguuucaauaaccucugccucccccaccccuuaacuuuaaaagaaugugaaaaacuagcaucauagugcauauaaauacucaaccacauuucagcuuaagcuucuaauuucucaagaaguugaaacaguuaguuauguaguugaagcaauuugugugcaaagccuacaucuaaacacuugagugaauaaucauuaacugcucaguaccagacguggcaaaucucaagugacaguggacccccccccccgcccagcuuaucaacucguccccucuuccacaccuucccagcaucacagucagaggucagcaggaagcugagucauuaccuucuuggugaccucccccaccaucuugucucacuuggaaaccguugucaguuuauuuacuaugcaauagacauucauuguuuuguauccagcuagcuuugguuuaauaugccacugcuuugucuuucuguuacacauacaguuuugauuuauuuacaauauaugcugugccauuuugauuuuuauauuguuugguugguugaauaaauuugcgacacucaaacacuugaggugauaguuguuaaaaacaauaccaguauuugauccaguuucaucucaacuauguuauaccuggacauuuuagauguuuaucaaaggucuuuuauggggaagaaaguaaauguaugaaauaauaaaugugugacauuuauagggaauguuggcucugaacaaacuuuugaaaacuugguugacaaaauuuuggaucaacugaaaaaagcaugacuuuugaaaucucugaaugccuugguucucaguauuaucauucuuuauugaauuuauuucuuauuaaaauauguaguuuuuaagacuuuuuuucugacaguauuauguaauuuuuuagcguggguagaugggagugucgcuuguauguuaucguacagcugacauguauuuuugucuauucuuuauuaucuuaguuucaugcuauguauguaccauaaccaaccuauugccuaugagaaacauguaagauaauguauuuacagccauuguuacaaguuuauaauguauuuuucuaucuuguuuuauauguauguuauauaacauucaaaaagaauuuuuuucuugauugagaaaaggauacaaaaugcaaaauccacaauuuugauaacugaaaauugccaauuguuuugcaguacuuuauuauauugggugucuugucuuuuuugggcuuuuaguuagcuaaugaaugaauacauuagacacuuuuggguuuuaguugggauuuuacauagcuugcauuuuaauucuuugguucuuugcuguuucuauuaacccacagcauuauuuuaauaaauguuauaauaccaaccuacuaacucuggacuuugucuguuuauuaaccuuuauauguuuaauuaaaauaaacaaauaaagacaaaagaauguaaagucuugaguuacuggacuaacccugaagaacgugaccacagauaacauagugugacuuguuuuuauguuguuugucagcugacaguuuugcacacuacuguuaaaauggcuuugguuauucuggucuuuuagcuugggggccggggcggguguggguguaggugcaguagagaugggaacaaaguagccuuuguuugaggcauucuucucuugccacuugccuuucagcaucugcauuacuaauuccacaccuuauuuuuccuucuccaaaauaaucacugucugcucacagcaucgaguaaaaagauacauugucaugguauauaccuggagaaaccaguuugauucuuuguugaaccauuucuuuuaauuucuguaaauaaguaauuuuauggucacauuguaaagcagagaaacuguugauauuuuaccuuuauuuagauuuuuguuuuaacuugaccuuuuuccuuuggaaagcguugaacuuguuuagcuaacuacuccuauuaggggagcuuaacuuggguaaagucaaaggcccacuggcuguuucucaugguggccugaagccccgcugcuuuccagugaggaaaagaacccucagcacguuacugagcuuuccucagguauuuuuuucucugucaccuacuuuacaaauacagucagacuaaaacauuaaacaagaaguuuaagggggaauaacauugggauuugaaaaguuguaugugauuaauuucuugcaugcaaaucaauuuaagauuucuuaaauaucuucaugcuuuucuuggcuggaacuuggguaaauuaaccuguaauuaugaauuauuuaguuuaacucaaggugcauuuuuauuucacagauugauacuugcuuuuuucugugaaacaguucuccuagauaccaaaauccucaggaugaauuuuugcauuuguaaauaacaauuuaucuuuguaaauuacauuuuauuuauuuagaguucuaaaauugagggcugaaaugaaugacauguaauagaagacaaaacuguaccaauacgcuguuaaccaaucagacuguccagguauucucauuucauaaauauguauacacacauauaaauacacagaguaguguuagcugaugugagugauaacagcgaaucaccauugagauggucauuuggaucauguaaacaaaccgugucaugucucugaugggagcgguggguaguguacuguuagguggacaggucuuuuaacaugcuacuauuuguaaaaguuuccguugccauaacaguucauguaguagucugugaaaauucgaagcugucugucaagcugcuaauauacaaguugucaugguaccauguaacuuaaauugaugaauuucuaugcaauuuacuauucucugaagccuaugaaugcagccucaucucauuuucaguuaaggguacuuuauguauaauuaugucauugcugccuuuuauguaggaaauguaaaaaaguuuuaaaaggcgaaaauaacauuacagauuucauuuuuauacagguuacaauauuaaacaugaaacuaccaaauuccaagaauaguaaaauucuagagucaguugauugauugaaacuauuugacaaaacaguugaaagcugagcuuucagucaagaaguuggugaaauuaauguccuuaaacuuuuccuuuaggcaaaaugucagggaaaacuuaacaugaaaguaguuacucccauacgcaccagugcaguagcuccagguguaagaggucacgaaggcacccugcccauaaaaccaggaugugcauacguacacacaaucggugucugguuaugguuuucuaaacacuacauaaggaucugaauuuuuuagugugccaaaagcaaacgauagguuuaaaugaaauugcucacuaccaaaucaagacucuucauauauauacguguguguauauauauaugucauaacacuuucacgugugacagccuaagguuucuuuaguuuuaggaugggguggggugggagugguuuugcauguuugaccaggaugugaugccugucuuuauuccuugauaagaaguagcuguuacagcauuucaagcaaauuauuuuagguagaauagaacuuacugccaaaugauuauuuauuuagcaaaaauuauucuccaacuuuucgaauucacaauuuuucuaagugcauagaguaguuuuuuacauugucacgaauuccuuaauaccuuuauuuaaaauggaaaaaauauggacaauauuuuagaaauaugugcuacacaucuaauuuuaucuguaguuuaagugaucuaaauugagaugccuuugauaugaagagauuuaccaacauuauaugcacucgugccuucaauguggaaucaaacugguuaaccucagcacaguccucucguuucuguguuucugcugcacuaauugccagggggugggggagaaggcgaaugaauuaauucauaguagaaggaggcgauaggugcagcaaagggcagccacaguguugauuccacauuauaauguuguugccucuucuuggcaaaagacaccacauugugggaagaucucagcuuccaggguaaaaguuaauuuauaacuuaaaagugcuauuaaguuuuuauuaccaaauauaucuuuuaugguuuauauuguagugguauguaugaaacauuuaaaauuuuacuguggaaauuguguauauauauauauauagucgaaauagguguucacaggucacaugugaacggagaacugcaugaccguacaugaaaugcaauaaaccaacuggaaaaagugcaugugcuucauccucucaagccaacugcagcuggaaagugcugcuuauccuccacccccagaaaaugcauguaucaauaugagaauaaagaacgcacacuuucaauuuuauugaggcuuucaacacuauuuaaaagaaaauguaagaauuugacauucuggaguuauuauaacauuagaaaaugagcauaacauucacucugauuuuagccauuaagggagauuaguaaacagacugcuacaguguuccauaguuggacugugcauccaaaacauuuuuuuaucuuuaauaaaugguacaguuuuuauguaguuuucgaauguaagaagaaaggaaugcugaccaaaacuugauuucaucagcuucaugaaaaggacuagugucauuaaccuguugaacagaauugguuuauuaaaaaaaucauuuccaguagugugaaaccuuuacgagucuuuaacaucuaaauguuaugacuccuuguaccuuaaguuuuccagucuuucuuauuuauaucaucuccaaguaccucuggcuccuuuccucuugcucaccggaaccuuaguuuuccucaacagaaugcuuuguuaaaguagcccacaguugcaggauccauagcaccgucgugcagacuagcagcccaaagguguguuugguuuggcuuauacgguguuuugcuuuuuaaacuacuugccauaauuuaaaaguggcaacacuagacuuaaaaaaaaaaaagucugauugcccauauuagauuuuuuuuuuaauucuucacaaaaucugcucuuccugaaagaucaaagugucuagaaagcccaaacauguauucuuaacauaguaggcaccagcugaaacugaguaauuaaacgguccccugaagccaaguauucccugguuagucgccaccccaccacuccuuccugucucuagugucacacuugggcuguugauuuucuuacucuucucuugcuuuaauccuuccccccggcucucuggcuccuguggauaucugugcuuguuuccugguccaggauggugaucugacuuucaaaccagcuucucaaaaggggugacauaaaaucaguuuugauguuuuuccuccugaaaaaucagaugaauauuuuagucacuguuacucaugcacauuguguucuuauguuuacagaagugcuuaagugaauggaagcacuagacauuuggauuuccuuccuaacguaauuuuuaaugauuaccccuuuuauacaguaauuuguggucuuuagaaagcaguuaaacuaauugaccaucuaauaguuguacuauacauaugucuaaaauaauagucaugguaaguuuggcaucauaucuuccccaaaaaauguuuauuaaaauuagauuauuccaguuuaaugcuauuuugugaacuguauacccucugaaagcgcuuauuuuuacaugcuacacaacaguuccaauuuuaaggagugucuccuaaaauugggaugaaaaucuacuguagucuguuuuaaaguaugcuauacuauguucauugguuacuuaacuggauauuaauauaaagguuauuacaagaaaaaugaugaagagcaaaaggagaagaaaauauuugcaagugaauccacaauucuugcagaacuauuugaguugauacuaaagauuuuauguucacuccuuuaccuuagaacugucaagcuugggaaugaggaaagugccuuggcugugcuugaaaccugaauuuuagugcuucccuuauuacauucauuguuuucaaugauugauuuauaaaauuaagacauacugguaguacaaguugaaaguugguuugaauacauuuuaauuaaaugauaauauguuaauaugcuuuuguucauugcuuucucacugagguaaaacagcauuaaaaaguuguccagaauuuaaacugaaaaaaaaaaaaaaaaa

SEQ ID NO: 65 - RNA, 15431 nt, Homo sapiens
ugaacugaggcgaacuccgccgccaagugagugaggaggaggaggaggagaaggaggaggaggaagaggaggagcgcagucggagcgcggcggcagcggcagaggcgccgcggcggggaccagcccagagagaccccccgagcccgcggcacaggcggcggcggcgcugagcgggcucgaccagccgagcgagcggcggccccggcuccuccucguccggcgccagcagcccgcgccccgcgcccgcacucggccagccgagacccgccucccgcccgcccgccggccucgugagggacgcgccgagccgggcggccgagagcggcggcggcugggagcgcgucgggcccgggcggaaagugccugcggggggcgggcgagcgcgcggugccggccgccccggggcucggcgcgggagccagagcgggagccggcgcggagcgggacgccgggucccgagcggcccgcggccggggcucgcccccgccccucccuccucuccggcggcggcggcggcggcggcgggcggagugagcugcggagccuggaauauggucggggaaauggaaacgaaggagaagccgaagcccaccccagauuaccugaugcagcugaugaacgacaagaagcucaugagcagccugcccaacuucugcgggaucuucaaccaccucgagcggcugcuggacgaagaaauuagcagaguacggaaagacauguacaaugacacauuaaauggcaguacagagaaaaggagugcagaauugccugaugcugugggaccuauuguucaguuacaagagaaacuuuaugugccuguaaaagaauacccagauuuuaauuuuguugggagaauccuuggaccuagaggacuuacagccaaacaacuugaagcagaaaccggauguaaaaucaugguccgaggcaaaggcucaaugagggauaaaaaaaaggaggagcaaaauagaggcaagcccaauugggagcaucuaaaugaagauuuacauguacuaaucacuguggaagaugcucagaacagagcagaaaucaaauugaagagagcaguugaagaagugaagaaauuauugguaccugcagcagaaggagaagacagccugaagaagaugcagcugauggagcuugcgauucugaauggcaccuacagagaugccaacauuaaaucaccagcccuugccuuuucucuugcagcaacagcccaggcugcuccaaggaucauuacugggccugcgccgguucucccaccagcugcccugcguacuccuacgccagcuggcccuaccauaaugccuuugaucagacaaauacagaccgcugucaugccaaacggaacuccucacccaacugcugcaauaguuccuccagggcccgaagcugguuuaaucuauacacccuaugaguaccccuacacauuggcaccagcuacaucaauccuugaguauccuauugaaccuagugguguauuagaguggauugaaaugccagucaugccugauauuucagcccauugacuugcuggaugaaggacuagaauacagcagcuguuauaacacgaccagucaauguggaacaaacuguuucugugcaaccccuuuguuuuaccagacaaaauuugaauacuuuuuuuccugaauuguauaugaccuuggugcugcaugcaugcuguugacuuuuaggacuuugaucuuuuaagguuuuuuuuccccagcauuaauauugauuuauaaagauuugaaaaucuuuaaugaacuggagaacacuaagauuuaaacucgaaaauucguuguucaaguaaagaaagccaugaugcucuguauguuaucugugugugugcaugcacucaggugcccuuuguuucaugaacaaauacauuucauuguacauguuuucuguuuaaaucauuguauaaaguaauugcaggucagaauuauaccacagaacuguuuaugagaggcuugugucuguugcacauuucuugaagcauuuuuaaaauaacauguaaccuguaaccuuguuguuuaaguuuucuuuucuauuaauacucuguccuguggucccgugcaugcuccuuuucccagaacuccucucugcugcacccacagcaucuguucccgaggaguuaugacucuugacuuccugcagggcuggggcucuuagccaccagcugcuguuccagcacuuucagcgcaagaucucccugauuuugccacguggaauuguacuuguauaugauuaccuuaucuaaaaugaauaagaggugauggaccaguuuacugcuuagaaauagcaagaggcacugcaguaaaacuuguuucucauuguaaagcuucaugucuuuuguuuguuggaaaauuuuuacuuauagaaacuuaauuauuagacugguaaaauaaagaccaaaauaugcagauuucuaauuggcauucauaaggugaauaauaauaagugcccaaugaaaaaaucuauuaugguuaauuucauuucuugcuuugccaccuaagcaguaaaacaugauauugaccacuuggagaacucagaaaauuauuuuaaauuucuaaguuauaauaaauuugcacacagauaacaugcaugcuauuuaugucacaucucacauuaaauuauuuuaaaauaagcagugcccuucaaaacagaugcagacauguguguugguaguagugaggagauugguauuagcaucaagucuucauugaugacuaauuuuuaauucccuuccuuuuaucuuuagguauggcuuucccaacgaaaggcuaagaauucaagaacggucuuaacugaacccucaucagaucugaauuuaacaaaugcuuagucucagcagccuccgggggaaaaaagcuuagccuagcagucagugacuuacuugcacuuuuugcacauagauauaaaguaaaauuauguuauuaauuugguuuagucuguaauauuacacaguaaugguaauuuauaaaggaguguauaguaguauacugacugcuaaguguacuauacuguuugcuuuacuaaucaccuaauucauugcaguucauacuuauugacucagaguuuaaaaccacaaucuguaggcuuuaagaauugcuuuuaaaccuuuuuaagugauaaacucuugaaguggucuuaagguuagcaaauaauugucuguauuaaacauggcguuauuuaaguaguccugcugcaagaaaggcacuucagugaauaugugacuaguaaagcuuaaauauguuuggaucuaauagagcucaugaaauacuguaacuuggggauuguaaaugaauggauacaauagguuaaggcccaagauguuugaaaugaaugcaauauuaauagaugcauauauacaugacauauugugguuaauuuuaaaacuacugugccuuaacguguuucuuaaacuuuuguaguaaaugaacauuugaaauccauuuugauaaaccugcuguuaauguuuuuuccccccuugugaauguuuucuaacuuugucuugguaauugcaauuuaacuaggugcgguggcuacuaaaguucgaaggcacgauaugcguguccauccuuaccaaaggauugugaccgcagaccgagguuaguuuaguucugcaguucuugucuauaagaaaugcguuggguguccauaaaauuugcaacaccacaccugauggaaaaauguaauugcuuaccugcacacaguuuuuuuuccuuuucuaaauuuguuugugaucauugacagauuuaaggguuggguuuuuuguuuugguuugguuuuuucucccuucauauuuuaacuuauuaaaucuucuauuauuauguacuaugauuuauuccuacuaaaacauaaauuagucaauuuggggagcuauuaaauuuugauguugccucaguuauuuucauuaauaacuuuugcuaaaauuuaccuuuuaaaaguucugaaauuugggguugguauugcaaaaugaauugaacggauggugcuuuaaugagagaaguaaaggaauauaugccuccuguguacuuuuggagaaaacugaagauuuaguuuccauuuuucuuuauaauaaaauauuugagccacuguauaaacaugagggugcacaugucaaguacuuagcuaaaaucaacaaagugaggacugugaagauaacuuucauguaguuggauuuuuuuauauaaauuagagcgucaauucccuuuuauucuuacauucucuugccaaaauggauccuuugauuuuugucauuugcugcuaaaauauauuaucugaagguaaacugucucuugaaguucuguggugguaugagauccagcugugugaauauaauuuuugacuaaccugugacuggaaaauaagucuucauuuuucuccuuuacaacuuuuauaauuuguguccuguauaugcccuuggacccuuuuauaaauuauuuuugcuuauugaggaaugguauuuucgguuuauugacauugaauuguuuucuaaguuuuuccaucacaagauaggaauaucaagcaugguaguaaaaaaggauuuuuuacaaguuucuauuauggcacauuuguuuccuacaaauuaaauugaucaaagucucaaauauuaguggaggaaaaagugcuggauuuguacuucaauuguacaucuucuaugaauuuaugaaggaccuaccugcuuuaugugauuaccuugaguacugaugauaaauacuagacacagcuguuguccacaucagaaauaacaugccuuucccaaagacauuguuaauauuuguuuagcauaauccauaaagaacguugaagacuuuuaaacuaauguauucaucacuaacagugauuguucagugaacaaaauagaccccagcuccguggucgucuaugaggaugguauuccuuguaaguguucuguaugagcacgucucauagggauggggaaguuuuuuggaaagucacuugaguuuuaaagaaacuggacaauuccaugaauaauuugagguuuuauucauauugcuaaugucuacaauuguucuaaagaguauuuuaaaguaguuuuuuucccccagcuaaucuuucccuauguaugcccccuuuuaggagauuucucaccauuuugcuuuaacacaauaauuauuggccuacuuaauacuccagugccuugagauaguuggacauauuuuaggaagauuuuauuguauuuggaaaauuacgacacauuccaugcauggaucuuuuguaguuacguguuuugauuaguccugucacuguuuuuauuuaaaaugcagugucagcaaaccgaaaugcucauuucuuuuuuaguaacagaguacucguauacuuaucacuguaugcacucauugggucuaagggagaugucugaaguccuucacuuaaauuauuuuaugauacuacuguuuucucuauuuuugagguuaaaauuauuuuaaaaaaauauuuugaagagaaguuuauuaguauaagcauucauuuaaugcaaaaaaacucagcccuuggacauauaaccauguaauucuauaugaacguuuagaguacuacaugguugaccaauaucuaguucuuuacuaaaaguaaacucaccucuuuauauauauuucaaaacugaauuugaagccuauuagagaccaacugaccuuuuaguuguaaugaacuguauggucuugcauggugacacaccuguguaggaggccaagacuuggcagaggauguagucuagguguccugguauauauaaauggucacucacuucguuuauauauuccacacucagaaaagaagucagaguuuuuucugaguaauacaaaaaccaucacuuacauaauuuguauaagaaacuuuaaacaguuuuaaucaaaacuaugaguaacaucaaaaaugcuuuaagauucuauauuuuggaauaccugugaaguaguaugacuacaucauguuaaauauuuauuuucuucuaugauuuuggagcagcaauuuuuauuuuaaauaaaauuccacuuuuaagaaauucagggaagauuuggucacauugaagauacaguauuuuuguaguauuuauaaacuguucuaaaugauagacuauagaaaacauuuuugucauaugaagguaaaucaguccauuauuuuggaucauuuaaacugaacauuacaccuucugggcuuugauuuaugaaguggcacagaaucuaacuuugcacugaauaucuuuucauucacaucuccucugccuuagucacaauggcaacaguacagaaaauucuaucaaaccucagaauauaguagaaauaauuaagcuguugaaugagucuuaaaaauuauacuacuguuaaguggaccaaguuuggugaagcagaaugugacaaagguugauuaaggaaggaacaacucaaggacauugggaaugauaacuuuuccacuugagaacuacuuuauguuuuacuguaauuuuuaaaguuuuuuuguccuuuuuguuauuuugcaaaagaaaauaguauuuacagguggcuucuuuuaaaauauaaaaauauaaagcaggaauguauaugaaaugucagauuuuauuguauuugcagaguauuagcuuugaaauugaauaaagaaagcuguuuguaguuuuaaaaugccuuauugguaucaauuagaaauuucuucuauuuuuugauguacuuagagcuuuuugaguguagaauuuuaaauggcaggauuuuacaguguuuacaugcaagugcauuuuauaaguguucuauauguguaaaauaguauuuucaacuggaaaguguuggcuagugcaaaaggccuggccauuuucugguucccaugauguugccuacacugcuagacuacaguuuaguaucgcuuuguaucaugaggccaagaaauuccauguuguuuguaaauagaauaauugaaaaagcaauaaacauuuauugaacaaaagaaaccuuguuuggcccagaguuuauguugaaccaaaugguucauagaauuuugaauuucuuuagauucauuucuguuaaucaucaguuauuaauaaucacauccaucauuaauaguugcuuuaaugcuugcaucuuagagaauacuaaugcuuaauagcagucagauacugauaacugcacugacuguuuaaaaauucuguauaccaaaccuauucauuguuguuuuucaauuuccuucauugacugcuacuaacuauuaguuguuaguuguauuuuauuucuauuuuuucuuguuaaccaguuuuuuuuuuuuuuuugugggagugggcucuucuucauuuguucaaaucaugauacgaaauugucaugauuuuaauuguauucuguagauggugucaaucaguuugucagaauuaaacaugcuuuuuuuuuuauuaguugugauuaguuuuucauguugucauuaagccgucacuagcugaaacuaaucucuucucuaucuuuucuuuucuuuuuuuuguuuuguuuuuuuuuguuucuaaccacccagccgccaccggcaacuaaccuaugaccuucugaccucugaacucuucacccaaugaugaccugaccaugccugccugcugaucaguuaacugguaaucgccuuugcuugccugucgucagugcagcgagcugaggcacuuguccguucgucuuaccaucuaaccaaacaaaagacaaagaaauuguuguccuccaacucagcuuuuuuuuuuuuuuuuuccuguuugggugaaagugguucuagaaacugcacugaauaguaguaaagcaauaaggcccaauucaucccacagcacugaucaucuuuuaauaucccacccuaagcgaacgguaagaaggccucucuuaagaaggggagacagaugguccuuaacuacucaaugacagaggcaguuacugugagagacuucuaggaaucuuuuucuucucauagcgaagucaaagcucucucugaauguacugugugaugaugcaucaugcaugaaccuucggucagggauaucauuggugaagugauuucaaaaaguauucaaaauuugauaugcuguuuagucacuacagugcccucaaagggcagaaguugcagccuuuuuuauauugccugccaaaauuugaaguauuagaagaaagugugccaugagagaaaaacuuaaggaguuuugaaaaguaaugcaaauaacaaaacugcaacacuauuuuuaaaaagauaaauaucugaguuaaaauuacugaaucuuuauuuuacaccuaaaaaaauaugagaacaagguacaugcauuaugugucacauuacugggcaaacuguucaaguauuuuuuuuuaaaccucccuguauagaaaaaaaucauuaaggauguaaaagccaugcuugccuauuugcuguauacauguaaugaaauuguagauaaaguguagugcauugaaacaaaugaacaaaaaguagauacuuuuacuauacaagggugcuggugcagaaaaaaauauauauauuuuuggaaauguagcauuuuauacuuucaaguguuauaaaaaaaaagaaaaagaacaaagaaacccuuuauuucauuaaaugauuuuuucuggugguugucaagaaacaaaagaccaaaagagccuuuuugucuuucuuuuuuauuuuuuaaguauuggaauaagucuaaagaaaagaaagugccuuauuuuccuucauggucauucagucaagugucucauaaagaucagcuccucccucagaauacaaguuaaugcauguuacucagucgcccaguaugugaaagaagauaugagggagacaaaugaguugauaguuugaauuauaggauuuuugccacguuaccuugaugcaaauuugccaaaucugauacugugaaaagauuugauaacuuuucuaaugccugacuaguaaguuuuaaaacaucauucuuuaaaagaauaguuuaauacuuuugguuauuccaaucuucaauguuaacccaaaggauucugagauaaaaauuaugugucaucuaaacuucaguuuacagacaaaauuagagcuuuguuuuauuuggagaagggggaugcauucauuugauuuuguuuuuuguuuuuaauuaauugaauauaauaggauaccuagcaaauguuuucacaaaagugaaucuuugugauuuucauucuagaaaacaauguuuuaaaucacccaaauuuaaucacaugucuuuauaucauaccagauaacugguguaguguauccuuuuacuguuucaauaaccucugccucccccaccccuuaacuuuaaaagaaugugaaaaacuagcaucauagugcauauaaauacucaaccacauuucagcuuaagcuucuaauuucucaagaaguugaaacaguuaguuauguaguugaagcaauuugugugcaaagccuacaucuaaacacuugagugaauaaucauuaacugcucaguaccagacguggcaaaucucaagugacaguggacccccccccccgcccagcuuaucaacucguccccucuuccacaccuucccagcaucacagucagaggucagcaggaagcugagucauuaccuucuuggugaccucccccaccaucuugucucacuuggaaaccguugucaguuuauuuacuaugcaauagacauucauuguuuuguauccagcuagcuuugguuuaauaugccacugcuuugucuuucuguuacacauacaguuuugauuuauuuacaauauaugcugugccauuuugauuuuuauauuguuugguugguugaauaaauuugcgacacucaaacacuugaggugauaguuguuaaaaacaauaccaguauuugauccaguuucaucucaacuauguuauaccuggacauuuuagauguuuaucaaaggucuuuuauggggaagaaaguaaauguaugaaauaauaaaugugugacauuuauagggaauguuggcucugaacaaacuuuugaaaacuugguugacaaaauuuuggaucaacugaaaaaagcaugacuuuugaaaucucugaaugccuugguucucaguauuaucauucuuuauugaauuuauuucuuauuaaaauauguaguuuuuaagacuuuuuuucugacaguauuauguaauuuuuuagcguggguagaugggagugucgcuuguauguuaucguacagcugacauguauuuuugucuauucuuuauuaucuuaguuucaugcuauguauguaccauaaccaaccuauugccuaugagaaacauguaagauaauguauuuacagccauuguuacaaguuuauaauguauuuuucuaucuuguuuuauauguauguuauauaacauucaaaaagaauuuuuuucuugauugagaaaaggauacaaaaugcaaaauccacaauuuugauaacugaaaauugccaauuguuuugcaguacuuuauuauauugggugucuugucuuuuuugggcuuuuaguuagcuaaugaaugaauacauuagacacuuuuggguuuuaguugggauuuuacauagcuugcauuuuaauucuuugguucuuugcuguuucuauuaacccacagcauuauuuuaauaaauguuauaauaccaaccuacuaacucuggacuuugucuguuuauuaaccuuuauauguuuaauuaaaauaaacaaauaaagacaaaagaauguaaagucuugaguuacuggacuaacccugaagaacgugaccacagauaacauagugugacuuguuuuuauguuguuugucagcugacaguuuugcacacuacuguuaaaauggcuuugguuauucuggucuuuuagcuugggggccggggcggguguggguguaggugcaguagagaugggaacaaaguagccuuuguuugaggcauucuucucuugccacuugccuuucagcaucugcauuacuaauuccacaccuuauuuuuccuucuccaaaauaaucacugucugcucacagcaucgaguaaaaagauacauugucaugguauauaccuggagaaaccaguuugauucuuuguugaaccauuucuuuuaauuucuguaaauaaguaauuuuauggucacauuguaaagcagagaaacuguugauauuuuaccuuuauuuagauuuuuguuuuaacuugaccuuuuuccuuuggaaagcguugaacuuguuuagcuaacuacuccuauuaggggagcuuaacuuggguaaagucaaaggcccacuggcuguuucucaugguggccugaagccccgcugcuuuccagugaggaaaagaacccucagcacguuacugagcuuuccucagguauuuuuuucucugucaccuacuuuacaaauacagucagacuaaaacauuaaacaagaaguuuaagggggaauaacauugggauuugaaaaguuguaugugauuaauuucuugcaugcaaaucaauuuaagauuucuuaaauaucuucaugcuuuucuuggcuggaacuuggguaaauuaaccuguaauuaugaauuauuuaguuuaacucaaggugcauuuuuauuucacagauugauacuugcuuuuuucugugaaacaguucuccuagauaccaaaauccucaggaugaauuuuugcauuuguaaauaacaauuuaucuuuguaaauuacauuuuauuuauuuagaguucuaaaauugagggcugaaaugaaugacauguaauagaagacaaaacuguaccaauacgcuguuaaccaaucagacuguccagguauucucauuucauaaauauguauacacacauauaaauacacagaguaguguuagcugaugugagugauaacagcgaaucaccauugagauggucauuuggaucauguaaacaaaccgugucaugucucugaugggagcgguggguaguguacuguuagguggacaggucuuuuaacaugcuacuauuuguaaaaguuuccguugccauaacaguucauguaguagucugugaaaauucgaagcugucugucaagcugcuaauauacaaguugucaugguaccauguaacuuaaauugaugaauuucuaugcaauuuacuauucucugaagccuaugaaugcagccucaucucauuuucaguuaaggguacuuuauguauaauuaugucauugcugccuuuuauguaggaaauguaaaaaaguuuuaaaaggcgaaaauaacauuacagauuucauuuuuauacagguuacaauauuaaacaugaaacuaccaaauuccaagaauaguaaaauucuagagucaguugauugauugaaacuauuugacaaaacaguugaaagcugagcuuucagucaagaaguuggugaaauuaauguccuuaaacuuuuccuuuaggcaaaaugucagggaaaacuuaacaugaaaguaguuacucccauacgcaccagugcaguagcuccagguguaagaggucacgaaggcacccugcccauaaaaccaggaugugcauacguacacacaaucggugucugguuaugguuuucuaaacacuacauaaggaucugaauuuuuuagugugccaaaagcaaacgauagguuuaaaugaaauugcucacuaccaaaucaagacucuucauauauauacguguguguauauauauaugucauaacacuuucacgugugacagccuaagguuucuuuaguuuuaggaugggguggggugggagugguuuugcauguuugaccaggaugugaugccugucuuuauuccuugauaagaaguagcuguuacagcauuucaagcaaauuauuuuagguagaauagaacuuacugccaaaugauuauuuauuuagcaaaaauuauucuccaacuuuucgaauucacaauuuuucuaagugcauagaguaguuuuuuacauugucacgaauuccuuaauaccuuuauuuaaaauggaaaaaauauggacaauauuuuagaaauaugugcuacacaucuaauuuuaucuguaguuuaagugaucuaaauugagaugccuuugauaugaagagauuuaccaacauuauaugcacucgugccuucaauguggaaucaaacugguuaaccucagcacaguccucucguuucuguguuucugcugcacuaauugccagggggugggggagaaggcgaaugaauuaauucauaguagaaggaggcgauaggugcagcaaagggcagccacaguguugauuccacauuauaauguuguugccucuucuuggcaaaagacaccacauugugggaagaucucagcuuccaggguaaaaguuaauuuauaacuuaaaagugcuauuaaguuuuuauuaccaaauauaucuuuuaugguuuauauuguagugguauguaugaaacauuuaaaauuuuacuguggaaauuguguauauauauauauauagucgaaauagguguucacaggucacaugugaacggagaacugcaugaccguacaugaaaugcaauaaaccaacuggaaaaagugcaugugcuucauccucucaagccaacugcagcuggaaagugcugcuuauccuccacccccagaaaaugcauguaucaauaugagaauaaagaacgcacacuuucaauuuuauugaggcuuucaacacuauuuaaaagaaaauguaagaauuugacauucuggaguuauuauaacauuagaaaaugagcauaacauucacucugauuuuagccauuaagggagauuaguaaacagacugcuacaguguuccauaguuggacugugcauccaaaacauuuuuuuaucuuuaauaaaugguacaguuuuuauguaguuuucgaauguaagaagaaaggaaugcugaccaaaacuugauuucaucagcuucaugaaaaggacuagugucauuaaccuguugaacagaauugguuuauuaaaaaaaucauuuccaguagugugaaaccuuuacgagucuuuaacaucuaaauguuaugacuccuuguaccuuaaguuuuccagucuuucuuauuuauaucaucuccaaguaccucuggcuccuuuccucuugcucaccggaaccuuaguuuuccucaacagaaugcuuuguuaaaguagcccacaguugcaggauccauagcaccgucgugcagacuagcagcccaaagguguguuugguuuggcuuauacgguguuuugcuuuuuaaacuacuugccauaauuuaaaaguggcaacacuagacuuaaaaaaaaaaaagucugauugcccauauuagauuuuuuuuuuaauucuucacaaaaucugcucuuccugaaagaucaaagugucuagaaagcccaaacauguauucuuaacauaguaggcaccagcugaaacugaguaauuaaacgguccccugaagccaaguauucccugguuagucgccaccccaccacuccuuccugucucuagugucacacuugggcuguugauuuucuuacucuucucuugcuuuaauccuuccccccggcucucuggcuccuguggauaucugugcuuguuuccugguccaggauggugaucugacuuucaaaccagcuucucaaaaggggugacauaaaaucaguuuugauguuuuuccuccugaaaaaucagaugaauauuuuagucacuguuacucaugcacauuguguucuuauguuuacagaagugcuuaagugaauggaagcacuagacauuuggauuuccuuccuaacguaauuuuuaaugauuaccccuuuuauacaguaauuuguggucuuuagaaagcaguuaaacuaauugaccaucuaauaguuguacuauacauaugucuaaaauaauagucaugguaaguuuggcaucauaucuuccccaaaaaauguuuauuaaaauuagauuauuccaguuuaaugcuauuuugugaacuguauacccucugaaagcgcuuauuuuuacaugcuacacaacaguuccaauuuuaaggagugucuccuaaaauugggaugaaaaucuacuguagucuguuuuaaaguaugcuauacuauguucauugguuacuuaacuggauauuaauauaaagguuauuacaagaaaaaugaugaagagcaaaaggagaagaaaauauuugcaagugaauccacaauucuugcagaacuauuugaguugauacuaaagauuuuauguucacuccuuuaccuuagaacugucaagcuugggaaugaggaaagugccuuggcugugcuugaaaccugaauuuuagugcuucccuuauuacauucauuguuuucaaugauugauuuauaaaauuaagacauacugguaguacaaguugaaaguugguuugaauacauuuuaauuaaaugauaauauguuaauaugcuuuuguucauugcuuucucacugagguaaaacagcauuaaaaaguuguccagaauuuaaacugaaaaaaaaaaaaaaaaa

SEQ ID NO: 66 - RNA, 16378 nt, Homo sapiens
ugaacugaggcgaacuccgccgccaagugagugaggaggaggaggaggagaaggaggaggaggaagaggaggagcgcagucggagcgcggcggcagcggcagaggcgccgcggcggggaccagcccagagagaccccccgagcccgcggcacaggcggcggcggcgcugagcgggcucgaccagccgagcgagcggcggccccggcuccuccucguccggcgccagcagcccgcgccccgcgcccgcacucggccagccgagacccgccucccgcccgcccgccggccucgugagggacgcgccgagccgggcggccgagagcggcggcggcugggagcgcgucgggcccgggcggaaagugccugcggggggcgggcgagcgcgcggugccggccgccccggggcucggcgcgggagccagagcgggagccggcgcggagcgggacgccgggucccgagcggcccgcggccggggcucgcccccgccccucccuccucuccggcggcggcggcggcggcggcgggcggagugagcugcggagccuggaauauggucggggaaauggaaacgaaggagaagccgaagcccaccccagauuaccugaugcagcugaugaacgacaagaagcucaugagcagccugcccaacuucugcgggaucuucaaccaccucgagcggcugcuggacgaagaaauuagcagaguacggaaagacauguacaaugacacauuaaauggcaguacagagaaaaggagugcagaauugccugaugcugugggaccuauuguucaguuacaagagaaacuuuaugugccuguaaaagaauacccagauuuuaauuuuguugggagaauccuuggaccuagaggacuuacagccaaacaacuugaagcagaaaccggauguaaaaucaugguccgaggcaaaggcucaaugagggauaaaaaaaaggaggagcaaaauagaggcaagcccaauugggagcaucuaaaugaagauuuacauguacuaaucacuguggaagaugcucagaacagagcagaaaucaaauugaagagagcaguugaagaagugaagaaauuauugguaccugcagcagaaggagaagacagccugaagaagaugcagcugauggagcuugcgauucugaauggcaccuacagagaugccaacauuaaaucaccagcccuugccuuuucucuugcagcaacagcccaggcugcuccaaggaucauuacugggccugcgccgguucucccaccagcugcccugcguacuccuacgccagcuggcccuaccauaaugccuuugaucagacaaauacagaccgcugucaugccaaacggaacuccucacccaacugcugcaauaguuccuccagggcccgaagcugguuuaaucuauacacccuaugaguaccccuacacauuggcaccagcuacaucaauccuugaguauccuauugaaccuagugguguauuagguaaguucuucuccccaugggguuaacaacauucucuuuauaaauauuuuugcucccucagauuuugaaaugauuuuaucaguuuuagagaggcaagacaaguuugcauuagggaagaccgaaaacuaaauuuuguuuuauuucagccucucauaaggguuccccuuugaaauaauugcaccuuaaauuuauucagauccacuuugguaacugagguucaguuugguuggguaaaucuguuguguacauuucaacaaauacuuuuacauuugacauuacugaggucauucugaguuuuaggucccaccccauuggcccgucaccuccaucagcuccacuucucaaacuuuauuugagauuuauuuuaucucacagguuuguguaauuguuggaaaauuuuuaaacuucuaagugaaaguagucugagcgcuuuauaaaauucauauuauuguguguguuuuguaauauuugcgaucacuuaaaauaucagauaaauacagucaugcaucgcuuaacaacggggauacagucuaagaauugccuugucuggcaguuucaucauuugguguacaucauagaguguacuuacacagacauagaugauaucguuuacuacacaccuaggcugugugguauagccauuauaaucuuaugggaccacauaguauaagugguccguuguugaccaaaauguuguuaugcaggcaugacuguaguuguauucuuaagauuuuucaguucuuguguuuuggaguccuagucauauuaaacaugagagauacucuuaauuucaguaguacaguauggaauaguuaaauaaauagcaaaucacugaauucagaaucucacuuaaggcagaaauuaaaaucauaauauaaacgcuugguuuuacaugaacaaaaaguggugugcucgaaaaagacauuacugaugccuuuuuuuuauagaguggauugaaaugccagucaugccugauauuucagcccauugacuugcuggaugaaggacuagaauacagcagcuguuauaacacgaccagucaauguggaacaaacuguuucugugcaaccccuuuguuuuaccagacaaaauuugaauacuuuuuuuccugaauuguauaugaccuuggugcugcaugcaugcuguugacuuuuaggacuuugaucuuuuaagguuuuuuuuccccagcauuaauauugauuuauaaagauuugaaaaucuuuaaugaacuggagaacacuaagauuuaaacucgaaaauucguuguucaaguaaagaaagccaugaugcucuguauguuaucugugugugugcaugcacucaggugcccuuuguuucaugaacaaauacauuucauuguacauguuuucuguuuaaaucauuguauaaaguaauugcaggucagaauuauaccacagaacuguuuaugagaggcuugugucuguugcacauuucuugaagcauuuuuaaaauaacauguaaccuguaaccuuguuguuuaaguuuucuuuucuauuaauacucuguccuguggucccgugcaugcuccuuuucccagaacuccucucugcugcacccacagcaucuguucccgaggaguuaugacucuugacuuccugcagggcuggggcucuuagccaccagcugcuguuccagcacuuucagcgcaagaucucccugauuuugccacguggaauuguacuuguauaugauuaccuuaucuaaaaugaauaagaggugauggaccaguuuacugcuuagaaauagcaagaggcacugcaguaaaacuuguuucucauuguaaagcuucaugucuuuuguuuguuggaaaauuuuuacuuauagaaacuuaauuauuagacugguaaaauaaagaccaaaauaugcagauuucuaauuggcauucauaaggugaauaauaauaagugcccaaugaaaaaaucuauuaugguuaauuucauuucuugcuuugccaccuaagcaguaaaacaugauauugaccacuuggagaacucagaaaauuauuuuaaauuucuaaguuauaauaaauuugcacacagauaacaugcaugcuauuuaugucacaucucacauuaaauuauuuuaaaauaagcagugcccuucaaaacagaugcagacauguguguugguaguagugaggagauugguauuagcaucaagucuucauugaugacuaauuuuuaauucccuuccuuuuaucuuuagguauggcuuucccaacgaaaggcuaagaauucaagaacggucuuaacugaacccucaucagaucugaauuuaacaaaugcuuagucucagcagccuccgggggaaaaaagcuuagccuagcagucagugacuuacuugcacuuuuugcacauagauauaaaguaaaauuauguuauuaauuugguuuagucuguaauauuacacaguaaugguaauuuauaaaggaguguauaguaguauacugacugcuaaguguacuauacuguuugcuuuacuaaucaccuaauucauugcaguucauacuuauugacucagaguuuaaaaccacaaucuguaggcuuuaagaauugcuuuuaaaccuuuuuaagugauaaacucuugaaguggucuuaagguuagcaaauaauugucuguauuaaacauggcguuauuuaaguaguccugcugcaagaaaggcacuucagugaauaugugacuaguaaagcuuaaauauguuuggaucuaauagagcucaugaaauacuguaacuuggggauuguaaaugaauggauacaauagguuaaggcccaagauguuugaaaugaaugcaauauuaauagaugcauauauacaugacauauugugguuaauuuuaaaacuacugugccuuaacguguuucuuaaacuuuuguaguaaaugaacauuugaaauccauuuugauaaaccugcuguuaauguuuuuuccccccuugugaauguuuucuaacuuugucuugguaauugcaauuuaacuaggugcgguggcuacuaaaguucgaaggcacgauaugcguguccauccuuaccaaaggauugugaccgcagaccgagguuaguuuaguucugcaguucuugucuauaagaaaugcguuggguguccauaaaauuugcaacaccacaccugauggaaaaauguaauugcuuaccugcacacaguuuuuuuuccuuuucuaaauuuguuugugaucauugacagauuuaaggguuggguuuuuuguuuugguuugguuuuuucucccuucauauuuuaacuuauuaaaucuucuauuauuauguacuaugauuuauuccuacuaaaacauaaauuagucaauuuggggagcuauuaaauuuugauguugccucaguuauuuucauuaauaacuuuugcuaaaauuuaccuuuuaaaaguucugaaauuugggguugguauugcaaaaugaauugaacggauggugcuuuaaugagagaaguaaaggaauauaugccuccuguguacuuuuggagaaaacugaagauuuaguuuccauuuuucuuuauaauaaaauauuugagccacuguauaaacaugagggugcacaugucaaguacuuagcuaaaaucaacaaagugaggacugugaagauaacuuucauguaguuggauuuuuuuauauaaauuagagcgucaauucccuuuuauucuuacauucucuugccaaaauggauccuuugauuuuugucauuugcugcuaaaauauauuaucugaagguaaacugucucuugaaguucuguggugguaugagauccagcugugugaauauaauuuuugacuaaccugugacuggaaaauaagucuucauuuuucuccuuuacaacuuuuauaauuuguguccuguauaugcccuuggacccuuuuauaaauuauuuuugcuuauugaggaaugguauuuucgguuuauugacauugaauuguuuucuaaguuuuuccaucacaagauaggaauaucaagcaugguaguaaaaaaggauuuuuuacaaguuucuauuauggcacauuuguuuccuacaaauuaaauugaucaaagucucaaauauuaguggaggaaaaagugcuggauuuguacuucaauuguacaucuucuaugaauuuaugaaggaccuaccugcuuuaugugauuaccuugaguacugaugauaaauacuagacacagcuguuguccacaucagaaauaacaugccuuucccaaagacauuguuaauauuuguuuagcauaauccauaaagaacguugaagacuuuuaaacuaauguauucaucacuaacagugauuguucagugaacaaaauagaccccagcuccguggucgucuaugaggaugguauuccuuguaaguguucuguaugagcacgucucauagggauggggaaguuuuuuggaaagucacuugaguuuuaaagaaacuggacaauuccaugaauaauuugagguuuuauucauauugcuaaugucuacaauuguucuaaagaguauuuuaaaguaguuuuuuucccccagcuaaucuuucccuauguaugcccccuuuuaggagauuucucaccauuuugcuuuaacacaauaauuauuggccuacuuaauacuccagugccuugagauaguuggacauauuuuaggaagauuuuauuguauuuggaaaauuacgacacauuccaugcauggaucuuuuguaguuacguguuuugauuaguccugucacuguuuuuauuuaaaaugcagugucagcaaaccgaaaugcucauuucuuuuuuaguaacagaguacucguauacuuaucacuguaugcacucauugggucuaagggagaugucugaaguccuucacuuaaauuauuuuaugauacuacuguuuucucuauuuuugagguuaaaauuauuuuaaaaaaauauuuugaagagaaguuuauuaguauaagcauucauuuaaugcaaaaaaacucagcccuuggacauauaaccauguaauucuauaugaacguuuagaguacuacaugguugaccaauaucuaguucuuuacuaaaaguaaacucaccucuuuauauauauuucaaaacugaauuugaagccuauuagagaccaacugaccuuuuaguuguaaugaacuguauggucuugcauggugacacaccuguguaggaggccaagacuuggcagaggauguagucuagguguccugguauauauaaauggucacucacuucguuuauauauuccacacucagaaaagaagucagaguuuuuucugaguaauacaaaaaccaucacuuacauaauuuguauaagaaacuuuaaacaguuuuaaucaaaacuaugaguaacaucaaaaaugcuuuaagauucuauauuuuggaauaccugugaaguaguaugacuacaucauguuaaauauuuauuuucuucuaugauuuuggagcagcaauuuuuauuuuaaauaaaauuccacuuuuaagaaauucagggaagauuuggucacauugaagauacaguauuuuuguaguauuuauaaacuguucuaaaugauagacuauagaaaacauuuuugucauaugaagguaaaucaguccauuauuuuggaucauuuaaacugaacauuacaccuucugggcuuugauuuaugaaguggcacagaaucuaacuuugcacugaauaucuuuucauucacaucuccucugccuuagucacaauggcaacaguacagaaaauucuaucaaaccucagaauauaguagaaauaauuaagcuguugaaugagucuuaaaaauuauacuacuguuaaguggaccaaguuuggugaagcagaaugugacaaagguugauuaaggaaggaacaacucaaggacauugggaaugauaacuuuuccacuugagaacuacuuuauguuuuacuguaauuuuuaaaguuuuuuuguccuuuuuguuauuuugcaaaagaaaauaguauuuacagguggcuucuuuuaaaauauaaaaauauaaagcaggaauguauaugaaaugucagauuuuauuguauuugcagaguauuagcuuugaaauugaauaaagaaagcuguuuguaguuuuaaaaugccuuauugguaucaauuagaaauuucuucuauuuuuugauguacuuagagcuuuuugaguguagaauuuuaaauggcaggauuuuacaguguuuacaugcaagugcauuuuauaaguguucuauauguguaaaauaguauuuucaacuggaaaguguuggcuagugcaaaaggccuggccauuuucugguucccaugauguugccuacacugcuagacuacaguuuaguaucgcuuuguaucaugaggccaagaaauuccauguuguuuguaaauagaauaauugaaaaagcaauaaacauuuauugaacaaaagaaaccuuguuuggcccagaguuuauguugaaccaaaugguucauagaauuuugaauuucuuuagauucauuucuguuaaucaucaguuauuaauaaucacauccaucauuaauaguugcuuuaaugcuugcaucuuagagaauacuaaugcuuaauagcagucagauacugauaacugcacugacuguuuaaaaauucuguauaccaaaccuauucauuguuguuuuucaauuuccuucauugacugcuacuaacuauuaguuguuaguuguauuuuauuucuauuuuuucuuguuaaccaguuuuuuuuuuuuuuuugugggagugggcucuucuucauuuguucaaaucaugauacgaaauugucaugauuuuaauuguauucuguagauggugucaaucaguuugucagaauuaaacaugcuuuuuuuuuuauuaguugugauuaguuuuucauguugucauuaagccgucacuagcugaaacuaaucucuucucuaucuuuucuuuucuuuuuuuuguuuuguuuuuuuuuguuucuaaccacccagccgccaccggcaacuaaccuaugaccuucugaccucugaacucuucacccaaugaugaccugaccaugccugccugcugaucaguuaacugguaaucgccuuugcuugccugucgucagugcagcgagcugaggcacuuguccguucgucuuaccaucuaaccaaacaaaagacaaagaaauuguuguccuccaacucagcuuuuuuuuuuuuuuuuuccuguuugggugaaagugguucuagaaacugcacugaauaguaguaaagcaauaaggcccaauucaucccacagcacugaucaucuuuuaauaucccacccuaagcgaacgguaagaaggccucucuuaagaaggggagacagaugguccuuaacuacucaaugacagaggcaguuacugugagagacuucuaggaaucuuuuucuucucauagcgaagucaaagcucucucugaauguacugugugaugaugcaucaugcaugaaccuucggucagggauaucauuggugaagugauuucaaaaaguauucaaaauuugauaugcuguuuagucacuacagugcccucaaagggcagaaguugcagccuuuuuuauauugccugccaaaauuugaaguauuagaagaaagugugccaugagagaaaaacuuaaggaguuuugaaaaguaaugcaaauaacaaaacugcaacacuauuuuuaaaaagauaaauaucugaguuaaaauuacugaaucuuuauuuuacaccuaaaaaaauaugagaacaagguacaugcauuaugugucacauuacugggcaaacuguucaaguauuuuuuuuuaaaccucccuguauagaaaaaaaucauuaaggauguaaaagccaugcuugccuauuugcuguauacauguaaugaaauuguagauaaaguguagugcauugaaacaaaugaacaaaaaguagauacuuuuacuauacaagggugcuggugcagaaaaaaauauauauauuuuuggaaauguagcauuuuauacuuucaaguguuauaaaaaaaaagaaaaagaacaaagaaacccuuuauuucauuaaaugauuuuuucuggugguugucaagaaacaaaagaccaaaagagccuuuuugucuuucuuuuuuauuuuuuaaguauuggaauaagucuaaagaaaagaaagugccuuauuuuccuucauggucauucagucaagugucucauaaagaucagcuccucccucagaauacaaguuaaugcauguuacucagucgcccaguaugugaaagaagauaugagggagacaaaugaguugauaguuugaauuauaggauuuuugccacguuaccuugaugcaaauuugccaaaucugauacugugaaaagauuugauaacuuuucuaaugccugacuaguaaguuuuaaaacaucauucuuuaaaagaauaguuuaauacuuuugguuauuccaaucuucaauguuaacccaaaggauucugagauaaaaauuaugugucaucuaaacuucaguuuacagacaaaauuagagcuuuguuuuauuuggagaagggggaugcauucauuugauuuuguuuuuuguuuuuaauuaauugaauauaauaggauaccuagcaaauguuuucacaaaagugaaucuuugugauuuucauucuagaaaacaauguuuuaaaucacccaaauuuaaucacaugucuuuauaucauaccagauaacugguguaguguauccuuuuacuguuucaauaaccucugccucccccaccccuuaacuuuaaaagaaugugaaaaacuagcaucauagugcauauaaauacucaaccacauuucagcuuaagcuucuaauuucucaagaaguugaaacaguuaguuauguaguugaagcaauuugugugcaaagccuacaucuaaacacuugagugaauaaucauuaacugcucaguaccagacguggcaaaucucaagugacaguggacccccccccccgcccagcuuaucaacucguccccucuuccacaccuucccagcaucacagucagaggucagcaggaagcugagucauuaccuucuuggugaccucccccaccaucuugucucacuuggaaaccguugucaguuuauuuacuaugcaauagacauucauuguuuuguauccagcuagcuuugguuuaauaugccacugcuuugucuuucuguuacacauacaguuuugauuuauuuacaauauaugcugugccauuuugauuuuuauauuguuugguugguugaauaaauuugcgacacucaaacacuugaggugauaguuguuaaaaacaauaccaguauuugauccaguuucaucucaacuauguuauaccuggacauuuuagauguuuaucaaaggucuuuuauggggaagaaaguaaauguaugaaauaauaaaugugugacauuuauagggaauguuggcucugaacaaacuuuugaaaacuugguugacaaaauuuuggaucaacugaaaaaagcaugacuuuugaaaucucugaaugccuugguucucaguauuaucauucuuuauugaauuuauuucuuauuaaaauauguaguuuuuaagacuuuuuuucugacaguauuauguaauuuuuuagcguggguagaugggagugucgcuuguauguuaucguacagcugacauguauuuuugucuauucuuuauuaucuuaguuucaugcuauguauguaccauaaccaaccuauugccuaugagaaacauguaagauaauguauuuacagccauuguuacaaguuuauaauguauuuuucuaucuuguuuuauauguauguuauauaacauucaaaaagaauuuuuuucuugauugagaaaaggauacaaaaugcaaaauccacaauuuugauaacugaaaauugccaauuguuuugcaguacuuuauuauauugggugucuugucuuuuuugggcuuuuaguuagcuaaugaaugaauacauuagacacuuuuggguuuuaguugggauuuuacauagcuugcauuuuaauucuuugguucuuugcuguuucuauuaacccacagcauuauuuuaauaaauguuauaauaccaaccuacuaacucuggacuuugucuguuuauuaaccuuuauauguuuaauuaaaauaaacaaauaaagacaaaagaauguaaagucuugaguuacuggacuaacccugaagaacgugaccacagauaacauagugugacuuguuuuuauguuguuugucagcugacaguuuugcacacuacuguuaaaauggcuuugguuauucuggucuuuuagcuugggggccggggcggguguggguguaggugcaguagagaugggaacaaaguagccuuuguuugaggcauucuucucuugccacuugccuuucagcaucugcauuacuaauuccacaccuuauuuuuccuucuccaaaauaaucacugucugcucacagcaucgaguaaaaagauacauugucaugguauauaccuggagaaaccaguuugauucuuuguugaaccauuucuuuuaauuucuguaaauaaguaauuuuauggucacauuguaaagcagagaaacuguugauauuuuaccuuuauuuagauuuuuguuuuaacuugaccuuuuuccuuuggaaagcguugaacuuguuuagcuaacuacuccuauuaggggagcuuaacuuggguaaagucaaaggcccacuggcuguuucucaugguggccugaagccccgcugcuuuccagugaggaaaagaacccucagcacguuacugagcuuuccucagguauuuuuuucucugucaccuacuuuacaaauacagucagacuaaaacauuaaacaagaaguuuaagggggaauaacauugggauuugaaaaguuguaugugauuaauuucuugcaugcaaaucaauuuaagauuucuuaaauaucuucaugcuuuucuuggcuggaacuuggguaaauuaaccuguaauuaugaauuauuuaguuuaacucaaggugcauuuuuauuucacagauugauacuugcuuuuuucugugaaacaguucuccuagauaccaaaauccucaggaugaauuuuugcauuuguaaauaacaauuuaucuuuguaaauuacauuuuauuuauuuagaguucuaaaauugagggcugaaaugaaugacauguaauagaagacaaaacuguaccaauacgcuguuaaccaaucagacuguccagguauucucauuucauaaauauguauacacacauauaaauacacagaguaguguuagcugaugugagugauaacagcgaaucaccauugagauggucauuuggaucauguaaacaaaccgugucaugucucugaugggagcgguggguaguguacuguuagguggacaggucuuuuaacaugcuacuauuuguaaaaguuuccguugccauaacaguucauguaguagucugugaaaauucgaagcugucugucaagcugcuaauauacaaguugucaugguaccauguaacuuaaauugaugaauuucuaugcaauuuacuauucucugaagccuaugaaugcagccucaucucauuuucaguuaaggguacuuuauguauaauuaugucauugcugccuuuuauguaggaaauguaaaaaaguuuuaaaaggcgaaaauaacauuacagauuucauuuuuauacagguuacaauauuaaacaugaaacuaccaaauuccaagaauaguaaaauucuagagucaguugauugauugaaacuauuugacaaaacaguugaaagcugagcuuucagucaagaaguuggugaaauuaauguccuuaaacuuuuccuuuaggcaaaaugucagggaaaacuuaacaugaaaguaguuacucccauacgcaccagugcaguagcuccagguguaagaggucacgaaggcacccugcccauaaaaccaggaugugcauacguacacacaaucggugucugguuaugguuuucuaaacacuacauaaggaucugaauuuuuuagugugccaaaagcaaacgauagguuuaaaugaaauugcucacuaccaaaucaagacucuucauauauauacguguguguauauauauaugucauaacacuuucacgugugacagccuaagguuucuuuaguuuuaggaugggguggggugggagugguuuugcauguuugaccaggaugugaugccugucuuuauuccuugauaagaaguagcuguuacagcauuucaagcaaauuauuuuagguagaauagaacuuacugccaaaugauuauuuauuuagcaaaaauuauucuccaacuuuucgaauucacaauuuuucuaagugcauagaguaguuuuuuacauugucacgaauuccuuaauaccuuuauuuaaaauggaaaaaauauggacaauauuuuagaaauaugugcuacacaucuaauuuuaucuguaguuuaagugaucuaaauugagaugccuuugauaugaagagauuuaccaacauuauaugcacucgugccuucaauguggaaucaaacugguuaaccucagcacaguccucucguuucuguguuucugcugcacuaauugccagggggugggggagaaggcgaaugaauuaauucauaguagaaggaggcgauaggugcagcaaagggcagccacaguguugauuccacauuauaauguuguugccucuucuuggcaaaagacaccacauugugggaagaucucagcuuccaggguaaaaguuaauuuauaacuuaaaagugcuauuaaguuuuuauuaccaaauauaucuuuuaugguuuauauuguagugguauguaugaaacauuuaaaauuuuacuguggaaauuguguauauauauauauauagucgaaauagguguucacaggucacaugugaacggagaacugcaugaccguacaugaaaugcaauaaaccaacuggaaaaagugcaugugcuucauccucucaagccaacugcagcuggaaagugcugcuuauccuccacccccagaaaaugcauguaucaauaugagaauaaagaacgcacacuuucaauuuuauugaggcuuucaacacuauuuaaaagaaaauguaagaauuugacauucuggaguuauuauaacauuagaaaaugagcauaacauucacucugauuuuagccauuaagggagauuaguaaacagacugcuacaguguuccauaguuggacugugcauccaaaacauuuuuuuaucuuuaauaaaugguacaguuuuuauguaguuuucgaauguaagaagaaaggaaugcugaccaaaacuugauuucaucagcuucaugaaaaggacuagugucauuaaccuguugaacagaauugguuuauuaaaaaaaucauuuccaguagugugaaaccuuuacgagucuuuaacaucuaaauguuaugacuccuuguaccuuaaguuuuccagucuuucuuauuuauaucaucuccaaguaccucuggcuccuuuccucuugcucaccggaaccuuaguuuuccucaacagaaugcuuuguuaaaguagcccacaguugcaggauccauagcaccgucgugcagacuagcagcccaaagguguguuugguuuggcuuauacgguguuuugcuuuuuaaacuacuugccauaauuuaaaaguggcaacacuagacuuaaaaaaaaaaaagucugauugcccauauuagauuuuuuuuuuaauucuucacaaaaucugcucuuccugaaagaucaaagugucuagaaagcccaaacauguauucuuaacauaguaggcaccagcugaaacugaguaauuaaacgguccccugaagccaaguauucccugguuagucgccaccccaccacuccuuccugucucuagugucacacuugggcuguugauuuucuuacucuucucuugcuuuaauccuuccccccggcucucuggcuccuguggauaucugugcuuguuuccugguccaggauggugaucugacuuucaaaccagcuucucaaaaggggugacauaaaaucaguuuugauguuuuuccuccugaaaaaucagaugaauauuuuagucacuguuacucaugcacauuguguucuuauguuuacagaagugcuuaagugaauggaagcacuagacauuuggauuuccuuccuaacguaauuuuuaaugauuaccccuuuuauacaguaauuuguggucuuuagaaagcaguuaaacuaauugaccaucuaauaguuguacuauacauaugucuaaaauaauagucaugguaaguuuggcaucauaucuuccccaaaaaauguuuauuaaaauuagauuauuccaguuuaaugcuauuuugugaacuguauacccucugaaagcgcuuauuuuuacaugcuacacaacaguuccaauuuuaaggagugucuccuaaaauugggaugaaaaucuacuguagucuguuuuaaaguaugcuauacuauguucauugguuacuuaacuggauauuaauauaaagguuauuacaagaaaaaugaugaagagcaaaaggagaagaaaauauuugcaagugaauccacaauucuugcagaacuauuugaguugauacuaaagauuuuauguucacuccuuuaccuuagaacugucaagcuugggaaugaggaaagugccuuggcugugcuugaaaccugaauuuuagugcuucccuuauuacauucauuguuuucaaugauugauuuauaaaauuaagacauacugguaguacaaguugaaaguugguuugaauacauuuuaauuaaaugauaauauguuaauaugcuuuuguucauugcuuucucacugagguaaaacagcauuaaaaaguuguccagaauuuaaacugaaaaaaaaaaaaaaaaa

SEQ ID NO: 67 - RNA, 2648 nt, Homo sapiens
cagguacgcaugccccagaggcgcggcgcuuggcgggaagcugaguccuggccuugcgucgcacugucuguccucagcucgcguagccgcgcucgcgacucccuuucccggcaugccaggcggugcggccgcccucugggccguguaaaggccccucggucuaaggcuucccuauuuccugguucgccggcggccauuuuggguggaagcgauagcugaguggcggcggcugcugauuguguucuaggggacggaguaggggaagacguuugcucucccggaacagccuaucucauuccuuucuuucgauuacccguggcgcggagagucagggcggcggcugcggcagcaagggcggcgguggcggcggcggcagcugcagugacauguccagcaugaaucccgaauaugauuauuuauucaaguuacuucugauuggcgacucagggguuggaaagucuugccuucuucuuagguuugcagaugauacauauacagaaagcuacaucagcacaauugguguggauuucaaaauaagaacuauagaguuagacgggaaaacaaucaagcuucaaauaugggacacagcaggccaggaaagauuucgaacaaucaccuccaguuauuacagaggagcccauggcaucauaguuguguaugaugugacagaucaggaguccuucaauaauguuaaacaguggcugcaggaaauagaucguuaugccagugaaaaugucaacaaauuguugguagggaacaaaugugaucugaccacaaagaaaguaguagacuacacaacagcgaaggaauuugcugauucccuuggaauuccguuuuuggaaaccagugcuaagaaugcaacgaauguagaacagucuuucaugacgauggcagcugagauuaaaaagcgaaugggucccggagcaacagcugguggugcugagaaguccaauguuaaaauucagagcacuccagucaagcagucagguggagguugcugcuaaaauuugccuccauccuuuucucacagcaaugaauuugcaaucugaacccaagugaaaaaacaaaauugccugaauuguacuguauguagcugcacuacaacagauucuuaccgucuccacaaaggucagagauuguaaauggucaauacugacuuuuuuuuuauucccuugacucaagacagcuaacuucauuuucagaacuguuuuaaaccuuugugugcugguuuauaaaauaauguguguaauccuuguugcuuuccugauaccagacuguuucccgugguugguuagaauauauuuuguuuugauguuuauauuggcauguuuagaugucagguuuagucuucugaagaugaaguucagccauuuuguaucaaacagcacaagcagugucugucacuuuccaugcauaaaguuuagugagauguuauauguaagaucugauuugcuaguucuuccuuguagaguuauaaauggaaagauuacacuaucugauuaauaguuucuucauacucugcauauaauuuguggcugcagaauauuguaauuuguugcacacuauguaacaaaacaacugaagauauguuuaauaaauauuguacuuauuggaaguaauaucaaacuguauggugauaaguauuguuuugauucuuaugguuaaagggaaauagagccuugcauuauauucaacacagccauuugugugugcacaaugcaaacuaagguauucuagaccuaucuuagagcagcauccaguauuugcuuucuagauaauaugcccaauaacaugaccuagaggggcuucugugcuguguagggauuuaaccaacuucagugguucagggagcucaaacuauauguaaaacaaguuuagaauguaugcuaucuagcccguuaucucugauccuucucuaaaaccauuugaaauagcuucauugaucaacauuucauaaaugcaucugugguagagguagaaagcagcaccuuuccuaauuggcaaaugaucagacuaaugugugcuaauguuuuucuuccaugcuuucagucagauucaacuauuuuauccuccacaguugcuuaacuugguguuggaggaggguuuaagcauuaagauaggaagcaggaaauuugauugcucuaaauuuagaaauuauaucccuaaaaauuaaaacaugaauacugggugguaaugauaauugaggcaaauguauuuauuuuggugacauuuugcauauaugaagauuuucugaaauaggaccuucaagauccuaggggguuuuguuugguuuuuaauugugaggaauaaaaaaucuucugcccacacuggcauuuuaaggugacugaggucaaacguuguuuccuuagguugaaauagcagccaaaacauucuucacgcaggggcuugggauauggcugcuggcaacacauuuuguugugggcuccuuaauuuaaugauaaaauuuaagcuaaacacaagccaaaaaugaauagguuuuuuuaauuuuuauuuuucacuaaacaggcaauugaaauacaugguacaaaaauaagugguaagauaauuguaaaaugaaauggacagaauauucaauuuuccaucuaugaaaauuucacaauaaaaaucauaguuuacuuuguaaaaaa

SEQ ID NO: 68 - RNA, 2420 nt, Homo sapiens
cagguacgcaugccccagaggcgcggcgcuuggcgggaagcugaguccuggccuugcgucgcacugucuguccucagcucgcguagccgcgcucgcgacucccuuucccggcaugccaggcggugcggccgcccucugggccguguaaaggccccucggucuaaggcuucccuauuuccugguucgccggcggccauuuuggguggaagcgauagcugaguggcggcggcugcugauuguguucuaggggacggaguaggggaagacguuugcucucccggaacagccuaucucauuccuuucuuucgauuacccguggcgcggagagucagggcggcggcugcggcagcaagggcggcgguggcggcggcggcagcugcagugacauguccagcaugaaucccgaauaugauuauuuauucaaguuacuucugauuggcgacucagggguuggaaagucuugccuucuucuuagguuugcagaugauacauauacagaaagcuacaucagcacaauugguguggauuucaaaauaagaacuauagaguuagacgggaaaacaaucaagcuucaaauagaauuugcugauucccuuggaauuccguuuuuggaaaccagugcuaagaaugcaacgaauguagaacagucuuucaugacgauggcagcugagauuaaaaagcgaaugggucccggagcaacagcugguggugcugagaaguccaauguuaaaauucagagcacuccagucaagcagucagguggagguugcugcuaaaauuugccuccauccuuuucucacagcaaugaauuugcaaucugaacccaagugaaaaaacaaaauugccugaauuguacuguauguagcugcacuacaacagauucuuaccgucuccacaaaggucagagauuguaaauggucaauacugacuuuuuuuuuauucccuugacucaagacagcuaacuucauuuucagaacuguuuuaaaccuuugugugcugguuuauaaaauaauguguguaauccuuguugcuuuccugauaccagacuguuucccgugguugguuagaauauauuuuguuuugauguuuauauuggcauguuuagaugucagguuuagucuucugaagaugaaguucagccauuuuguaucaaacagcacaagcagugucugucacuuuccaugcauaaaguuuagugagauguuauauguaagaucugauuugcuaguucuuccuuguagaguuauaaauggaaagauuacacuaucugauuaauaguuucuucauacucugcauauaauuuguggcugcagaauauuguaauuuguugcacacuauguaacaaaacaacugaagauauguuuaauaaauauuguacuuauuggaaguaauaucaaacuguauggugauaaguauuguuuugauucuuaugguuaaagggaaauagagccuugcauuauauucaacacagccauuugugugugcacaaugcaaacuaagguauucuagaccuaucuuagagcagcauccaguauuugcuuucuagauaauaugcccaauaacaugaccuagaggggcuucugugcuguguagggauuuaaccaacuucagugguucagggagcucaaacuauauguaaaacaaguuuagaauguaugcuaucuagcccguuaucucugauccuucucuaaaaccauuugaaauagcuucauugaucaacauuucauaaaugcaucugugguagagguagaaagcagcaccuuuccuaauuggcaaaugaucagacuaaugugugcuaauguuuuucuuccaugcuuucagucagauucaacuauuuuauccuccacaguugcuuaacuugguguuggaggaggguuuaagcauuaagauaggaagcaggaaauuugauugcucuaaauuuagaaauuauaucccuaaaaauuaaaacaugaauacugggugguaaugauaauugaggcaaauguauuuauuuuggugacauuuugcauauaugaagauuuucugaaauaggaccuucaagauccuaggggguuuuguuugguuuuuaauugugaggaauaaaaaaucuucugcccacacuggcauuuuaaggugacugaggucaaacguuguuuccuuagguugaaauagcagccaaaacauucuucacgcaggggcuugggauauggcugcuggcaacacauuuuguugugggcuccuuaauuuaaugauaaaauuuaagcuaaacacaagccaaaaaugaauagguuuuuuuaauuuuuauuuuucacuaaacaggcaauugaaauacaugguacaaaaauaagugguaagauaauuguaaaaugaaauggacagaauauucaauuuuccaucuaugaaaauuucacaauaaaaaucauaguuuacuuuguaaaaaa

SEQ ID NO: 69 - RNA, 6239 nt, Homo sapiens
acuucggggccgcuccugcccccuggggauacucugggcucaggcggauguccacucugguacccccgggugcggacuccaaagccugccagugccggcccccuuccccuacgcauuccucuaccguaaggcagggcgccuccaccccgcccccuccgccaguccuggggaggggaagggggcgggcagcgcagggggugugacagcucccgcccggaccggugccagcgcagccuugcuucagcgcccggugccagcuggcuccuucgucugagacgaaggcgcaggcucggggcucccugucgccgguggcuggguggcguccccccaccacgcccccaccccucuacucacccaagaggagccucuccaccgagcccugcauccugcugcccuggcagccaguccagcuaggcacuggcccaguucacuguaccuggacuuacccacuggcacccuccggauccccuuaucaaagcugaugggggucgcuggggaccccccagccuuuugcuagagccugcacggcugugcgagcuugaaaagaaacaugaaggugggcuggccaggugagagcugcuggcaggugggccuggcuguggaggauagcccagcucugggagcaccgcgggugggagcccucccugacguggugccggaggggacacuacucaacaugguguugagaaggaugcaccggccccgaagcugcuccuaccagcugcugcuggagcaccagcguccgagcugcauccaggggcugcgcuggacaccacucaccaacagcgaggagucccuggauuucagcgagagccuggagcaggccuccacagagcgggugcucagggcugggaggcagcugcaucggcaucugcuggccaccugcccaaaccucauccgagaccggaaguaccaccuuaggcucuaucggcagugcugcucuggccgggagcugguggaugggaucuuggcccugggacuugggguccauucccggagccaaguugugggaaucugccaggugcugcuggaugaaggugcccucugccaugugaaacacgacugggccuuccaggaccgagaugcccaauucuaccgguuccccgggcccgagcccgagcccgugggaacucaugagauggaggaggaguuggccgaagcuguggcccugcucucccagcgggggccugacgcccugcucacuguggcacuucgaaagcccccaggucagcgcacggaugaagagcuggaccucaucuuugaggagcugcugcacaucaaggcuguggcccaccucuccaacucggugaagcgagaauuagcggcuguucugcucuuugaaccacacagcaaggcagggaccguguuguucagccagggggacaagggcacuucgugguacauuaucuggaagggaucugucaacguggugacccauggcaaggggcuggugaccacccugcaugagggagaugauuuuggacagcuggcucuggugaaugaugcaccccgggcagccaccaucauccugcgagaagacaacugucauuuccugcguguggacaagcaggacuucaaccguaucaucaaggauguggaggcaaagaccaugcggcuggaagaacauggcaaaguggugcuggugcuggagagagccucucagggcgccggcccuucccgacccccaaccccaggcaggaaccgguauacagugaugucuggcaccccagagaagauccuagagcuucuguuggaggccaugggaccagauuccagugcucaugacccaacagagacauuccucagcgacuuccuccugacccacagggucuucaugcccagcgcccaacucugcgcugcccuucugcaccacuuccauguggagccugcggguggcagcgagcaggagcgcagcaccuacgucugcaacaagaggcagcagaucuugcggcuggucagccaguggguggcccuguauggcuccaugcuccacacugacccuguggccaccagcuuccuccagaaacucucagaccuggugggcagggacacccgacucagcaaccugcugagggagcaguggccagagaggcggcgaugccacagguuggagaauggcugugggaaugcaucuccucagaugaaggcccggaacuugccuguuuggcuccccaaccaggacgagccccuuccuggcagcagcugugccauccaaguuggggauaaaguccccuaugacaucugccggccagaccacucaguguugacccugcagcugccugugacagccuccgugagagaggugauggcagcguuggcccaggaggauggcuggaccaaggggcaggugcuggugaaggucaauucugcaggugaugccauuggccugcagccagaugcccgugguguggccacaucucuggggcucaaugagcgucucuuuguugucaacccacaggaagugcaugagcugaucccacacccugaccagcuggggcccacugugggcucugcugaggggcuggaccuggugagugccaaggaccuggcaggccagcugacggaccacgacuggagccucuucaacaguauccaccagguggagcugauccacuaugugcugggcccccagcaucugcgggaugucaccaccgccaaccuggagcgcuucaugcgccgcuucaaugagcugcaguacuggguggccaccgagcugugucucugccccgugcccggcccccgggcccagcugcucaggaaguucauuaagcuggcggcccaccucaaggagcagaagaaucucaauuccuucuuugccgucauguuuggccucagcaacucggccaucagccgccuagcccacaccugggagcggcugccucacaaaguccggaagcuguacuccgcccucgagaggcugcuggaucccucauggaaccaccggguauaccgacuggcccucgccaagcucuccccuccugucauccccuucaugccccuucuucucaaagacaugaccuucauucaugagggaaaccacacacuaguggagaaucucaucaacuuugagaagaugagaaugauggccagagccgcgcggaugcugcaccacugccgaagccacaacccugugccucucucaccacucagaagccgaguuucccaccuccacgaggacagccagguggcgaggauuuccacaugcucggagcagucccugagcacccggaguccagccagcaccugggcuuauguccagcagcugaaggucauugacaaccagcgggaacucucccgccucucccgagagcuggagccaugaggaggggcugggacuggagcuggagcaggcacuugcagccgggaaagccagggugugccgggccaagauacucacaggcuggccacagcugggcaaggcucuccguggaguggacucgagucccuggagcaggcaguguggaggcagccauccccugugaugacuggcagcuaaggaggaccucggaguggacccgagccaggaauaacgaaugacccaaggccaaggaagggaggacagagaggccccaggaguggguggagaguggagugcgcugggacguugugugcaauagagaggucuccacaccagaugucuuccagauucugugccucuggcuuuguuguccagccaggccugcaguuuauuuucacaguggacagagagagagagagaggcugcauguguguaccguguguggcaagggcagggccuuggccuggggcaggggcccccugcuuucuuuccacagcuuucuuccaacagcaggcaguggggcugcgggccugugcgguggucccugaggagcuuuauuguuacugucaacauaguaguauguuccaggccagcccagagugggcguggcagggaggcugaggccccgcccucugggaaggagcuguggaggagaagcagccccaugcugggcacgggccagacuggguccaagauccugaugccacccagaugugccuccagggcucuggccaaagggcgggcuggagccuggagccccugucagcagccagccagggugaccaucagcuccccguggugguccaauaggacuuuacaguccagaugcaacugacagcuucuucccaacugcaauccccgcuuuuccacacuugagcucguccuggccuguugggccaggcuucuggggucuucuggggguggcuugugauagaaacaaugcugaagcuuccgcggagagcccgucgcuuguugaggcccaacagacuuccaggaaaucaggguuuaucaucaguguugagaauggucccagcaagcucuggucauacgaaguccucucuucccugccccuucagcacuuccccuaggaaccagcuacugccaggacgccucuugcgaggcagagcucacccaggaagcuccagggacuccacccucuugccuugcuauugcccugaguccuggcagcaggaggcacuugucuggccagagagagaacucauggccccuggcaugacuccugaguagguauugaggcugcagggccuugggcguccagaggcagcugguauggccccucugcacuucagccccauggauguggauacacaggccugcugcuuccagaaagggucagagccuuuagcagggaggcagcacagcugggcuuagaccucaggucacuaagugaaucugagcaagucagugaacaguucucuuccuucauuucuccaccuguaagccagguaugauaaacgccauacagcgcuuagugacaaacacauaaaacuguagcuccugccucuguagcagaggugacagccaagggcaguccugugcuauccaggccugguaggggggcagccucgccucaguugagggcugagggagagacugaggugcagcagauggcagugguaaccaaaccaguucccuugccguggguggggacacaggagaagcacggaagcaucuggaacugagcaaucaggacaggaggcucaagccugggugagucaggaguccgggggcuuucuuuucauccaacccauugucugcuggugucucggcaaaagccagccagccagccguggcccuucaccguucuucagacucuagggugagcccagcccagcgacacaacagaaaccaagugccacgggggccgguguccuccaccagcauuucuccucuggucacaucuacguccugaugggacuucucccuccuccuuggaccaucagcuuuacaguccagaccacccugcagccacccgguggucucccagcugccccagucucgggcaggauggcuuuugagccuguugggagagaccuugccaggagaauggugcccagggcaggcaagaggacacagacucugggagcuagaagaguugcggcccagggggccaggccacucccagaggacaggaggcccaaaagcggggaaaggcuccauguuacgguggccccaugcugggaguuugugcugccuucugucucccugacugcccaagcguggggugguguggggcaggaggcauccucugguguucccuagcagcccaggcugugugcaggggcucccauuuugugucucagcugcaagaaggccacaaagggaggaggggcucaugggacugcugagaagaaggugugcagaaagggaguguggcccuagaucaguccuuuaaaaagccucuguuaccugaaaggaggucaagcagagaggaauggcugcguguguguccuaccgggcaggacaggacagagggaggucauuguuacacacacauccccccuccuuuccuccuuccccuccacuucccugugaaggccugaggcaggcuguggccaggcucaggcccugacugugggguacacaccagggagggccacgccuccccacagacgggguggaguggucaguggagccagcacccucugcugcuucagcaucuccgccuggagccagcuguccccucucucucaggcuccauccgaacaaaaggaaggcagagccugagauccggcaccccaucccccuccacucagcacugaguuagagcugggugggggcaugucugccuucacacgcugccuuauggcuucugugucuccaacagcccucagccucuggcugcucuccuucccaauagucacaagauuuaccaugauaauaauagguuaagacuugaaaaaaaaaaaaaaaaaaaaaa

SEQ ID NO: 70 - RNA, 6003 nt, Homo sapiens
agugaaucuauagcuucauacugggagguaguggagaucccugcaggggcaugggcagugagaugggacagaauuccaaagaggacuaagaagggccugaucuuuuccaagagccagcuggagaccccuaagugcgccagaccggcaggccccagugaaggucuuccuguuccuccccccagcuccuggcaggguugcagcaccuccgccugaccaguccagaggacucuaaggaaguaguucugccuggggcaguuguacuguggcccagccgccccagucgugucccauggcuguccacuuuccuccccgugucacaggugggcuggccaggugagagcugcuggcaggugggccuggcuguggaggauagcccagcucugggagcaccgcgggugggagcccucccugacguggugccggaggggacacuacucaacaugguguugagaaggaugcaccggccccgaagcugcuccuaccagcugcugcuggagcaccagcguccgagcugcauccaggggcugcgcuggacaccacucaccaacagcgaggagucccuggauuucagcgagagccuggagcaggccuccacagagcgggugcucagggcugggaggcagcugcaucggcaucugcuggccaccugcccaaaccucauccgagaccggaaguaccaccuuaggcucuaucggcagugcugcucuggccgggagcugguggaugggaucuuggcccugggacuugggguccauucccggagccaaguugugggaaucugccaggugcugcuggaugaaggugcccucugccaugugaaacacgacugggccuuccaggaccgagaugcccaauucuaccgguuccccgggcccgagcccgagcccgugggaacucaugagauggaggaggaguuggccgaagcuguggcccugcucucccagcgggggccugacgcccugcucacuguggcacuucgaaagcccccaggucagcgcacggaugaagagcuggaccucaucuuugaggagcugcugcacaucaaggcuguggcccaccucuccaacucggugaagcgagaauuagcggcuguucugcucuuugaaccacacagcaaggcagggaccguguuguucagccagggggacaagggcacuucgugguacauuaucuggaagggaucugucaacguggugacccauggcaaggggcuggugaccacccugcaugagggagaugauuuuggacagcuggcucuggugaaugaugcaccccgggcagccaccaucauccugcgagaagacaacugucauuuccugcguguggacaagcaggacuucaaccguaucaucaaggauguggaggcaaagaccaugcggcuggaagaacauggcaaaguggugcuggugcuggagagagccucucagggcgccggcccuucccgacccccaaccccaggcaggaaccgguauacagugaugucuggcaccccagagaagauccuagagcuucuguuggaggccaugggaccagauuccagugcucaugacccaacagagacauuccucagcgacuuccuccugacccacagggucuucaugcccagcgcccaacucugcgcugcccuucugcaccacuuccauguggagccugcggguggcagcgagcaggagcgcagcaccuacgucugcaacaagaggcagcagaucuugcggcuggucagccaguggguggcccuguauggcuccaugcuccacacugacccuguggccaccagcuuccuccagaaacucucagaccuggugggcagggacacccgacucagcaaccugcugagggagcaguggccagagaggcggcgaugccacagguuggagaauggcugugggaaugcaucuccucagaugaaggcccggaacuugccuguuuggcuccccaaccaggacgagccccuuccuggcagcagcugugccauccaaguuggggauaaaguccccuaugacaucugccggccagaccacucaguguugacccugcagcugccugugacagccuccgugagagaggugauggcagcguuggcccaggaggauggcuggaccaaggggcaggugcuggugaaggucaauucugcaggugaugccauuggccugcagccagaugcccgugguguggccacaucucuggggcucaaugagcgucucuuuguugucaacccacaggaagugcaugagcugaucccacacccugaccagcuggggcccacugugggcucugcugaggggcuggaccuggugagugccaaggaccuggcaggccagcugacggaccacgacuggagccucuucaacaguauccaccagguggagcugauccacuaugugcugggcccccagcaucugcgggaugucaccaccgccaaccuggagcgcuucaugcgccgcuucaaugagcugcaguacuggguggccaccgagcugugucucugccccgugcccggcccccgggcccagcugcucaggaaguucauuaagcuggcggcccaccucaaggagcagaagaaucucaauuccuucuuugccgucauguuuggccucagcaacucggccaucagccgccuagcccacaccugggagcggcugccucacaaaguccggaagcuguacuccgcccucgagaggcugcuggaucccucauggaaccaccggguauaccgacuggcccucgccaagcucuccccuccugucauccccuucaugccccuucuucucaaagacaugaccuucauucaugagggaaaccacacacuaguggagaaucucaucaacuuugagaagaugagaaugauggccagagccgcgcggaugcugcaccacugccgaagccacaacccugugccucucucaccacucagaagccgaguuucccaccuccacgaggacagccagguggcgaggauuuccacaugcucggagcagucccugagcacccggaguccagccagcaccugggcuuauguccagcagcugaaggucauugacaaccagcgggaacucucccgccucucccgagagcuggagccaugaggaggggcugggacuggagcuggagcaggcacuugcagccgggaaagccagggugugccgggccaagauacucacaggcuggccacagcugggcaaggcucuccguggaguggacucgagucccuggagcaggcaguguggaggcagccauccccugugaugacuggcagcuaaggaggaccucggaguggacccgagccaggaauaacgaaugacccaaggccaaggaagggaggacagagaggccccaggaguggguggagaguggagugcgcugggacguugugugcaauagagaggucuccacaccagaugucuuccagauucugugccucuggcuuuguuguccagccaggccugcaguuuauuuucacaguggacagagagagagagagaggcugcauguguguaccguguguggcaagggcagggccuuggccuggggcaggggcccccugcuuucuuuccacagcuuucuuccaacagcaggcaguggggcugcgggccugugcgguggucccugaggagcuuuauuguuacugucaacauaguaguauguuccaggccagcccagagugggcguggcagggaggcugaggccccgcccucugggaaggagcuguggaggagaagcagccccaugcugggcacgggccagacuggguccaagauccugaugccacccagaugugccuccagggcucuggccaaagggcgggcuggagccuggagccccugucagcagccagccagggugaccaucagcuccccguggugguccaauaggacuuuacaguccagaugcaacugacagcuucuucccaacugcaauccccgcuuuuccacacuugagcucguccuggccuguugggccaggcuucuggggucuucuggggguggcuugugauagaaacaaugcugaagcuuccgcggagagcccgucgcuuguugaggcccaacagacuuccaggaaaucaggguuuaucaucaguguugagaauggucccagcaagcucuggucauacgaaguccucucuucccugccccuucagcacuuccccuaggaaccagcuacugccaggacgccucuugcgaggcagagcucacccaggaagcuccagggacuccacccucuugccuugcuauugcccugaguccuggcagcaggaggcacuugucuggccagagagagaacucauggccccuggcaugacuccugaguagguauugaggcugcagggccuugggcguccagaggcagcugguauggccccucugcacuucagccccauggauguggauacacaggccugcugcuuccagaaagggucagagccuuuagcagggaggcagcacagcugggcuuagaccucaggucacuaagugaaucugagcaagucagugaacaguucucuuccuucauuucuccaccuguaagccagguaugauaaacgccauacagcgcuuagugacaaacacauaaaacuguagcuccugccucuguagcagaggugacagccaagggcaguccugugcuauccaggccugguaggggggcagccucgccucaguugagggcugagggagagacugaggugcagcagauggcagugguaaccaaaccaguucccuugccguggguggggacacaggagaagcacggaagcaucuggaacugagcaaucaggacaggaggcucaagccugggugagucaggaguccgggggcuuucuuuucauccaacccauugucugcuggugucucggcaaaagccagccagccagccguggcccuucaccguucuucagacucuagggugagcccagcccagcgacacaacagaaaccaagugccacgggggccgguguccuccaccagcauuucuccucuggucacaucuacguccugaugggacuucucccuccuccuuggaccaucagcuuuacaguccagaccacccugcagccacccgguggucucccagcugccccagucucgggcaggauggcuuuugagccuguugggagagaccuugccaggagaauggugcccagggcaggcaagaggacacagacucugggagcuagaagaguugcggcccagggggccaggccacucccagaggacaggaggcccaaaagcggggaaaggcuccauguuacgguggccccaugcugggaguuugugcugccuucugucucccugacugcccaagcguggggugguguggggcaggaggcauccucugguguucccuagcagcccaggcugugugcaggggcucccauuuugugucucagcugcaagaaggccacaaagggaggaggggcucaugggacugcugagaagaaggugugcagaaagggaguguggcccuagaucaguccuuuaaaaagccucuguuaccugaaaggaggucaagcagagaggaauggcugcguguguguccuaccgggcaggacaggacagagggaggucauuguuacacacacauccccccuccuuuccuccuuccccuccacuucccugugaaggccugaggcaggcuguggccaggcucaggcccugacugugggguacacaccagggagggccacgccuccccacagacgggguggaguggucaguggagccagcacccucugcugcuucagcaucuccgccuggagccagcuguccccucucucucaggcuccauccgaacaaaaggaaggcagagccugagauccggcaccccaucccccuccacucagcacugaguuagagcugggugggggcaugucugccuucacacgcugccuuauggcuucugugucuccaacagcccucagccucuggcugcucuccuucccaauagucacaagauuuaccaugauaauaauagguuaagacuugaaaaaaaaaaaaaaaaaaaaaa

SEQ ID NO: 71 - RNA, 5773 nt, Homo sapiens
guuagucagggcugaggaggagaggagaaggguuuaagccgaagcugcauuucugggagaccuagugaaucuauagcuucauacugggaggugggcuggccaggugagagcugcuggcaggugggccuggcuguggaggauagcccagcucugggagcaccgcgggugggagcccucccugacguggugccggaggggacacuacucaacaugguguugagaaggaugcaccggccccgaagcugcuccuaccagcugcugcuggagcaccagcguccgagcugcauccaggggcugcgcuggacaccacucaccaacagcgaggagucccuggauuucagcgagagccuggagcaggccuccacagagcgggugcucagggcugggaggcagcugcaucggcaucugcuggccaccugcccaaaccucauccgagaccggaaguaccaccuuaggcucuaucggcagugcugcucuggccgggagcugguggaugggaucuuggcccugggacuugggguccauucccggagccaaguugugggaaucugccaggugcugcuggaugaaggugcccucugccaugugaaacacgacugggccuuccaggaccgagaugcccaauucuaccgguuccccgggcccgagcccgagcccgugggaacucaugagauggaggaggaguuggccgaagcuguggcccugcucucccagcgggggccugacgcccugcucacuguggcacuucgaaagcccccaggucagcgcacggaugaagagcuggaccucaucuuugaggagcugcugcacaucaaggcuguggcccaccucuccaacucggugaagcgagaauuagcggcuguucugcucuuugaaccacacagcaaggcagggaccguguuguucagccagggggacaagggcacuucgugguacauuaucuggaagggaucugucaacguggugacccauggcaaggggcuggugaccacccugcaugagggagaugauuuuggacagcuggcucuggugaaugaugcaccccgggcagccaccaucauccugcgagaagacaacugucauuuccugcguguggacaagcaggacuucaaccguaucaucaaggauguggaggcaaagaccaugcggcuggaagaacauggcaaaguggugcuggugcuggagagagccucucagggcgccggcccuucccgacccccaaccccaggcaggaaccgguauacagugaugucuggcaccccagagaagauccuagagcuucuguuggaggccaugggaccagauuccagugcucaugacccaacagagacauuccucagcgacuuccuccugacccacagggucuucaugcccagcgcccaacucugcgcugcccuucugcaccacuuccauguggagccugcggguggcagcgagcaggagcgcagcaccuacgucugcaacaagaggcagcagaucuugcggcuggucagccaguggguggcccuguauggcuccaugcuccacacugacccuguggccaccagcuuccuccagaaacucucagaccuggugggcagggacacccgacucagcaaccugcugagggagcaguggccagagaggcggcgaugccacagguuggagaauggcugugggaaugcaucuccucagaugaaggcccggaacuugccuguuuggcuccccaaccaggacgagccccuuccuggcagcagcugugccauccaaguuggggauaaaguccccuaugacaucugccggccagaccacucaguguugacccugcagcugccugugacagccuccgugagagaggugauggcagcguuggcccaggaggauggcuggaccaaggggcaggugcuggugaaggucaauucugcaggugaugccauuggccugcagccagaugcccgugguguggccacaucucuggggcucaaugagcgucucuuuguugucaacccacaggaagugcaugagcugaucccacacccugaccagcuggggcccacugugggcucugcugaggggcuggaccuggugagugccaaggaccuggcaggccagcugacggaccacgacuggagccucuucaacaguauccaccagguggagcugauccacuaugugcugggcccccagcaucugcgggaugucaccaccgccaaccuggagcgcuucaugcgccgcuucaaugagcugcaguacuggguggccaccgagcugugucucugccccgugcccggcccccgggcccagcugcucaggaaguucauuaagcuggcggcccaccucaaggagcagaagaaucucaauuccuucuuugccgucauguuuggccucagcaacucggccaucagccgccuagcccacaccugggagcggcugccucacaaaguccggaagcuguacuccgcccucgagaggcugcuggaucccucauggaaccaccggguauaccgacuggcccucgccaagcucuccccuccugucauccccuucaugccccuucuucucaaagacaugaccuucauucaugagggaaaccacacacuaguggagaaucucaucaacuuugagaagaugagaaugauggccagagccgcgcggaugcugcaccacugccgaagccacaacccugugccucucucaccacucagaagccgaguuucccaccuccacgaggacagccagguggcgaggauuuccacaugcucggagcagucccugagcacccggaguccagccagcaccugggcuuauguccagcagcugaaggucauugacaaccagcgggaacucucccgccucucccgagagcuggagccaugaggaggggcugggacuggagcuggagcaggcacuugcagccgggaaagccagggugugccgggccaagauacucacaggcuggccacagcugggcaaggcucuccguggaguggacucgagucccuggagcaggcaguguggaggcagccauccccugugaugacuggcagcuaaggaggaccucggaguggacccgagccaggaauaacgaaugacccaaggccaaggaagggaggacagagaggccccaggaguggguggagaguggagugcgcugggacguugugugcaauagagaggucuccacaccagaugucuuccagauucugugccucuggcuuuguuguccagccaggccugcaguuuauuuucacaguggacagagagagagagagaggcugcauguguguaccguguguggcaagggcagggccuuggccuggggcaggggcccccugcuuucuuuccacagcuuucuuccaacagcaggcaguggggcugcgggccugugcgguggucccugaggagcuuuauuguuacugucaacauaguaguauguuccaggccagcccagagugggcguggcagggaggcugaggccccgcccucugggaaggagcuguggaggagaagcagccccaugcugggcacgggccagacuggguccaagauccugaugccacccagaugugccuccagggcucuggccaaagggcgggcuggagccuggagccccugucagcagccagccagggugaccaucagcuccccguggugguccaauaggacuuuacaguccagaugcaacugacagcuucuucccaacugcaauccccgcuuuuccacacuugagcucguccuggccuguugggccaggcuucuggggucuucuggggguggcuugugauagaaacaaugcugaagcuuccgcggagagcccgucgcuuguugaggcccaacagacuuccaggaaaucaggguuuaucaucaguguugagaauggucccagcaagcucuggucauacgaaguccucucuucccugccccuucagcacuuccccuaggaaccagcuacugccaggacgccucuugcgaggcagagcucacccaggaagcuccagggacuccacccucuugccuugcuauugcccugaguccuggcagcaggaggcacuugucuggccagagagagaacucauggccccuggcaugacuccugaguagguauugaggcugcagggccuugggcguccagaggcagcugguauggccccucugcacuucagccccauggauguggauacacaggccugcugcuuccagaaagggucagagccuuuagcagggaggcagcacagcugggcuuagaccucaggucacuaagugaaucugagcaagucagugaacaguucucuuccuucauuucuccaccuguaagccagguaugauaaacgccauacagcgcuuagugacaaacacauaaaacuguagcuccugccucuguagcagaggugacagccaagggcaguccugugcuauccaggccugguaggggggcagccucgccucaguugagggcugagggagagacugaggugcagcagauggcagugguaaccaaaccaguucccuugccguggguggggacacaggagaagcacggaagcaucuggaacugagcaaucaggacaggaggcucaagccugggugagucaggaguccgggggcuuucuuuucauccaacccauugucugcuggugucucggcaaaagccagccagccagccguggcccuucaccguucuucagacucuagggugagcccagcccagcgacacaacagaaaccaagugccacgggggccgguguccuccaccagcauuucuccucuggucacaucuacguccugaugggacuucucccuccuccuuggaccaucagcuuuacaguccagaccacccugcagccacccgguggucucccagcugccccagucucgggcaggauggcuuuugagccuguugggagagaccuugccaggagaauggugcccagggcaggcaagaggacacagacucugggagcuagaagaguugcggcccagggggccaggccacucccagaggacaggaggcccaaaagcggggaaaggcuccauguuacgguggccccaugcugggaguuugugcugccuucugucucccugacugcccaagcguggggugguguggggcaggaggcauccucugguguucccuagcagcccaggcugugugcaggggcucccauuuugugucucagcugcaagaaggccacaaagggaggaggggcucaugggacugcugagaagaaggugugcagaaagggaguguggcccuagaucaguccuuuaaaaagccucuguuaccugaaaggaggucaagcagagaggaauggcugcguguguguccuaccgggcaggacaggacagagggaggucauuguuacacacacauccccccuccuuuccuccuuccccuccacuucccugugaaggccugaggcaggcuguggccaggcucaggcccugacugugggguacacaccagggagggccacgccuccccacagacgggguggaguggucaguggagccagcacccucugcugcuucagcaucuccgccuggagccagcuguccccucucucucaggcuccauccgaacaaaaggaaggcagagccugagauccggcaccccaucccccuccacucagcacugaguuagagcugggugggggcaugucugccuucacacgcugccuuauggcuucugugucuccaacagcccucagccucuggcugcucuccuucccaauagucacaagauuuaccaugauaauaauagguuaagacuugaaaaaaaaaaaaaaaaaaaaaa

SEQ ID NO: 72 - RNA, 4576 nt, Homo sapiens
uggcagccagugucgggguggcggcugggaaugggggccgcuccggacuuccgcugccaacuacaagggggcggguccgagggggguuagccgaaguuguaggcggggcgcgagguucuaguacccgagcucauacuagggacgggaagucgcgaccagagccauuggagggcgcggggacugcaacccuaaucagagcccaaauggcgcagugggaaaugcugcagaaucuugacagccccuuucaggaucagcugcaccagcuuuacucgcacagccuccugccuguggacauucgacaguacuuggcugucuggauugaagaccagaacuggcaggaagcugcacuugggagugaugauuccaaggcuaccaugcuauucuuccacuucuuggaucagcugaacuaugaguguggccguugcagccaggacccagaguccuuguugcugcagcacaauuugcggaaauucugccgggacauucagcccuuuucccaggauccuacccaguuggcugagaugaucuuuaaccuccuucuggaagaaaaaagaauuuugauccaggcucagagggcccaauuggaacaaggagagccaguucucgaaacaccuguggagagccagcaacaugagauugaaucccggauccuggauuuaagggcuaugauggagaagcugguaaaauccaucagccaacugaaagaccagcaggaugucuucugcuuccgauauaagauccaggccaaagggaagacacccucucuggacccccaucagaccaaagagcagaagauucugcaggaaacucucaaugaacuggacaaaaggagaaaggaggugcuggaugccuccaaagcacugcuaggccgauuaacuacccuaaucgagcuacugcugccaaaguuggaggaguggaaggcccagcagcaaaaagccugcaucagagcucccauugaccacggguuggaacagcuggagacaugguucacagcuggagcaaagcuguuguuucaccugaggcagcugcugaaggagcugaagggacugaguugccugguuagcuaucaggaugacccucugaccaaagggguggaccuacgcaacgcccaggucacagaguugcuacagcgucugcuccacagagccuuugugguagaaacccagcccugcaugccccaaacuccccaucgaccccucauccucaagacuggcagcaaguucaccguccgaacaaggcugcuggugagacuccaggaaggcaaugagucacugacuguggaagucuccauugacaggaauccuccucaauuacaaggcuuccggaaguucaacauucugacuucaaaccagaaaacuuugacccccgagaaggggcagagucaggguuugauuugggacuuugguuaccugacucugguggagcaacguucaggugguucaggaaagggcagcaauaaggggccacuaggugugacagaggaacugcacaucaucagcuucacggucaaauauaccuaccagggucugaagcaggagcugaaaacggacacccucccuguggugauuauuuccaacaugaaccagcucucaauugccugggcuucaguucucugguucaauuugcucagcccaaaccuucagaaccagcaguucuucuccaacccccccaaggcccccuggagcuugcugggcccugcucucaguuggcaguucuccuccuauguuggccgaggccucaacucagaccagcugagcaugcugagaaacaagcuguucgggcagaacuguaggacugaggauccauuauuguccugggcugacuucacuaagcgagagagcccuccuggcaaguuaccauucuggacauggcuggacaaaauucuggaguugguacaugaccaccugaaggaucucuggaaugauggacgcaucaugggcuuugugagucggagccaggagcgccggcugcugaagaagaccaugucuggcaccuuucuacugcgcuucagugaaucgucagaagggggcauuaccugcuccuggguggagcaccaggaugaugacaaggugcucaucuacucugugcaaccguacacgaaggaggugcugcagucacucccgcugacugaaaucauccgccauuaccaguugcucacugaggagaauauaccugaaaacccacugcgcuuccucuauccccgaaucccccgggaugaagcuuuugggugcuacuaccaggagaaaguuaaucuccaggaacggaggaaauaccugaaacacaggcucauuguggucucuaauagacagguggaugaacugcaacaaccgcuggagcuuaagccagagccagagcuggagucauuagagcuggaacuagggcuggugccagagccagagcucagccuggacuuagagccacugcugaaggcagggcuggaucuggggccagagcuagagucugugcuggaguccacucuggagccugugauagagcccacacuaugcaugguaucacaaacagugccagagccagaccaaggaccuguaucacagccagugccagagccagauuugcccugugaucugagacauuugaacacugagccaauggaaaucuucagaaacuguguaaagauugaagaaaucaugccgaauggugacccacuguuggcuggccagaacaccguggaugagguuuacgucucccgccccagccacuucuacacugauggacccuugaugccuucugacuucuaggaaccacauuuccucuguucuuuucauaucucuugcccuuccuacuccucauagcaugauauuguucuccaaggaugggaaucaggcaugugucccuuccaagcuguguuaacuguucaaacucaggccugugugacuccauuggggugagaggugaaagcauaacauggguacagaggggacaacaaugaaucagaacagaugcugagccauaggucuaaauaggauccuggaggcugccugcugugcugggagguauagggguccugggggcaggccagggcaguugacagguacuuggagggcucagggcaguggcuucuuuccaguauggaaggauuucaacauuuuaauaguugguuaggcuaaacuggugcauacuggcauuggcccuugguggggagcacagacacaggauaggacuccauuucuuucuuccauuccuucaugucuaggauaacuugcuuucuucuuuccuuuacuccuggcucaagcccugaauuucuucuuuuccugcagggguugagagcuuucugccuuagccuaccaugugaaacucuacccugaagaaagggauggauaggaaguagaccucuuuuucuuaccagucuccuccccuacucugccccuaagcuggcuguaccuguuccucccccauaaaaugauccugccaaucuaaugugagugugaagcuuugcacacuaguuuaugcuaccuagucuccacuuucucaaugcuuaggagacagaucacuccuggaggcuggggaugguaggauugcuggggauuuuuuuuuuuuuaaacagggucucacucuguugcccaggcuagagugcaauggugcaaucacagcucacugcagccucaaccuccuggguucaagcaauccuccuaccucagccuccuggguagcuagcaccauggcaugcgccaccaugcccuauuuuuuuuuuuuaaagacagggucuugcuauauugcccaggcuggucuugaacugggcucaagugauccucacgccuuggccucccaaagugcugggauuauaggcaugagccacugugcuuggccaggauuuuuuuuuuuuuuuuuuugagauggaguuucucucuuguuguccaggcuggagugcaauggugugaucucggcucacugcaaccuccgccuuccggguucaagugacucuccugccucagccuccccaguagcugggauuacagaucugcaccaccaugcccagcuaauuuuguauuuuuaguagagacgggguuucuccauguuggucaggcuggucucgaacuccugaccucaagugaucuguccaccucggccucccagagugcugggauuacaggcgugagccacuguucccagcaggaauuucuuuuuuauaguauuggauaaaguuugguguuuuuacagaggagaagcaaugggucuuagcucuuucucuauuauguuaucauccucccuuuuuuguacaauauguuguuuaccugaaaggaagguuucuauucguugguuguggaccuggacaaaguccaagucuguggaacuuaaaaccuugaaggucugucauaggacucuggacaaucucacaccuuagcuauucccagggaaccccagggggcaacugacauugcuccaagauguucuccugauguagcuugagauauaaaggaaaggcccugcacagguggcuguuucuugucuguuaugucagaggaacaguccuguucagaaaggggcucuucugagcagaaauggcuaauaaacuuugugcugaucuggaaaaaaaaaaaaaaaaaaaaaaaaaaaa

SEQ ID NO: 73 - RNA, 4564 nt, Homo sapiens
uggcagccagugucgggguggcggcugggaaugggggccgcuccggacuuccgcugccaacuacaagggggcggguccgagggggguuagccgaaguuguaggcggggcgcgagguucuaguacccgagcucauacuagggacgggaagucgcgaccagagccauuggagggcgcggggacugcaacccuaaucagagcccaaauggcgcagugggaaaugcugcagaaucuugacagccccuuucaggaucagcugcaccagcuuuacucgcacagccuccugccuguggacauucgacaguacuuggcugucuggauugaagaccagaacuggcaggaagcugcacuugggagugaugauuccaaggcuaccaugcuauucuuccacuucuuggaucagcugaacuaugaguguggccguugcagccaggacccagaguccuuguugcugcagcacaauuugcggaaauucugccgggacauucaggauccuacccaguuggcugagaugaucuuuaaccuccuucuggaagaaaaaagaauuuugauccaggcucagagggcccaauuggaacaaggagagccaguucucgaaacaccuguggagagccagcaacaugagauugaaucccggauccuggauuuaagggcuaugauggagaagcugguaaaauccaucagccaacugaaagaccagcaggaugucuucugcuuccgauauaagauccaggccaaagggaagacacccucucuggacccccaucagaccaaagagcagaagauucugcaggaaacucucaaugaacuggacaaaaggagaaaggaggugcuggaugccuccaaagcacugcuaggccgauuaacuacccuaaucgagcuacugcugccaaaguuggaggaguggaaggcccagcagcaaaaagccugcaucagagcucccauugaccacggguuggaacagcuggagacaugguucacagcuggagcaaagcuguuguuucaccugaggcagcugcugaaggagcugaagggacugaguugccugguuagcuaucaggaugacccucugaccaaagggguggaccuacgcaacgcccaggucacagaguugcuacagcgucugcuccacagagccuuugugguagaaacccagcccugcaugccccaaacuccccaucgaccccucauccucaagacuggcagcaaguucaccguccgaacaaggcugcuggugagacuccaggaaggcaaugagucacugacuguggaagucuccauugacaggaauccuccucaauuacaaggcuuccggaaguucaacauucugacuucaaaccagaaaacuuugacccccgagaaggggcagagucaggguuugauuugggacuuugguuaccugacucugguggagcaacguucaggugguucaggaaagggcagcaauaaggggccacuaggugugacagaggaacugcacaucaucagcuucacggucaaauauaccuaccagggucugaagcaggagcugaaaacggacacccucccuguggugauuauuuccaacaugaaccagcucucaauugccugggcuucaguucucugguucaauuugcucagcccaaaccuucagaaccagcaguucuucuccaacccccccaaggcccccuggagcuugcugggcccugcucucaguuggcaguucuccuccuauguuggccgaggccucaacucagaccagcugagcaugcugagaaacaagcuguucgggcagaacuguaggacugaggauccauuauuguccugggcugacuucacuaagcgagagagcccuccuggcaaguuaccauucuggacauggcuggacaaaauucuggaguugguacaugaccaccugaaggaucucuggaaugauggacgcaucaugggcuuugugagucggagccaggagcgccggcugcugaagaagaccaugucuggcaccuuucuacugcgcuucagugaaucgucagaagggggcauuaccugcuccuggguggagcaccaggaugaugacaaggugcucaucuacucugugcaaccguacacgaaggaggugcugcagucacucccgcugacugaaaucauccgccauuaccaguugcucacugaggagaauauaccugaaaacccacugcgcuuccucuauccccgaaucccccgggaugaagcuuuugggugcuacuaccaggagaaaguuaaucuccaggaacggaggaaauaccugaaacacaggcucauuguggucucuaauagacagguggaugaacugcaacaaccgcuggagcuuaagccagagccagagcuggagucauuagagcuggaacuagggcuggugccagagccagagcucagccuggacuuagagccacugcugaaggcagggcuggaucuggggccagagcuagagucugugcuggaguccacucuggagccugugauagagcccacacuaugcaugguaucacaaacagugccagagccagaccaaggaccuguaucacagccagugccagagccagauuugcccugugaucugagacauuugaacacugagccaauggaaaucuucagaaacuguguaaagauugaagaaaucaugccgaauggugacccacuguuggcuggccagaacaccguggaugagguuuacgucucccgccccagccacuucuacacugauggacccuugaugccuucugacuucuaggaaccacauuuccucuguucuuuucauaucucuugcccuuccuacuccucauagcaugauauuguucuccaaggaugggaaucaggcaugugucccuuccaagcuguguuaacuguucaaacucaggccugugugacuccauuggggugagaggugaaagcauaacauggguacagaggggacaacaaugaaucagaacagaugcugagccauaggucuaaauaggauccuggaggcugccugcugugcugggagguauagggguccugggggcaggccagggcaguugacagguacuuggagggcucagggcaguggcuucuuuccaguauggaaggauuucaacauuuuaauaguugguuaggcuaaacuggugcauacuggcauuggcccuugguggggagcacagacacaggauaggacuccauuucuuucuuccauuccuucaugucuaggauaacuugcuuucuucuuuccuuuacuccuggcucaagcccugaauuucuucuuuuccugcagggguugagagcuuucugccuuagccuaccaugugaaacucuacccugaagaaagggauggauaggaaguagaccucuuuuucuuaccagucuccuccccuacucugccccuaagcuggcuguaccuguuccucccccauaaaaugauccugccaaucuaaugugagugugaagcuuugcacacuaguuuaugcuaccuagucuccacuuucucaaugcuuaggagacagaucacuccuggaggcuggggaugguaggauugcuggggauuuuuuuuuuuuuaaacagggucucacucuguugcccaggcuagagugcaauggugcaaucacagcucacugcagccucaaccuccuggguucaagcaauccuccuaccucagccuccuggguagcuagcaccauggcaugcgccaccaugcccuauuuuuuuuuuuuaaagacagggucuugcuauauugcccaggcuggucuugaacugggcucaagugauccucacgccuuggccucccaaagugcugggauuauaggcaugagccacugugcuuggccaggauuuuuuuuuuuuuuuuuuugagauggaguuucucucuuguuguccaggcuggagugcaauggugugaucucggcucacugcaaccuccgccuuccggguucaagugacucuccugccucagccuccccaguagcugggauuacagaucugcaccaccaugcccagcuaauuuuguauuuuuaguagagacgggguuucuccauguuggucaggcuggucucgaacuccugaccucaagugaucuguccaccucggccucccagagugcugggauuacaggcgugagccacuguucccagcaggaauuucuuuuuuauaguauuggauaaaguuugguguuuuuacagaggagaagcaaugggucuuagcucuuucucuauuauguuaucauccucccuuuuuuguacaauauguuguuuaccugaaaggaagguuucuauucguugguuguggaccuggacaaaguccaagucuguggaacuuaaaaccuugaaggucugucauaggacucuggacaaucucacaccuuagcuauucccagggaaccccagggggcaacugacauugcuccaagauguucuccugauguagcuugagauauaaaggaaaggcccugcacagguggcuguuucuugucuguuaugucagaggaacaguccuguucagaaaggggcucuucugagcagaaauggcuaauaaacuuugugcugaucuggaaaaaaaaaaaaaaaaaaaaaaaaaaaa

SEQ ID NO: 74 - RNA, 6030 nt, Homo sapiens
gccccugcgcgccgcccgagccggucccgcugagccgcgggccccgugcccugcgauggcucggcuggugcagcgcggcgccaggugccagccguccucccgcugagacgcgcccgaguggggacccgcugggccucggggcucgcagccuucgccuccccgccgcgcccgcucccuuucuggggacuccgccgcuguuucuggggacgaggggacaggggacccagacaaagcccacuuugugcagggaguuggccgcaggcggggaaugugcgcgucggcgcgcgcccccuccccgcucccggccagcugcgagucuuggcucccggacuugucucgucgcgucggagaaaucgccccccagcgccgcucucccgcccgggggucuugguuccgagcucgcgcggccgggagucgccucggucuuccuuggggcgcgcgcagaugugagcgugcgagaguuguguaggggauuuuguucccuccgaaacugagacccagggcgcccagugggcacccgugccuugacucuguccuuucugcagccgcugguccgagcugucuggccucaguuucccuccgacuuuucuccgcucugccagcccucacugcugcccgucauuguucucgcaguuagaugggggugcuuugugacggcugccaaguugggguguguucucuuuauuccguuuuucaaacagaacaaggccuccaaggcugaccccagacaacccacccccucggacccuaauucaccuuauugcacugauuuuuuuuaucaagucguauuuuauuguacaggagccacgcccugauuucuuaaaggcgccuugcacucuggccauguguuaucucugcagccggugugugggaggccucuugugagccaguuguuuucccgccuccaccaccccccucgaagauuuagggaugccaggggggaagaagguggcuggggguggcagcagcggugccacuccaacguccgcugcggccaccgcccccucuggggucaggcguuuggagaccagcgaaggaaccucagcccagagagaugaggagccagaagaggaaggggaagaggaccugcgagacggaggcguccccuucuuugucaaccggggugggcuaccuguggaugaggccaccugggaaaggauguggaaacacguggccaagauccaccccgauggagagaagguggcgcaacggauccguggggccacagaccugcccaagauccccauaccgagugugccuacguuccagccgucuacaccugucccugagcgccuggaagcugugcagcgcuacaucagagagcugcaguacaaucacacagggacacaguucuuugaaauuaagaagagcagaccucugacagggcugauggaccuggccaaggaaaugaccaaagaggcccugccaaucaaaugccuggaagccgugauccugggaauuuaccucaccaacagcaugcccacccuggagcgcuuccccaucagcuucaagaccuacuucucagggaacuacuuccgccacaucgugcugggggugaacuucgcgggccgcuacggugcgcugggcaugagucggcgcgaggaccugauguacaagccgcccgccuuccgcacgcucagcgagcucgugcuggacuucgaggccgccuacggccgcugcuggcacgugcucaagaaggugaagcugggccagagcgugucacacgacccgcacagcguggagcagaucgaguggaagcacucggugcuggacguggagcgccugggccgcgaugacuuccgcaaggagcuggagcgccacgcccgcgacaugcggcucaagauuggcaaagggacgggcccucccucucccaccaaggaccggaagaaggauguuucuuccccgcagcgggcccaguccagcccccaccgcaggaacagccgcagugaaagacggcccucgggugacaagaagacuuccgagcccaaagccaugccagaccuuaacggguaccagauccgggucugaggcggaugccagcaccccaggccccacccacucuugggggccaggauccaccugcuggaaccagccuuaugcauggggaaggcggggcuggugacaaggcagggcaagaggcugcaggaagaguguguuccagcucagccccccaagcugcucucgcucccacugagccaagcccccuaacuuugggccuagaggccguuaguauuuuauuuggaguuuuuaacucuacaacugaaguuuaagguauuuggggaaaacuuaguccaaauggaucugcugauggugggaaggccagugcuuaacaaauccaugugucauggggccaggugagggaaacugcugguucugcuggugccucugccccuggcuucucucugggaguugggugcaucuuaucagugggaaaucucccagccuuaccaggccugugauggggggugggggugggguggagauguuucuccaguucugccugcccuggcagaaucuugacccagggaaagggaagcaggguaggaguccuucugagaaaggucuguguagcccauuaaccaggagcuuggcacaggccacaucugccccaagagcaugagcucguggcucaggagagccuucaggcccuugaggcccccaugggcagugcugugugggcagaggaggggugauaugagagcgagcccagggaaggaccucugggcaaaaaguucccaggcccuaacugcgucuacuugcucagucccagcucugccuguugcucuagcccacaggcucccugcggagggucugggcuuggcggaggacccagaauggcacugaggccagcauggcugugggaguugagcaaacccugggugccaguccaggagcuguggcuggacauggggugauggggcgggaugcuugggccugggucucuccgccagcagugccaggagcccuugcuggggaaaucaagaccagacuaggaugcuucugccccaggccugccuuccauucuuuaacagcccaucuuggcuugggguugcaaugauggcugggccagucacuuguggcagggcaucagggcccuggcagggaagaaccuaggcaccugggguuguccccagccugcccgucagcaugagauacccagugggaaagugagaggaugcggagagguuggcagagccagggguagguucuggaggcucaagcaacaaggaggugcagguaaagggugcagugcagccacugagggacagcugggaacugggggaugcaagugagaaagggaugugggggagaguucaggaucaggcugcuugaggaguaauggguuaccuacagcagagacgagauggcuguuugucaggaggcgguagaaagguagaaaggauucagauuguggaaggguaaacggaugagaugaccauuaagguuuuguuuuacugagaggcuguaagucugccagggacagcugucaguaaggcugcagaagggcuggggggcuacacaaggaagagcagaacuaggguuguaagcucaaaugacgagcaaacggugagaaggaggccugaagggcuguguggggacuauggcuaccugaagagggcacacccuguuaaaggggccacagcugcucagcuguucuuacagugccggcccuguguugccagauugucugcuuugucagaggccagaauucugacuuuuuaugugaaauaggauuuuuaaaugcuggugaucacuuaaaaaaaacuuaaaaacccaauacuggcaaaagaggaugccaguuugcaaucccugaaguagagagagcucgugcuggggagaaguccaccaagaugcuuugaggcgggcuguagcaaaguccuguuucucagagcugggcugggcugggguaggauccuugcagcugaggaggaggaaaagccacugagucuccucccagagcgggacaaaccagaggcccuuugcaguugcugggucaccagcggggguggcgcauggaguacagacaguguagcucuuggccugccagggagacggauggugccuuugaagcaaagaggaagggaaggcagaaccagggaugccuuugcugaugagagugccugucagggaaggcgccacaggcuggcagcucuucaaaccagcagcgcuugacccagagccagauccagcauggccuggccagaggcacccugggaggccagcuggucagucccuugccuccccaaguucccccuggggucaaugagcccugggaggaugccuaaccuaacuccagccagacuaauaggggcauggugacccuugacucaccaucccaucccagcuuucagggaguggggguaguguggucuccauguuccuacuaugccuaagaagagauggcucaccuugggaggugccaggcugaaacuagguccuuuccgggucuggaugcugccgcucaggagcaggggcguggccucagcugccucugggagcuucccgggaaugacaggguuugaggggaguagauaugagagggagccgcuccugguucuggagucuuaggagguuccaacuugcaggauccuuucccagagcccuccauggagaaaacagcaaaaugaagcccuuaccugcuugcugucugcaagggagggagccgagccccagcugauaaucccccagcacucacccuuccugagcugagacuucggggcuguggagaccagcacaggacauaguggugcuuuuuaaauuuauuuuuaacuguuucucauauguagcaaccccuccuccccuccugggcauguuuacacaggcucugcucugggggcuggccuggcugugagguuucuggggaggcagagaggcagggacuuuggggccuuagucaccauccaugguaucaccucaucucacuuccugugagggacagggccuggcugaugugaucccagcuccccccaguucaggacugucuuucagcuccuuugccccuggaggugggggcugcuggcugaggaggggucaaggugaguucaagaaagcuaccuguggaaaauggaccagguugggggggugauugcaaagucuccccaaagccuggcuccucaugcucagugccaggggcagaacacuggggagccagguauagagagccuuccugucauaacugccaguccucuuccuccaaggccucugcauauucucauguuccccucacccaucaugccagccaccccuaucccucuucuagcagggccaagauggggacagcagcagcccucuggccuugggaugugaugauaaagcaagccuagggccaggguuuggggagcagagagagccaagaaguugaccacgugugauuuccagcccuucccacugggacuugacuucccaggucaaggaguccgucucauucuggcuggucgagugaccagaggccugugugaaugugugcaccugcuuuuccugccuggaauguuuucuggcucagcugcagcaacaucugugagcccagugucugcccugugucccugggcucgcuccaagugcaggaacauacaugcagggcccaacaugaugauggugugaagggcaggaaacaguccucugaaggaguggggaggugggcagucugcccccgccagguaccaucgccuccugccagcuuccuuagaccaggcagggcugccauggugcuagcugcaaguccaucaguauugaccgucucgcuccaucuugguccuccggagucccaaguuuccuuuucaucaaaucugacaagagagaagaaacaugggugugcuuggcccacagggccugguggugauggaccuccccgcucccucaagcucuggauggcugcaguguuguacuagacuuuguucaggcuguucucaucucaguauugccccuuccuuucacuuucacacuucaucucauuccuguugucacuuuccccgaaacgaauaaagucuccccagcuccugcuguguaggcugggcagaaaccacaacacgu

SEQ ID NO: 75 - RNA, 23 nt, Homo sapiens
ccugcagcgacuugauggcuucc

SEQ ID NO: 76 - RNA, 20 nt, Homo sapiens
cggcucugggucugugggga

SEQ ID NO: 77 - RNA, 21 nt, Homo sapiens
uggcagggaggcugggagggg